(19) United States
(12) Patent Application Publication
(10) Pub. No.: US 2019/0350929 A1
(43) Pub. Date: Nov. 21, 2019

(54) CANCER TREATMENT MODALITIES

(71) Applicant: Epizyme, Inc., Cambridge, MA (US)

(72) Inventor: Scott RIBICH, Lexington, MA (US)

(21) Appl. No.: 16/482,748

(22) PCT Filed: Feb. 2, 2018
(86) PCT No.: PCT/US2018/016562

§ 371 Date: Aug. 1, 2019

Related U.S. Application Data
(60) Provisional application No. 62/453,929, filed on Feb. 2, 2017.
(60) Provisional application No. 62/479,878, filed on Mar. 31, 2017.

(51) Int. Cl. A61K 31/501; A61K 31/4468; A61K 31/4436; A61K 31/496; A61K 31/5377; A61K 31/4412; A61K 9/00; A61K 9/20; G01N 33/68

(52) U.S. Cl. CPC A61K 31/501; A61K 31/4468; A61K 31/4436; A61K 31/496; C07K 16/2818; A61K 31/4412; A61K 9/0053; A61K 9/20; G01N 33/6818; A61K 31/5377

(57) ABSTRACT

The present disclosure provides treatment modalities, e.g., strategies, treatment methods, patient stratification methods, combinations, and compositions that are useful for the treatment of disorders, e.g., proliferative disorders, such as certain cancer. Some aspects of this disclosure provide treatment modalities, methods, strategies, compositions, combinations, and dosage forms for the treatment of cell proliferative disorders, e.g., cancers, dependent upon EZH2 (enhancer of zeste 2 polycomb repressive complex 2) function with an EZH2 inhibitor.

### CROSS-REFERENCE TO RELATED APPLICATIONS

This application claims the benefit of U.S. Provisional Patent Application Ser. No, 62/453,929, filed Feb. 2, 2017, and of U.S. Provisional Patent Application Ser. No. 62/479,878, filed Mar. 31, 2017. The entire contents of each the above-mentioned applications are herein incorporated by reference in their entireties.

### SEQUENCE LISTING

The instant application contains a Sequence Listing which has been submitted in ASCII format via EFS-Web and is hereby incorporated by reference in its entirety. Said ASCII copy, created on Jan. 17, 2018, is named EPIZ-074001WO_ST_{25.}txt and is 196,906 bytes in size.

### SUMMARY

The present disclosure provides treatment modalities, e.g., strategies, treatment methods, patient stratification methods, combinations, and compositions that are useful for the treatment of disorders, e.g., proliferative disorders, such as certain cancer. Some aspects of this disclosure provide treatment modalities, methods, strategies, compositions, combinations, and dosage forms for the treatment of cell proliferative disorders, e.g., cancers, dependent upon EZH2 (enhancer of zeste 2 polycomb repressive complex 2) function with an EZH2 inhibitor.

Some aspects of this disclosure provide treatment modalities for treating cell proliferative disorders characterized by the presence of a hyperproliferative cell or cell population, e.g., a cancer cell or cancer cell population, originating from a stem cell, stem-like cell, progenitor cell, or an immature cell, wherein the hyperproliferative cell or cell population comprises a genetic and/or an epigenetic lesion conferring dependence of the cancer cell on an EZH2 function. In some embodiments, the cell proliferative disorder, e.g., a cancer, is characterized by a combination of a stem-, stem-like, or progenitor cell of origin, and one or more genetic and/or epigenetic lesions in at least one gene that regulates polycomb signaling. In some embodiments, the cell proliferative disorder, e.g., a cancer, is characterized by one or more genetic and/or epigenetic lesions resulting in loss of function of one or more SWI/SNF complex members, e.g., INI-1 (also known as SMARCB1, SWI/SNF related, matrix associated, actin dependent regulator of chromatin, subfamily b, member 1), SMARCA2 (SWI/SNF related, matrix associated, actin dependent regulator of chromatin, subfamily a, member 2), and/or SMARCA4 (SWI/SNF related, matrix associated, actin dependent regulator of chromatin, subfamily a, member 4). For example, in some embodiments, the cell proliferative disorder is characterized by one or more genetic and/or epigenetic lesions resulting in loss of function of SMARCA2 and/or SMARCA4. In some embodiments, the cell proliferative disorder is a cell proliferative disorder of the lung, e.g., lung cancer. In certain embodiments the EZH2 inhibitor is tazemetostat. In some embodiments, the cell proliferative disorder is a cancer. In some embodiments, the cell proliferative disorder is characterized by a solid tumor. In some embodiments, the cell proliferative disorder is a cell proliferative disorder of the lung, e.g., lung cancer, such as, for example, non-small cell lung cancer, small cell lung cancer, or mesothelioma. In certain embodiments, treatment modalities, e.g., certain strategies, treatment methods, and patient stratification methods provided herein include administering the EZH2 inhibitor in temporal proximity to the administration of one or more additional therapeutics to a subject in need thereof, e.g., a subject having a cell proliferative disorder described herein. In some embodiments, the one or more additional therapeutics comprise a standard-of-care agent, e.g., an agent commonly used in the clinic for first-line, second-line, or third-line treatment of the cell proliferative disorder. In some embodiments, the one or more additional agents comprise an immune checkpoint inhibitor, e.g., a PD-1 or PDL-1 inhibitor.

Some aspects of this disclosure provide methods comprising administering an EZH2 inhibitor to a subject having or diagnosed with a cell proliferative disorder characterized by a cell or a population of cells that exhibits a loss of function of SMARCA2 and/or SMARCA4. Some aspects of this disclosure provide methods of treating a cell proliferative disorder in a subject in need thereof comprising administering to the subject a therapeutically effective amount of an enhancer of a zeste homolog 2 (EZH2) inhibitor, wherein the cell proliferative disorder is characterized by a cell or a population of cells that exhibits a loss of function of SMARCA2 and/or SMARCA4.

In some embodiments, the cell proliferative disorder is a cell proliferative disorder of the lung. Some aspects of this disclosure provide methods of treating a cell proliferative disorder of the lung in a subject in need thereof comprising administering to the subject a therapeutically effective amount of an enhancer of a zeste homolog 2 (EZH2) inhibitor. In some embodiments, the cell proliferative disorder comprises or is characterized by a cell or a population of cells that exhibits a loss of function of SMARCA2 and/or a loss of function of SMARCA4. In some embodiments, the cell proliferative disorder comprises or is characterized by a cell or a population of cells that exhibits a loss of function of SMARCA2. and SMARCA4. In some embodiments, the cell proliferative disorder is characterized by a stem-, stem-like, or progenitor cell of origin. In some embodiments, the cell proliferative disorder of the lung is characterized by a malignant growth or lesion in the lung. In some embodiments, the malignant growth or lesion is a primary lesion. In some embodiments, the malignant growth or lesion is, or is characterized by, a secondary or metastatic lesion. In some embodiments, the lung cancer is a malignant lung neoplasm, a carcinoma, or a carcinoid tumor. In some embodiments, the cell proliferative disorder of the lung is asbestos-induced hyperplasia, squamous metaplasia, and benign reactive mesothelial metaplasia. In some embodiments, the cell proliferative disorder of the lung is lung cancer. In some embodiments, the lung cancer is small cell lung cancer. In some embodiments, the lung cancer is non-small cell lung cancer. In some embodiments, the lung cancer is a squamous cell carcinoma. In sonic embodiments, the lung cancer is an adenocarcinoma. In some embodiments, the lung cancer is a small cell carcinoma. In some embodiments, the lung cancer is a large cell carcinoma. In some embodiments, the lung cancer is an adenosquamous cell carcinoma. In some embodiments, the lung cancer is mesothelioma.

In some embodiments, the cell proliferative disease is characterized by a primary tumor, wherein the primary tumor (A) exhibits SMARCA2/SMARCA4 dual loss; and (B) is poorly differentiated and/or exhibits epithelial to mesenchymal transition (EPvIT) features. In some embodiments, the primary tumor exhibits low E-cadherin and high vimentin expression levels.

In some embodiments, the subject has been or is being administered an additional therapeutic agent concurrently or in temporal proximity with the administration of the EZH2 inhibitor. In some embodiments, the additional therapeutic agent is a standard-of-care agent. In some embodiments, the additional agent is or comprises an agent listed in Schematic 1, or is or comprises a combination of two or more agents listed in Schematic 1. In some embodiments, the additional therapeutic agent is an immune checkpoint inhibitor. In some embodiments, the immune checkpoint inhibitor is a CTLA4 inhibitor, a PD-1 inhibitor and/or a PD-L1 inhibitor, a LAG3 inhibitor, a B7-H3 inhibitor, or a Tim3 inhibitor. In some embodiments, the immune checkpoint inhibitor comprises Ipilimumab, Ticilimumab, AGEN-1884, Nivolumab, Pembrolizumab, Atezolizumab, Durvalumab, Avelumab, BMS-936559, AMP-224, MEDI-0680, TSR-042, BGB-108, STI-1014, KY-1003, ALN-PDL, BGB-A317, KD-033, REGN-2810, PDR-001, SHR-1210, MGD-013, PF-06801591, CX-072, IMP-731, LAG-525, BMS-986016, GSK-2831781, Enoblituzumab, 1241-8H9, DS-5573, MBG-453, or a combination thereof. In some embodiments, the EZH2 inhibitor and the additional therapeutic agent are administered sequentially to the subject. In some embodiments, the EZH2 inhibitor and the additional therapeutic agent are administered via different administration routes and at different intervals. In some embodiments, the EZH2 inhibitor is administered orally twice a day.

In some embodiments, the method further comprises detecting SMARCA2 and/or SMARCA4 protein expression and/or a function of a SMARCA2 and/or of a SMARCA4 protein. In some embodiments, the expression and/or function of the SMARCA2 and/or the SMARCA4 protein is evaluated by a method comprising: (a) obtaining a biological sample from the subject; (b) contacting the biological sample or a portion thereof with an antibody that specifically binds SMARCA2 or SMARCA4; and (c) detecting an amount of the antibody that is bound to SMARCA2 or SMARCA4.

In some embodiments, the method further comprises detecting a genomic mutation in the gene encoding the SMARCA2 and/or the gene encoding the SMARCA4 protein in a biological sample obtained from the subject. In some embodiments, the genomic mutation is detected by a method comprising: (a) obtaining a biological sample from the subject; (b) sequencing at least one DNA sequence encoding a SMARCA2 protein or a portion hereof, and/or at least one DNA sequence encoding a SMARCA4 protein or a portion thereof, in the biological sample; and (c) determining if the at least one DNA sequence encoding a SMARCA2 protein or a portion thereof, and/or the at least one DNA sequence encoding a SMARCA4 protein or a portion thereof, comprises a mutation affecting the expression and/or function of the SMARCA2 protein or the SMARCA4 protein.

In some embodiments, the EZH2 inhibitor inhibits tri-methylation of lysine 27 of histone 3 (H3K27).

In some embodiments, the treatment modalities, e.g., treatment methods, compositions, or combinations comprise or use a small molecule EZH2 inhibitor of Formula (VIa) below or a pharmaceutically acceptable salt or ester thereof.

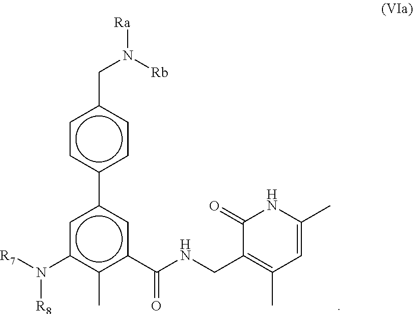

In some embodiments, the compounds of Formula (VIa) can include one or more of the following features:

Each of Rₐ and R_{b}, independently, is H or C₁-C₆ alkyl optionally substituted with one or more -Q₃-T₃.

Rₐ and R_{b}, together with the N atom to which they are attached, form a 4- to 12-membered heterocycloalkyl ring having 0 or 1 additional heteroatoms, wherein the 4- to 12-membered heterocycloalkyl ring is optionally substituted with one or more -Q₃-T₃.

Rₐ and R_{b}, together with the N atom to which they are attached, is a 4 to 7-membered heterocycloalkyl ring having 0 or 1 additional heteroatom, wherein the 4 to 7-membered heterocycloalkyl ring is optionally substituted with one or more -Q₃-T₃.

Each Q₃ is independently a bond or unsubstituted or substituted C₁-C₃ alkyl linker.

Each T₃ is independently H, halo, C₁-C₃ alkyl, OR_{d}, COOR_{d}, S(O)₂R_{d}, NR_{d}Rₑ, or 4 to 7-membered heterocycloalkyl, wherein each of R_{d} and Rₑ, independently, is H or C₁-C₆ alkyl.

R₇ is C₁-C₆ alkyl, C₃-C₈ cycloalkyl, or 4 to 12-membered heterocycloalkyl, each optionally substituted with one or more -Q₅-T₅.

R₇ is C₁-C₆ alkyl, C₃-C₈ cycloalkyl, or 4 to 12-membered (e.g., 4 to 7-membered) heterocycloalkyl, each optionally substituted with one or more -Q₅-T₅. For example, R₇ is not H.

R₇ is 4 to 7-membered heterocycloalkyl optionally substituted with one or more -Q5-T5.

R₇ is piperidinyl, tetrahydropyran, cyclopentyl, or cyclohexyl, each optionally substituted with one -Q₅-T₅.

Each Q₅ is independently a bond, CO, S(O)₂, NHC(O), or C₁-C₃ alkyl linker.

Each T₅ is independently H, halo, S(O)_{q}R_{q}, C₁-C₆ alkyl, C₁-C₆ alkoxy, C₃-C₈ cycloalkyl, 4 to 12-membered heterocycloalkyl, or C₆-C₁₀ aryl, wherein q is 0, 1, or 2 and R_{q} is C₁-C₆ alldyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl.

Each T₅ is independently H, halo, C₁-C₆ alkyl, C₁-C₆ alkoxy, C₃-C₈ cyclaalkyl, C₆-C₁₀ aryl, or 4 to 12-membered (e.g., 4 to 7-membered) heterocycloalkyl.

Q₅ is a bond and T₅ is C₁-C₆ alkyl C₃-C₈ cycloalkyl, or 4 to 12-membered (e.g., 4 to 7-membered) heterocycloalkyl.

Q₅ is CO, S(O)₂, or NHC(O); and T₅ is C₁-C₆ alkyl, C₁-C₆ alkoxy, C₃-C₈ cycloalkyl, or 4 to 12-membered (e.g., 4 to 7-membered) heterocycloalkyl.

Q₅ is C₁-C₃ alkyl linker and T₅ is H or C₆-C₁₀ aryl.

Q₅ is C₁-C₃ alkyl linker and T₅ is C3-C8 cycloalkyl, 4 to 7-membered heterocycloalkyl, or S(O)_{q}R_{q}.

R₇ is cyclopentyl or cyclohexyl, each optionally substituted with one -Q₅-T₅.

Q₅ is NHC(O) and T₅ is C₁-C₆ alkyl or C₁-C₆ alkoxy.

R₇ is isopropyl.

R₈ is H, C₁-C₆ alkyl, or 4 to 7-membered heterocycloalkyl, wherein C₁-C₆ alkyl and heterocycloalkyl are each optionally substituted with one or more substituents selected from the group consisting of halo, hydroxyl, COOH, C(O)O—C₁-C₆ alkyl, cyano, C₁-C₆ alkoxyl, amino, mono-C₁-C₆ alkylamino, and di-C₁-C₆ alkylamino.

R₈ is H, methyl, or ethyl.

R₈ is methyl.

R₈ is ethyl.

R₈ is 4 to 7-heterocycloalkyl, e.g., tetrahydropyran.

In some embodiments, the EZH2 inhibitor is

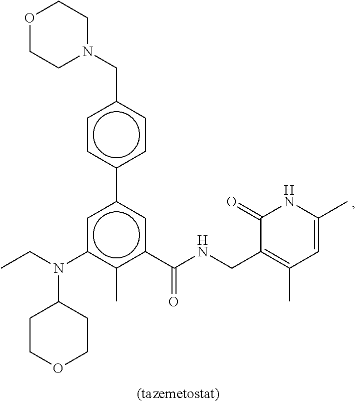

or a pharmaceutically acceptable salt thereof.

In some embodiments, the EZH2 inhibitor is

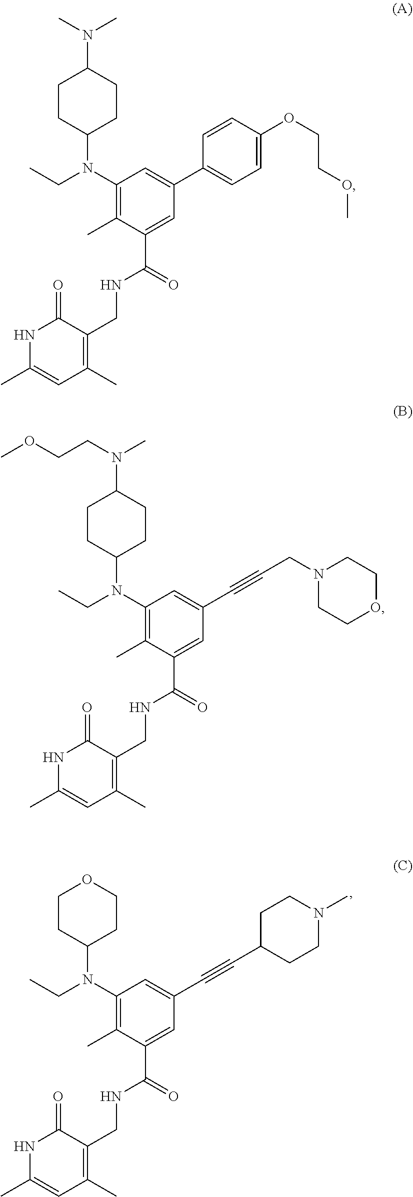

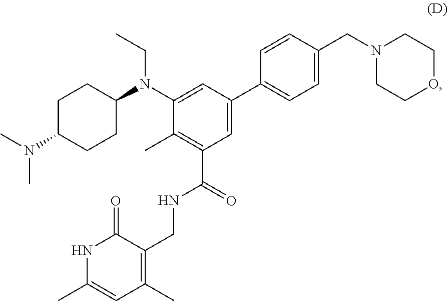 [SMILES: CCN(c1cc(-c2ccc(CN3CCOCC3)cc2)cc(C(=O)NCc2c(C)cc(C)[nH]c2=O)c1C)[C@H]1CC[C@H](N(C)C)CC1]

or a stereoisomer, a pharmaceutically acceptable salt and/or a solvate thereof.

In some embodiments, the EZH2 inhibitor is

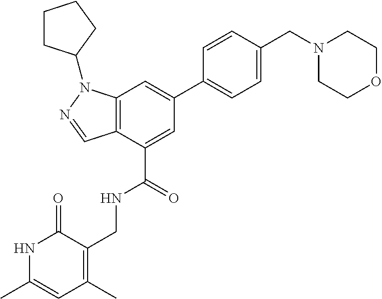 [SMILES: Cc1cc(C)c(CNC(=O)c2cc(-c3ccc(CN4CCOCC4)cc3)cc3c2cnn3C2CCCC2)c(=O)[nH]1]

or a pharmaceutically acceptable salt thereof.

In some embodiments, the EZH2 inhibitor is

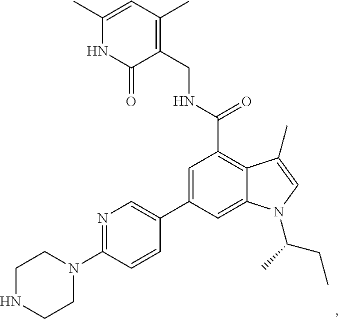 [SMILES: CC[C@H](C)n1cc(C)c2c(C(=O)NCc3c(C)cc(C)[nH]c3=O)cc(-c3ccc(N4CCNCC4)nc3)cc21]

or a stereoisomer, a pharmaceutically acceptable salt and/or a solvate thereof.

In some embodiments, the EZH2 inhibitor is

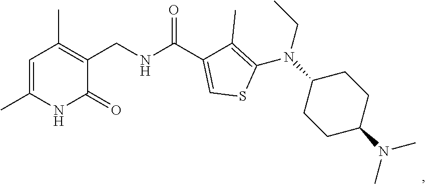

or a stereoisomer, a pharmaceutically acceptable salt and/or a solvate thereof.

In some embodiments, the EZH2 inhibitor is

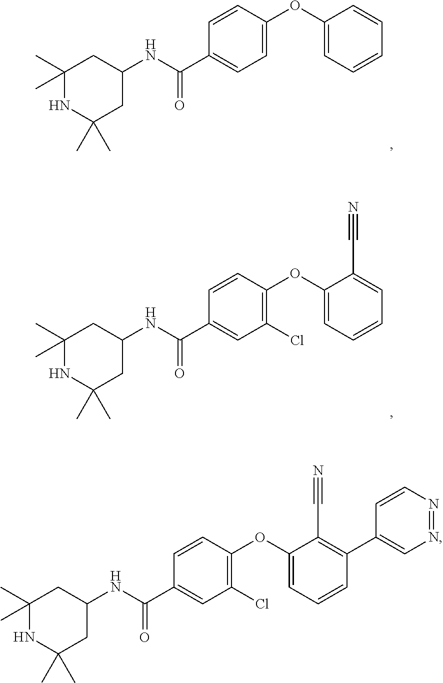 [SMILES: CC1(C)CC(NC(=O)c2ccc(Oc3ccccc3)cc2)CC(C)(C)N1 ; CC1(C)CC(NC(=O)c2ccc(Oc3ccccc3C#N)c(Cl)c2)CC(C)(C)N1 ; CC1(C)CC(NC(=O)c2ccc(Oc3cccc(-c4ccnnc4)c3C#N)c(Cl)c2)CC(C)(C)N1]

or a stereoisomer, a pharmaceutically acceptable salt and/or a solvate thereof.

In some embodiments, the EZH2 inhibitor may comprise, consist essentially of or consist of CPI-I205 or GSK343.

In some embodiments, the EZH2 inhibitor is administered orally. In some embodiments, the EZH2 inhibitor is formulated as an oral tablet. In some embodiments, the EZH2 inhibitor is administered at a dose of between 10 mg/kg/day and 1600 mg/kg/day. In some embodiments, the EZH2 inhibitor is administered at a dose of about 100, 200, 400, 800, or 1600 mg. In some embodiments, the EZH2 inhibitor is administered at a dose of about 800 mg. In some embodiments, the EZH2 inhibitor is administered twice per day (BID).

Some aspects of this disclosure provide methods comprising detecting a SMARCA2 and/or a SMARCA4 loss of function in a sample obtained from a subject. In some embodiments, the subject has cancer. In some embodiments, the method further comprises administering an EZH2 inhibitor to the subject, if a SMARCA2 and/or SMARCA4 loss of function is detected in the subject. In some embodiments, the SMARCA2 loss of function is not associated with a genomic mutation in a gene encoding SMARCA2 protein, and/or wherein the SMARCA4 loss of function is associated with a genomic mutation in a gene encoding SMARCA4. In some embodiments, wherein the subject has NSCLC.

In some embodiments, the treatment modalities provided herein comprise or use a compound selected from Table 1 or a pharmaceutically acceptable salt or ester thereof and one or more other therapeutic agents.

In some embodiments, the treatment modalities provided herein comprise or use the compound provided below:

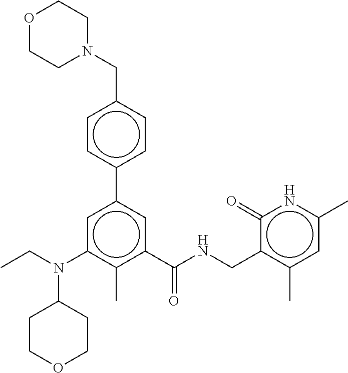

or a pharmaceutically acceptable salt or ester thereof and one or more other therapeutic agents.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed. Description, the Drawings, the Examples, and the Claims.

### BRIEF DESCRIPTION OF THE DRAWINGS

The patent or application file contains at least one drawing executed in color. Copies of this patent or patent application publication with color drawings will be provided by the Office upon request and payment of the necessary fee.

The above and further features will be more clearly appreciated from the following detailed description when taken in conjunction with the accompanying drawings.

FIG. 1. Subunits of SWI/SNF complexes are mutated across various indications.

FIG. 2. Sensitivity of SMARCA2/SMARCA4 and SWI/SNF-mutant lung cancer cells to EZH2 inhibition in vitro.

FIG. 3. Effect of EZH2 inhibition on tumor growth in SMARCA4 single-loss NSCLC cell line xenografts in vivo.

FIG. 4. Effect of EZH2 inhibition on tumor growth in SMARCA2/SMARCA4 dual-loss NSCLC cell line xenografts in vivo.

### DETAILED DESCRIPTION

Some aspects of this disclosure provide treatment modalities, e.g., methods, strategies, compositions, combinations, and dosage forms that are useful in the context of treating cell proliferative disorders, e.g., cancers, dependent upon EZH2 (enhancer of zeste 2 polycomb repressive complex 2) function with an EZH2 inhibitor. Some aspects of this disclosure are based on the recognition that a subtype of cell proliferative disorder conditions, a subtype of certain cancers, is dependent on EZH2 function and can thus effectively be treated with an EZH2 inhibitor. In some embodiments, the EZH2-dependent subtype is characterized by the presence of a hyperproliferative cell or cell population, e.g., a cancer cell or cancer cell population, originating from a stem cell, stem-like cell, progenitor cell, or an immature cell, wherein the at least one hyperproliferative cell or cell population, e.g., at least one cancer cell, comprises a genetic and/or an epigenetic lesion conferring dependence of the cancer cell on an EZH2 function. In some embodiments, the genetic or epigenetic lesion results in loss of function of one or more SWI/SNF complex members, e.g., INT-1 (also known as SMARCB1, SWI/SNF related, matrix associated, actin dependent regulator of chromatin, subfamily b, member 1), SMARCA2 (SWI/SNF related, matrix associated, actin dependent regulator of chromatin, subfamily a, member 2; also sometimes referred to as BRM, SNF2L2, or SNF2LA), and/or SMARCA4 (SWI/SNF related, matrix associated, actin dependent regulator of chromatin, subfamily a, member 4; also sometimes referred to as brahma homologue, BRG1, CSS4, MRD16, RTPS2, SNF2L4, or SNF2LB). For example, in some embodiments, the cell proliferative disorder is characterized by a genetic or epigenetic lesion resulting in loss of function of SMARCA2 and/or SMARCA4.

Some aspects of this disclosure are based on the recognition that certain cell proliferative disorders, e.g., some cancers that exhibit loss of function of SMARCA2 and/or SMARCA4 depend on EZH2 function and are thus sensitive to treatment with an EZH2 inhibitor. For example, some aspects of this disclosure provide treatment modalities, e.g., methods, strategies, compositions, combinations, and dosage forms for the treatment of solid tumors characterized by a stem-, stem-like, or progenitor cell of origin and loss of function in SMARCA2 or SMARCA4.

Genomic, mRNA, and protein sequences of SWI/SNF complex members, including sequence variants and isoforms not associated with loss of function or states of disease or disorder are known to those of skill in the art. Exemplary, non-limiting sequences for SMARCA2 and SMARCA4 are provided herein, e.g., in the “Exemplary Sequences” section below. Additional suitable sequences, e.g., sequences of other species as well as functional sequence variants will be known to those of skill in the art, and the disclosure is not limited in this respect.

Some aspects of this disclosure are based on the recognition that, in certain cell proliferative disorders characterized by loss of function of SMARCA4 and SMARCA2, SMARCA4 function is lost as a result of a genetic mutation, typically biallelic mutation of the SMARCA4 gene, while loss of function of SMARCA2 is not associated with a genetic mutation but with epigenetic silencing. Accordingly, some aspects of this disclosure provide that in some embodiments of cell proliferative disorders sensitive to treatment with an EZH2 inhibitor, loss of SMARCA2 function is a result of epigenetic downregulation or silencing of SMARCA2 gene expression, by hypermethylation of SMARCA2 regulatory sequences. Some aspects of the present disclosure provide methods comprising reactivating epigenetically repressed SMARCA2 expression in hyperproliferative cells, e.g., in malignant cells also exhibiting loss of function of SMARCA4 mediated by genetic mutations, by contacting the cells with an EZH2 inhibitor, for example, with tazemetostat. Typically, EZH2 inhibition and SMARCA2 reactivation in such hyperproliferative cells results in an inhibition of cell survival and/or proliferation. In some clinical embodiments, treatment of a patient having a hyperproliferative disease characterized by loss of function of SMARCA2 and SMARCA4 with an EZH2 inhibitor results in inhibition of hyperproliferation and/or ablation of hyperproliferative cells.

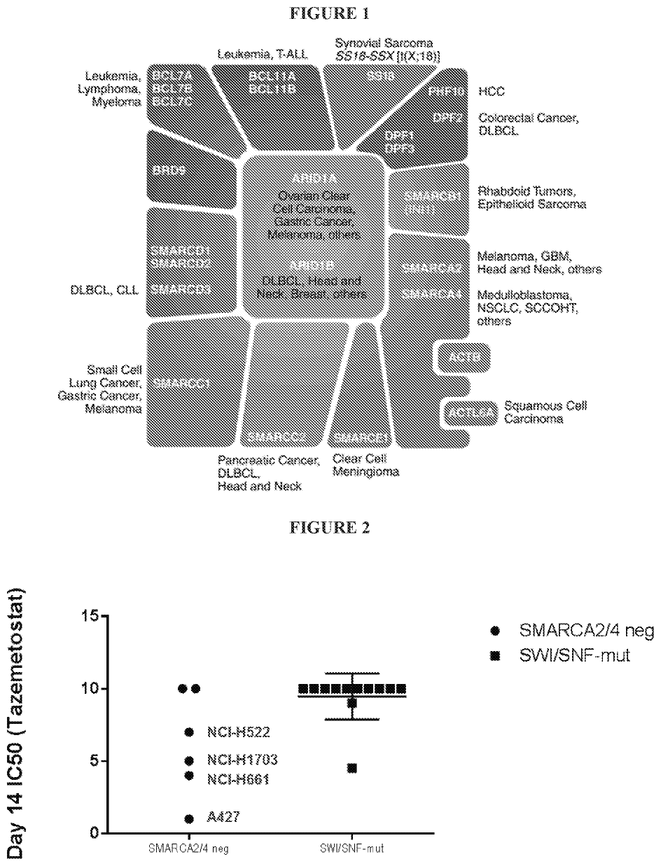

Lesions in genes encoding members of the SWI/SNF complex have previously been reported in a variety of cancers. FIG. 1 lists some exemplary malignant indications in which such lesions were reported. Loss of SMARCA2 and/or SMARCA4, e.g., based on genetic lesions, has been observed in various cell proliferative diseases including, for example, some solid tumor indications, such as, e.g., certain malignant rhabdoid tumors (e.g., malignant rhabdoid tumor of the ovary (MRTO), small cell cancer of the ovary of the hypercalcemic type (SCCOHT); see, e.g., PCT Application PCT/US2016/053673, filed Sep. 26, 2016, the entire contents of which are incorporated herein by reference), and certain lung cancer subtype (e.g., non-small cell lung cancer, small cell lung cancer, adenosarcoma, squamous cell sarcoma). Other cell proliferative disorders characterized by SMARCA2 and/or SMARCA4 loss of function will be known to the person of skill in the art, or will be ascertainable to the skilled artisan based on the present disclosure with no more than routine experimentation. The disclosure is not limited in this respect.

Table 1A below provides a summary of the frequency of SMARCA2/SMARCA4 loss in NSCLC primary tumors.

TABLE 1A

| | | Dual | | SMARCA4 single | | SMARCA4 | SMARCA2 |
|---|---|---|---|---|---|---|---|
| Reference | subtype | loss (IHC) | | Loss (IHC) | | mutation | mutation |
| Matsubara | adeno | 5/93 | (5%) | 11/93 | (12%) | No data | No data |
| et al. 2013 | | | | | | | |
| Resimann | Squamous | 6/60 | (10%) | 6/60 | (10%) | No data | No data |
| et al. 2003 | and adeno | | | | | | |
| Oike | Squamous | 6/103 | (6%) | 16/103 | (18%) | 1/6 | 0/6 |
| et al. 2013 | and adeno | | | | | | |
| NIH Atlas | Squamous | | | 15-30% | | 0.56-3.31% | 0% |
| database | and adeno | | | | | | |
| Epizyme | NSCLC | 6/226 | (3%) | 19/272 | (7%) | No data | No data |
| Internal Data* | | | | | | | |

The observed dual SMARCA2/SMARCA4 loss frequency of 3-10% equates to 7,000-23,000 cases of NSCLC per year in the U.S. alone. Some aspects of this disclosure are based on the surprising discovery that SMARCA4 and SMARCA2 protein loss is significantly higher in certain cancers, e.g., in NSCLC, than the frequency at which the encoding genes comprise a loss-of-function mutation.

Loss of protein function without underlying genomic mutation cannot be detected by genomic sequence analysis. Accordingly, conventional methods for classifying hyperproliferative diseases that are associated with SMARCA2 and/or SMARCA4 loss of function based on DNA sequence analysis are prone to false negative results, and typically underestimate the frequency of dual SMARCA2/SMARCA4 loss of function. Some aspects of this disclosure provide methods for accurately determining SMARCA2 and SMARCA4 status in hyperproliferative cells or cell populations, e.g., in a tumor biopsy obtained from a subject having cancer, by analyzing protein expression levels or protein function of SMARCA2 and/or SMARCA4. For example, in some embodiments, a patient stratification method is provided that comprises detecting the level of SMARCA2 and/or SMARCA4 protein in a biological sample obtained from a subject having cancer, e.g., lung cancer, and comparing the level to a reference or control level, e.g., a level observed or expected in healthy, non-malignant cells.

In some embodiments, the method comprises detecting the level of SMARCA2 and/or of SMARCA4 protein in the sample obtained from the subject by an immunology-based method, e.g., by immunohistochemistry, westem blot, ELISA, or other suitable assay. In some embodiments, the method comprises detecting the level of SMARCA2 and/or SMARCA4 activity based on a protein dynamics assay, e.g., by an assay determining the enzymatic activity of SMARCA2 and/or SMARCA4 in the sample. In some embodiments, the methods provided herein can detect hyperproliferative cells or cell populations exhibiting SMARCA2/SMARCA4 dual loss, e.g., in malignant cells obtained from a subject, with greater accuracy than conventional, DNA-sequencing-based methods.

In some embodiments, the method comprises classifying a cancer, e.g., a lung cancer, such as NSCLC, as sensitive to treatment with an EZH2 inhibitor, if the protein level of SMARCA2 and/or of SMARCA4 is decreased as compared to the reference or control level. For example, in some embodiments, the method comprises classifying the cancer as sensitive to treatment with an EZH2 inhibitor, if the protein level of SMARCA2 and/or of SMARCA4 protein is decreased as compared to the reference or control level. In some embodiments, a cancer is classified as sensitive to treatment with an EZH2 inhibitor, if the cancer exhibits dual SMARCA2/SMARCA4 loss, and if SMARCA2 function or SMARCA4 function, or both, are lost without a loss-of-function mutation in the respective encoding gene. For example, in some embodiments, the method comprises classifying a cancer characterized by SMARCA4 loss of function based on a genomic mutation in the SMARCA4 gene, and SMARCA2 loss of function not associated with a genomic mutation in the SMARCA2 gene as sensitive to treatment with an EZH2 inhibitor. For another example, in some embodiments, the method comprises classifying a cancer characterized by SMARCA2 loss of function based on a genomic mutation in the SMARCA2 gene, and SMARCA4 loss of function not associated with a genomic mutation in the SMARCA4 gene as sensitive to treatment with an EZH2 inhibitor.

In some embodiments, a method is provided that comprises administering an EZH2 inhibitor, e.g., tazemetostat, to a subject harboring hyperproliferative cells exhibiting SMARCA2/SMARCA4 dual loss. In some embodiments, the subject harbors a solid tumor having a stem-, stem-like, or progenitor cell of origin, and exhibiting a SMARCA2/SMARCA4 dual loss, wherein the loss of SMARCA2 and/or SMARCA4 is not associated with a loss-of-function mutation in the respective encoding gene. For example, in some embodiments, the method comprises administering an EZH2 inhibitor to a subject having a cancer, e.g., lung cancer, such as, e.g., NSCLC, characterized by SMARCA4 loss of function based on a genomic mutation in the SMARCA4 gene, and SMARCA2 loss of function not associated with a genomic mutation in the SMARCA2 gene. For another example, in some embodiments, the method comprises administering the EZH2 inhibitor to a subject having a cancer characterized by SMARCA2 loss of function based on a genomic mutation in the SMARCA2 gene, and SMARCA4 loss of function not associated with a genomic mutation in the SMARCA4 gene.

Some aspects of the present disclosure provide that EZH2 inhibition can inhibit or abolish a hyperproliferative state of a cell that is characterized loss of function of SMARCA2 and/or SMARCA4, e.g., dual loss of SMARCA2. and SMARCA4, where at least one of the loss-of-function lesions in the cell is an epigenetic lesion. A hyperproliferative state of a cell in a subject is typically associated with a cell proliferative disorder, e.g., with a cancerous or precancerous condition. Cell proliferative disorders that can be treated with the treatment modalities provided herein include all forms of cell proliferative disorders, e.g., cancer, precancer or precancerous conditions, benign growths or lesions, malignant growths or lesions, and metastatic lesions. In some embodiments, the cell proliferative disorder is characterized by hyperplasia, metaplasia, or dysplasia. In some embodiments, the cell proliferative disease is characterized by a primary tumor. In some embodiments, the primary tumor is a solid tumor. In some embodiments, the primary tumor is a liquid tumor. In some embodiments, the cell proliferative disease is characterized by a malignant growth or tumor. In some embodiments, the cell proliferative disease is characterized by a secondary or metastatic tumor.

Some aspects of the present disclosure provide treatment modalities suitable for the treatment of a cell proliferative disorder of the lung that is characterized by loss of function of SMARCA2 and/or SMARCA4, e.g., dual loss of function of SMARCA2 and SMARCA4, where at least one of the loss-of-function lesions in the cell is an epigenetic lesion.: cell proliferative disorder of the lung is a cell proliferative disorder involving cells of the lung. Cell proliferative disorders of the lung can include all forms of cell proliferative disorders affecting lung cells, Cell proliferative disorders of the lung can include lung cancer, a precancer or precancerous condition of the lung, benign growths or lesions of the lung, and malignant growths or lesions of the lung, and metastatic lesions in tissue and organs in the body other than the lung. In one aspect, compositions of the present disclosure may be used to treat lung cancer or cell proliferative disorders of the lung, or used to identify suitable candidates for such purposes. Lung cancer can include all forms of cancer of the lung. Lung cancer can include malignant lung neoplasms, carcinoma in situ, typical carcinoid tumors, and atypical carcinoid tumors. Lung cancer can include small cell lung cancer (“SCLC”), non-small cell lung cancer (“NSCLC”), squamous cell carcinoma, adenocarcinoma, small cell carcinoma, large cell carcinoma, adenosquamous cell carcinoma, and mesothelioma. Lung cancer can include “scar carcinoma,” bronchioalveolar carcinoma, giant cell carcinoma, spindle cell carcinoma, and large cell neuroendocrine carcinoma. Lung cancer can include lung neoplasms having histologic and ultrastructural heterogeneity (e.g., mixed cell types).

Cell proliferative disorders of the lung can include all forms of cell proliferative disorders affecting lung cells. Cell proliferative disorders of the lung can include lung cancer, precancerous conditions of the lung. Cell proliferative disorders of the lung can include hyperplasia, metaplasia, and dysplasia of the lung. Cell proliferative disorders of the lung can include asbestos-induced hyperplasia, squamous metaplasia, and benign reactive mesothelial metaplasia. Cell proliferative disorders of the lung can include replacement of columnar epithelium with stratified squamous epithelium, and mucosal dysplasia. Individuals exposed to inhaled injurious environmental agents such as cigarette smoke and asbestos may be at increased risk for developing cell proliferative disorders of the lung. Prior lung diseases that may predispose individuals to development of cell proliferative disorders of the lung can include chronic interstitial lung disease, necrotizing pulmonary disease, scleroderma, rheumatoid disease, sarcoidosis, interstitial pneumonitis, tuberculosis, repeated pneumonias, idiopathic pulmonary fibrosis, granulomata, asbestosis, fibrosing alveolitis, and Hodgkin's disease.

Some aspects of the present disclosure provide treatment modalities suitable for the treatment of lung cancer, e.g., lung cancer characterized by loss of function of SMARCA2 and/or SMARCA4, e.g., dual loss of SMARCA2 and SMARCA4 function, where at least one of the loss-of-function lesions in the cell is an epigenetic lesion. Lung cancer is the most common cause of cancer-related death worldwide. There are about 225.000 new cases of lung cancer diagnoses per year in the U.S. alone. About 85-90% of lung cancers are characterized as non-small-cell lung cancer (NSCLC), which display a diverse range of genetic driver mutations. Treatment for lung cancers has evolved from chemotherapy to targeted therapies. However, there remains a large unmet clinical need for new treatment modalities, e.g., methods, strategies, compositions, combinations, and dosage forms, as well as for efficient patient stratification. This is particularly true for patients receiving later-line chemotherapy. The more recently developed molecular targeted therapies are most suitable for treating adenocarcinomas (e.g., non-squamous carcinomas), while effective targeted treatments are not available for other lung cancer subtypes.

An overview of an exemplary paradigm for patient stratification and clinical management of NSCLC is described in Thomas et al. Nature Reviews 2016, the entire contents of which are incorporated herein by reference. Schematic 1 below was adapted from Thomas et al. to outline some exemplary treatment modalities in first-, second-, and third-line treatment. It will be understood that the schematic below is included here to illustrate certain exemplary treatment modalities used by clinicians, that it is not limiting the scope of the present disclosure, and that other suitable patient stratification and treatment modalities will be known to those of skill in the art.

While good responses are often observed in initial treatment regimen of conventional and targeted treatment modalities, resistance to such therapeutics ultimately develops in the majority of cases and treatment options for those patients who develop resistant or refractory disease are limited. New targeted treatment modalities, e.g. immune-checkpoint inhibitors, are being developed for certain lung cancer indications, but there remains a need for effective treatment options for first-line treatment and treatment of lung cancers resistant to standard-of-care treatment strategies.

Some aspects of the present disclosure provide treatment modalities suitable for the treatment of a cell proliferative disorder of the hematologic system that is characterized by loss of function of SMARCA2 and/or SMARCA4, e.g., dual loss of SMARCA2 and SMARCA4, where at least one of the loss-of-function lesions in the cell is an epigenetic lesion. A cell proliferative disorder of the hematologic system is a cell proliferative disorder involving cells of the hematologic system. A cell proliferative disorder of the hematologic system suitable for the strategies, treatment modalities, methods, combinations, and compositions provided herein can include lymphoma, leukemia, myeloid neoplasms, mast cell neoplasms, myelodysplasia, benign monoclonal gammopathy, lymphomatoid granulomatosis, lymphomatoid papulosis, polycythemia vera, chronic myelocytic leukemia, agnogenic myeloid metaplasia, and essential thrombocythemia. A cell proliferative disorder of the hematologic system can include hyperplasia, dysplasia, and metaplasia of cells of the hematologic system. In some embodiments, the strategies, treatment modalities, methods, combinations, and compositions provided herein are used to treat a cancer selected from the group consisting of a hematologic cancer of the disclosure or a hematologic cell proliferative disorder of the disclosure. A hematologic cancer of the disclosure can include multiple myeloma, lymphoma (including Hodgkin's lymphoma, non-Hodgkin's lymphoma, childhood lymphomas, and lymphomas of lymphocytic and cutaneous origin), leukemia (including childhood leukemia, hairy-cell leukemia, acute lymphocytic leukemia, acute myelocytic leukemia, chronic lymphocytic leukemia, chronic myelocytic leukemia, chronic myelogenous leukemia, and mast cell leukemia), myeloid neoplasms and mast cell neoplasms.

Some aspects of the present disclosure provide treatment modalities suitable for the treatment of a cancer. In some embodiments, the cancer is characterized by loss of function of SMARCA2 and/or SMARCA4, e.g., dual loss of SMARCA2 and SMARCA4, where at least one of the loss-of-function lesions in the cell is an epigenetic lesion. In some embodiments, the cancer is characterized by a cell of origin that is a stem cell, a stem-like cell, or a progenitor cell. In some embodiments, the cancer is a poorly-differentiated cancer. In some embodiments, the cancer is characterized by a solid tumor. In some embodiments, the cancer is characterized by a secondary or metastatic tumor. In some embodiments, the cancer is resistant or refractory to chemotherapy. In some embodiments, the cancer is resistant or refractory to first-, second-, and/or third-line treatment. In some embodiments, the cancer is derived from an immune cell. In some embodiments, the cancer is a form of lymphoma, e.g., a B-cell lymphoma, Non-Hodgkin's Lymphoma or Diffuse Large B-cell Lymphoma (DLBCL). In some embodiments, the cancer is adrenocortical carcinoma, AIDS-related cancers, AIDS-related lymphoma, anal cancer, anorectal cancer, cancer of the anal canal, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, AIDS-related lymphoma, non-Hodgkin lymphoma, primary central nervous system lymphoma, Waldenstroem macroglobulinemia, medulloblastoma, melanoma, intraocular (eye) melanoma, merkel cell carcinoma, mesothdioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, Ewing family of sarcoma tumors, Kaposi Sarcoma, soft tissue sarcoma, epithelioid sarcoma, synovial sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, or Wilm's Tumor.

In some embodiments, a cancer that can be treated with the strategies, treatment modalities, methods, combinations, and compositions of the disclosure comprise a solid tumor. In some embodiments, a cancer that can be treated with the strategies, treatment modalities, methods, combinations, and compositions of the disclosure comprises or is derived from a cell of epithelial origin. In some embodiments, cancers that can be treated with the strategies, treatment modalities, methods, combinations, and compositions of the disclosure are primary tumors. In some embodiments, cancers that can be treated with the strategies, treatment modalities, methods, combinations, and compositions of the disclosure are secondary tumors. In some embodiments, the cancer is metastatic.

Some aspects of the present disclosure provide treatment modalities suitable for the treatment of a cancer staged according to the American Joint Committee on Cancer (MCC) TNM classification system, where the tumor (T) has been assigned a stage of TX, T1, T1mic, T1a, T1b, T1c, T2, T3, T4, T4a, T4b, T4c, or T4d; and where the regional lymph nodes (N) have been assigned a stage of NX, N0, N1, N2, N2a, N2b, N3, N3a, N3b, or N3c; and where distant metastasis (M) can be assigned a stage of MX, M0, or M1. In some embodiments, a cancer suitable for treated with the modalities provided herein is a cancer staged according to an American Joint Committee on Cancer (AJCC) classification as Stage I, Stage IIA, Stage IIB, Stage IIIA, Stage IIIB, Stage IIIC, or Stage IV. In some embodiments, a cancer suitable for treatment with the modalities provided herein can be assigned a grade according to an AJCC classification as Grade GX (e.g., grade cannot be assessed), Grade 1, Grade 2, Grade 3 or Grade 4. In some embodiments, the cancer that is to be treated is staged according to an AJCC pathologic classification (pN) of pNX, pN0, PNO (I−), PNO (I+), PN0 (mol−), PN0 (mol+), PN1, PN1(mi), PN1a, PN1b, PN1c, pN2, pN2a, pN2b, pN3, pN3a, pN3b, or pN3c.

Some aspects of the present disclosure provide treatment modalities suitable for the treatment of a cancer that includes a tumor that has been determined to be less than or equal to about 2 centimeters in diameter. In some embodiments, the cancer that is to be treated can include a tumor that has been determined to be from about 2 to about 5 centimeters in diameter. In some embodiments, a cancer that is to be treated can include a tumor that has been determined to be greater than or equal to about 3 centimeters in diameter. In some embodiments, a cancer that is to be treated can include a tumor that has been determined to be greater than 5 centimeters in diameter. In some embodiments, a cancer that is to be treated can be classified by microscopic appearance as well differentiated, moderately differentiated, poorly differentiated, or undifferentiated. In some embodiments, a cancer that is to be treated can be classified by microscopic appearance with respect to mitosis count (e.g., amount of cell division) or nuclear pleioniorphism (e.g., change in cells). In some embodiments, a cancer that is to be treated can be classified by microscopic appearance as being associated with areas of necrosis (e.g., areas of dying or degenerating cells). In some embodiments, a cancer that is to be treated can be classified as having an abnormal karyotype, having an abnormal number of chromosomes, or having one or more chromosomes that are abnormal in appearance. In some embodiments, a cancer that is to be treated can be classified as being aneuploid, triploid, tetraploid, or as having an altered ploidy. In some embodiments, a cancer that is to be treated can be classified as having a chromosomal translocation, or a deletion or duplication of an entire chromosome, or a region of deletion, duplication or amplification of a portion of a chromosome.

In some embodiments, a cancer that is to be treated can be evaluated by DNA cytometry, flow cytometry, or image cytometry. In some embodiments, a cancer that is to be treated can be typed as having 10%, 20%, 30%, 40%, 50%, 60%, 70%, 80%, or 90% of cells in the synthesis stage of cell division (e.g., in S phase of cell division). In some embodiments, a cancer that is to be treated can be typed as having a low S-phase fraction or a high S-phase fraction.

In some embodiments, the present disclosure provides treatment modalities that are useful for the treatment of cancer. Treating cancer can result in a reduction in size of a tumor. A reduction in size of a tumor may also be referred to as “tumor regression”. Preferably, after treatment with the strategies, treatment modalities, methods, combinations, and compositions provided herein, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer can result in a reduction in tumor volume. Preferably, after treatment with the strategies, treatment modalities, methods, combinations, and compositions provided herein, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

In some embodiments, treating cancer results in a decrease in the number of tumors. Preferably, after treatment with the strategies, treatment modalities, methods, combinations, and compositions provided herein, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

In some embodiments, treating cancer can result in a decrease in number of metastatic lesions in other tissues or organs distant from the primary tumor site. Preferably, after treatment with the strategies, treatment modalities, methods, combinations, and compositions provided herein, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

In some embodiments, treating cancer can result in an increase in average survival time of a population of treated subjects in comparison to a population receiving carrier alone. Preferably, after treatment with the strategies, treatment modalities, methods, combinations, and compositions provided herein, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

In some embodiments, treating cancer can result in an increase in average survival time of a population of treated subjects in comparison to a population of untreated subjects. Preferably, after treatment with the strategies, treatment modalities, methods, combinations, and compositions provided herein, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

In some embodiments, treating cancer can result in increase in average survival time of a population of treated subjects in comparison to a population receiving monotherapy with a drug that is not a compound of the disclosure, or a pharmaceutically acceptable salt, solvate, analog or derivative thereof. Preferably, after treatment with the strategies, treatment modalities, methods, combinations, and compositions provided herein, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

In some embodiments, treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the disclosure, or a pharmaceutically acceptable salt, solvate, analog or derivative thereof. Preferably, after treatment with the strategies, treatment modalities, methods, combinations, and compositions provided herein, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

In some embodiments, treating cancer can result in a decrease in tumor growth rate. Preferably, after treatment with the strategies, treatment modalities, methods, combinations, and compositions provided herein, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

In some embodiments, treating cancer can result in a decrease in tumor regrowth. Preferably, after treatment with the strategies, treatment modalities, methods, combinations, and compositions provided herein, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

In some embodiments, treating a cell proliferative disorder can result in a reduction in the rate of cellular proliferation. Preferably, after treatment with the strategies, treatment modalities, methods, combinations, and compositions provided herein, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

In some embodiments, treating a cell proliferative disorder can result in a reduction in the proportion of proliferating cells. Preferably, after treatment with the strategies, treatment modalities, methods, combinations, and compositions provided herein, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

In some embodiments, treating or preventing a cell proliferative disorder can result in a decrease in size of an area or zone of cellular proliferation. Preferably, after treatment with the strategies, treatment modalities, methods, combinations, and compositions provided herein, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

In some embodiments, treating or preventing a cell proliferative disorder can result in a decrease in the number or proportion of cells having an abnormal appearance or morphology. Preferably, after treatment with the strategies, treatment modalities, methods, combinations, and compositions provided herein, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism

In some embodiments, treating a cell proliferative disorder can result in death of hyperproliferative cells, and preferably, cell death results in a decrease of at least 10% in number of cells in a hyperproliferative cell population. More preferably, cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. A number of cells in a population can be measured by fluorescence activated cell sorting (FACS), immunofluorescence microscopy and light microscopy. Methods of measuring cell death are as shown in Li et al., Proc Acad Sci USA. 100(5): 2674-8, 2003. In some embodiments, cell death occurs by apoptosis.

In some embodiments, treating a cell proliferative disorder, e.g., cancer, by administering an EZH2 inhibitor to a subject in need thereof results in one or more of the following: prevention of cancer cell proliferation by accumulation of cells in one or more phases of the cell cycle (e.g. G1, G1/S, G2/M), or induction of cell senescence, or promotion of tumor cell differentiation; promotion of cell death in cancer cells via cytotoxicity, necrosis or apoptosis, preferably without a significant amount of cell death in normal cells.

In certain embodiments of the methods of the disclosure, the treatmentmodalities, e.g., treatment strategies, treatment methods, molecular assays, compositions, and combinations provided herein are applied or administered to a subject in need thereof, e.g., a subject having a cell proliferative disorder. I some embodiments, the subject has been diagnosed with cancer. In some embodiments, the subject is an adult. In some embodiments, the subject is a pediatric subject. In some embodiments, the subject is a human.

In certain embodiments, the subject is an adult, and a therapeutically effective amount of an EZH2 inhibitor, e.g., of tazemetostat, is administered to the subject, wherein the therapeutically effective amount is about 100 mg to about 1600 mg. In certain embodiments, the subject is an adult, and the therapeutically effective amount of the EZH2 inhibitor is about 100 mg, 200 mg, 400 mg, 800 mg, or about 1600 mg. In certain embodiments, the subject is an adult, and the therapeutically effective amount of the EZH2 inhibitor is about 800 mg, e.g., 800 mg/day administered at a dose of 400mg orally twice a day.

In certain embodiments, the subject is pediatric, and the EZH2 inhibitor, e.g., tazemetostat, may be administered at a dose of between 230 mg/m² and 600 mg/m² twice per day (BID), inclusive of the endpoints. In certain embodiments, the subject is pediatric, and the EZH2 inhibitor is administered at a dose of between 230 mg/m² and 305 mg/m² twice per day (BID), inclusive of the endpoints. In certain embodiments, the subject is pediatric, and the EZH2 inhibitor is administered at a dose of 240 mg/m² twice per day (BID). In certain embodiments, the subject is pediatric, and the EZH2 inhibitor is administered at a dose of 300 mg/m² twice per day (BID). In certain embodiments, the subject is pediatric, and the EZH2 inhibitor is administered at a dose of about 60% of the area under the curve (AUC) at steady state (AUCss) following administration of 1600 mg twice a day to an adult subject. In certain embodiments, the subject is pediatric, and the EZH2 inhibitor is administered at a dose of about 600 mg/m² per day. In certain embodiments, the subject is pediatric, and the EZH2 inhibitor is administered at a dose of at least 600 mg/m² per day. In certain embodiments, the subject is pediatric, and the EZH2 inhibitor is administered at a dose of about 80% of the area under the curve (AUC) at steady state (AUCss) following administration of 800 mg twice a day to an adult subject. In certain embodiments, the subject is pediatric, and the EZH2 inhibitor is administered at a dose of about 390 mg/m² twice per day (BID). In certain embodiments, the subject is pediatric, and the EZH2 inhibitor is administered at a dose of at least 390 mg/m² twice per day (BID) In certain embodiments, the subject is pediatric, and the EZH2 inhibitor is administered at a dose of between 300 mg/m² and 600 mg/m² twice per day (BID).

In some embodiments, e.g., in some embodiments where the subject s pediatric, the EZH2 inhibitor is formulated as an oral suspension.

Some aspects of the present disclosure provide combination treatment modalities suitable for the treatment of a cell proliferative disorder, e.g., a cancer described herein by administering to a subject in need thereof a therapeutically effective dose of an EZH2 inhibitor. In some such combination treatment embodiments, the treatment modalities provided herein include methods that comprise administering an EZH2 inhibitor to a subject in need thereof, e.g., a subject having a cell proliferative disorder, wherein the subject has been or is being administered an additional therapeutic agent in temporal proximity to the administration of the EZH2 inhibitor. In some embodiments, treatment modalities are provided that comprise administering the EZH2 inhibitor and the additional therapeutic agent to the subject. In some embodiments, administration in temporal proximity refers consecutive administration of the EZH2 inhibitor and the additional therapeutic agent, in any order, within hours or days of each other, or to an overlap in administration regimens of the EZH2 inhibitor (e.g. twice daily) and the additional therapeutic agent (e.g., once every week) for a certain period of time (e.g., at least one week, at least one month, at least one round of treatment, etc.).

In some embodiments, the present disclosure provides combination therapy strategies, treatment modalities, methods, combinations, and compositions that are useful for improving the clinical outcome and/or the prognosis of a subject having a cell proliferative disease, e.g., a cancer characterized by a loss of SMARCA2 and/or SMARCA4, as compared to monotherapeutic approaches. In some embodiments, the combination therapy approaches provided herein result in a shorter time period being required to achieve a desired clinical outcome (e.g., partial or complete disease remission, inhibition of tumor growth, stable disease), as compared to monotherapy. In some embodiments, the combination therapy approaches provided herein result in a better clinical outcome as compared to monotherapy (e.g., complete vs. partial remission, stable vs. progressive disease, lower recurrence risk).

As used herein, the terms “combination treatment,” “combination therapy,” and “co-therapy” are used interchangeably and generally refer to treatment modalities featuring an EZH2 inhibitor as provided herein and an additional therapeutic agent. Typically, combination treatment modalities are part of a specific treatment regimen intended to provide a beneficial effect from the concurrent action of the therapeutic agent combination. The beneficial effect of the combination may include, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). In some embodiments, combination treatment comprises administration of two or more therapeutic agents in a sequential manner, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single dosage form having a fixed ratio of each therapeutic agent or in multiple, separate dosage forms for the therapeutic agents. Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. The therapeutic agents can be administered according to the same or to a different administration interval. For example, a first therapeutic agent of the combination selected may be administered by intravenous injection while the other therapeutic agents of the combination may be administered orally. Alternatively, for example, all therapeutic agents may be administered orally or all therapeutic agents may be administered by intravenous injection.

In some embodiments, combination therapy also embraces the administration of the therapeutic agents as described above in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, perhaps by days or even weeks.

In some embodiments, the additional therapeutic agent is a chemotherapeutic agent (also referred to as an anti-neoplastic agent or anti-proliferative agent), e.g., an alkylating agent; an antibiotic; an anti-metabolite; a detoxifying agent; an interferon; a polyclonal or monoclonal antibody; an EGFR inhibitor; a HER₂ inhibitor; a histone deacetylase inhibitor; a hormone; a mitotic inhibitor; an MTOR inhibitor; a multi-kinase inhibitor; a serine/threonine kinase inhibitor; a tyrosine kinase inhibitors; a VEGF/VEGFR inhibitor; a taxane or taxane derivative, an aromatase inhibitor, an anthracycline, a microtubule targeting drug, a topoisomerase poison drug, an inhibitor of a molecular target or enzyme (e.g., a kinase or a protein methyltransferase), a cytidine analogue drug or any chemotherapeutic, an immune checkpoint inhibitor, or any anti-neoplastic or anti-proliferative agent known to those of skill in the art.

Exemplary alkylating agents suitable for use according to the combination treatment modalities provided herein include, but are not limited to, cyclophosphamide (Cytoxan; Neosar); chlorambucil (Leukeran); melphalan (Alkeran); carmustine (BiCNU); busulfan (Busulfex); lomustine (CeeNU); dacarbazine (DTIC-Dome); oxaliplatin (Eloxatin); carmustine (Gliadel); ifosfamide (Hex); mechlorethamine (Mustargen); busulfan (Myleran); carboplatin (Paraplatin); cisplatin (CDDP; Platinol); temozolomide (Temodar); thiotepa (Thioplex); bendamustine (Treanda); or streptozocin (Zanosar).

Exemplary suitable antibiotics include, but are not limited to, doxorubicin (Adriamycin); doxorubicin liposomal (Doxil); mitoxantrone (Novantrone); bleomycin (Blenoxane); daunorubicin (Cerubidine); daunorubicin liposomal (DaunoXome); dactinomycin (Cosmegen); epirubicin (Ellence); idarubicin (Idamycin); plicamycin (Mithracin); mitomycin (Mutamycin); pentostatin (Nipent); or valrubicin (Valstar).

Exemplary anti-metabolites include, but are not limited to, fluorouracil (Adrucil); capecitabine (Xeloda); hydroxyurea (Hydrea); mercaptopurine (Purinethol); pemetrexed (Alimta); fludarabine (Fludara); nelarabine (Arranon); cladribine (Cladribine Novaplus); clofarabine (Clolar); cytarabine (Cytosar-U); decitabine (Dacogen); cytarabine liposomal (DepoCyt); hydroxyurea (Droxia); pralatrexate (Folotyn); floxuridine (FUDR); gemcitabine (Gemzar); cladribine (Leustatin); fludarabine (Oforta); methotrexate (MTX; Rheumatrex); methotrexate (Trexall); thioguanine (Tabloid); TS-1 or cytarabine (Tarabine PFS).

Exemplary detoxifying agents include, but are not limited to, amifostine (Ethyol) or mesna (Mesnex).

Exemplary interferons include, but are not limited to, interferon alfa-2b (Intron A) or interferon alfa-2a (Roferon-A).

Exemplary polyclonal or monoclonal antibodies include, but are not limited to, trastuzumab (Herceptin); ofatumumab (Arzerra); bevacizumab (Avastin); rituximab (Rituxan); cetuximab (Erbitux); panitumumab (Vectibix); tositumomab/iodine-131 tositumomab (Bexxar); alemtuzumab (Campath); ibritumomab (Zevalin; In-111; Y-90 Zevalin); gemtuzumab (Mylotarg); eculizumab (Soliris) or denosumab.

Exemplary EGFR inhibitors include, but are not limited to, gefitinib (Iressa); lapatinib (Tykerb); cetuximab (Erbitux); erlotinib (Tarceva); panitumumab (Vectibix); PKI-166; canertinib (C1-1033); matuzumab (EMD 72000) or EKB-569.

Exemplary HER₂ inhibitors include, but are not limited to, trastuzumab (Herceptin); lapatinib (Tykerb) or AC-480.

Histone Deacetylase Inhibitors include, but are not limited to, vorinostat(Zolinza)

Exemplary hormones include, but are not limited to, tamoxifen (Soltamox; Nolvadex); raloxifene (Evista); megestrol (Megace); leuprolide (Lupron; Lupron Depot; Eligard; Viadur); fulvestrant (Faslodex); letrozole (Femara); triptorelin (Trelstar LA; Trelstar Depot) ; exemestane (Aromasin) ; goserelin (Zoladex) ; bicalutamide (Casodex); anastrozole (Arimidex); fluoxymesterone (Androxy; Halotestin); medroxyprogesterone (Provera; Depo-Provera); estramustine (Emcyt); flutamide (Eulexin); toremifene (Fareston); degarelix (Firmagon); nilutamide (Nilandron); abarelix (Plenaxis); or testolactone (Teslac).

Exemplary mitotic inhibitors include, but are not limited to, paclitaxel (Taxol; Onxol; Abraxane); docetaxel (Taxotere); vincristine (Oncovin; Vincasar PFS); vinblastine (Velban); etoposide (Toposar; Etopophos; VePesid); teniposide (Vumon); ixabepilone (Ixempra); nocodazole; epothilone; vinorelbine (Navelbine); camptothecin (CPT); irinotecan (Camptosar); topotecan (Hycamtin); amsacrine or lamellarin D (LAM-D).

Exemplary MTOR inhibitors include, but are not limited to, everolimus (Afinitor) or temsirolimus (Torisel); rapamune, ridaforolimus; or AP23573.

Exemplary multi-kinase inhibitors include, but are not limited to, sorafenib (Nexavar); sunitinib (Sutent); BMW 2992; E7080; Zd6474; PKC-412; motesanib; or AP24534.

Exemplary serine/threonine kinase inhibitors include, but are not limited to, ruboxistaurin; eril/fasudil hydrochloride; flavopiridol; seliciclib (CYC202; Roscovitine); SNS-032 (BMS-387032); Pkc412; bryostatin; KAI-9803; SF1126; VX-680; Azd1152; Arry-142886 (AZD-6244); SCIO-469; GW681323; CC-401; CEP-1347 or PD 332991.

Exemplary tyrosine kinase inhibitors include, but are not limited to, erlotinib (Tarceva); gefitinib (Iressa); imatinib (Gleevec); sorafenib (Nexavar); sunitinib (Sutent); trastuzumab (Herceptin); bevacizumab (Avastin); rituximab (Rituxan); lapatinib (Tykerb); cetuximab (Erbitux); panitumumab (Vectibix); everolimus (Afinitor); alemtuzumab (Campath); gemtuzumab (Mylotarg); temsirolimus (Torisel); pazopanib (Votrient); dasatinib (Sprycel); nilotinib (Tasigna); vatalanib (Ptk787; ZK222584); CEP-701; SU5614; MLN518; XL999; VX-322; Azd0530; BMS-354825; SKI-606 CP-690; AG-490; WHI-P154; WHI-P131; AC-220; or AMG888.

Exemplary VEGF/VEGFR inhibitors include, but are not limited to, bevacizumab (Avastin); sorafenib (Nexavar); sunitinib (Sutent); ranibizumab; pegaptanib; or vandetinib.

Exemplary microtubule targeting drugs include, but are not limited to, paclitaxel, docetaxel, vincristin, vinblastin, nocodazole, epothilones and navelbine.

Exemplary topoisomerase poison drugs include, but are not limited to, teniposide, etoposide, adriamycin, camptothecin, daunorubicin, dactinomycin, mitoxantrone, amsacrine, epirubicin and idarubicin.

Exemplary taxanes or taxane derivatives include, but are not limited to, paclitaxel and docetaxel.

Exemplary general chemotherapeutic, anti-neoplastic, anti-proliferative agents include, but are not limited to, altretamine (Hexalen); isotretinoin (Accutane; Amnesteem; Claravis; Sotret), tretinoin (Vesanoid); azacitidine (Vidaza); bortezomib (Velcade) asparaginase (Elspar); levamisole (Ergamisol); mitotane (Lysodren); procarbazine (Matulane); pegaspargase (Oncaspar); denileukin diftitox (Ontak); porfimer (Photofrin); aldesleukin (Proleukin); lenalidomide (Revlimid); bexarotene (Targretin); thalidomide (Thalomid); temsirolimus (Torisel); arsenic trioxide (Trisenox); verteporfin (Visudyne); mimosine (Leucenol); (1M tegafur-0.4 M 5-chloro-2,4-dihydroxypyrimidine-1 M potassium oxonate) or lovastatin.

In some embodiments, combination treatment modalities are provided in which the additional therapeutic agent is a cytokine, e.g., G-CST (granulocyte colony stimulating factor). In another aspect, an EZH2 inhibitor provided herein may be administered in combination with radiation therapy. Radiation therapy can also be administered in combination with an EZH2 inhibitor provided herein and another chemotherapeutic agent described herein as part of a multi-agent therapy. In yet another aspect, an EZH2 inhibitor provided herein may be administered in combination with standard chemotherapy combinations such as, but not restricted to, CMF (cyclophosphamide, methotrexate and 5-fluorouracil), CAF (cyclophosphamide, adriamycin and 5-fluorouracil), AC (adriamycin and cyclophosphamide), FEC (5-fluorouracil, epirubicin, and cyclophosphamide), ACT or ATC (adriamycin, cyclophosphamide, and paclitaxel), rituximab, Xeloda (capecitabine), Cisplatin (CDDP), Carboplatin, TS-1 (tegafur, gimestat and otastat potassium at a molar ratio of 1:0,4:1), Camptothecin-I1 (CPT-11, Irinotecan or Camptosar™), CHOP (cyclophosphamide, hydroxydaunorubicin, oncovin, and prednisone or prednisolone), R-CHOP (rituximab, cyclophosphamide, hydroxydaunorubicin, oncovin, prednisone or prednisolone), or CMFP (cyclophosphamide, methotrexate, 5-fluorouracil and prednisone).

In some preferred embodiments, an EZH2 inhibitor provided herein may be administered with an inhibitor of an enzyme, such as a receptor or non-receptor kinase. Receptor and non-receptor kinases are, for example, tyrosine kinases or serine/threonine kinases. Kinase inhibitors described herein are small molecules, polynucleic acids, polypeptides, or antibodies.

Exemplary kinase inhibitors include, but are not limited to, Bevacizumab (targets VEGF), BIBW 2992 (targets EGFR and Erb2), Cetuximab/Erbitux (targets Erb1), Imatinib/Gleevec (targets Bcr-Abl), Trastuzumab (targets Erb2), Gefitinib/Iressa (targets EGFR), Ranibizumab (targets VEGF), Pegaptanib (targets VEGF), Erlotinib/Tarceva (targets Erb1), Nilotinib (targets Bcr-Abl), Lapatinib (targets Erb1, and Erb2/Her2), GW-572016/lapatinib ditosylate (targets HER_{2/}Erb2), Panitumumab/Vectibix (targets EGFR), Vandetinib (targets RET/VEGFR), E7080 (multiple targets including RET and VEGFR), Herceptin (targets HER₂/Erb2), PKI-166 (targets EGFR), Canertinib/CI-1033 (targets EGFR), Sunitinib/SU-11464/Sutent (targets EGER and FLT3), Matuzumab/Emd7200 (targets EGFR), EKB-569 (targets EGFR), Zd6474 (targets EGFR and VEGFR), PKC-412 (targets VEGR and FLT3), Vatalanib/Ptk787/ZK222584 (targets VEGR), CEP-701 (targets FLT3), SU5614 (targets FLT3), MLN518 (targets FLT3), XL999 (targets FLT3), VX-322 (targets FLT3), Azd0530 (targets SRC), BMS-354825 (targets SRC), SKI-606 (targets SRC), CP-690 (targets JAK), AG-490 (targets JAK), WHI-P154 (targets JAK), WHI-P131 (targets JAK), sorafenib/Nexavar (targets RAF kinase, VEGFR-1, VEGFR-2, VEGFR-3, PDGFR-β, KIT, FLT-3, and RET), Dasatinib/Sprycel (BCR/ABL and Src), AC-220 (targets Flt3), AC-480 (targets all HER proteins, “panHER”), Motesanib diphosphate (targets VEGF1-3, PDGFR, and c-kit), Denosumab (targets RANKL, inhibits SRC), AMG888 (targets HER₃), and AP24534 (multiple targets including Flt3).

Exemplary serine/threonine kinase inhibitors include, but are not limited to, Rapamune (targets mTOR/FRAP1), Deforolimus (targets mTOR), Certican/Everolimus (targets mTOR/FRAP1), AP23573 (targets mTOR/FRAP1), Eril/Fasudil hydrochloride (targets RHO), Flavopiridol (targets CDK), Seliciclib/CYC202/Roscovitrine (targets CDK), SNS-032/BMS-387032 (targets CDK), Ruboxistaurin (targets PKC), Pkc412 (targets PKC), Bryostatin (targets PKC), KAI-9803 (targets PKC), SF1126 (targets PI3K), VX-680 (targets Aurora kinase), Azd1152 (targets Aurora kinase), Arry-142886/AZD-6244 (targets MAP/MEK), SCIO-469 (targets MAP/MEK), GW681323 (targets MAP/MEK), CC-401 (targets JNK), CEP-1347 (targets JNK), and PD 332991 (targets CDK).

In some embodiments, combination treatment modalities are provided that include an EZH2 inhibitor as provided herein and an immune checkpoint inhibitor. Immune checkpoint proteins inhibit the action of the immune cells (e.g., T cells) against certain cells. Immune checkpoint signaling plays an important role in balancing a subject's immune response against cells targeted by the immune system (e.g., infected or malignant cells), and cells that are not targeted by immune system effectors (e.g., healthy cells). Without wishing to be bound by any particular theory, it is believed that evasion of some cancer cells from immune system surveillance and destruction is mediated by aberrant immune checkpoint signaling, wherein cancer cells modulate or abolish the host's immune response by activating one or more immune checkpoint signaling pathways in the host's immune cells. Various immune checkpoint signaling proteins have been identified, for example, and without limitation, CTLS4, PD-1, PD-L1, LAG3, B7-H3, and Tim3, and immune checkpoint inhibitors targeting such immune checkpoint proteins have been developed. Such immune checkpoint inhibitors decrease or abolish the activity of the immune checkpoint signaling pathway they target and can thus boost the subject's immune response, e.g., against pathologic cells that otherwise escape proper immune system surveillance. For example, some immune checkpoint inhibitors have been reported to effectively inhibit immune checkpoint signaling that prevented a T-cell mediated attack of an infected or cancerous cell. Accordingly, the immune checkpoint inhibitors described herein enable or support immune system surveillance and effector functions (e.g., in the form of a T-cell attack) targeted at malignant or infective cells. Some of the immune checkpoint inhibitors referred to herein include monoclonal antibodies that specifically bind and inhibit an activity of one or more checkpoint protein(s) on an immune cell (e.g. a T cell). Immune checkpoint inhibitors of the disclosure may be used to boost the subject's immune response against any type of cancer cell.

While any checkpoint protein may be targeted, exemplary immune checkpoint inhibitors of the disclosure may target, bind, and/or inhibit an activity of a protein including, but not limited to, CTLA4, PD-1, PD-L1, LAG3, B7-H3, Tim3 or any combination thereof. Immune checkpoint inhibitors that target, bind, and/or inhibit an activity of CTLA4 may comprise Ipilimumab, Ticilimumab, AGEN-1884 or a combination thereof. Immune checkpoint inhibitors that target, bind, and/or inhibit an activity of PD-1 and/or PD-L1 may comprise Nivolumab, Pembrolizumab, Atezolizumab, Durvalumab, Avelumab, BMS-936559, AMP-224, MEDI-0680, TSR-042, BCiB-108, STI-1014, KY-1003, ALN-PDL, BGB-A317, KD-033, REGN-2810, PDR-001, SHR-1210, MGD-013, PF-06801591, CX-072 or a combination thereof. Immune checkpoint inhibitors that target, bind, and/or inhibit an activity of LAG3 may comprise IMP-731, LAG-525, BMS-986016, GSK-2831781 or a combination thereof. Immune checkpoint inhibitors that target, bind, and/or inhibit an activity of B7-H3 may comprise Enoblituzumab, 1241-8H9, DS-5573 or a combination thereof. Immune checkpoint inhibitors that target, bind, and/or inhibit an activity of Tim3 may comprise MBG-453,

Exemplary immune checkpoint inhibitors suitable for use in the combination treatment modalities provided herein include, but are not limited to, Ipilimumab, Ticilimumab, AGEN-1884, Nivolumab, Pembrolizumab, Atezolizumab, Durvalumab, Avelumab, BMS-936559, AMP-224, MEDI-0680, TSR-042, BGB-108, STI-1014, KY-1003, ALN-PDL, BGB-A317, KD-033, REGN-2810, PDR-001, SHR-1210, MGD-013, PF-06801591, CX-072, IMP-731, LAG-525, BMS-986016, GSK-2831781, Enoblituzumab, 1241-8H9, DS-5573, or a combination thereof.

For example, in some embodiments, combination therapy strategies, treatment modalities, and methods for the treatment of cell proliferative diseases, e.g., certain cancers, are provided, wherein the ELI-I2 inhibitor is tazemetostat, or a pharmaceutically acceptable salt thereof, and the immune checkpoint inhibitor is Atezolizumab. For example, in some embodiments, a method is provided that comprises administering to a subject in need thereof, e.g., a subject having or being diagnosed with a proliferative disease (e.g., a cancer), a therapeutically effective amount of tazemetostat, or a pharmaceutically acceptable salt thereof, and a therapeutically effective amount of Atezolizumab. In some embodiments, the cell proliferative disease is a cell proliferative disease of the lung. In some embodiments, the cell proliferative disease of the lung is lung cancer. In some embodiments, the lung cancer is NSCLC. In some embodiments, the lung cancer is SCLC. In some embodiments, the lung cancer is metastatic lung cancer. In some embodiments, the lung cancer is resistant or refractory to first-, second-, or third-line lung cancer treatment, e.g., as described herein or otherwise known or used in the art. In some embodiments, the lung cancer is characterized by SMARCA2 and/or SMARCA4 loss of function. In some embodiments, the lung cancer is characterized by SMARCA2 loss of function mediated by an epigenetic lesion. In some embodiments, the lung cancer is characterized by SMARCA4 loss of function mediated by a genetic lesion. In some embodiments, the lung cancer is characterized by SMARCA2 loss of function mediated by an epigenetic lesion and SMARCA4 loss of function mediated by a genetic lesion. In some embodiments, the lung cancer is characterized by a poorly-differentiated tumor or lesion. In some embodiments, the lung cancer is characterized by features of an epithelial-to-mesenchymal transition.

In certain embodiments, this disclosure provides a method of treating a cell proliferative disorder, e.g., a cancer, in a subject in need thereof comprising administering to the subject a combination of an EZH2 inhibitor provided herein and an immune checkpoint inhibitor. In some embodiments, the EZH2 inhibitor is tazemetostat. In some embodiments, the EZH2 inhibitor is administered at an oral dose of 800 mg twice per day. In some embodiments, the immune checkpoint inhibitor is atezolizumab (TECENTRIQ™). In some embodiments, the immune checkpoint inhibitor, e.g., atezolizumab, is administered at a dose of 1200 mg as an intravenous infusion over about 60 minutes every 3 weeks (see, accessdata.fda.gov/drugsatfda_docs/label/2016/761034s000lbl.pdf, the contents of which are incorporated herein for additional information about atezolizumab).

In certain embodiments, this disclosure provides a method of treating lung cancer, e.g., NSCLC, SCLC, mesothelioma, or any other form of lung cancer, in a subject in need thereof comprising administering to the subject a combination of tazemetostat at an oral dose of 800 mg twice per day and atezolizumab (TECENTRIQ™) at a dose of 1200 mg as an intravenous infusion over about 60 minutes every 3 weeks.

In certain embodiments, this disclosure provides a method of treating Non-Hodgkin's Lymphoma (or any other form of heme cancer) in a subject in need thereof comprising administering to the subject a combination of tazemetostat at an oral dose of 800 mg twice per day and atezolizumab (TECENTRIQ™) at a dose of 1200 mg as an intravenous infusion over 60 minutes every 3 weeks.

In some embodiments, the treatment modalities provided herein comprise monitoring the methylation status in a target cell or tissue in the subject, e.g., by methods described herein or otherwise known to those in the art, e.g., by methods described herein or otherwise known in the art. In some embodiments, the treatment modalities provided herein comprise monitoring the status of SMARCA2 and/or SMARCA4 protein expression or protein function in a target cell or tissue in the subject, e.g., by methods described herein or otherwise known to those in the art. In some embodiments, the treatment modalities provided herein comprise monitoring the immune response status in the subject, e.g., by methods described herein or otherwise known to those in the art.

Various small molecule EZH2 inhibitors suitable for use with the treatment modalities provided herein have previously been described. Some non-limiting examples of EZH2 inhibitors that are suitable for use in the treatment modalities provided herein are those described in U.S. Pat. Nos. 8,410,088, 8,765,732, 9,090,562, 8,598,167, 8,962,620, US-2015/0065483, U.S. Pat Nos. 9,206,157, 9,006,242, 9,089,575, US 2015-0352119, WO 2014/062733, US-2015/0065503, WO2015/057859, U.S. Pat. No. 8,536,179, WO 2011/140324,PCT/US2014/015706, published as WO2014/124418, in PCT/US2013/025639, published as WO/2013/120104, and in U.S. Ser. No. 14/839,273, published as US 2015/0368229, the entire contents of each of which are incorporated herein by, reference.

In some embodiments, an EZH2 inhibitor suitable for use in the strategies, treatment modalities, methods, combinations, and compositions described herein has the following :Formula (1):

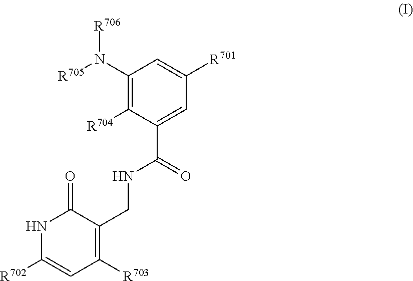

or a pharmaceutically acceptable salt thereof; wherein

R⁷⁰¹ is H, F, OR⁷⁰⁷, NHR⁷⁰⁷, —(C≡C)—(CH₂)ₙ₇-R⁷⁰⁸, phenyl, 5- or 6-membered heteroaryl, C₃₋₈ cycloalkyl, or 4-7 membered heterocycloalkyl containing 1-3 heteroatoms, wherein the phenyl, 5- or 6-membered heteroaryl, C₃₋₈ cycloalkyl or 4-7 membered heterocycloalkyl each independently is optionally substituted with one or more groups selected from halo, C₁₋₃ alkyl, OH, O—C₁₋₆ alkyl, NH—C₁₋₆ alkyl, and, C₁₋₃ alkyl substituted with C₃₋₈ cycloalkyl or 4-7 membered heterocycloalkyl containing 1-3 heteroatoms, wherein each of the O—C₁₋₆ alkyl and NH—C₁₋₆ alkyl is optionally substituted with hydroxyl, O—C₁₋₃ alkyl or NH—C₁₋₃ alkyl, each of the O—C₁₋₃ alkyl and NH—C₁₋₃ alkyl being optionally further substituted with O—C₁₋₃ alkyl or NH—C₁₋₃ alkyl;

each of R⁷⁰² and R⁷⁰³, independently is H, halo, C₁₋₄ alkyl, C₁₋₆ alkoxyl or C₆₋₁₀ aryloxy, each optionally substituted with one or more halo;

each of R⁷⁰⁴ and R⁷⁰⁵, independently is C₁₋₄ alkyl;

R⁷⁰⁶ is cyclohexyl substituted by N(C₁₋₄ alkyl)₂ wherein one or both of the C₁₋₄ alkyl is optionally substituted with C₁₋₆ alkoxy, or R⁷⁰⁶ is tetrahydropyranyl;

R⁷⁰⁷ is C₁₋₄ alkyl optionally substituted with one or more groups selected from hydroxyl, C₁₋₄ alkoxy, amino, mono- or di-C₁₋₄ alkylamino, C₃₋₈ cycloalkyl, and 4-7 membered heterocycloalkyl containing 1-3 heteroatoms, wherein the C₃₋₈ cycloalkyl or 4-7 membered heterocycloalkyl each independently is further optionally substituted with C₁₋₃ alkyl;

R⁷⁰⁸ is C₁₋₄ alkyl optionally substituted with one or more groups selected from OH, halo, and C₁₋₄ alkoxy, 4-7 membered heterocycloalkyl containing 1-3 heteroatoms, or O—C₁₋₆ alkyl, wherein the 4-7 membered heterocycloalkyl can be optionally further substituted with OH or C₁₋₆ alkyl; and

n₇ is 0, 1 or 2.

In some embodiments, R⁷⁰⁶ is cyclohexyl substituted by N(C₁₋₄ alkyl)₂ wherein one of the C₁₋₄ alkyl is unsubstituted and the other is substituted with methoxy.

In some embodiments, R⁷⁰⁶ is

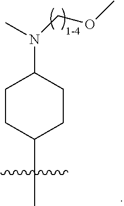

In some embodiments, the compound is of Formula II:

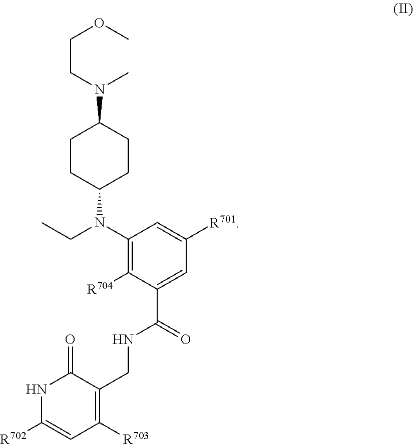

In some embodiments, R⁷⁰² is methyl or isopropyl and R⁷⁰³ is methyl or methoxyl

In some embodiments, R⁷⁰⁴ is methyl,

In some embodiments, R⁷⁰¹ and OR⁷⁰⁷ and R⁷⁰⁷ is C₁₋₃ alkyl optionally substituted with OCH₃ or morpholine.

In some embodiments, R⁷⁰¹ is H or F.

In some embodiments, R⁷⁰¹ is tetrahydropyranyl, phenyl, pyridyl, pyrimidyl, pyrazinyl, imidazolyl, or pyrazolyl, each of which is optionally substituted with methyl, methoxy, ethyl substituted with morpholine, or —OCH₂CH₂OCH₃.

In some embodiments, R⁷⁰⁸ is morpholine, piperidine, piperazine, pyrrolidine, diazepane, or azetidine, each of which is optionally substituted with OH or C₁₋₆ alkyl.

In some embodiments, R₇₀₈ is morpholine

In some embodiments, R⁷⁰⁸ is piperazine substituted with C₁₋₆ alkyl.

In some embodiments, R⁷⁰⁸ is methyl, t-butyl or C(CH₃)₂OH.

In some embodiments, an EZH2 inhibitor that can be used in the strategies, treatment modalities, methods, combinations, and compositions described herein may have the following Formula III:

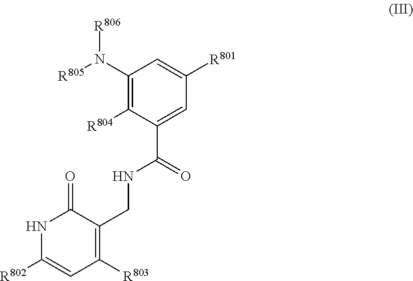

or a pharmaceutically acceptable salt thereof.
In this Formula:

R⁸⁰¹ is C₁₋₆ alkyl, C₂₋₆ alkenyl, C₂₋₆ alkynyl, C₃₋₈ cycloalkyl, 4-7 membered heterocycloalkyl containing 1-3 heteroatoms, phenyl or 5- or 6-membered heteroaryl, each of which is substituted with O—C₁₋₆ alkyl-Rₓ or NH—C₁₋₆ alkyl-Rₓ, wherein Rₓ is hydroxyl, O—C₁₋₃ alkyl or NH—C₁₋₃ alkyl, and Rₓ is optionally further substituted with O—C₁₋₃ alkyl or NH—C₁₋₃ alkyl except when Rₓ is hydroxyl; or R₈₀₁ is phenyl substituted with -Q₂-T₂, wherein Q₂ is a bond or C₁-C₃ alkyl linker optionally substituted with halo, cyano, hydroxyl or C₁-C₆ alkoxy, and T₂ is optionally substituted 4- to 12-membered heterocycloalkyl; and R⁸⁰¹ is optionally further substituted;

each of R⁸⁰² and R⁸⁰³, independently is H, halo, C₁₋₄ alkyl, C₁₋₆ alkoxyl or C₆-C₁₀ aryloxy, each optionally substituted with one or more halo;

each of R⁸⁰⁴ and R⁸⁰⁵, independently is C₁₋₄ alkyl; and

R⁸⁰⁶ is -Qₓ-Tₓ, wherein Qₓ is a bond or C₁₋₄ alkyl linker, Tₓ is H, optionally substituted. C₁₋₄ alkyl, optionally substituted C₃-C₈ cycloalkyl or optionally substituted 4- to 14-membered heterocycloalkyl.

In some embodiments, each of Qₓ and Q₂ independently is a bond or methyl linker, and each of Tₓ and T₂ independently is tetrahydropyranyl, piperidinyl substituted by 1, 2, or 3 C₁₋₄ alkyl groups, or cyclohexyl substituted by N(C₁₋₄ alkyl)₂ wherein one or both of the C₁₋₄ alkyl is optionally substituted with C₁₋₆ alkoxy;

In some embodiments, R⁸⁰⁶ is cyclohexyl substituted by N(C₁₋₄ alkyl)₂ or R⁸⁰⁶ is tetrahydropyranyl.

In some embodiments, R⁸⁰⁶ is

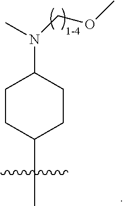

In some embodiments, R⁸⁰¹ is phenyl or 5- or 6-membered heteroaryl substituted with O—C₁₋₆ alkyl-Rₓ, or R⁸⁰¹ is phenyl substituted with CH₂-tetrahydropyranyl.

In some embodiments, in some embodiments, a compound according to some aspects of the present disclosure is of Formula IVa or IVb:

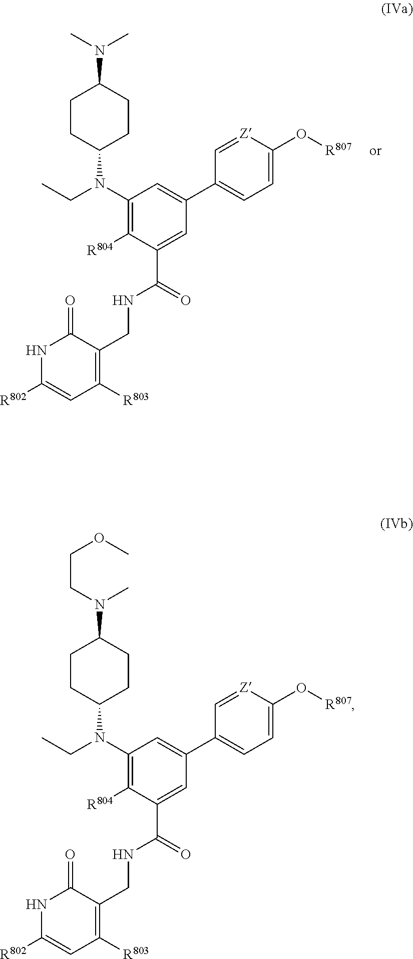

wherein Z′ is CH or N, and R⁸⁰⁷ is C₂₋₃ alkyl-Rₓ.

In some embodiments, R⁸⁰⁷ is —CH₂CH₂OH, —CH₂CH₂OCH₃, or —CH₂CH₂OCH₂CH₂OCH₃.

In some embodiments, R⁸⁰² is methyl or isopropyl and R⁸⁰³ is methyl or methoxyl.

In some embodiments, R⁸⁰⁴ is methyl.

In some embodiments, a compound of the present disclosure may have the following Formula (V):

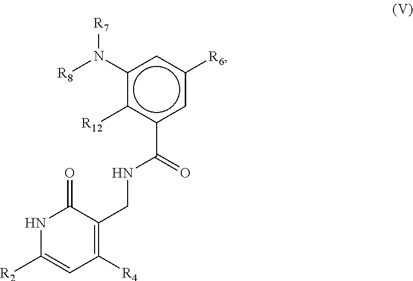

or a pharmaceutically acceptable salt or ester thereof.
In this Formula:

R₂, R₄ and R₁₂ are each, independently C₁₋₆ alkyl;

R₆ is C₆-C₁₀ aryl or 5- or 6-membered heteroaryl, each of which is optionally substituted with one or more -Q₂-T₂, wherein Q₂ is a bond or C₁-C₃ alkyl linker optionally substituted with halo, cyano, hydroxyl or C₁-C₆ alkoxy, and T₂ is H, halo, cyano, —ORₐ, —NRₐR_{b}, —(NRₐR_{b}R_{c})⁺A⁻, —C(O)Rₐ, —C(O)ORₐ, —C(O)NRₐR_{b}, —NR_{b}C(O)Rₐ, —NR_{b}C(O)ORₐ, —S(O)₂Rₐ, —S(O)₂NRₐR_{b}, or R_{S2}, in which each of Rₐ, R_{b}, and R_{c}, independently is H or R_{S3}, A⁻ is a pharmaceutically acceptable anion, each of R_{S2} and R_{S3}, independently, is C₁-C₆ alkyl, C₃-C₈ cycloalkyl, C₆-C₁₀ aryl, 4 to 12-membered heterocycloalkyl, or 5- or 6-membered heteroaryl, or Rₐ and R_{b}, together with the N atom to which they are attached, form a 4 to 12-membered heterocycloalkyl ring having 0 or 1 additional heteroatom, and each of R_{S2}, R_{S3}, and the 4 to 12-membered heterocycloalkyl ring formed by Rₐ and R_{b}, is optionally substituted with one or more -Q₃-T₃, wherein Q₃ is a bond or C₁-C₂ alkyl linker each optionally substituted with halo, cyano, hydroxyl or C₁-C₆ alkoxy, and T₃ is selected from the group consisting of halo, cyano, C₁-C₆ alkyl, C₃-C₈ cycloalkyl, C₆-C₁₀ aryl, 4 to 12-membered heterocycloalkyl, 5- or 6-membered heteroaryl, OR_{d}, COOR_{d}, —S(O)₂R_{d}, —NR_{d}Rₑ, and —C(O)NR_{d}Rₑ, each of R_{d} and Rₑ independently being H or C₁-C₆ alkyl, or —Q₃-T₃ is oxo; or any two neighboring -Q₂-T₂, together with the atoms to which they are attached form a 5- or 6-membered ring optionally containing 1-4 heteroatoms selected from N, O and S and optionally substituted with one or more substituents selected from the group consisting of halo, hydroxyl, COOK C(O)O—C₁-C₆ alkyl, cyano, C₁-C₆ alkoxyl, amino, mono-C₁-C₆ alkylamino di-C₁-C₆ alkylamino, C₃-C₈ cycloalkyl, C₆-C₁₀ aryl, 4 to 12-membered heterocycloalkyl, and 5- or 6-membered heteroaryl;

R₇ is -Q₄-T₄, in which Q₄ is a bond, C₁-C₄ alkyl linker, or C₂-C₄ alkenyl linker, each linker optionally substituted with halo, cyano, hydroxyl or C₁-C₆ alkoxy, and T₄ is H, halo, cyano, NR_{f}R_{g}, —OR_{f}, —C(O)R_{f}, —C(O)OR_{f}, —C(O)NR_{f}R_{g}, —C(O)NR_{f}OR_{g}, —NR_{f}C(O)R_{g}, —S(O)₂R_{f}, or R_{S4}, in which each of R_{f} and R_{g}, independently is H or R_{S}, each of R_{S4} and R_{S5}, independently is C₁-C₆ alkyl, C₂-C₆ alkenyl, C₂-C₆ alkenyl, C₃-C₈ cycloalkyl, C₆-C₁₀ aryl, 4 to 12-membered heterocycloalkyl, or 5- or 6-membered heteroaryl, and each of R_{S4} and R_{S5} is optionally substituted with one or more -Q₅-T₅, wherein Q₅ is a bond, C(O), C(O)NRk, NRₖC(O), S(O)₂, or C₁-C₃ alkyl linker, Rₖ being H or C₁-C₆ alkyl, and T₅ is H, halo, C₁-C₆ alkyl, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, 5- or 6-membered heteroaryl, or S(O)_{q}R_{q} in which q is 0, 1, or 2 and R_{q} is C₂-C₆ alkyl, C₂-C₆ alkenyl, C₂-C₆ alkynyl, C₃-C₈ cycloalkyl, C₆-C₁₀ aryl, 4 to 12-membered heterocycloalkyl, or 5- or 6-membered heteroaryl, and T₅ is optionally substituted with one or more substituents selected from the group consisting of halo, C₁-C₆ alkyl, hydroxyl, cyano, C₁-C₆ alkoxyl, amino, mono-C₁-C₆ alkylamino, di-C₁-C₆ alkylamino, C₃-C₈cycloalkyl, C₆-C₁₀ to aryl, 4 to 12-membered heterocycloalkyl, and 5- or 6-membered heteroaryl except when T₅ is H, halo, hydroxyl, or cyano; or -Q₅-T₅ is oxo; and

R₅ is H, halo, hydroxyl, COOH, cyano, R_{S6}, OR_{S6}, or COOR_{S6}, in which R_{S6} is C₁-C₆ alkyl, C₂-C₆ alkenyl, C₂-C₆ alkynyl, C₃-C₈ cycloalkyl, 4 to 12-membered heterocycloalkyl, amino, mono-C₁-C₆ alkylamino, or di-C₁-C₆ alkylamino, and R_{S6} is optionally substituted with one or more substituents selected from the group consisting of halo, hydroxyl, COOH, C(O)O-C₁-C₆ alkyl, cyano, C₁-C₆ alkoxyl, amino, mono-C₁-C₆ alkylamino, and di-C₁-C₆ alkylamino; or R₇ and R₈, together with the N atom to which they are attached, form a 4 to 11-membered heterocycloalkyl ring having 0 to 2 additional heteroatoms, and the 4 to 11-membered heterocycloalkyl ring formed by R₇ and R₈ is optionally substituted with one or more -Q₆-T₆, wherein Q₆ is a bond, C(O), C(O)NRₘ, NRₘC(O), S(O)₂, or C₁-C₃ alkyl linker, Rₘ being H or C₁-C₆ alkyl, and T₆ is H, halo, C₁-C₆ alkyl, hydroxyl, cyano, C₁-C₆ alkoxyl, amino, mono-C₁-C₆ alkylamino, di-C₁-C₆ alkylamino, C₃-C₈ cycloalkyl, C₆-C₁₀ aryl, 4 to 12-membered heterocycloalkyl, 5- or 6-membered heteroaryl, or S(O)ₚRₚ in which p is 0, 1, or 2 and Rₚ is C₁-C₆ alkyl, C₂-C₆ alkenyl, C₂-C₆ alkynyl, C₃-C₈ cycloalkyl, C₆-C₁₀ to aryl, 4 to 12-membered heterocycloalkyl, or 5- or 6-membered heteroaryl, and T₆ is optionally substituted with one or more substituents selected from the group consisting of halo, C₁-C₆ alkyl, hydroxyl, cyano, C₁-C₆ alkoxyl, amino, mono-C₁-C₆ alkylamino, alkylamino, C₃-C₈ cycloalkyl, C₆-C₁₀ aryl, 4 to 12-membered heterocycloalkyl, and 5- or 6-membered heteroaryl except when T₆ is H, halo, hydroxyl, or cyano; or -Q₆-T₆ is oxo.

In some embodiments, R₆ is C₆-C₁₀ aryl or 5- or 6-membered heteroaryl, each of which is optionally, independently substituted with one or more -Q₂-T₂, wherein Q₂ is a bond or C₁-C₃ alkyl linker, and T₂ is H, halo, cyano, —ORₐ, —NRₐR_{b}, —(NRₐR_{b}R_{c})⁺A⁻, —C(O)NRₐR_{b}, —NR_{b}C(O)Rₐ, —S(O)₂Rₐ, or R_{S2}, in which each of Rₐ and R_{b}, independently is H or R_{S3}, each of R_{S2} and R_{S3}, independently, is C₁-C₆ alkyl, or Rₐ and R_{b}, together with the N atom to which they are attached, form a 4 to 7-membered heterocycloalkyl ring having 0 or 1 additional heteroatom, and each of R_{S2}, R_{S3}, and the 4 to 7-membered heterocycloalkyl ring formed by Rₐ and R_{b}, is optionally, independently substituted with one or more -Q₃-T₃, wherein Q₃ is a bond or C₁-C₃ alkyl linker and T₃ is selected from the group consisting of halo, C₁-C₆ alkyl, 4 to 7-membered heterocycloalkyl, OR_{d}, —S(O)₂R_{d}, and —NR_{d}Rₑ, each of R_{d} and Rₑ independently being H or C₁-C₆ alkyl, or -Q₃-T₃ is oxo; or any two neighboring -Q₂-T₂, together with the atoms to which they are attached form a 5- or 6-membered ring optionally containing 1-4 heteroatoms selected from N, O and S.

In some embodiments, the compound is of Formula (VI):

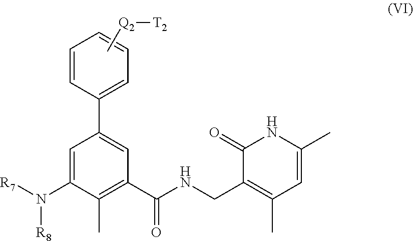

or a pharmaceutically acceptable salt thereof, wherein Q₂ is a bond or methyl linker, T₂ is H, halo, —ORₐ, —NRₐR_{b}, —(NRₐR_{b}R_{c})⁺A⁻, or —S(O)₂NRₐR_{b}, R₇ is piperidinyl, tetrahydropyran, cyclopentyl, or cyclohexyl, each optionally substituted with one -Q₅-T₅ and R₈ is ethyl.

Some aspects of the present disclosure provide the compounds of Formula (VIa):

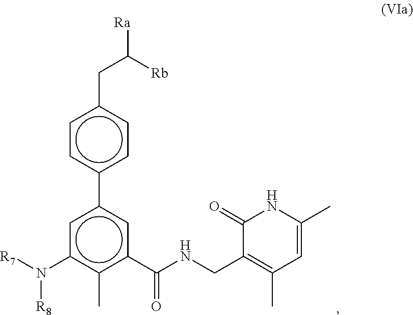

and pharmaceutically acceptable salts or esters thereof, wherein R₇, R₈, Rₐ, and R_{b} are defined herein.

The compounds of Formula (VIa) can include one or more of the following features:

In some embodiments, each of Rₐ and R_{b} independently is H or C₁-C₆ alkyl optionally substituted with one or more -Q₃-T₃.

In some embodiments, one of Rₐ and R_{b} is H.

In some embodiments, Rₐ and R_{b}, together with the N atom to which they are attached, form a 4 to 7-membered heterocycloalkyl ring having 0 or 1 additional heteroatoms to the N atom (e.g., azetidinyl, pyrrolidinyl, imidazolidinyl, pyrazolidinyl, oxazolidinyl, isoxazolidinyl, triazolidinyl, piperidinyl, 1,2,3,6-tetrahydropyridinyl, piperazinyl, morpholinyl, 1,4-diazepanyl, 1,4-oxazepanyl, 2-oxa-5-azabicyclo[2.2.1]heptanyl, 2,5-diazabicyclo[2.2.1]heptanyl, and the like) and the ring is optionally substituted with one or more -Q₃-T₃.

In some embodiments, Rₐ and R_{b}, together with the N atom to which they are attached, form azetidinyl, pyrrolidinyl, imidazolidinyl, pyrazolidinyl, oxazolidinyl, isoxazolidinyl, triazolidinyl, tetrahyrofuranyl, piperidinyl, 1,2,3,6-tetrahydropyridinyl, piperazinyl, or morpholinyl, and the ring is optionally substituted with one or more -Q₃-T₃.

In some embodiments, one or more -Q₃-T₃ are oxo.

In some embodiments, Q₃ is a bond or unsubstituted or substituted C₁-C₃ alkyl linker.

In some embodiments, T₃ is H, halo, 4 to 7-membered heterocycloalkyl, C₁-C₃ alkyl, OR_{d}, COOR_{d},—S(O)₂R_{d}, or —NR_{d}Rₑ.

In some embodiments, each of R_{d} and Rₑ independently being H or C₁-C₆ alkyl.

In some embodiments, R₇ is C₃-C₈ cycloalkyl or 4 to 7-membered heterocycloalkyl, each optionally substituted with one or more -Q₅-T₅.

In some embodiments, R₇ is piperidinyl, tetrahydropyran, tetrahydro-2H-thiopyranyl, cyclopentyl, cyclohexyl, pyrrolidinyl, or cycloheptyl, each optionally substituted with one or more -Q₅-T₅.

In some embodiments, R₇ is cyclopentyl cyclohexyl or tetrahydro-2H-thiopyranyl, each of which is optionally substituted with one or more -Q₅-Q₅.

In some embodiments, Q₅ is NHC(O) and T₅ is C₁-C₆ alkyl or C₁-C₆ alkoxy, each

In some embodiments, one or more -Q₅-T₅ are oxo.

In some embodiments, R₇ is 1-oxide-tetrahydro-2H-thiopyranyl or 1,1-dioxide-tetrahydro-2H-thiopyranyl.

In some embodiments, Q₅ is a bond and T₅ is amino, mono-C₁-C₆ alkylamino, di-C₁-C₆ alkylamino.

In some embodiments, Q₅ is CO, S(O)₂, or NHC(O); and T₅ is C₁-C₆ alkyl, C₁-C₆ alkoxyl, C₃-C₈ cycloalkyl, or 4 to 7-membered heterocycloalkyl.

In some embodiments, R₈ is H or C₁-C₆ alkyl which is optionally substituted with one or more substituents selected from the group consisting of halo, hydroxyl, COOH, C(O)O—C₁-C₆ alkyl, cyano, C₁-C₆ alkoxyl, amino, mono-C₁-C₆ alkylamino, and di-C₁-C₆ alkylamino.

In some embodiments, R₈ is H, methyl, or ethyl.

Other compounds of Formulae (I)-(VIa) suitable for use in the strategies, treatment modalities, methods, combinations, and compositions provided herein are described in U.S. Publication 20120264734, the contents of which are hereby incorporated by reference in their entireties. The compounds of Formulae (i)-(VIa) are suitable for administration as part of a combination therapy with one or more other therapeutic agents, e.g., with an immune checkpoint inhibitor as provided herein.

In some embodiments of the strategies, treatment modalities, methods, combinations, and compositions provided herein, the EZH2 inhibitor is Compound 44

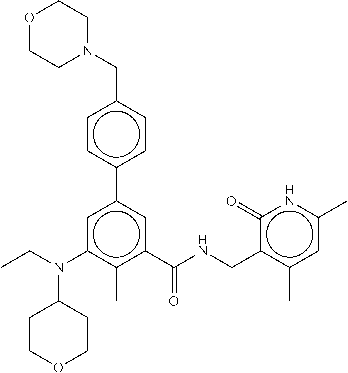

or a pharmaceutically acceptable salt thereof. Compound 44 is also referred to as tazemetostat, EPZ006438 or 6438.

Compound 44 or a pharmaceutically acceptable salt thereof, as described herein, is potent in targeting both wild type and mutant EZH2. Compound 44 is orally bioavailable and has high selectivity to EZH2 compared with other histone methyltransferases (i.e. >20,000 fold selectivity by Ki). Importantly, Compound 44 has target methyl mark inhibition that results in the killing of genetically defined cancer cells in vitro. Animal models have also shown sustained in vivo efficacy following inhibition of target methyl mark.

In some embodiments, Compound 44 or a pharmaceutically acceptable salt thereof is administered to the subject at a dose of approximately 100 mg to approximately 3200 mg daily, such as about 100 mg BID to about 1600 mg BID (e.g., 100 mg BID, 200 mg BID, 400 mg BID, 800 mg BID, or 1600 mg BID), for treating a germinal center-derived lymphoma.

In some embodiments, Compound 44 or a pharmaceutically acceptable salt thereof is administered to a subject in combination (either simultaneously or sequentially) with an immune checkpoint inhibitor provided herein.

In some embodiments, a compound that can be used in the strategies, treatment modalities, methods, combinations, and compositions presented here is:

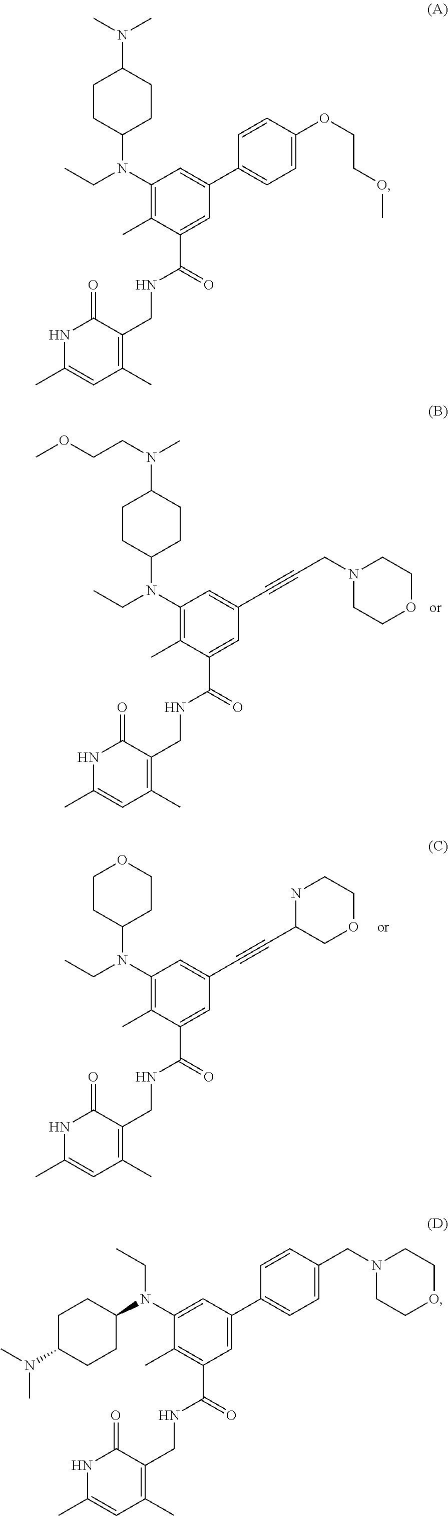

stereoisomers thereof or pharmaceutically acceptable salts and solvates thereof.

In some embodiments, the EZH2 inhibitor may comprise, consist essentially of or consist of GSK-126, having the following formula:

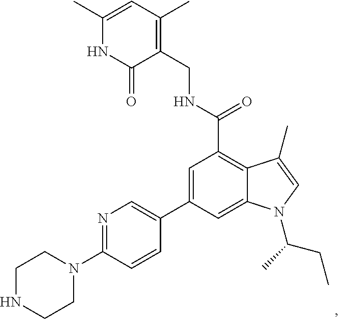 [SMILES: CC[C@H](C)n1cc(C)c2c(C(=O)NCc3c(C)cc(C)[nH]c3=O)cc(-c3ccc(N4CCNCC4)nc3)cc21]

stereoisomers thereof, pharmaceutically acceptable salts or solvates thereof. In some embodiments of the strategies, treatment modalities, methods, combinations, and compositions provided herein, the EZH2 inhibitor is an EZH2 inhibitor described in U.S. Pat. No. 8,536,179 (describing GSK-126 among other compounds and corresponding to WO 2011/140324), the entire contents of each of which are incorporated herein by reference.

In some embodiments of the strategies, treatment modalities, methods, combinations, and compositions provided herein, the EZH2 inhibitor is an EZH2 inhibitor described in PCT/US2014/015706, published as WO/2014/124418, in PCT/US2013/025639, published as WO/2013/120104, and in U.S. Ser. No. 14/839,273, published as US 2015/0368229, the entire contents of each of which are incorporated herein by reference. In some embodiments of the strategies, treatment modalities, methods, combinations, and compositions provided herein, the EZH2 inhibitor is a compound of the formula:

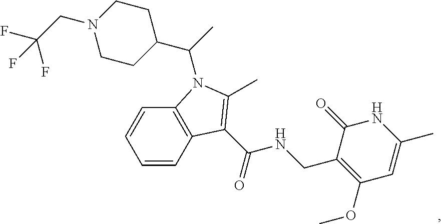 [SMILES: COc1cc(C)[nH]c(=O)c1CNC(=O)c1c(C)n(C(C)C2CCN(CC(F)(F)F)CC2)c2ccccc12]

or a pharmaceutically acceptable salt thereof (see, for example, US 2015/0368229, the contents of which are incorporated herein)

In some embodiments, the EZH2 inhibitor is a small molecule that is used as the compound itself, i.e., as the free base or “naked” molecule. In some embodiments, the EZH2 inhibitor is a salt thereof, e.g., a mono-HCl or tri-HCl salt, mono-HBr or tri-HBr salt of the naked molecule.

Representative compounds that are suitable for the strategies, treatment modalities, methods, combinations, and compositions provided herein include compounds listed in Table 1. In the table below, each occurrence of

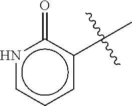

should be construed as

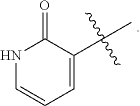 [SMILES: *c1ccc[nH]c1=O]

TABLE 1

| Compound | | |
|---|---|---|
| Number | Structure | MS (M + 1)⁺ |
| 1 | 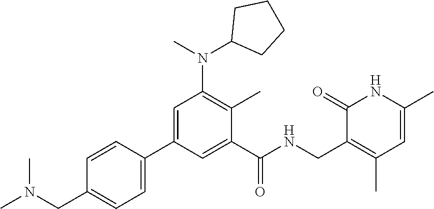 | 501.39 |
| 2 | 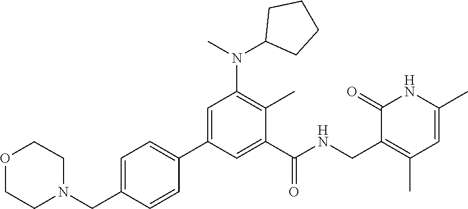 [SMILES: Cc1cc(C)c(CNC(=O)c2cc(-c3ccc(CN4CCOCC4)cc3)cc(N(C)C3CCCC3)c2C)c(=O)[nH]1] | 543.22 |
| 3 | 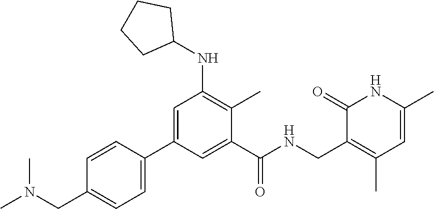 | 486.21 |
| 4 | 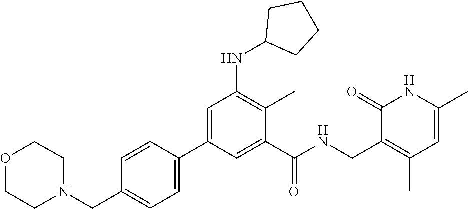 [SMILES: Cc1cc(C)c(CNC(=O)c2cc(-c3ccc(CN4CCOCC4)cc3)cc(NC3CCCC3)c2C)c(=O)[nH]1] | 529.30 |
| 11 | 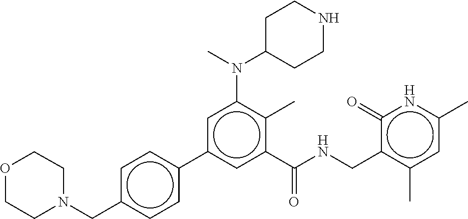 | 558.45 |
| 12 | 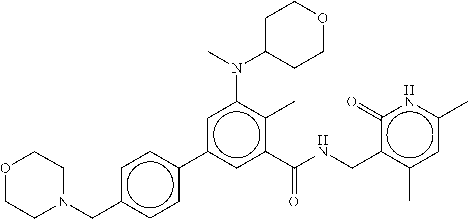 | 559.35 |
| 13 | 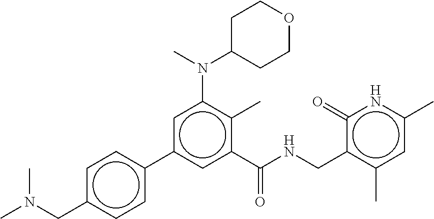 | 517.3 |
| 14 | 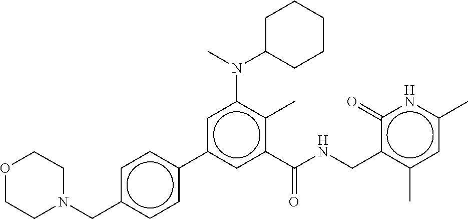 | 557.4 |
| 16 | 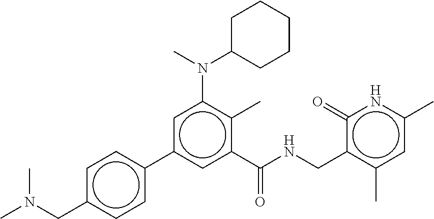 | 515.4 |
| 20 | 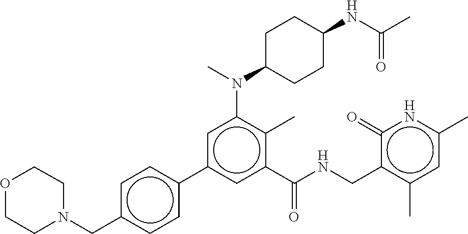 | 614.4 |
| 21 | 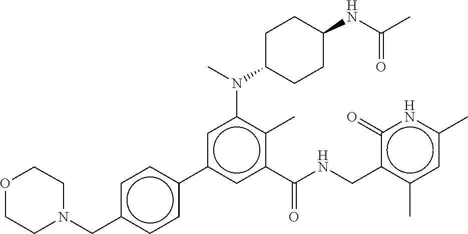 | 614.4 |
| 27 | 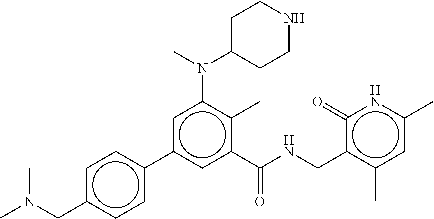 | 516.35 |
| 36 | 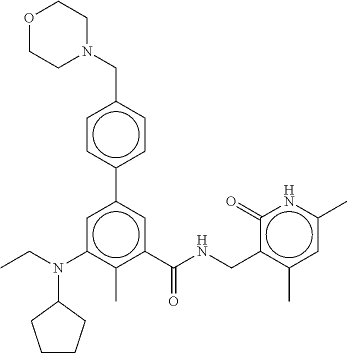 | 557.35 |
| 39 | 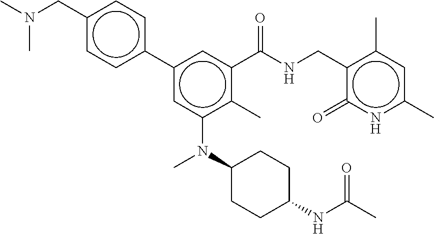 | 572.35 |
| 40 | 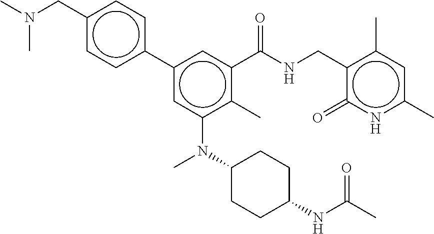 | 572.35 |
| 42 | 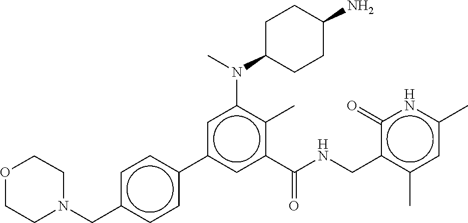 | 572.4 |
| 43 | 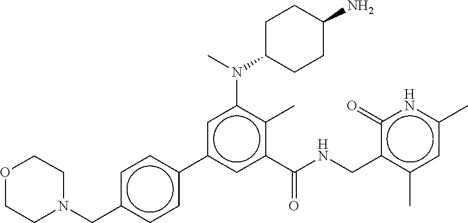 | 572.6 |
| 44 | 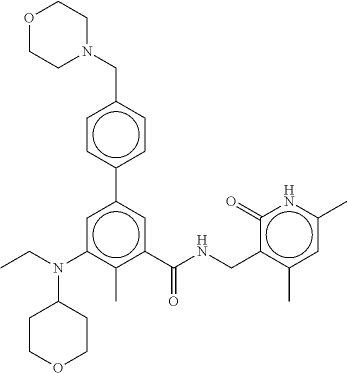 | 573.40 |
| 47 | 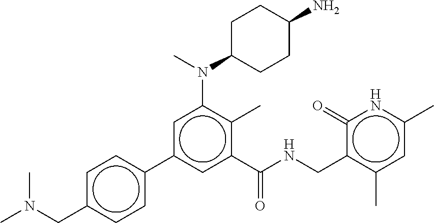 | 530.35 |
| 59 | 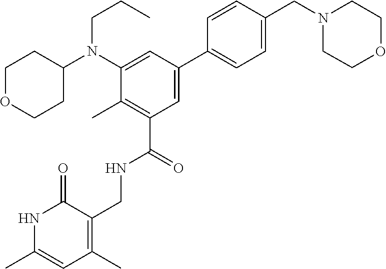 [SMILES: CCCN(c1cc(-c2ccc(CN3CCOCC3)cc2)cc(C(=O)NCc2c(C)cc(C)[nH]c2=O)c1C)C1CCOCC1] | 587.40 |
| 60 | 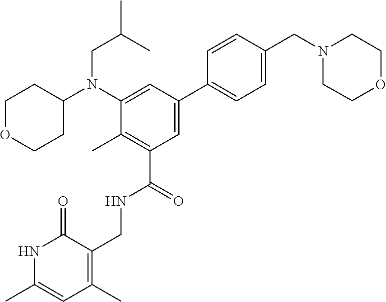 [SMILES: Cc1cc(C)c(CNC(=O)c2cc(-c3ccc(CN4CCOCC4)cc3)cc(N(CC(C)C)C3CCOCC3)c2C)c(=O)[nH]1] | 601.30 |
| 61 | 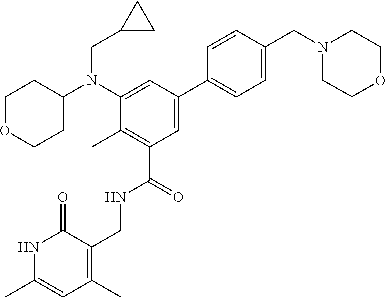 [SMILES: Cc1cc(C)c(CNC(=O)c2cc(-c3ccc(CN4CCOCC4)cc3)cc(N(CC3CC3)C3CCOCC3)c2C)c(=O)[nH]1] | 599.35 |
| 62 | 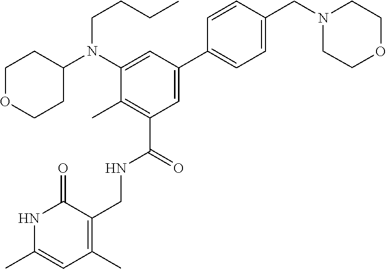 [SMILES: CCCCN(c1cc(-c2ccc(CN3CCOCC3)cc2)cc(C(=O)NCc2c(C)cc(C)[nH]c2=O)c1C)C1CCOCC1] | 601.35 |
| 63 | 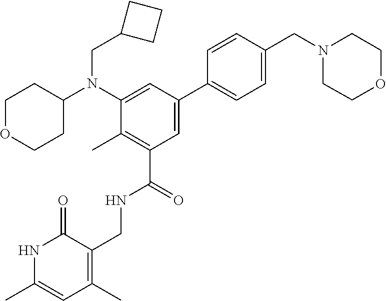 [SMILES: Cc1cc(C)c(CNC(=O)c2cc(-c3ccc(CN4CCOCC4)cc3)cc(N(CC3CCC3)C3CCOCC3)c2C)c(=O)[nH]1] | 613.35 |
| 65 | 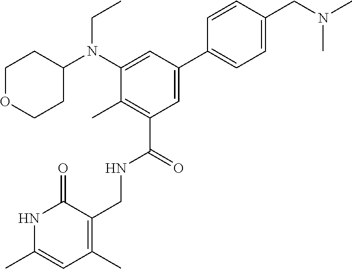 [SMILES: CCN(c1cc(-c2ccc(CN(C)C)cc2)cc(C(=O)NCc2c(C)cc(C)[nH]c2=O)c1C)C1CCOCC1] | 531.30 |
| 66 | 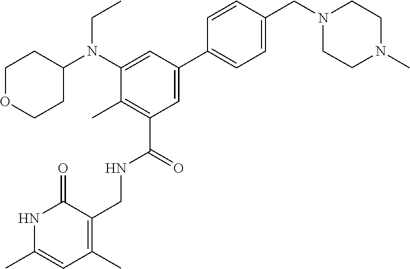 [SMILES: CCN(c1cc(-c2ccc(CN3CCN(C)CC3)cc2)cc(C(=O)NCc2c(C)cc(C)[nH]c2=O)c1C)C1CCOCC1] | 586.40 |
| 67 | 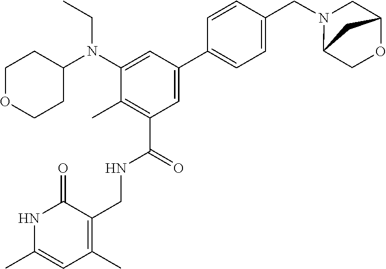 [SMILES: CCN(c1cc(-c2ccc(CN3C[C@H]4C[C@@H]3CO4)cc2)cc(C(=O)NCc2c(C)cc(C)[nH]c2=O)c1C)C1CCOCC1] | 585.25 |
| 68 | 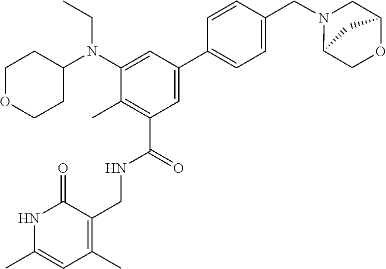 [SMILES: CCN(c1cc(-c2ccc(CN3C[C@@H]4C[C@H]3CO4)cc2)cc(C(=O)NCc2c(C)cc(C)[nH]c2=O)c1C)C1CCOCC1] | 585.35 |
| 69 | 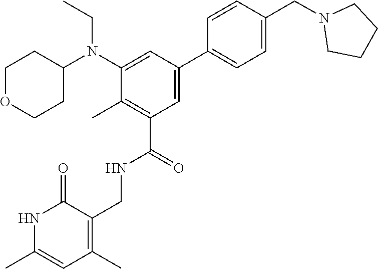 [SMILES: CCN(c1cc(-c2ccc(CN3CCCC3)cc2)cc(C(=O)NCc2c(C)cc(C)[nH]c2=O)c1C)C1CCOCC1] | 557.25 |
| 70 | 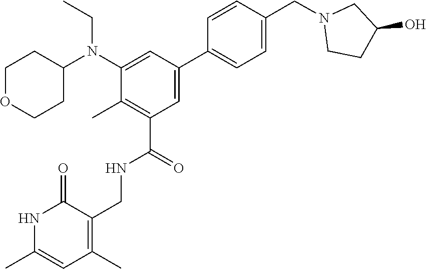 [SMILES: CCN(c1cc(-c2ccc(CN3CC[C@H](O)C3)cc2)cc(C(=O)NCc2c(C)cc(C)[nH]c2=O)c1C)C1CCOCC1] | 573.40 |
| 71 | 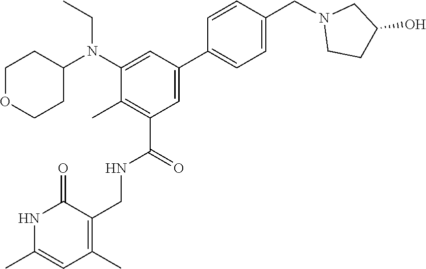 [SMILES: CCN(c1cc(-c2ccc(CN3CC[C@@H](O)C3)cc2)cc(C(=O)NCc2c(C)cc(C)[nH]c2=O)c1C)C1CCOCC1] | 573.40 |
| 72 | 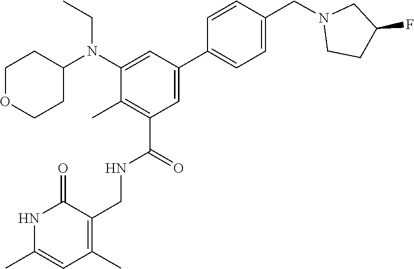 [SMILES: CCN(c1cc(-c2ccc(CN3CC[C@H](F)C3)cc2)cc(C(=O)NCc2c(C)cc(C)[nH]c2=O)c1C)C1CCOCC1] | 575.35 |
| 73 | 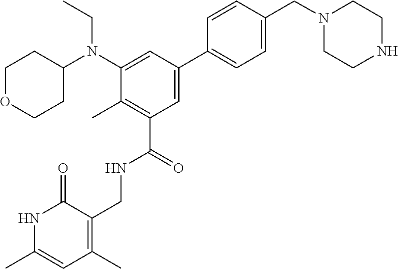 [SMILES: CCN(c1cc(-c2ccc(CN3CCNCC3)cc2)cc(C(=O)NCc2c(C)cc(C)[nH]c2=O)c1C)C1CCOCC1] | 572.10 |
| 74 | 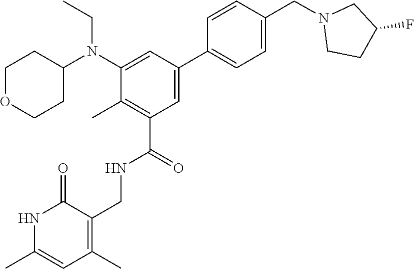 [SMILES: CCN(c1cc(-c2ccc(CN3CC[C@@H](F)C3)cc2)cc(C(=O)NCc2c(C)cc(C)[nH]c2=O)c1C)C1CCOCC1] | 575.35 |
| 75 | 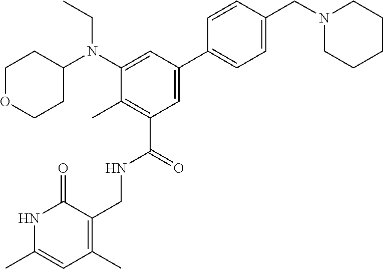 [SMILES: CCN(c1cc(-c2ccc(CN3CCCCC3)cc2)cc(C(=O)NCc2c(C)cc(C)[nH]c2=O)c1C)C1CCOCC1] | 571.25 |
| 76 | 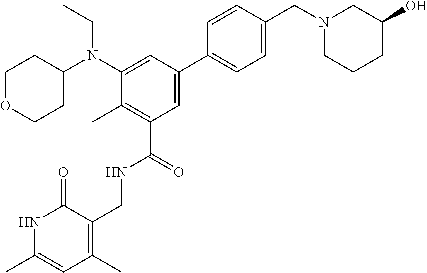 [SMILES: CCN(c1cc(-c2ccc(CN3CCC[C@H](O)C3)cc2)cc(C(=O)NCc2c(C)cc(C)[nH]c2=O)c1C)C1CCOCC1] | 587.40 |
| 77 | 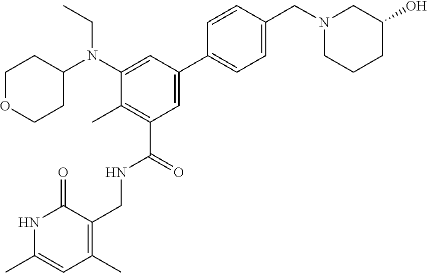 [SMILES: CCN(c1cc(-c2ccc(CN3CCC[C@@H](O)C3)cc2)cc(C(=O)NCc2c(C)cc(C)[nH]c2=O)c1C)C1CCOCC1] | 587.45 |
| 78 | 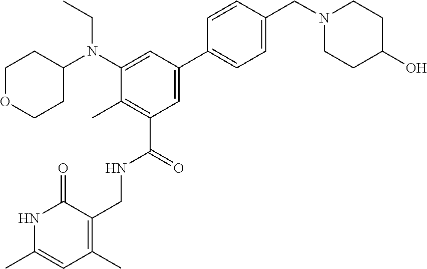 [SMILES: CCN(c1cc(-c2ccc(CN3CCC(O)CC3)cc2)cc(C(=O)NCc2c(C)cc(C)[nH]c2=O)c1C)C1CCOCC1] | 587.20 |
| 79 | 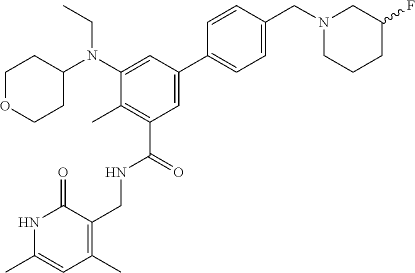 [SMILES: CCN(c1cc(-c2ccc(CN3CCCC(F)C3)cc2)cc(C(=O)NCc2c(C)cc(C)[nH]c2=O)c1C)C1CCOCC1] | 589.35 |
| 80 | 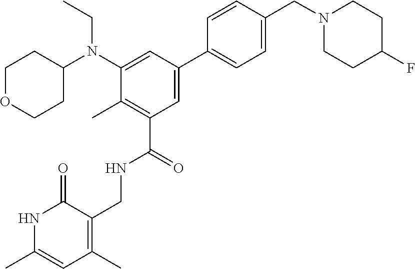 [SMILES: CCN(c1cc(-c2ccc(CN3CCC(F)CC3)cc2)cc(C(=O)NCc2c(C)cc(C)[nH]c2=O)c1C)C1CCOCC1] | 589.30 |
| 81 | 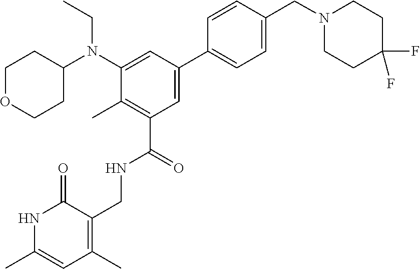 [SMILES: CCN(c1cc(-c2ccc(CN3CCC(F)(F)CC3)cc2)cc(C(=O)NCc2c(C)cc(C)[nH]c2=O)c1C)C1CCOCC1] | 607.35 |
| 82 | 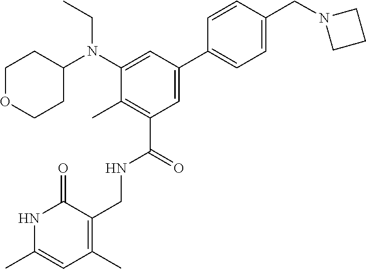 [SMILES: CCN(c1cc(-c2ccc(CN3CCC3)cc2)cc(C(=O)NCc2c(C)cc(C)[nH]c2=O)c1C)C1CCOCC1] | 543.40 |
| 83 | 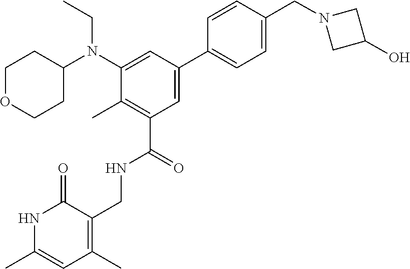 [SMILES: CCN(c1cc(-c2ccc(CN3CC(O)C3)cc2)cc(C(=O)NCc2c(C)cc(C)[nH]c2=O)c1C)C1CCOCC1] | 559.80 |
| 84 | 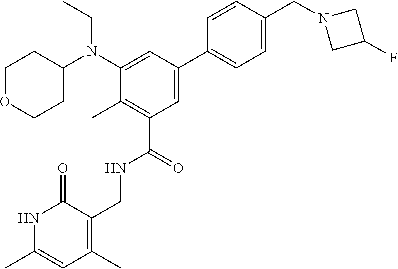 [SMILES: CCN(c1cc(-c2ccc(CN3CC(F)C3)cc2)cc(C(=O)NCc2c(C)cc(C)[nH]c2=O)c1C)C1CCOCC1] | 561.25 |
| 85 | 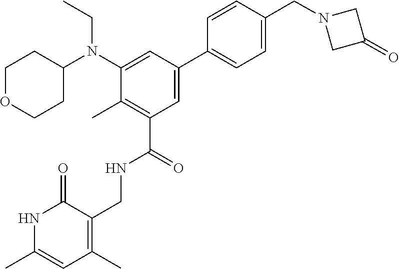 [SMILES: CCN(c1cc(-c2ccc(CN3CC(=O)C3)cc2)cc(C(=O)NCc2c(C)cc(C)[nH]c2=O)c1C)C1CCOCC1] | |
| 86 | 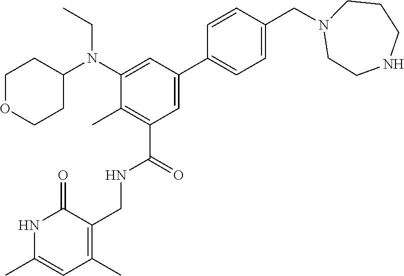 [SMILES: CCN(c1cc(-c2ccc(CN3CCCNCC3)cc2)cc(C(=O)NCc2c(C)cc(C)[nH]c2=O)c1C)C1CCOCC1] | 585.37 |
| 87 | 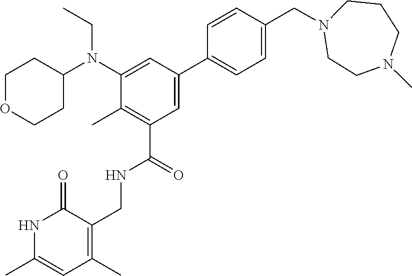 [SMILES: CCN(c1cc(-c2ccc(CN3CCCN(C)CC3)cc2)cc(C(=O)NCc2c(C)cc(C)[nH]c2=O)c1C)C1CCOCC1] | 600.30 |
| 88 | 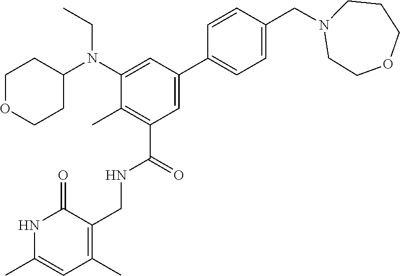 [SMILES: CCN(c1cc(-c2ccc(CN3CCCOCC3)cc2)cc(C(=O)NCc2c(C)cc(C)[nH]c2=O)c1C)C1CCOCC1] | 587.40 |
| 89 | 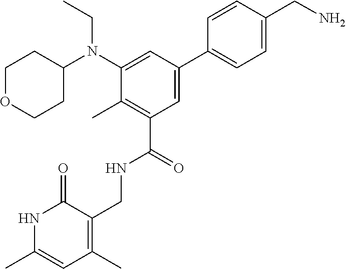 [SMILES: CCN(c1cc(-c2ccc(CN)cc2)cc(C(=O)NCc2c(C)cc(C)[nH]c2=O)c1C)C1CCOCC1] | 503.40 |
| 90 | 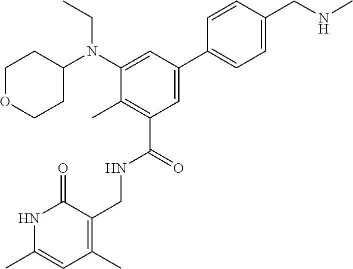 [SMILES: CCN(c1cc(-c2ccc(CNC)cc2)cc(C(=O)NCc2c(C)cc(C)[nH]c2=O)c1C)C1CCOCC1] | 517.30 |
| 91 | 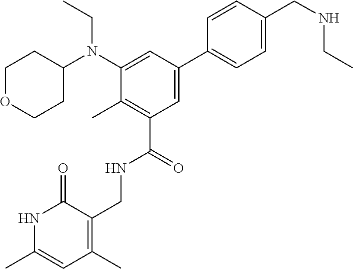 [SMILES: CCNCc1ccc(-c2cc(C(=O)NCc3c(C)cc(C)[nH]c3=O)c(C)c(N(CC)C3CCOCC3)c2)cc1] | 531.35 |
| 92 | 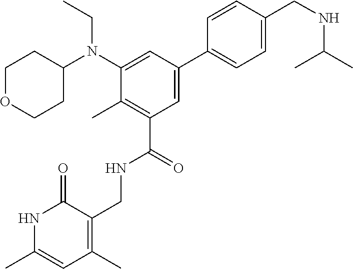 [SMILES: CCN(c1cc(-c2ccc(CNC(C)C)cc2)cc(C(=O)NCc2c(C)cc(C)[nH]c2=O)c1C)C1CCOCC1] | 545.40 |
| 93 | 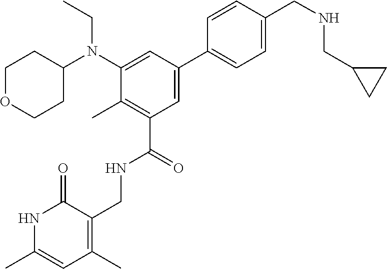 [SMILES: CCN(c1cc(-c2ccc(CNCC3CC3)cc2)cc(C(=O)NCc2c(C)cc(C)[nH]c2=O)c1C)C1CCOCC1] | 557.35 |
| 94 | 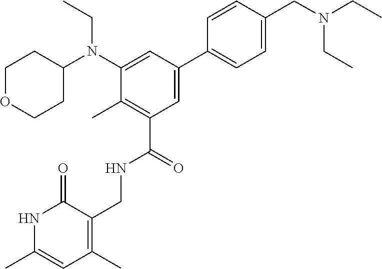 [SMILES: CCN(CC)Cc1ccc(-c2cc(C(=O)NCc3c(C)cc(C)[nH]c3=O)c(C)c(N(CC)C3CCOCC3)c2)cc1] | 559.20 |
| 95 | 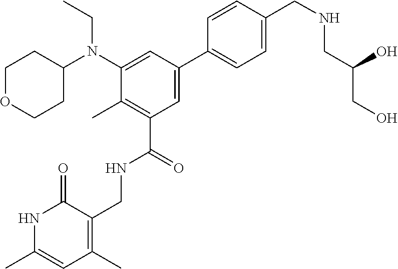 [SMILES: CCN(c1cc(-c2ccc(CNC[C@@H](O)CO)cc2)cc(C(=O)NCc2c(C)cc(C)[nH]c2=O)c1C)C1CCOCC1] | 599.35 (M + Na) |
| 96 | 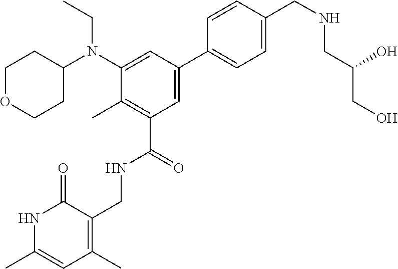 [SMILES: CCN(c1cc(-c2ccc(CNC[C@H](O)CO)cc2)cc(C(=O)NCc2c(C)cc(C)[nH]c2=O)c1C)C1CCOCC1] | 577.25 |
| 97 | 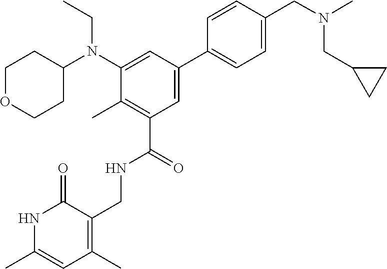 [SMILES: CCN(c1cc(-c2ccc(CN(C)CC3CC3)cc2)cc(C(=O)NCc2c(C)cc(C)[nH]c2=O)c1C)C1CCOCC1] | 571.40 |
| 98 | 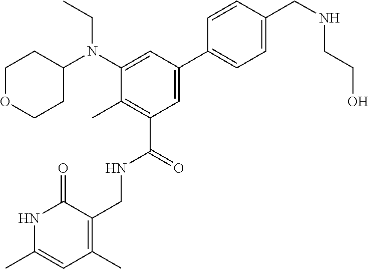 [SMILES: CCN(c1cc(-c2ccc(CNCCO)cc2)cc(C(=O)NCc2c(C)cc(C)[nH]c2=O)c1C)C1CCOCC1] | 547.35 |
| 99 | 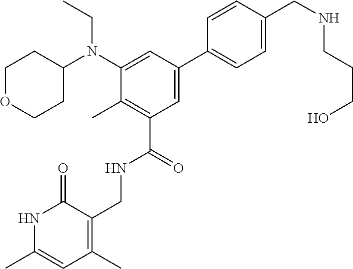 [SMILES: CCN(c1cc(-c2ccc(CNCCCO)cc2)cc(C(=O)NCc2c(C)cc(C)[nH]c2=O)c1C)C1CCOCC1] | 561.30 |
| 100 | 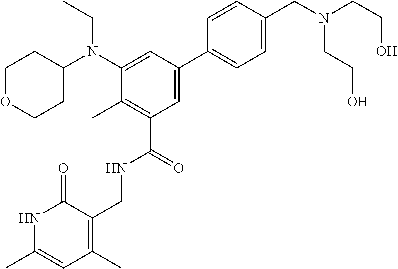 [SMILES: CCN(c1cc(-c2ccc(CN(CCO)CCO)cc2)cc(C(=O)NCc2c(C)cc(C)[nH]c2=O)c1C)C1CCOCC1] | 591.25 |
| 101 | 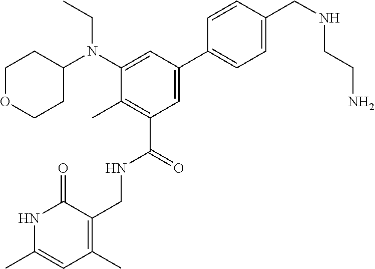 [SMILES: CCN(c1cc(-c2ccc(CNCCN)cc2)cc(C(=O)NCc2c(C)cc(C)[nH]c2=O)c1C)C1CCOCC1] | 546.35 |
| 102 | 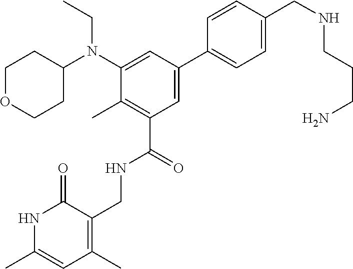 [SMILES: CCN(c1cc(-c2ccc(CNCCCN)cc2)cc(C(=O)NCc2c(C)cc(C)[nH]c2=O)c1C)C1CCOCC1] | 560.20 |
| 103 | 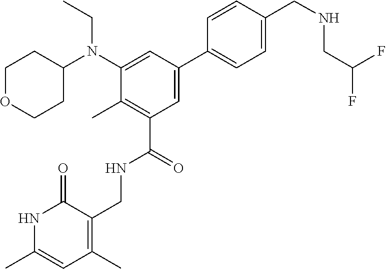 [SMILES: CCN(c1cc(-c2ccc(CNCC(F)F)cc2)cc(C(=O)NCc2c(C)cc(C)[nH]c2=O)c1C)C1CCOCC1] | 567.30 |
| 104 | 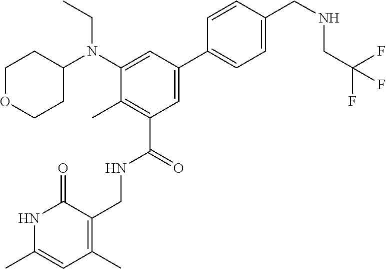 [SMILES: CCN(c1cc(-c2ccc(CNCC(F)(F)F)cc2)cc(C(=O)NCc2c(C)cc(C)[nH]c2=O)c1C)C1CCOCC1] | 585.25 |
| 105 | 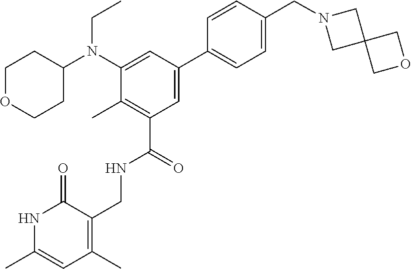 [SMILES: CCN(c1cc(-c2ccc(CN3CC4(COC4)C3)cc2)cc(C(=O)NCc2c(C)cc(C)[nH]c2=O)c1C)C1CCOCC1] | 585.40 |
| 107 | 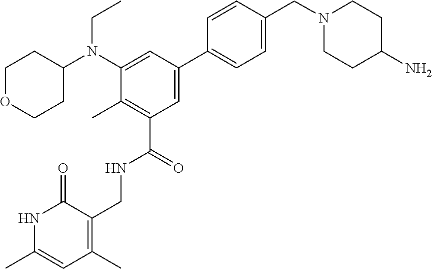 [SMILES: CCN(c1cc(-c2ccc(CN3CCC(N)CC3)cc2)cc(C(=O)NCc2c(C)cc(C)[nH]c2=O)c1C)C1CCOCC1] | |
| 108 | 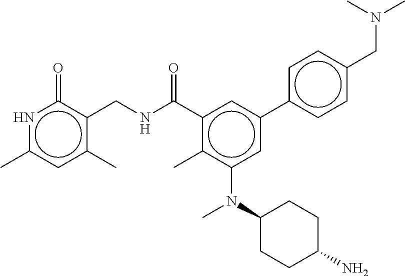 | 530.35 |
| 114 | 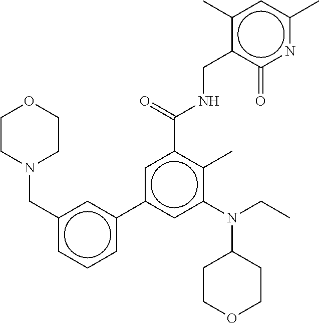 | 573.25 |
| 115 | 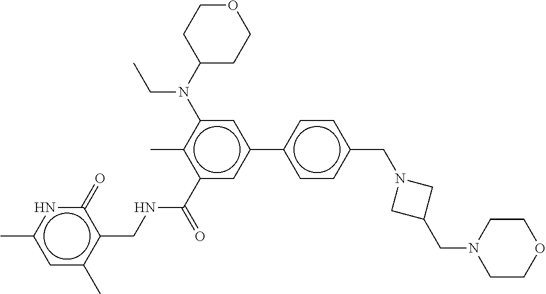 | 642.45 |
| 116 | 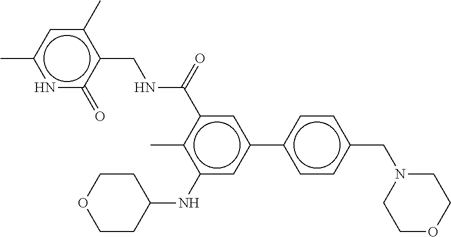 | 545.15 |
| 117 | 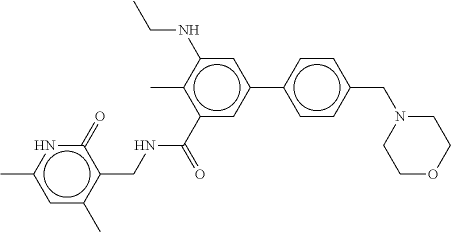 | 489.20 |
| 119 | 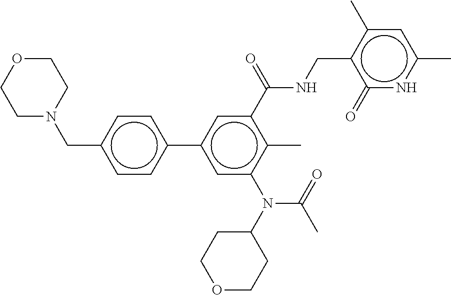 | 609.35 |
| 122 | 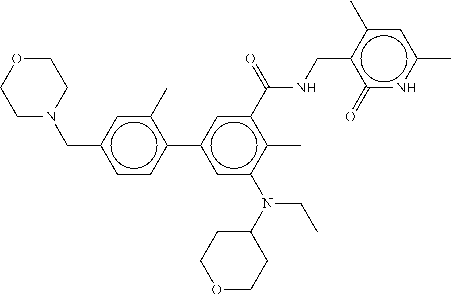 | 587.55 |
| 124 | 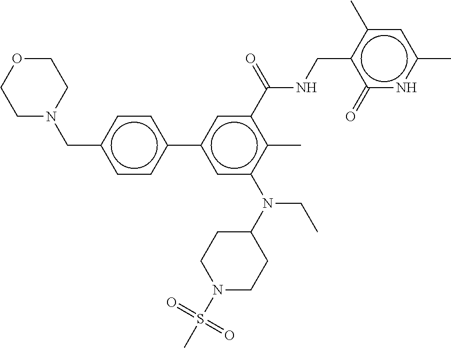 | 650.85 |
| 125 | 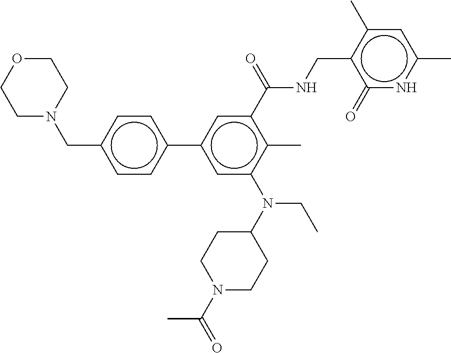 | 614.75 |
| 126 | 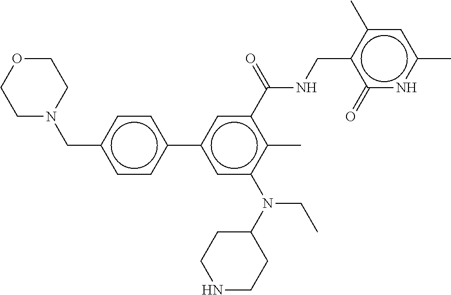 | 572.35 |
| 127 | 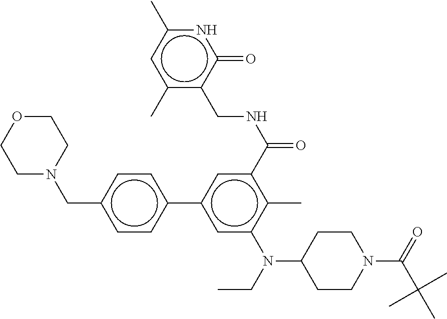 | 656.65 |
| 128 | 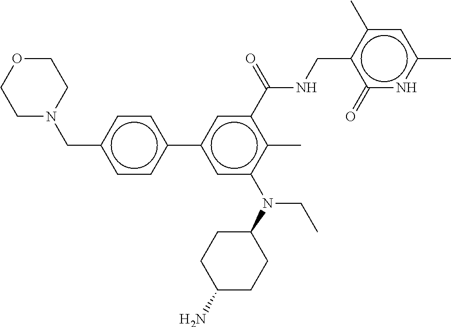 | 586.45 |
| 129 | 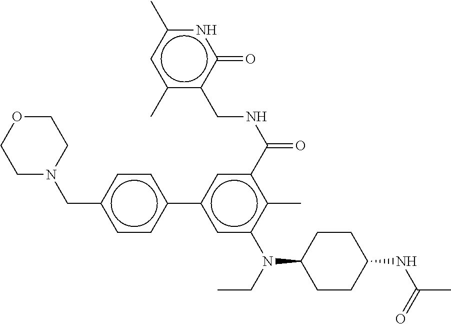 | 628.35 |
| 130 | 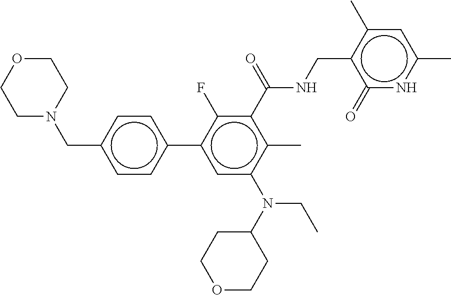 | 591.2 |
| 131 | 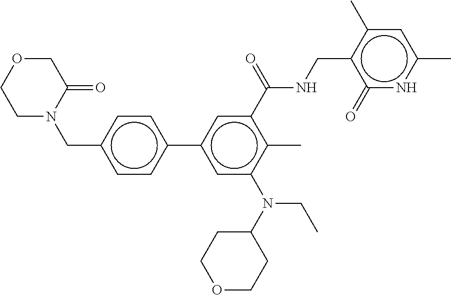 | 587.35 |
| 132 | 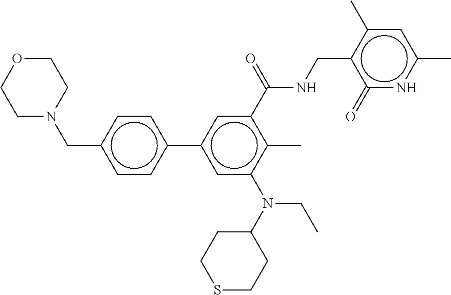 | 589.25 |
| 133 | 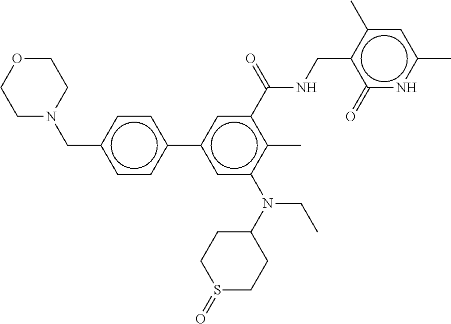 | 605.25 |
| 135 | 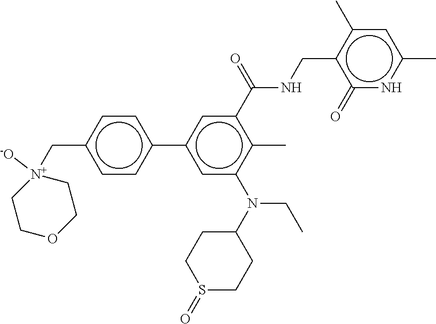 | 621.40 |
| 136 | 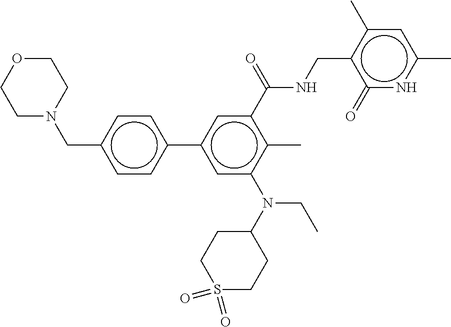 | 621.45 |
| 137 | 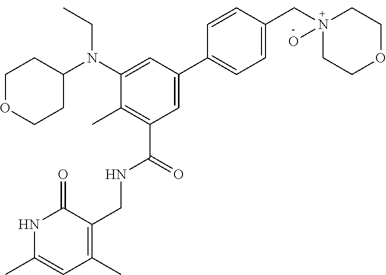 | 589.35 |
| 138 | 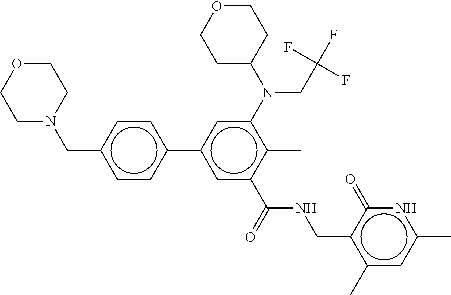 | 627.5 |
| 141 | 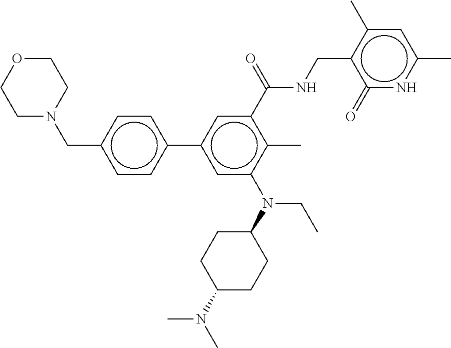 | 614.65 |
| 142 | 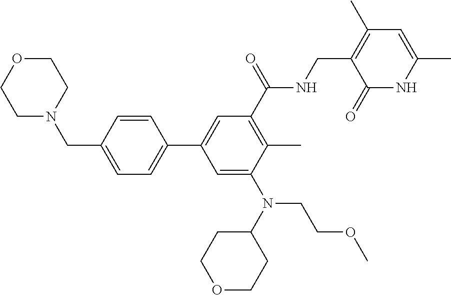 [SMILES: COCCN(c1cc(-c2ccc(CN3CCOCC3)cc2)cc(C(=O)NCc2c(C)cc(C)[nH]c2=O)c1C)C1CCOCC1] | 603.45 |
| 143 | 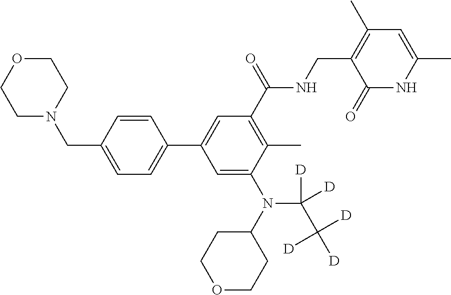 [SMILES: [2H]C([2H])([2H])C([2H])([2H])N(c1cc(-c2ccc(CN3CCOCC3)cc2)cc(C(=O)NCc2c(C)cc(C)[nH]c2=O)c1C)C1CCOCC1] | 578.35 |
| 144 | 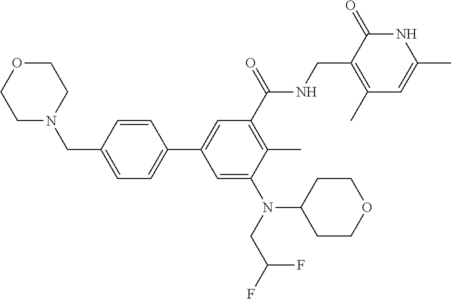 [SMILES: Cc1cc(C)c(CNC(=O)c2cc(-c3ccc(CN4CCOCC4)cc3)cc(N(CC(F)F)C3CCOCC3)c2C)c(=O)[nH]1] | 609.15 |
| 146 | 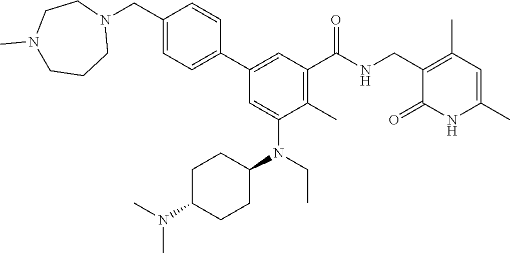 | 641.50 |
| 178 | 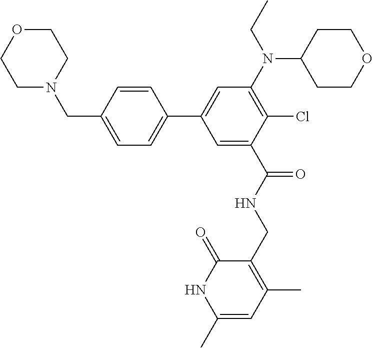 [SMILES: CCN(c1cc(-c2ccc(CN3CCOCC3)cc2)cc(C(=O)NCc2c(C)cc(C)[nH]c2=O)c1Cl)C1CCOCC1] | 593.60 |

As used herein, “alkyl”, “C₁, C₂, C₃, C₄, C₅ or C₆ alkyl” or “C₁-C₆ alkyl” is intended to include C₁, C₂, C₃, C₄, C₅ or C₆ straight chain (linear) saturated aliphatic hydrocarbon groups and C₃, C₄, C₅ or C₆ branched saturated aliphatic hydrocarbon groups. For example, C₁-C₆ alkyl is intended to include C₁, C₂, C₃, C₄, C₅ and C₆ alkyl groups. Examples of alkyl include, moieties having from one to six carbon atoms, such as, but not limited to, methyl, ethyl, n-propyl, i-propyl, n-butyl, s-butyl, t-butyl, n-pentyl, s-pentyl or n-hexyl.

In certain embodiments, a straight chain or branched alkyl has six or fewer carbon atoms (e.g., C₁-C₆ for straight chain, C₃-C₆ for branched chain), and in some embodiments, a straight chain or branched alkyl has four or fewer carbon atoms.

As used herein, the term “cycloalkyl” refers to a saturated or unsaturated nonaromatic hydrocarbon mono-or multi-ring (e.g., fused, bridged, or Spiro rings) system having 3 to 30 carbon atoms (e.g., C₃-C₁₀). Examples of cycloalkyl include, but are not limited to, cyclopropyl, cyclobutyl, cyclopentyl, cyclohexyl, cycloheptyl, cyclooctyl, cyclopentenyl, cyclohexenyl, cycloheptenyl, and adamantyl. The term “heterocycloalkyl” refers to a saturated or unsaturated nonaromatic 3-8 membered monocyclic, 7-12 membered bicyclic (fused, bridged, or Spiro rings), or 11-14 membered tricyclic ring system (fused, bridged, or Spiro rings) having one or more heteroatoms (such as O, N, S, or Se), unless specified otherwise. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, triazolidinyl, tetrahyrofuranyl, oxiranyl, azetidinyl, oxetanyl, thietanyl, 3,6-tetrahydropyridinyl, tetrahydropyranyl, dihydropyranyl, pyranyl, morpholinyl, 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl and the like.

The term “optionally substituted alkyl” refers to unsubstituted alkyl or alkyl having designated substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulthydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

An “arylalkyl” or an “aralkyl” moiety is an alkyl substituted with an aryl (e.g., phenylmethyl (benzyl)). An “alkylaryl” moiety is an aryl substituted with an alkyl (e.g., methylphenyl).

As used herein, “alkyl linker” is intended to include C₁, C₂, C₃, C₄, C₅ or C₆ straight chain (linear) saturated divalent aliphatic hydrocarbon groups and C₃, C₄, C₅ or C₆ branched saturated aliphatic hydrocarbon groups. For example, C₁-C₆ alkyl linker is intended to include C₁, C₂, C₃, C₄, C₅ and C₆ alkyl linker groups. Examples of alkyl linker include, moieties having from one to six carbon atoms, such as, but not limited to, methyl (—CH₂—), ethyl (—CH₂CH₂—), n-propyl (—CH₂CH₂CH₂—), i-propyl (—CHCH₃CH₂—), n-butyl (—CH₂CH₂CH₂CH₂—), s-butyl (—CHCH₃CH₂CH₂—), i-butyl (—C(CH₃)₂CH₂) n-pentyl (—CH₂CH₂CH₂CH₂CH₂—), s-pentyl (—CHCH₃CH₂CH₂CH₂—) or n-hexyl (—CH₂CH₂CH₂CH₂CH₂CH₂—).

“Alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₂-C₆ for straight chain, C₃-C₆ for branched chain). The term “C₂-C₆” includes alkenyl groups containing two to six carbon atoms. The term “C₃-C₆” includes alkenyl groups containing three to six carbon atoms.

The terra “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, amino, sulthydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

“Alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C₂-C₆ for straight chain, C₃-C₆ for branched chain). The term “C₂-C₆” includes alkynyl groups containing two to six carbon atoms. The term “C₃-C₆” includes alkynyl groups containing three to six carbon atoms.

The term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarhonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, amino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylatyl, or an aromatic or heteroaromatic moiety.

Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3, 6-tetrahydropyridinyl.

“Aryl” includes groups with aromaticity, including “conjugated,” or multicyclic systems with at least one aromatic ring and do not contain any heteroatom in the ring structure. Examples include phenyl, benzyl, 1, 2,3,4-tetrahydronaphthalenyl, etc.

“Heteroaryl” groups are aryl groups, as defined above, except having from one to four heteroatoms in the ring structure, and may also be referred to as “aryl heterocycles” or “heteroaromatics.” As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted N or NR wherein R is H or other substituents, as defined). The nitrogen and sulfur heteroatoms may optionally be oxidized (i.e., N→O and S(O)ₚ, where p=1 or 2). It is to be noted that total number of S and O atoms in the aromatic heterocycle is not more than 1.

Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like.

Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, methyl enedioxyphenyl, quinoline, isoquinoline, naphthrydine, indole, benzofuran, purine, benzofuran, deazapurine, indolizine.

In the case of multicyclic aromatic rings, only one of the rings needs to be aromatic (e.g., 2,3-dihydroindole), although all of the rings may be aromatic (e.g., quinoline). The second ring can also be fused or bridged.

The cycloalkyl, heterocycloalkyl, aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkylcarbonyloxy, arylcarbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, aryl amino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamide, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl).

As used herein, “carbocycle” or “carbocyclic ring” is intended to include any stable monocyclic, bicyclic or tricyclic ring having the specified number of carbons, any of which may be saturated, unsaturated, or aromatic. Carbocycle includes cycloalkyl and aryl. For example, a C₃-C₁₄ carbocycle is intended to include a monocyclic, bicyclic or tricyclic ring having 3, 4, 5, 6, 7, 8, 9, 10, 11, 12, 13 or 14 carbon atoms. Examples of carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, fluorenyl, phenyl, naphthyl, indanyl, adamantyl and tetrahydronaphthyl. Bridged rings are also included in the definition of carbocycle, including, for example, [3.3.0]bicyclooctane, [4.3.0]bicyclononane, [4.4.0]bicyclodecane and [2.2.2]bicyclooctane. A bridged ring occurs when one or more carbon atoms link two non-adjacent carbon atoms. In some embodiments, bridge rings are one or two carbon atoms. It is noted that a bridge always converts a monocyclic ring into a tricyclic ring. When a ring is bridged, the substituents recited for the ring may also be present on the bridge. Fused (e.g., naphthyl, tetrahydronaphthyl) and spino rings are also included.

As used herein, “heterocycle” or “heterocyclic group” includes any ring structure (saturated, unsaturated, or aromatic) which contains at least one ring heteroatom (e.g., N, O or S). Heterocycle includes heterocycloalkyl and heteroaryl. Examples of heterocycles include, but are not limited to, morpholine, pyrrolidine, tetrahydrothiophene, piperidine, piperazine, oxetane, pyran, tetrahydropyran, azetidine, and tetrahydrofuran.

Examples of heterocyclic groups include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl imidazolyl, 1H-indazolyl, indolenyl, indolizinyl, indolyl 3H-indolyl isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, 1,2,4-oxadiazol5(4H)-one, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl and xanthenyl.

The term “substituted,” as used herein, means that any one or more hydrogen atoms on the designated atom is replaced with a selection from the indicated groups, provided that the designated atom's normal valency is not exceeded, and that the substitution results in a stable compound. When a substituent is oxo or keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R₁) occurs more than one time in any constituent or formula for a compound, its definition at each occurrence is independent of its definition at every other occurrence. Thus, for example, if a group is shown to be substituted with 0-2 R₁ moieties, then the group may optionally be substituted with up to two R₁ moieties and R₁ at each occurrence is selected independently from the definition of R₁. Also, combinations of substituents and/or variables are permissible, but only if such combinations result in stable compounds.

The term “hydroxy” or “hydroxyl” includes groups with an —OH or —O⁻.

As used herein, “halo” or “halogen” refers to fluoro, chloro, bromo and iodo. The term “perhalogenated” generally refers to a moiety wherein all hydrogen atoms are replaced by halogen atoms. The term “haloalkyl” or “haloalkoxyl” refers to an alkyl or alkoxyl substituted with one or more halogen atoms.

The term “carbonyl” includes compounds and moieties which contain a carbon connected with a double bond to an oxygen atom. Examples of moieties containing a carbonyl include, but are not limited to, aldehydes, ketones, carboxylic acids, amides, esters, anhydrides, etc.

The term “carboxyl” refers to —COOH or its C₁-C₆ alkyl ester.

“Acyl” includes moieties that contain the acyl radical (R—C(O)—) or a carbonyl group. “Substituted acyl” includes acyl groups where one or more of the hydrogen atoms are replaced by, for example, alkyl groups, alkynyl groups, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano azido, heterocyclyl, alkylaryl, or an aromatic or heteroaromatic moiety.

“Aroyl” includes moieties with an aryl or heteroaromatic moiety bound to a carbonyl group. Examples of aroyl groups include phenylcarboxy, naphthyl carboxy, etc.

“Alkoxyalkyl,” “alkylaminoalkyl,” and “thioalkoxyalkyl” include alkyl groups, as described above, wherein oxygen, nitrogen, or sulfur atoms replace one or more hydrocarbon backbone carbon atoms.

The term “alkoxy” or “alkoxyl” includes substituted and unsubstituted alkyl, alkenyl and alkynyl groups covalently linked to an oxygen atom. Examples of alkoxy groups or alkoxyl radicals include, but are not limited to, methoxy, ethoxy, isopropyloxy, propoxy, butoxy and pentoxy groups. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy and trichloromethoxy.

The term “ether” or “alkoxy” includes compounds or moieties which contain an oxygen bonded to two carbon atoms or heteroatoms. For example, the term includes “alkoxyalkyl,” which refers to an alkyl, alkenyl, or alkynyl group covalently bonded to an oxygen atom which is covalently bonded to an alkyl group.

The term “ester” includes compounds or moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc.

The term “thioalkyl” includes compounds or moieties which contain an alkyl group connected with a sulfur atom. The thioalkyl groups can be substituted with groups such as alkyl, alkenyl, alkynyl, halogen, hydroxyl, alkylcarbonyloxy, arylcarbonyloxy, alkoxycarbonyloxy, ary, oxycarbonyloxy, carboxylate, carboxyacid, alkylcarbonyl, aryl carbonyl, alkoxycarbonyl, aminocarbonyl, alkylaminocarbonyl, dialkylaminocarbonyl, alkylthiocarbonyl, alkoxyl, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsultinyl, sulfonato, sulfamoyl, sulfonamide, nitro, trifluoromethyl, cyano, azido, heterocyclyl, alkylaryl, or an aromatic or heteroaromatic moieties.

The term “thiocarbonyl” or “thiocarboxy” includes compounds and moieties which contain a carbon connected with a double bond to a sulfur atom.

The term “thioether” includes moieties which contain a sulfur atom bonded to two carbon atoms or heteroatoms. Examples of thioethers include, but are not limited to alkthioalkyls, alkthioalkenyls, and alkthioalkynyls. The term “alkthioalkyls” include moieties with an alkyl, alkenyl, or alkynyl group bonded to a sulfur atom which is bonded to an alkyl group. Similarly, the term “alkthioalkenyls” refers to moieties wherein an alkyl, alkenyl or alkynyl group is bonded to a sulfur atom which is covalently bonded to an alkenyl group; and alkthioalkynyls” refers to moieties wherein an alkyl, alkenyl or alkynyl group is bonded to a sulfur atom which is covalently bonded to an alkynyl group.

As used herein, “amine” or “amino” refers to unsubstituted or substituted —NH₂. “Alkylamino” includes groups of compounds wherein nitrogen of —NH₂ is bound to at least one alkyl group. Examples of alkylamino groups include benzylamino, methylamino, ethylamino, phenethylamino, etc. “Dialkylamino” includes groups wherein the nitrogen of —NH₂ is bound to at least two additional alkyl groups. Examples of dialkylamino groups include, but are not limited to, dimethylamino and diethylamino. “Arylamino” and “diarylamino” include groups wherein the nitrogen is bound to at least one or two aryl groups, respectively. “Aminoaryl” and “aminoaryloxy” refer to aryl and aryloxy substituted with amino. “Alkylarylamino,” “alkylaminoaryl” or “arylaminoalkyl” refers to an amino group which is bound to at least one alkyl group and at least one aryl group. “Alkaminoalkyl” refers to an alkyl, alkenyl, or alkynyl group bound to a nitrogen atom which is also bound to an alkyl group. “Acylamino” includes groups wherein nitrogen is bound to an acyl group. Examples of acylamino include, but are not limited to, alkylcarbonylamino, arylcarbonylamino, carbamoyl and ureide groups.

The term “amide” or “aminocarboxy” includes compounds or moieties that contain a nitrogen atom that is bound to the carbon of a carbonyl or a thiocarbonyl group. The term includes “alkaminocarboxy” groups that include alkyl, alkenyl or alkynyl groups bound to an amino group which is bound to the carbon of a carbonyl or thiocarbonyl group. It also includes “arylaminocarboxy” groups that include aryl or heteroaryl moieties bound to an amino group that is bound to the carbon of a carbonyl or thiocarbonyl group. The terms “alkylaminocarboxy”, “alkenylaminocarboxy”, “alkynylaminocarboxy” and “arylaminocarboxy” include moieties wherein alkyl, alkenyl, alkynyl and aryl moieties, respectively, are bound to a nitrogen atom which is in turn bound to the carbon of a carbonyl group. Amides can be substituted with substituents such as straight chain alkyl, branched alkyl, cycloalkyl, aryl, heteroaryl or heterocycle. Substituents on amide groups may be further substituted.

Compounds of the present disclosure that contain nitrogens can be converted to N-oxides by treatment with an oxidizing agent (e.g., 3-chloroperoxybenzoic acid (mCPBA) and/or hydrogen peroxides) to afford other compounds of the present disclosure. Thus, all shown and claimed nitrogen-containing compounds are considered, when allowed by valency and structure, to include both the compound as shown and its N-oxide derivative (which can be designated as N→O or N⁺-O⁻). Furthermore, in other instances, the nitrogens in the compounds of the present disclosure can be converted to N-hydroxy or N-alkoxy compounds. For example, N-hydroxy compounds can be prepared by oxidation of the parent amine by an oxidizing agent such as m-CPBA. All shown and claimed nitrogen-containing compounds are also considered, when allowed by valency and structure, to cover both the compound as shown and its N-hydroxy (i.e., N—OH) and N-alkoxy (i.e., N—OR, wherein R is substituted or unsubstituted C₁-C₆ alkyl, C₁-C₆ alkenyl, C₁-C₆ alkynyl, 3-14-membered carbocycle or 3-14-membered heterocycle) derivatives.

“Isomerism” means compounds that have identical molecular formulae but differ in the sequence of bonding of their atoms or in the arrangement of their atoms in space, Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

A carbon atom bonded to four nonidentical substituents is termed a “chiral center.”

“Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem, Sac, 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds or a cycloalkyl linker (e.g., 1, 3-cylcobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the small molecule EZH2 inhibitors provided herein may be depicted as different chiral isomers or geometric isomers. It should also be understood that when compounds have chiral isomeric or geometric isomeric forms, all isomeric forms are intended to be included in the scope of the present disclosure, and the naming of the compounds does not exclude any isomeric forms.

Furthermore, the structures and other compounds discussed in this disclosure include all atropic isomers thereof. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertable by tautomerizations is called tautomerism.

Of the various types of tautomerism that are possible, two are commonly observed in keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), imine-enamine and enamine-enamine. An example of keto-enol equilibria is between pyridin-2(1H)-ones and the corresponding pyridin-2-ols, as shown below.

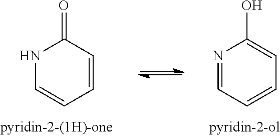 [SMILES: O=c1cccc[nH]1 ; Oc1ccccn1]

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form.

The EZH2 inhibitors of Formulae (I)-(VIa) disclosed herein include the compounds themselves, as well as their salts and their solvates, if applicable. A salt, for example, can be formed between an anion and a positively charged group (e.g., amino) on an aryl- or heteroaryl-substituted benzene compound. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate). The term “pharmaceutically acceptable anion” refers to an anion suitable for forming a pharmaceutically acceptable salt. Likewise, a salt can also be formed between a cation and a negatively charged group (e.g., carboxylate) on an aryl- or heteroaryl-substituted benzene compound. Suitable cations include sodium ion, potassium ion, magnesium ion, calcium ion, and an ammonium cation such as tetramethylammonium ion. The aryl- or heteroaryl-substituted benzene compounds also include those salts containing quaternary nitrogen atoms. In the salt form, it is understood that the ratio of the compound to the cation or anion of the salt can be 1:1, or any ration other than 1:1, e.g., 3:1, 2:1, 1:2, or 1:3.

Additionally, the EZH2 inhibitory compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure, and are substituted with various groups as described herein. For example, all of the compounds represented by Formula (I) are aryl- or heteroaryl-substituted benzene compounds, and have Formula (I) as a common core.

Some embodiments of the present disclosure embrace some or all isotopes of atoms occurring in the present EZH2 inhibitory compounds. Isotopes include those atoms having the same atomic number but different mass numbers. By way of general example and without limitation, isotopes of hydrogen include tritium and deuterium, and isotopes of carbon include C-13 and C-14.

In certain aspects of the disclosure an inhibitor of EZH2 “selectively inhibits” histone methyltransferase activity of the mutant EZH2 when it inhibits histone methyltransferase activity of the mutant EZH2 more effectively than it inhibits histone methyltransferase activity of wild-type EZH2. For example, in some embodiments the selective inhibitor has an IC₅₀ for the mutant EZH2 that is at least 40 percent lower than the IC₅₀ for wild-type EZH2. In some embodiments, the selective inhibitor has an IC50 for the mutant EZH2 that is at least 50 percent lower than the IC50 for wild-type EZH2. In some embodiments, the selective inhibitor has an IC50 for the mutant EZH2 that is at least 60 percent lower than the IC50 for wild-type EZH2. In some embodiments, the selective inhibitor has an IC50 for the mutant EZH2 that is at least 70 percent lower than the IC50 for wild-type EZH2. In some embodiments, the selective inhibitor has an IC50 for the mutant EZH2 that is at least 80 percent lower than the IC50 for wild-type EZH2. In some embodiments, the selective inhibitor has an IC50 for the mutant EZH2 that is at least 90 percent lower than the IC50 for wild-type EZH2.

In some embodiments, the selective inhibitor of a mutant EZH2 exerts essentially no inhibitory effect on wild-type EZH2.

In certain aspects of the disclosure the inhibitor (e.g. compound disclosed herein) inhibits conversion of H3-K27me2 to H3-K27me3. In some embodiments the inhibitor is said to inhibit trimethylation of H3-K27. Since conversion of H3-K27me1 to H3-K27me2 precedes conversion of H3-K27me2 to H3-K27me3, an inhibitor of conversion of H3-K27me1 to H3-K27me2 naturally also inhibits conversion of H3-K27me2 to H3-K27me3, i.e., it inhibits trimethylation of H3-K27. It is also possible to inhibit conversion of H3-K27me2 to H3-K27me3 without inhibition of conversion of H3-K27me1 to H3-K27me2. Inhibition of this type would also result in inhibition of trimethylation of H3-K27, albeit without inhibition of dimethylation of H3-K27.

In some embodiments the inhibitor (e.g. compound disclosed herein) inhibits conversion of H3-K27me1 to H3-K27me2 and the conversion of H3-K27me2 to H3-K27me3. Such inhibitor may directly inhibit the conversion of H3-K27me1 to H3-K27me2 alone. Alternatively, such inhibitor may directly inhibit both the conversion of H3-K27me1 to H3-K27me2 and the conversion of H3-K27me2 to 113-K 27me3.

In certain aspects of the disclosure, the EZH2 inhibitor (e.g. compound disclosed herein) inhibits histone methyltransferase activity. Inhibition of histone methyltransferase activity can be detected using any suitable method. The inhibition can be measured, for example, either in terms of rate of histone methyltransferase activity or as product of histone methyltransferase activity.

In some embodiments, strategies, treatment modalities, methods, combinations, and compositions are provided that are characterized by a measurable inhibition of EZH2 activity, for example, a measureable EZH2 inhibition as compared to a suitable control. In some embodiments, EZH2 inhibition is at least 10 percent inhibition compared to a suitable control, e.g., an EZH2 activity observed or expected in an untreated control cell, tissue, or subject. In some embodiments, the rate of EZH2 enzymatic activity in the presence of the EZH2 inhibitor is less than or equal to 90 percent of the corresponding enzymatic activity in the absence of the EZH2 inhibitor in some embodiments, EZH2 inhibition in the presence of the EZH2 inhibitor is at least 20, 25, 30, 40, 50, 60, 70, 75, 80, 90, or 95 percent inhibition as compared to a suitable control, e.g., to activity in the absence of the inhibitor . In some embodiments, inhibition is at least 99 percent inhibition compared to a suitable control. That is, the rate of enzymatic activity in the presence of the inhibitor is less than or equal to 1 percent of the corresponding activity in the absence of the inhibitor.

In some embodiments, the therapeutic agents provided herein, e.g., the EZH2 inhibitor, and, where applicable, any additional therapeutic agents, e.g., an immune checkpoint inhibitor, are provided in pharmaceutical formulations suitable for administration to a human subject. In embodiments where more than one therapeutic agent is used, each therapeutic agent may be formulated separately into a pharmaceutical formulation, and administered to the subject independently, e.g., sequentially in some such embodiments, the different pharmaceutical compositions may be administered via the same route, e.g., a parenteral route, or, alternatively, via different routes, e.g., an enteral and a parenteral route. For example, in some embodiments of combination treatment modalities provided herein, the EZH2 inhibitor may be formulated for oral administration and an additional therapeutic agent, e.g., an immune checkpoint inhibitor, is formulated for parenteral administration.

Suitable pharmaceutical compositions comprising EZH2 inhibitors have previously been described, and include, for example, and without limitation, those listed in U.S. Pat. Nos. 8,410,088, 8,765,732, 9,090,562, 8,598,167, 8,962,620, US-2015/0065483, U.S. Pat. No. 9,206,157, 9,006,242, 9,089,575, US 2015-0352119, WO 2014/062733, US-2015/0065503, WO2015/057859, U.S. Pat. No. 8,536,179, WO2011/140324,PCT/US2014/015706, published as WO/2014/124418, in PCT/US2013/025639, published as WO/2013/120104, and in U.S. Ser. No. 14/839,273, published as US 2015/0368229, the entire contents of each of which are incorporated herein by reference. Additional suitable pharmaceutical compositions will be apparent to those of skill in the art based on the present disclosure and the general knowledge in the art.

The disclosure also provides pharmaceutical compositions and combinations comprising a compound of Formulae (I)-(VIa) or pharmaceutically acceptable salts thereof, and one or more other therapeutic agents disclosed herein, e.g., one or more immune checkpoint inhibitors, mixed with pharmaceutically suitable carriers or excipient(s) at doses to treat or prevent a disease or condition as described herein. In one aspect, the disclosure also provides pharmaceutical compositions comprising any compound of Table I or pharmaceutically acceptable salts thereof, and one or more therapeutic agents, mixed with pharmaceutically suitable carriers or excipient (s) at doses to treat or prevent a disease or condition as described herein. In another aspect, the disclosure also provides pharmaceutical compositions comprising Compound 44

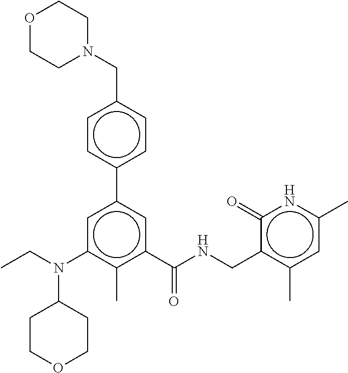

or pharmaceutically acceptable salts thereof, and one or more therapeutic agents, mixed with pharmaceutically suitable carriers or excipient(s) at doses to treat or prevent a disease or condition as described herein. The pharmaceutical compositions of the disclosure can also be administered in combination with other therapeutic agents or therapeutic modalities simultaneously, sequentially, or in alternation.

Mixtures or combinations of compositions of the disclosure can also be administered to the patient as a simple mixture or in suitable formulated pharmaceutical compositions. For example, one aspect of the disclosure relates to a pharmaceutical composition or combination comprising a therapeutically effective dose of an EZH2 inhibitor of Formulae (I)-(VIa), or a pharmaceutically acceptable salt, hydrate, enantiomer or stereoisomer thereof; one or more other therapeutic agents, and a pharmaceutically acceptable diluent or carrier.

A “pharmaceutical composition” is a formulation containing the compounds of the disclosure in a form suitable for administration to a subject. A compound of Formulae (i)-(VIa) and, where applicable, one or more other therapeutic agents described herein each can be formulated individually or in multiple pharmaceutical compositions in any combinations of the active ingredients. Accordingly, one or more administration routes can be properly elected based on the dosage form of each pharmaceutical composition. Alternatively, a compound of Formulae (I)-(VIa) and one or more other therapeutic agents described herein can be formulated as one pharmaceutical composition.

In some embodiments, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skill led in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In some embodiments, the active compound is mixed under sterile conditions with a pharmaceutically acceptable carrier, and with any preservatives, buffers, or propellants that are required.

As used herein, the phrase “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable excipient” refers to an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intraderrnal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A composition of the disclosure, e.g., a formulation comprising an EZH2 inhibitor, can be administered to a subject in many of the well-known methods currently used for chemotherapeutic treatment. For example, for treatment of cancers, a formulation comprising an EZH2 inhibitor may be injected directly into tumors, injected into the blood stream or body cavities or taken orally or applied through the skin with patches. The dose chosen for the EZH2 inhibitor and, where applicable, for any additional therapeutic agent, should be sufficient to constitute effective treatment but not so high as to cause unacceptable side effects. The state of the disease condition (e.g., cancer, precancer, and the like) and the health of the patient should preferably be closely monitored during and for a reasonable period after treatment.

The term “therapeutically effective amount”, as used herein, refers to an amount of a pharmaceutical agent to treat, ameliorate, or prevent an identified disease or condition, or to exhibit a desired clinical effect, e.g., a detectable therapeutic or inhibitory effect. Exemplary, non-limiting effective amounts and effective dosage ranges of EZH2 inhibitors and some exemplary additional therapeutic agents are provided herein. In some embodiments, the desired clinical effect can be detected directly, e.g., by any suitable assay method known in the art. In some embodiments, the desired clinical effect can be measured by a proxy measurement. For example, in some embodiments, reactivation of epigenetically repressed SMARCA2 and/or SMARCA4 expression can be monitored to determine a suitable, therapeutically effective amount of an EZH2 inhibitor. The precise effective amount for a subject will depend upon the subject's body weight, size, and health; the nature and extent of the condition; and the therapeutic or combination of therapeutics selected for administration Therapeutically effective amounts for a given situation can be determined by routine experimentation that is within the skill and judgment of the clinician. In a preferred aspect, the disease or condition to be treated is cancer. In another aspect, the disease or condition to be treated is a cell proliferative disorder.

In certain embodiments the therapeutically effective amount of each pharmaceutical agent used in combination will be lower when used in combination in comparison to monotherapy with each agent alone. Such lower therapeutically effective amount could afford for lower toxicity of the therapeutic regimen.

For many of the compounds described herein, e.g., various EZH2 inhibitors and various additional therapeutic agents, a therapeutically effective amount or an effective dosage range has been reported. In some embodiments, an effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and. frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and. antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin,

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In some embodiments of therapeutic applications, the dosages of the therapeutic agents provided herein vary depending on the specific agent(s) used, the age, weight, and clinical condition of the recipient patient, and the experience and judgment of the clinician or practitioner administering the therapy, among other factors affecting the selected dosage. Generally, the dose of the active ingredient(s) should be sufficient to result in slowing, and preferably regressing, the growth of the tumors and also preferably causing complete regression of the cancer. In some embodiments, dosages can range from about 0.01 mg/kg per day to about 5000 mg/kg per day. In preferred aspects, dosages can range from about 1 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m², and age in years). Additional suitable dosages are provided elsewhere herein. For example, regression of a tumor in a patient may be measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

As used herein, “pharmaceutically acceptable salts” refer to derivatives of the compounds of the disclosure, e.g., of the small molecule EZH2 inhibitors described herein, wherein the parent compound is modified by making acid or base salts thereof. Examples of pharmaceutically acceptable salts, e.g., of the EZH2 inhibitors provided herein, include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates), of the same salt.

The composition of the disclosure may also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., acetate, propionate or other ester.

The composition, or pharmaceutically acceptable salts or solvates thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In some embodiments, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the disclosure can be found in Remington: the Science and Practice of Pharmacy, 19^{th} edition, Mack Publishing Co., Easton, Pa. (1995). In some embodiments, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the disclosure are apparent from the different examples. The provided examples illustrate different components and methodology useful in practicing the disclosure. The examples do not limit the claimed disclosure. Based on the present disclosure the skilled artisan can identify and employ other components and methodology useful for practicing the disclosure.

In some embodiments, a “subject in need thereof” is a subject having a disorder in which EZH2-mediated protein methylation plays a part, or a subject having an increased risk of developing such disorder relative to the population at large. In some embodiments, a subject in need thereof has a cell proliferative disease, e.g., a cancer. In some embodiments, the subject has a cancer characterized by SMARCA2 and/or SMARCA4 loss of function. In Some embodiments, the subject has a cancer characterized by SMARCA2/SMARCA4 dual loss of function, wherein the SMARCA2 loss of function is mediated by an epigenetic lesion. In some embodiments, the subject has a disorder in which immune system evasion also plays a role, e.g., immune system evasion of cancer cells via immune checkpoint signaling. A “subject” includes a mammal. The mammal can be e.g., any mammal, e.g., a human, primate, bird, mouse, rat, fowl, dog, cat, cow, horse, goat, camel, sheep or a pig. Preferably, the mammal is a human.

In some embodiments, the subject is a human subject who has been diagnosed with, has symptoms of or is at risk of developing a cancer or a precancerous condition. In some embodiments, the subject expresses a mutant EZH2 protein. For example, a mutant EZH2 comprising one or more mutations, wherein the mutation is a substitution, a point mutation, a nonsense mutation, a missense mutation, a deletion, or an insertion or any other EZH2 mutation described herein. In some embodiments, the subject expresses a wild type EZH2 protein.

A subject in need thereof may have refractory or resistant cancer. “Refractory or resistant cancer” means cancer that does not respond to treatment, e.g., to treatment with a monotherapy, e.g., a monotherapy with a chemotherapeutic agent alone. In some embodiments, the cancer may be refractory or resistant to the standard of care treatment for that particular type of cancer. The cancer may be resistant at the beginning of treatment or it may become resistant during treatment. In some embodiments, the subject in need thereof has cancer recurrence following remission on most recent therapy. In some embodiments, the subject in need thereof received and failed all known effective therapies for cancer treatment. In some embodiments, the subject in need thereof received at least one prior therapy. In certain embodiments the prior therapy is monotherapy. In certain embodiments the prior therapy is combination therapy.

In some embodiments, a subject in need thereof may have a secondary cancer as a result of a previous therapy. “Secondary cancer” means cancer that arises due to or as a result from previous carcinogenic therapies, such as chemotherapy.

The subject may also exhibit resistance to EZH2 histone methyltransferase inhibitors or any other therapeutic agent.

As used herein, the term “responsiveness” is interchangeable with terms “responsive”, “sensitive”, and “sensitivity”, and it is meant that a subject is showing therapeutic responses when administered a composition of the disclosure, e.g., tumor cells or tumor tissues of the subject undergo apoptosis and/or necrosis, and/or display reduced growing, dividing, or proliferation. This term is also meant that a subject will or has a higher probability, relative to the population at large, of showing therapeutic responses when administered a composition of the disclosure, e.g., tumor cells or tumor tissues of the subject undergo apoptosis and/or necrosis, and/or display reduced growing, dividing, or proliferation.

The term “sample” refers to any biological sample derived from the subject, includes but is not limited to, cells, tissues samples, body fluids (including, but not limited to, mucus, blood, plasma, serum, urine, saliva, and semen), tumor cells, and tumor tissues. Preferably, the sample is selected from bone marrow, peripheral blood cells, blood, plasma and serum. Samples can be provided by the subject under treatment or testing. Alternatively samples can be obtained by the physician according to routine practice in the art.

As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell possesses normally functioning cell cycle checkpoint control mechanisms.

As used herein, “contacting a cell” refers to a condition in which a compound or other composition of matter is in direct contact with a cell, or is close enough to induce a desired biological effect in a cell.

As used herein, “treating” or “treat” describes the management and care of a patient for the purpose of combating a disease, condition, or disorder and includes the administration of an EZH2 inhibitor and/or an immune checkpoint inhibitor, to alleviate the symptoms or complications of a disease, condition or disorder, or to eliminate the disease, condition or disorder.

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXAMPLES

### Sensitivity to EZH2 Inhibition in Lung Cancer Cell Lines In Vitro

The status of SWI/SNF complex proteins was determined in various lung cancer cell lines. About ⅓ of all tested lung cancer cell lines exhibited SWI/SNF member protein aberrations. Table 2A below shows the SMARCA2 and SMARCA4 protein status in 31 lung cancer cell lines identified to harbor one or more SWI-SNF alterations. Dark gray color denotes loss of function, light gray color denotes normal function. As shown in Table 2A, 10 out of the 31 SWI/SNF loss of function lung cancer cell lines exhibited single SMARCA4 loss, while 8 of the 31 lines, listed at the top of the table, exhibited dual SMARCA2/SMARCA4 loss.

Table 2B below shows the SMARCA2 and SMARCA4 protein status in 33 lung cancer cell lines identified to harbor one or more SWI-SNF alterations. Dark gray color denotes mutation, light gray color denotes loss of function, and blank denotes normal function.

SWI/SNF-altered cell lines were treated with the EZH2 inhibitor tazemetostat in vitro, and cell proliferation was assessed after 14 days of treatment (see FIG. 2). SMARCA2/SMARCA4 dual-loss lung cancer cell lines were found to be more sensitive to EZH2 inhibition than lung cancer cell lines with other SWI/SNF aberrations.

### Sensitivity to EZH2 Inhibition in Lung Cancer Xenografts In Vivo

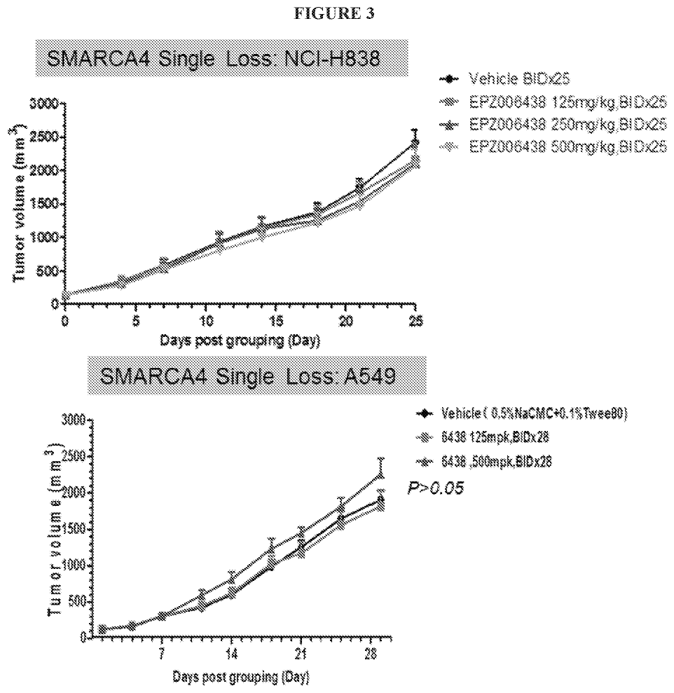

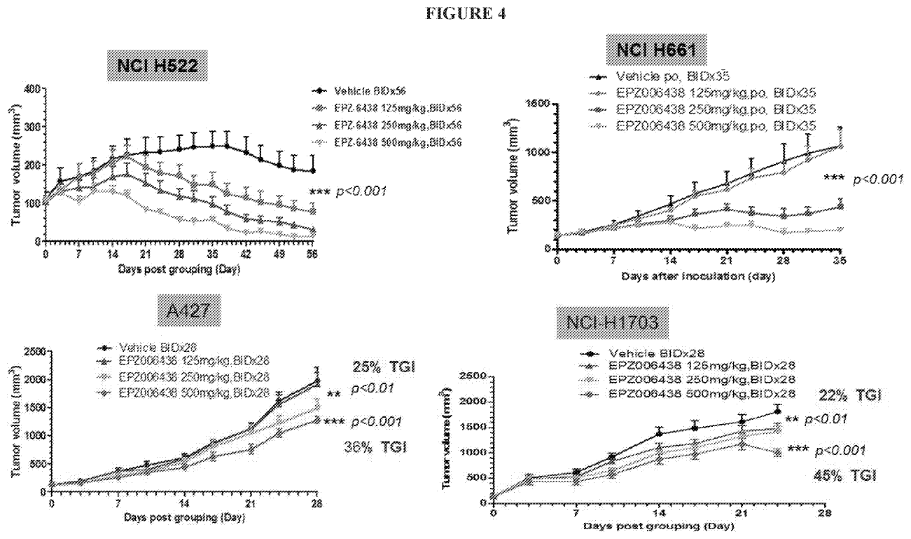

Both SMARCA4 single-loss and SMARCA2/SMARCA4 dual loss NSCLC cell lines were treated with the EZH2 inhibitor tazemetostat in vivo at clinically achievable dosage (˜250 mg/kg body weight) in the context of an NSCLC xenograft model (FIGS. 3 and 4). Consistent with the in vitro data, tumor growth inhibition was more prominent in SMARCA2/SMARCA4 dual loss xenografts than in SMARCA4 single loss xenografts. In two of the four SMARCA2/SMARCA4 dual loss cell lines, tumor regression was observed (FIG. 3).

### Discussion

The data provided herein demonstrate that a subtype of lung cancer, SMARCA2/SMARCA4 double loss NSCLC, can effectively be treated by EZH2 inhibition. Primary NSCLC tumors, including those exhibiting SMARCA2/SMARCA4 dual loss, are typically of the poorly-differentiated adenocarcinoma type (e.g., solid adenocarcinoma), and frequently exhibit epithelial to mesenchymal transition (EMT) features (e.g., low E-cadherin and high vimentin expression levels). These characteristics are consistent with features of rhabdoid tumors (e.g., poorly differentiated and mesenchymal-like), and thus point to a previously unrecognized rhabdoid-like subtype of NSCLC characterized by SMARCA2/SMARCA4 dual loss. Dual loss of SMARCA2 and SMARCA4 correlate with reduced survival in NSCLC patients (see, e.g., Reisman et al. Cancer Res 2003, incorporated herein by reference). In addition, dual loss tumors are frequently negative for other mutations associated with NSCLC (e.g., EGFR, KRAS, ALK fusions), thus limiting the available options for therapy. Accordingly, the SMARCA2/SMARCA4 double loss NSCLC tumor class represents a subtype of lung cancer with high unmet medical need. The present disclosure demonstrates that EZH2 inhibition is effective in inhibiting tumor growth and/or eliciting a desirable clinical outcome in such tumors.

### Exemplary Sequences

### SMARCA2

>NM_001289396.1 Homo sapiens SWI/SNF related, matrix associated, actin dependent regulator of chromatin, subfamily a, member 2 (SMARCA2), transcript variant 3, mRNA

(SEQ ID NO: 1)

TCAGAAGAAAGCCCCGAGATCACAGAGACCCGGCGAGATCACAGAGACCC
GGCCTGAAGGAACGTGGAAAGACCAATGTACCTGTTTTGACCGGTTGCCT
GGAGCAAGAAGTTCCAGTTGGGGAGAATTTTCAGAAGATAAAGTCGGAGA
TTGTGGAAAGACTTGACTTGCAGCATTAGTCTAGTGACTGGCAGAGACAG
GAGAGGTAGATGTCCACGCCCACAGACCCTGGTGCGATGCCCCACCCAGG
GCCTTCGCCGGGGCCTGGGCCTTCCCCTGGGCCAATTCTTGGGCCTAGTC
CAGGACCAGGACCATCCCCAGGTTCCGTCCACAGCATGATGGGGCCAAGT
GCTGGACCTCCAAGTGTCTCCCATCCTATGCCGACGATGGGGTCCACAGA
CTTCCCACAGGAAGGCATGCATCAAATGCATAAGCCCATCGATGGTATAC
ATGACAAGGGGATTGTAGAAGACATCCATTGTGGATCCATGAAGGGCACT
GGTATGCGACCACCTCACCCAGGCATGGGCCCTCCCCAGAGTCCAATGGA
TCAACACAGCCAAGGTTATATGTCACCACACCCATCTCCATTAGGAGCCC
CAGAGCACGTCTCCAGCCCTATGTCTGGAGGAGGCCCAACTCCACCTCAG
ATGCCACCAAGCCAGCCGGGGGCCCTCATCCCAGGTGATCCGCAGGCCAT
GAGCCAGCCCAACAGAGGTCCCTCACCTTTCAGTCCTGTCCAGCTGCATC
AGCTTCGAGCTCAGATTTTAGCTTATAAAATGCTGGCCCGAGGCCAGCCC
CTCCCCGAAACGCTGCAGCTTGCAGTCCAGGGGAAAAGGACGTTGCCTGG
CTTGCAGCAACAACAGCAGCAGCAACAGCAGCAGCAGCAGCAGCAGCAGC
AGCAGCAGCAGCAGCAACAGCAGCCGCAGCAGCAGCCGCCGCAACCACAG
ACGCAGCAACAACAGCAGCCGGCCCTTGTTAACTACAACAGACCATCTGG
CCCGGGGCCGGAGCTGAGCGGCCCGAGCACCCCGGAGAAGCTGCCGGTGC
CCGCGCCCGGCGGCCGGCCCTCGCCCGCGCCCCCCGCAGCCGCGCAGCCG
CCCGCGGCCGCAGTGCCCGGGCCCTCAGTGCCGCAGCCGGCCCCGGGGCA
GCCCTCGCCCGTCCTCCAGCTGCAGCAGAAGCAGAGCCGCATCAGCCCCA
TCCAGAAACCGCAAGGCCTGGACCCCGTGGAAATTCTGCAAGAGCGGGAA
TACAGACTTCAGGCCCGCATAGCTCATAGGATACAAGAACTGGAAAATCT
GCCTGGCTCTTTGCCACCAGATTTAAGAACCAAAGCAACCGTGGAACTAA
AAGCACTTCGGTTAGTCAATTTCCAGCGTCAGCTGAGACAGGAGGTGGTG
GCCTGCATGCGCAGGGACACGACCCTGGAGACGGCTCTCAACTCCAAAGC
ATACAAACGGAGCAAGCGCCAGACTCTGAGAGAAGCTCGCATGACCGAGA
AGCTGGAGAAGCAGCAGAAGATTGAGCAGGAGAGGAAACGCCGTCAGAAA
CACCAGGAATACCTGAACAGTATTTTGCAACATGCAAAAGATTTTAAGGA
ATATCATCGGTCTGTGGCCGGAAAGATCCAGAAGCTCTCCAAAGCAGTGG
CAACTTGGCATGCCAACACTGAAAGAGAGCAGAAGAAGGAGACAGAGCGG
ATTGAAAAGGAGAGAATGCGGCGACTGATGGCTGAAGATGAGGAGGGTTA
TAGAAAACTGATTGATCAAAAGAAAGACAGGCGTTTAGCTTACCTTTTGC
AGCAGACCGATGAGTATGTAGCCAATCTGACCAATCTGGTTTGGGAGCAC
AAGCAAGCCCAGGCAGCCAAAGAGAAGAAGAAGAGGAGGAGGAGGAAGAA
GAAGGCTGAGGAGAATGCAGAGGGTGGGGAGTCTGCCCTGGGACCGGATG
GAGAGCCCATAGATGAGAGCAGCCAGATGAGTGACCTCCCTGTCAAAGTG
ACTCACACAGAAACCGGCAAGGTTCTGTTCGGACCAGAAGCACCCAAAGC
AAGTCAGCTGGACGCCTGGCTGGAAATGAATCCTGGTTATGAAGTTGCCC
CTAGATCTGACAGTGAAGAGAGTGATTCTGATTATGAGGAAGAGGATGAG
GAAGAAGAGTCCAGTAGGCAGGAAACCGAAGAGAAAATACTCCTGGATCC
AAATAGCGAAGAAGTTTCTGAGAAGGATGCTAAGCAGATCATTGAGACAG
CTAAGCAAGACGTGGATGATGAATACAGCATGCAGTACAGTGCCAGGGGC
TCCCAGTCCTACTACACCGTGGCTCATGCCATCTCGGAGAGGGTGGAGAA
ACAGTCTGCCCTCCTAATTAATGGGACCCTAAAGCATTAGCAGCTCCAGG
GCCTGGAATGGATGGTTTCCCTGTATAATAACAACTTGAACGGAATCTTA
GCCGATGAAATGGGGCTTGGAAAGACCATACAGACCATTGCACTCATCAC
TTATCTGATGGAGCACAAAAGACTCAATGGCCCCTATCTCATCATTGTTC
CCCTTTCGACTCTATCTAACTGGACATATGAATTTGACAAATGGGCTCCT
TCTGTGGTGAAGATTTCTTACAAGGGTACTCCTGCCATGCGTCGCTCCCT
TGTCCCCCAGCTACGGAGTGGCAAATTCAATGTCCTCTTGACTACTTATG
AGTATATTATAAAAGACAAGCACATTCTTGCAAAGATTCGGTGGAAATAC
ATGATAGTGGACGAAGGCCACCGAATGAAGAATCACCACTGCAAGCTGAC
TCAGGTCTTGAACACTCACTATGTGGCCCCCAGAAGGATCCTCTTGACTG
GGACCCCGCTGCAGAATAAGCTCCCTGAACTCTGGGCCCTCCTCAACTTC
CTCCTCCCAACAATTTTTAAGAGCTGCAGCACATTTGAACAATGGTTCAA
TGCTCCATTTGCCATGACTGGTGAAAGGGTGGACTTAAATGAAGAAGAAA
CTATATTGATCATCAGGCGTCTACATAAGGTGTTAAGACCATTTTTACTA
AGGAGACTGAAGAAAGAAGTTGAATCCCAGCTTCCCGAAAAAGTGGAATA
TGTGATCAAGTGTGACATGTCAGCTCTGCAGAAGATTCTGTATCGCCATA
TGCAAGCCAAGGGGATCCTTCTCACAGATGGTTCTGAGAAAGATAAGAAG
GGGAAAGGAGGTGCTAAGACACTTATGAACACTATTATGCAGTTGAGAAA
AATCTGCAACCACCCATATATGTTTCAGCACATTGAGGAATCCTTTGCTG
AACACCTAGGCTATTCAAATGGGGTCATCAATGGGGCTGAACTGTATCGG
GCCTCAGGGAAGTTTGAGCTGCTTGATCGTATTCTGCCAAAATTGAGAGC
GACTAATCACCGAGTGCTGCTTTTCTGCCAGATGACATCTCTCATGACCA
TCATGGAGGATTATTTTGCTTTTCGGAACTTCCTTTACCTACGCCTTGAT
GGCACCACCAAGTCTGAAGATCGTGCTGCTTTGCTGAAGAAATTCAATGA
ACCTGGATCCCAGTATTTCATTTTCTTGCTGAGCACAAGAGCTGGTGGCC
TGGGCTTAAATCTTCAGGCAGCTGATACAGTGGTCATCTTTGACAGCGAC
TGGAATCCTCATCAGGATCTGCAGGCCCAAGACCGAGCTCACCGCATCGG
GCAGCAGAACGAGGTCCGGGTACTGAGGCTCTGTACCGTGAACAGCGTGG
AGGAAAAGATCCTCGCGGCCGCAAAATACAAGCTGAACGTGGATCAGAAA
GTGATCCAGGCGGGCATGTTTGACCAAAAGTCTTCAAGCCACGAGCGGAG
GGCATTCCTGCAGGCCATCTTGGAGCATGAGGAGGAAAATGAGGAAGAAG
ATGAAGTACCGGACGATGAGACTCTGAACCAAATGATTGCTCGACGAGAA
GAAGAATTTGACCTTTTTATGCGGATGGACATGGACCGGCGGAGGGAAGA
TGCCCGGAACCCGAAACGGAAGCCCCGTTTAATGGAGGAGGATGAGCTGC
CCTCCTGGATCATTAAGGATGACGCTGAAGTAGAAAGGCTCACCTGTGAA
GAAGAGGAGGAGAAAATATTTGGGAGGGGGTCCCGCCAGCGCCGTGACGT
GGACTACAGTGACGCCCTCACGGAGAAGCAGTGGCTAAGGGCCATCGAAG
ACGGCAATTTGGAGGAAATGGAAGAGGAAGTACGGCTTAAGAAGCGAAAA
AGACGAAGAAATGTGGATAAAGATCCTGCAAAAGAAGATGTGGAAAAAGC
TAAGAAGAGAAGAGGCCGCCCTCCCGCTGAGAAACTGTCACCAAATCCCC
CCAAACTGACAAAGCAGATGAACGCTATCATCGATACTGTGATAAACTAC
AAAGATAGGTGTAACGTGGAGAAGGTGCCCAGTAATTCTCAGTTGGAAAT
AGAAGGAAACAGTTCAGGGCGACAGCTCAGTGAAGTCTTCATTCAGTTAG
CTTCAAGGAAAGAATTACCAGAATACTATGAATTAATTAGGAAGCCAGTG
GATTTCAAAAAAATAAAGGAAAGGATTCGTAATCATAAGTACCGGAGCCT
AGGCCACCTGGAGAAGGATGTCATGCTTCTCTGTCACAACGCTCAGACGT
TCAACCTGGAGGGATCCCAGATCTATGAAGACTCCATCGTCTTACAGTCA
GTGTTTAAGAGTGCCCGGCAGAAAATTGCCAAAGAGGAAGAGAGTGAGGA
TGAAAGCAATGAAGAGGAGGAAGAGGAAGATGAAGAAGAGTCAGAGTCCG
AGGCAAAATCAGTCAAGGTGAAAATTAAGCTCAATAAAAAAGATGACAAA
GGCCGGGACAAAGGGAAAGGCAAGAAAAGGCCAAATCGAGGAAAAGCCAA
ACCTGTAGTGAGCGATTTTGACAGCGATGAGGAGCAGGATGAACGTGAAC
AGTCAGAAGGAAGTGGGACGGATGATGAGTGATCAGTATGGACCTTTTTC
CTTGGTAGAACTGAATTCCTTCCTCCCCTGTCTCATTTCTACCCAGTGAG
TTCATTTGTCATATAGGCACTGGGTTGTTTCTATATCATCATCGTCTATA
AACTAGCTTTAGGATAGTGCCAGACAAACATATGATATCATGGTGTAAAA
AACACACACATACACAAATATTTGTAACATATTGTGACCAAATGGGCCTC
AAAGATTCAGATTGAAACAAACAAAAAGCTTTTGATGGAAAATATGTGGG
TGGATAGTATATTTCTATGGGTGGGTCTAATTTGGTAACGGTTTGATTGT
GCCTGGTTTTATCACCTGTTCAGATGAGAAGATTTTTGTCTTTTGTAGCA
CTGATAACCAGGAGAAGCCATTAAAAGCCACTGGTTATTTTATTTTTCAT
CAGGCAATTTTCGAGGTTTTTATTTGTTCGGTATTGTTTTTTTACACTGT
GGTACATATAAGCAACTTTAATAGGTGATAAATGTACAGTAGTTAGATTT
CACCTGCATATACATTTTTCCATTTTATGCTCTATGATCTGAACAAAAGC
TTTTTGAATTGTATAAGATTTATGTCTACTGTAAACATTGCTTAATTTTT
TTGCTCTTGATTTAAAAAAAAGTTTTGTTGAAAGCGCTATTGAATATTGC
AATCTATATAGTGTATTGGATGGCTTCTTTTGTCACCCTGATCTCCTATG
TTACCAATGTGTATCGTCTCCTTCTCCCTAAAGTGTACTTAATCTTTGCT
TTCTTTGCACAATGTCTTTGGTTGCAAGTCATAAGCCTGAGGCAAATAAA
ATTCCAGTAATTTCGAAGAATGTGGTGTTGGTGCTTTCCTAATAAAGAAA
TAATTTAGCTTGACAAAAAAAAAAAAAAA.

>NM_139045.3 Homo sapiens SWI/SNF related, matrix associated, actin dependent regulator of chromatin, subfamily a, member 2 (SMARCA2), transcript variant 2, mRNA

(SEQ ID NO: 2)

GCGTCTTCCGGCGCCCGCGGAGGAGGCGAGGGTGGGACGCTGGGCGGAGC
CCGAGTTTAGGAAGAGGAGGGGACGGCTGTCATCAATGAAGTCATATTCA
TAATCTAGTCCTCTCTCCCTCTGTTTCTGTACTCTGGGTGACTCAGAGAG
GGAAGAGATTCAGCCAGCACACTCCTCGCGAGCAAGCATTACTCTACTGA
CTGGCAGAGACAGGAGAGGTAGATGTCCACGCCCACAGACCCTGGTGCGA
TGCCCCACCCAGGGCCTTCGCCGGGGCCTGGGCCTTCCCCTGGGCCAATT
CTTGGGCCTAGTCCAGGACCAGGACCATCCCCAGGTTCCGTCCACAGCAT
GATGGGGCCAAGTCCTGGACCTCCAAGTGTCTCCCATCCTATGCCGACGA
TGGGGTCCACAGACTTCCCACAGGAAGGCATGCATCAAATGCATAAGCCC
ATCGATGGTATACATGACAAGGGGATTGTAGAAGACATCCATTGTGGATC
CATGAAGGGCACTGGTATGCGACCACCTCACCCAGGCATGGGCCCTCCCC
AGAGTCCAATGGATCAACACAGCCAAGGTTATATGTCACCACACCCATCT
CCATTAGGAGCCCCAGAGCACGTCTCCAGCCCTATGTCTGGAGGAGGCCC
AACTCCACCTCAGATGCCACCAAGCCAGCCGGGGGCCCTCATCCCAGGTG
ATCCGCAGGCCATGAGCCAGCCCAACAGAGGTCCCTCACCTTTCAGTCCT
GTCCAGCTGCATCAGCTTCGAGCTCAGATTTTAGCTTATAAAATGCTGGC
CCGAGGCCAGCCCCTCCCCGAAACGCTGCAGCTTGCAGTCCAGGGGAAAA
GGACGTTGCCTGGCTTGCAGCAACAACAGCAGCAGCAACAGCAGCAGCAG
CAGCAGCAGCAGCAGCAGCAGCAGCAGCAACAGCAGCCGCAGCAGCAGCC
GCCGCAACCACAGACGCAGCAACAACAGCAGCCGGCCCTTGTTAACTACA
ACAGACCATCTGGCCCGGGGCCGGAGCTGAGCGGCCCGAGCACCCCGCAG
AAGCTGCCGGTGCCCGCGCCCGGCGGCCGGCCCTCGCCCGCGCCCCCCGC
AGCCGCGCAGCCGCCCGCGGCCGCAGTGCCCGGGCCCTCAGTGCCGCAGC
CGGCCCCGGGGCAGCCCTCGCCCGTCCTCCAGCTGCAGCAGAAGCAGAGC
CGCATCAGCCCCATCCAGAAACCGCAAGGCCTGGACCCCGTGGAAATTCT
GCAAGAGCGGGAATACAGACTTCAGGCCCGCATAGCTCATAGGATACAAG
AACTGGAAAATCTGCCTGGCTCTTTGCCACCAGATTTAAGAACCAAAGCA
ACCGTGGAACTAAAAGCACTTCGGTTACTCAATTTCCAGCGTCAGCTGAG
ACAGGAGGTGGTGGCCTGCATGCGCAGGGACACGACCCTGGAGACGGCTC
TCAACTCCAAAGCATACAAACGGAGCAAGCGCCAGACTCTGAGAGAAGCT
CGCATGACCGAGAAGCTGGAGAAGCAGCAGAAGATTGAGCAGGAGAGGAA
ACGCCGTCAGAAACACCAGGAATACCTGAACAGTATTTTGCAACATGCAA
AAGATTTTAAGGAATATCATCGGTCTGTGGCCGGAAAGATCCAGAAGCTC
TCCAAAGCAGTGGCAACTTGGCATGCCAACACTGAAAGAGAGCAGAAGAA
GGAGACAGAGCGGATTGAAAAGGAGAGAATGCGGCGACTGATGGCTGAAG
ATGAGGAGGGTTATAGAAAACTGATTGATCAAAAGAAAGACAGGCGTTTA
GCTTACCTTTTGCAGCAGACCGATGAGTATGTAGCCAATCTGACCAATCT
GGTTTGGGAGCACAAGCAAGCCCAGGCAGCCAAAGAGAAGAAGAAGAGGA
GGAGGAGGAAGAAGAAGGCTGAGGAGAATGCAGAGGGTGGGGAGTCTGCC
CTGGGACCGGATGGAGAGCCCATAGATGAGAGCAGCCAGATGAGTGACCT
CCCTGTCAAAGTGACTCACACAGAAACCGGCAAGGTTCTGTTCGGACCAG
AAGCACCCAAAGCAAGTCAGCTGGACGCCTGGCTGGAAATGAATCCTGGT
TATGAAGTTGCCCCTAGATCTGACAGTGAAGAGAGTGATTCTGATTATGA
GGAAGAGGATGAGGAAGAAGAGTCCAGTAGGCAGGAAACCGAAGAGAAAA
TACTCCTGGATCCAAATAGCGAAGAAGTTTCTGAGAAGGATGCTAAGCAG
ATCATTGAGACAGCTAAGCAAGACGTGGATGATGAATACAGCATGCAGTA
CAGTGCCAGGGGCTCCCAGTCCTACTACACCGTGGCTCATGCCATCTCGG
AGAGGGTGGAGAAACAGTCTGCCCTCCTAATTAATGGGACCCTAAAGCAT
TAGCAGCTCCAGGGCCTGGAATGGATGGTTTCCCTGTATAATAACAACTT
GAACGGAATCTTAGCCGATGAAATGGGGCTTGGAAAGACCATACAGACCA
TTGCACTCATCACTTATCTGATGGAGCACAAAAGACTCAATGGCCCCTAT
CTCATCATTGTTCCCCTTTCGACTCTATCTAACTGGACATATCAATTTGA
CAAATGGGCTCCTTCTGTGGTGAAGATTTCTTACAAGGGTACTCCTGCCA
TGCGTCGCTCCCTTGTCCCCCAGCTACGGAGTGGCAAATTCAATGTCCTC
TTGACTACTTATGAGTATATTATAAAAGACAAGCACATTCTTGCAAAGAT
TCGGTGGAAATACATGATAGTGGACGAAGGCCACCGAATGAAGAATCACC
ACTGCAAGCTGACTCAGGTCTTGAACACTCACTATGTGGCCCCCAGAAGG
ATCCTCTTGACTGGGACCCCGCTGCAGAATAAGCTCCCTGAACTCTGGGC
CCTCCTCAACTTCCTCCTCCCAACAATTTTTAAGAGCTGCAGCACATTTG
AACAATGGTTCAATGCTCCATTTGCCATCACTGGTGAAAGGGTGCACTTA
AATGAAGAAGAAACTATATTGATCATCAGGCGTCTACATAAGGTGTTAAG
ACCATTTTTACTAAGGAGACTGAAGAAAGAAGTTGAATCCCAGCTTCCCG
AAAAAGTGGAATATGTGATCAAGTGTGACATGTCAGCTCTGCAGAAGATT
CTGTATCGCCATATGCAAGCCAAGGGGATCCTTCTCACAGATGGTTCTGA
GAAAGATAAGAAGGGGAAAGGAGGTGCTAAGACACTTATGAACACTATTA
TGCAGTTGAGAAAAATCTGCAACCACCCATATATGTTTCAGCACATTGAG
GAATCCTTTGCTGAACACCTAGGCTATTCAAATGGGGTCATCAATGGGGC
TGAACTGTATCGGGCCTCAGGGAAGTTTGAGCTGCTTGATCGTATTCTGC
CAAAATTGAGAGCGACTAATCACCGAGTGCTGCTTTTCTGCCAGATGACA
TCTCTCATGACCATCATGGAGGATTATTTTGCTTTTCGGAACTTCCTTTA
CCTACGCCTTGATGGCACCACCAAGTCTGAAGATCGTGCTGCTTTGCTGA
AGAAATTCAATGAACCTGGATCCCAGTATTTCATTTTCTTGCTGAGCACA
AGAGCTGGTGGCCTGGGCTTAAATCTTCAGGCAGCTGATACAGTGGTCAT
CTTTGACAGCGACTGGAATCCTCATCAGCATCTGCAGGCCCAAGACCGAG
CTCACCGCATCGGGCAGCAGAACGAGGTCCGGGTACTGAGGCTCTGTACC
GTGAACAGCGTGGAGGAAAAGATCCTCGCGGCCGCAAAATACAAGCTGAA
CGTGGATCAGAAAGTGATCCAGGCGGGCATGTTTGACCAAAAGTCTTCAA
GCCACGAGCGGAGGGCATTCCTGCAGGCCATCTTGGAGCATGAGGAGGAA
AATGAGGAAGAAGATGAAGTACCGGACGATGAGACTCTGAACCAAATGAT
TGCTCGACGAGAAGAAGAATTTGACCTTTTTATGCGGATGGACATGGACC
GGCGGAGGCAAGATGCCCGGAACCCGAAACGGAAGCCCCGTTTAATGGAG
GAGGATGAGCTGCCCTCCTGGATCATTAAGGATGACGCTGAAGTAGAAAG
GCTCACCTGTGAAGAAGAGGAGGAGAAAATATTTGGGAGGGGGTCCCGCC
AGCGCCGTGACGTGGACTACAGTGACGCCCTCACGGAGAAGCAGTGGCTA
AGGGCCATCGAAGACGGCAATTTGGAGGAAATGGAAGAGGAAGTACGGCT
TAAGAAGCGAAAAAGACGAAGAAATGTGGATAAAGATCCTGCAAAAGAAG
ATGTGGAAAAAGCTAAGAAGAGAAGAGGCCGCCCTCCCGCTGAGAAACTG
TCACCAAATCCCCCCAAACTGACAAAGCAGATGAACGCTATCATCGATAC
TGTGATAAACTACAAAGATAGTTCAGGGCGACAGCTCAGTGAAGTCTTCA
TTCAGTTACCTTCAAGGAAAGAATTACCAGAATACTATGAATTAATTAGG
AAGCCAGTGGATTTCAAAAAAATAAAGGAAAGGATTCGTAATCATAAGTA
CCGGAGCCTAGGCGACCTGGAGAAGGATGTCATGCTTCTCTGTCACAACG
CTCAGACGTTCAACCTGGAGGGATCCCAGATCTATGAAGACTCCATCGTC
TTACAGTCAGTGTTTAAGAGTGCCCGGCAGAAAATTGCCAAAGAGGAAGA
GAGTGAGGATGAAAGCAATGAAGAGGAGGAAGAGGAAGATGAAGAAGAGT
CAGAGTCCGAGGCAAAATCAGTCAAGGTGAAAATTAAGCTCAATAAAAAA
GATGACAAAGGCCGGGACAAAGGGAAAGGCAAGAAAAGGCCAAATCGAGG
AAAAGCCAAACCTGTAGTGAGCGATTTTGACAGCGATGAGGAGCAGGATG
AACGTGAACAGTCAGAAGGAAGTGGGACGGATGATGAGTGATCAGTATGG
ACCTTTTTCCTTGGTAGAACTGAATTCCTTCCTCCCCTGTCTCATTTCTA
CCCAGTGAGTTCATTTGTCATATAGGCACTGGGTTGTTTCTATATCATCA
TCGTCTATAAACTAGCTTTAGGATAGTGCCAGACAAACATATGATATCAT
GGTGTAAAAAACACACACATACACAAATATTTGTAACATATTGTGACCAA
ATGGGCCTCAAAGATTCAGATTGAAACAAACAAAAAGCTTTTGATGGAAA
ATATGTGGGTGGATAGTATATTTCTATGGGTGGGTCTAATTTGGTAACGG
TTTGATTGTGCCTGGTTTTATCACCTGTTCAGATGAGAAGATTTTTGTCT
TTTGTAGCACTGATAACCAGGAGAAGCCATTAAAAGCCACTGGTTATTTT
ATTTTTCATCAGGCAATTTTCGAGGTTTTTATTTGTTCGGTATTGTTTTT
TTACACTGTGGTACATATAAGCAACTTTAATAGGTGATAAATGTACAGTA
GTTAGATTTCACCTGCATATACATTTTTCCATTTTATGCTCTATGATCTG
AACAAAAGCTTTTTGAATTGTATAAGATTTATGTCTACTGTAAACATTGC
TTAATTTTTTTGCTCTTGATTTAAAAAAAAGTTTTGTTGAAAGCGCTATT
GAATATTGCAATCTATATAGTGTATTGGATGGCTTCTTTTGTCACCCTGA
TCTCCTATGTTACCAATGTGTATCGTCTCCTTCTCCCTAAAGTGTACTTA
ATCTTTGCTTTCTTTGCACAATGTCTTTGGTTGCAAGTCATAAGCCTGAG
GCAAATAAAATTCCAGTAATTTCGAAGAATGTGGTGTTGGTGCTTTCCTA
ATAAAGAAATAATTTAGCTTGACAAAAAAAAAAAAAAA.

>NM_001289397.1 Homo sapiens SWI/SNF related, matrix associated, actin dependent regulator of chromatin, subfamily a, member 2 (SMARCA2), transcript variant 4, mRNA

(SEQ ID NO: 3)

GCGTCTTCCGGCGCCCGCGGAGGAGGCGAGGGTGGGACGCTGGGCGGAGC
CCGAGTTTAGGAAGAGGAGGGGACGGCTGTCATCAATGAAGTCATATTCA
TAATCTAGTCCTCTCTCCCTCTGTTTCTGTACTCTGGGTGACTCAGAGAG
GGAAGAGATTCAGCCAGCACACTCCTCGCGAGCAAGCATTAGTCTACTGA
CTGGCAGAGACAGGAGAGGTAGATGTCCACGCCCACAGACCCTGGTGCGA
TGCCCCACCCAGGGCCTTCGCCGGGGCCTGGGCCTTCCCCTGGGCCAATT
CTTGGGCCTAGTCCAGGACCAGGACCATCCCCAGGTTCCGTCCACAGCAT
GATGGGGCCAAGTCCTGGACCTCCAAGTGTCTCCCATCCTATGCCGACGA
TGGGGTCCACAGACTTCCCACAGGAAGGCATGCATCAAATGCATAAGCCC
ATCGATGGTATACATGACAAGGGGATTGTAGAAGACATCCATTGTGGATC
CATGAAGGGCACTGGTATGCGACCACCTCACCCAGGCATGGGCCCTCCCC
AGAGTCCAATGGATCAACACAGCCAAGGTTATATGTCACCACACCCATCT
CCATTAGGAGCCCCAGAGCACGTCTCCAGCCCTATGTCTGGAGGAGGCCC
AACTCCACCTCAGATGCCACCAAGCCAGCCGGGGGCCCTCATCCCAGGTG
ATCCGCAGGCCATGAGCCAGCCCAACAGAGGTCCCTCACCTTTCAGTCCT
GTCCAGCTGCATCAGCTTCGAGCTCAGATTTTAGCTTATAAAATGCTGGC
CCGAGGCCAGCCCCTCCCCGAAACGCTGCAGCTTGCAGTCCAGGGGAAAA
GGACGTTGCCTGGCTTGCAGCAACAACAGCAGCAGCAACAGCAGCAGCAG
CAGCAGCAGCAGCAGCAGCAGCAGCAGCAACAGCAGCCGCAGCAGCAGCC
GCCGCAACCACAGACGCAGCAACAACAGCAGCCGGCCCTTGTTAACTACA
ACAGACCATCTGGCCCGGGGCCGGAGCTGAGCGGCCCGAGCACCCCGCAG
AAGCTGCCGGTGCCCGCGCCCGGCGGCCGGCCCTCGCCCGCGCCCCCCGC
AGCCGCGCAGCCGCCCGCGGCCGCAGTGCCCGGGCCCTCAGTGCCGCAGC
CGGCCCCGGGGCAGCCCTCGCCCGTCCTCCAGCTGCAGCAGAAGCAGAGC
CGCATCAGCCCCATCCAGAAACCGCAAGGCCTGGACCCCGTGGAAATTCT
GCAAGAGCGGGAATACAGACTTCAGGCCCGCATAGCTCATAGGATACAAG
AACTGGAAAATCTGCCTGGCTCTTTGCCACCAGATTTAAGAACCAAAGCA
ACCGTGGAACTAAAAGCACTTCGGTTACTCAATTTCCAGCGTCAGCTGAG
ACAGGAGGTGGTGGCCTGCATGCGCAGGGACACGACCCTGGAGACGGCTC
TCAACTCCAAAGCATACAAACGGAGCAAGCGCCAGACTCTGAGAGAAGCT
CGCATGACCGAGAAGCTGGAGAAGCAGCAGAAGATTGAGCAGGAGAGGAA
ACGCCGTCAGAAACACCAGGAATACCTGAACAGTATTTTGCAACATGCAA
AAGATTTTAAGGAATATCATCGGTCTGTGGCCGGAAAGATCCAGAAGCTC
TCCAAAGCAGTGGCAACTTGGCATGCCAACACTGAAAGAGAGCAGAAGAA
GGAGACAGAGCGGATTGAAAAGGAGAGAATGCGGCGACTGATGGCTGAAG
ATGAGGAGGGTTATAGAAAACTGATTGATCAAAAGAAAGACAGGCGTTTA
GCTTACCTTTTGCAGCAGACCGATGAGTATGTAGCCAATCTGACCAATCT
GGTTTGGGAGCACAAGCAAGCCCAGGCAGCCAAAGAGAAGAAGAAGAGGA
GGAGGAGGAAGAAGAAGGCTGAGGAGAATGCAGAGGGTGGGGAGTCTGCC
CTGGGACCGGATGGAGAGCCCATAGATGAGAGCAGCCAGATGAGTGACCT
CCCTGTCAAAGTGACTCACACAGAAACCGGCAAGGTTCTGTTCGGACCAG
AAGCACCCAAAGCAAGTCAGCTGGACGCCTGGCTGGAAATGAATCCTGGT
TATGAAGTTGCCCCTAGATCTGACAGTGAAGAGAGTGATTCTGATTATGA
GGAAGAGGATGAGGAAGAAGAGTCCAGTAGGCAGGAAACCGAAGAGAAAA
TACTCCTGGATCCAAATAGCGAAGAAGTTTCTGAGAAGGATGCTAAGCAG
ATCATTGAGACAGCTAAGCAAGACGTGGATGATGAATACAGCATGCAGTA
CAGTGCCAGGGGCTCCCAGTCCTAGTACACCGTGGCTCATGCCATCTCGG
AGAGGGTGGAGAAACAGTCTGCCCTCCTAATTAATGGGACCCTAAAGCAT
TACCAGCTCCAGGGCCTGGAATGGATGGTTTCCCTGTATAATAACAACTT
GAACGGAATCTTAGCCGATGAAATGGGGCTTGGAAAGACCATACAGACCA
TTGCACTCATCACTTATCTGATGGAGCACAAAAGACTCAATGGCCCCTAT
CTCATCATTGTTCCCCTTTCGACTCTATCTAACTGGACATATGAATTTGA
CAAATGGGCTCCTTCTGTGGTGAAGATTTCTTACAAGGGTACTCCTGCCA
TGCGTCGCTCCCTTGTCCCCCAGCTAGGGAGTGGCAAATTCAATGTCCTC
TTGACTAGTTATGAGTATATTATAAAAGACAAGCACATTCTTGCAAAGAT
TCGGTGGAAATACATGATAGTGGACGAAGGCCACCGAATGAAGAATCACC
ACTGCAAGCTGACTCAGGTGGACTTAAATGAAGAAGAAACTATATTGATC
ATCAGGCGTCTACATAAGGTGTTAAGACCATTTTTACTAAGGAGACTGAA
GAAAGAAGTTGAATCCCAGCTTCCCGAAAAAGTGGAATATGTGATCAAGT
GTGACATGTCAGCTCTGCAGAAGATTCTGTATCGCCATATGCAAGCCAAG
GGGATCCTTCTCACAGATGGTTCTGAGAAAGATAAGAAGGGGAAAGGAGG
TGCTAAGACACTTATGAACACTATTATGCAGTTGAGAAAAATCTGCAACC
ACCCATATATGTTTCAGCACATTGAGGAATCCTTTGCTGAACACCTAGGC
TATTCAAATGGGGTCATCAATGGGGCTGAACTGTATCGGGCCTCAGGGAA
GTTTGAGCTGCTTGATCGTATTCTGCCAAAATTGAGAGCGACTAATCACC
GAGTGCTGCTTTTCTGCCAGATGACATCTCTCATGACCATCATGGAGGAT
TATTTTGCTTTTCGGAACTTCCTTTACCTAGGCCTTGATGGCACCACCAA
GTCTGAAGATCGTGCTGCTTTGCTGAAGAAATTCAATGAACCTGGATCCC
AGTATTTCATTTTCTTGCTGAGCACAAGAGCTGGTGGCCTGGGCTTAAAT
CTTCAGGCAGCTGATACAGTGGTCATCTTTGACAGCGACTGGAATCCTCA
TCAGGATCTGCAGGCCCAAGACCGAGCTCACCGCATCGGGCAGCAGAACG
AGGTCCGGGTACTGAGGCTCTGTACCGTGAACAGCGTGGAGGAAAAGATC
CTCGCGGCCGCAAAATACAAGCTGAACGTGGATCAGAAAGTGATCCAGGC
GGGCATGTTTGACCAAAAGTCTTCAAGCCACGAGCGGAGGGCATTCCTGC
AGGCCATCTTGGAGCATGAGGAGGAAAATGAGGAAGAAGATGAAGTACCG
GACGATGAGACTCTGAACCAAATGATTGCTCGACGAGAAGAAGAATTTGA
CCTTTTTATGCGGATGGACATGGACCGGCGGAGGGAAGATGCCCGGAACC
CGAAACGGAAGCCCCGTTTAATGGAGGAGGATGAGCTGCCCTCCTGGATC
ATTAAGGATGACGCTGAAGTAGAAAGGCTCACCTGTGAAGAAGAGGAGGA
GAAAATATTTGGGAGGGGGTCCCGCCAGCGCCGTGACGTGGACTACAGTG
ACGCCCTCACGGAGAAGCAGTGGCTAAGGGCCATCGAAGACGGCAATTTG
GAGGAAATGGAAGAGGAAGTACGGCTTAAGAAGCGAAAAAGACGAAGAAA
TGTGGATAAAGATCCTGCAAAAGAAGATGTGGAAAAAGCTAAGAAGAGAA
GAGGCCGCCCTCCCGCTGAGAAACTGTCACCAAATCCCCCCAAACTGACA
AAGCAGATGAACGCTATCATCGATACTGTGATAAACTACAAAGATAGTTC
AGGGCGACAGCTCAGTGAAGTCTTCATTCAGTTAGCTTCAAGGAAAGAAT
TACCAGAATACTATGAATTAATTAGGAAGCCAGTGGATTTCAAAAAAATA
AAGGAAAGGATTCGTAATCATAAGTACCGGAGCCTAGGCGACCTGGAGAA
GGATGTCATGCTTCTCTGTCACAACGCTCAGACGTTCAACCTGGAGGGAT
CCCAGATCTATGAAGACTCCATCGTCTTACAGTCAGTGTTTAAGAGTGCC
CGGCAGAAAATTGCCAAAGAGGAAGAGAGTGAGGATGAAAGCAATGAAGA
GGAGGAAGAGGAAGATGAAGAAGAGTCAGAGTCCGAGGCAAAATCAGTCA
AGGTGAAAATTAAGCTCAATAAAAAAGATGACAAAGGCCGGGACAAAGGG
AAAGGCAAGAAAAGGCCAAATCGAGGAAAAGCCAAACCTGTAGTGAGCGA
TTTTGACAGCGATGAGGAGCAGGATGAACGTGAACAGTCAGAAGGAAGTG
GGACGGATGATGAGTGATCAGTATGGACCTTTTTCCTTGGTAGAACTGAA
TTCCTTCCTCCCCTGTCTCATTTCTACCCAGTGAGTTCATTTGTCATATA
GGCACTGGGTTGTTTCTATATCATCATCGTCTATAAACTAGCTTTAGGAT
AGTGCCAGACAAACATATGATATCATGGTGTAAAAAACACACACATACAC
AAATATTTGTAACATATTGTGACCAAATGGGCCTCAAAGATTCAGATTGA
AACAAACAAAAAGCTTTTGATGGAAAATATGTGGGTGGATAGTATATTTC
TATGGGTGGGTCTAATTTGGTAACGGTTTGATTGTGCCTGGTTTTATCAC
CTGTTCAGATGAGAAGATTTTTGTCTTTTGTAGCACTGATAACCAGGAGA
AGCCATTAAAAGCCACTGGTTATTTTATTTTTCATCAGGCAATTTTCGAG
GTTTTTATTTGTTCGGTATTGTTTTTTTACACTGTGGTACATATAAGCAA
CTTTAATAGGTGATAAATGTACAGTAGTTAGATTTCACCTGCATATACAT
TTTTCCATTTTATGCTCTATGATCTGAACAAAAGCTTTTTGAATTGTATA
AGATTTATGTCTAGTGTAAACATTGCTTAATTTTTTTGCTCTTGATTTAA
AAAAAAGTTTTGTTGAAAGCGCTATTGAATATTGCAATCTATATAGTGTA
TTGGATGGCTTCTTTTGTCACCCTGATCTCCTATGTTACCAATGTGTATC
GTCTCCTTCTCCCTAAAGTGTACTTAATCTTTGCTTTCTTTGCACAATGT
CTTTGGTTGCAAGTCATAAGCCTGAGGCAAATAAAATTCCAGTAATTTCG
AAGAATGTGGTGTTGGTGCTTTCCTAATAAAGAAATAATTTAGCTTGACA
AAAAAAAAAAAAAA.

>NM_001289398.1 Homo sapiens SWI/SNF related, matrix associated, actin dependent regulator of chromatin, subfamily a, member 2 (SMARCA2), transcript variant 5, mRNA

(SEQ ID NO: 4)

CTTGGAGAGGCGGAGGTGGAAACGATGCGCAGGAGTTGGCTTGGGGCTTT
TTGTTTGCGTGTCCCTGTTTACCTATTCATAATCATGGATCCCCTCTGCT
TTGTGATACTGTGAACCACGCATAACAGCAATTCTTTAGACCACCGGGTT
GAGAAGAAGGCGCCTGAGGCTGACTTTCTGGACCTGCCGTCACGCAGTAA
AGATGTGGTTGGCCATCGAAGACGGCAATTTGCAGGAAATGGAAGAGGAA
GTACGGCTTAAGAAGCGAAAAAGACGAAGAAATGTGGATAAAGATGCTGC
AAAAGAAGATGTGGAAAAAGCTAAGAAGAGAAGAGGCCGCCCTCCCGCTG
AGAAACTGTCACCAAATCCCCCCAAACTGACAAAGCAGATGAACGCTATC
ATCGATACTGTGATAAACTACAAAGATAGTTCAGGGCGAGAGCTCAGTGA
AGTCTTCATTCAGTTAGCTTCAAGGAAAGAATTACCAGAATACTATGAAT
TAATTAGGAAGCCAGTGGATTTCAAAAAAATAAAGGAAAGGATTCGTAAT
CATAAGTAGCGGAGCCTAGGCGACCTGGAGAAGGATGTCATGCTTCTCTG
TCACAACGCTCAGACGTTCAACCTGGAGGGATCCCAGATCTATGAAGACT
CCATCGTCTTACAGTCAGTGTTTAAGAGTGCCCGGCAGAAAATTGCCAAA
GAGGAAGAGAGTGAGGATGAAAGCAATGAAGAGGAGGAAGAGGAAGATGA
AGAAGAGTCAGAGTCCGAGGGAAAATCAGTCAAGGTGAAAATTAAGCTCA
ATAAAAAAGATGACAAAGGCCGGGACAAAGGGAAAGGCAAGAAAAGGCCA
AATCGAGGAAAAGCCAAACCTGTAGTGAGCGATTTTGACAGCGATGAGGA
GCAGGATGAACGTGAACAGTCAGAAGGAAGTGGGACGGATGATGAGTGAT
CAGTATGGACCTTTTTCCTTGGTAGAACTGAATTCCTTCCTCCCCTGTCT
CATTTCTACCCAGTGAGTTCATTTGTCATATAGGCACTGGGTTGTTTCTA
TATCATCATCGTCTATAAACTAGCTTTAGGATAGTGCCAGACAAACATAT
GATATCATGGTGTAAAAAACACACACATACACAAATATTTGTAACATATT
GTGACCAAATGGGCCTCAAAGATTCAGATTGAAACAAACAAAAAGCTTTT
GATGGAAAATATGTGGGTGGATAGTATATTTCTATGGGTGGGTCTAATTT
GGTAACGGTTTGATTGTGCCTGGTTTTATCACCTGTTCAGATGAGAAGAT
TTTTGTCTTTTGTAGCACTGATAACCAGGAGAAGCCATTAAAAGCCACTG
GTTATTTTATTTTTCATCAGGCAATTTTCGAGGTTTTTATTTGTTCGGTA
TTGTTTTTTTACACTGTGGTACATATAAGCAACTTTAATAGGTGATAAAT
GTACAGTAGTTAGATTTCACCTGCATATACATTTTTCCATTTTATGCTCT
ATGATCTGAACAAAAGCTTTTTGAATTGTATAAGATTTATGTCTACTGTA
AACATTGCTTAATTTTTTTGCTCTTGATTTAAAAAAAAGTTTTGTTGAAA
GCGCTATTGAATATTGCAATCTATATAGTGTATTGGATGGCTTCTTTTGT
CACCCTGATCTCCTATGTTACCAATGTGTATCGTCTCCTTCTCCCTAAAG
TGTACTTAATCTTTGCTTTCTTTGCACAATGTCTTTGGTTGCAAGTCATA
AGCCTGAGGCAAATAAAATTCCAGTAATTTCGAAGAATGTGGTGTTGGTG
CTTTCCTAATAAAGAAATAATTTAGCTTGACAAAAAAAAAAAAAAA.

>NP_001276325.1 probable global transcription activator SNF2L2 isoform a [Homo sapiens]

(SEQ ID NO: 5)

MSTPTDPGAMPHPGPSPGPGPSPGPILGPSPGPGPSPGSVHSMMGPSPGP
PSVSHPMPTMGSTDFPQEGMHQMHKPIDGIHDKGIVEDIHCGSMKGTGMR
PPHPGMGPPQSPMDQHSQGYMSPHPSPLGAPEHVSSPMSGGGPTPPQMPP
SQPGALIPGDPQAMSQPNRGPSPFSPVQLHOLRAQILAYKMLARGQPLPE
TLQLAVQGKRTLPGLQQQQQQQQQQQQQQQQQQQQQQQPQQQPPQPQTQQ
QQQPALVNYNRPSGPGPELSGPSTPQKLPVPAPGGRPSPAPPAAAQPPAA
AVPGPSVPQPAPGQPSPVLQLQQKQSRISPIQKPQGLDPVEILQEREYRL
QARIAHRIQELENLPGSLPPDLRTKATVELKALRLLNFQRQLRQEVVACM
RRDTTLETALNSKAYKRSKRQTLREARMTEKLEKQQKIEQERKRRQKHQE
YLNSILQHAKDFKEYHRSVAGKIQKLSKAVATWHANTEREQKKETERIEK
ERMRRLMAEDEEGYRKLIDQKKDRRLAYLLQQTDEYVANLTNLVWEHKQA
QAAKEKKKRRRRKKKAEENAEGGESALGPDGEPIDESSQMSDLPVKVTHT
ETGKVLFGPEAPKASQLDAWLEMNPGYEVAPRSDSEESDSDYEEEDEEEE
SSRQETEEKILLDPNSEEVSEKDAKQIIETAKQDVDDEYSMQYSARGSQS
YYTVAHAISERVEKQSALLlNGTLKHYQLQGLEWMVSLYNNNLNGILADE
MGLGKTIQTIALITYLMEHKRLNGPYLIIVPLSTLSNWTYEFDKWAPSVV
KISYKGTPAMRRSLVPQLRSGKFNVLLTTYEYIIKDKHILAKIRWKYMIV
DEGHRMKNHHCKLTQVLNTHYVAPRRILLTGTPLQNKLPELWALLNFLLP
TIFKSCSTFEQWFNAPFAMTGERVDLNEEETILIIRRLHKVLRPFLLRRL
KKEVESQLPEKVEYVIKCDMSALQKILYRHMQAKGILLTDGSEKDKKGKG
GAKTLMNTIMQLRKICNHPYMFQHIEESFAEHLGYSNGVINGAELYRASG
KFELLDRILPKLRATNHRVLLFCQMTSLMTIMEDYFAFRNFLYLRLDGTT
KSEDRAALLKKFNEPGSQYFIFLLSTRAGGLGLNLQAADTVVIFDSDWNP
HQDLQAQDRAHRIGQQNEVRVLRLCTVNSVEEKILAAAKYKLNVDQKVIQ
AGMFDQKSSSHERRAFLQAILEHEEENEEEDEVPDDETLNQMIARREEEF
DLFMRMDMDRRREDARNPKRKPRLMEEDELPSWIIKDDAEVERLTCEEEE
EKIFGRGSRQRRDVDYSDALTEKQWLRAIEDGNLEEMEEEVRLKKRKRRR
NVDKDPAKEDVEKAKKRRGRPPAEKLSPNPPKLTKQMNAIIDTVINYKDR
CNVEKVPSNSQLEIEGNSSGRQLSEVFIQLPSRKELPEYYELIRKPVDFK
KIKERIRNHKYRSLGDLEKDVMLLCHNAQTFNLEGSQIYEDSIVLQSVFK
SARQKIAKEEESEDESNEEEEEEDEEESESEAKSVKVKIKLNKKDDKGRD
KGKGKKRPNRGKAKPVVSDFDSDEEQDEREQSEGSGTDDE.

>NP_620614.2 probable global transcription activator SNF2L2 isoform b [Homo sapiens]

(SEQ ID NO: 6)
MSTPTDPGAMPHPGPSPGPGPSPGPILGPSPGPGPSPGSVHSMMGPSPGP
PSVSHPMPTMGSTDFPQEGMHQMHKPIDGIHDKGIVEDIHCGSMKGTGMR
PPHPGMGPPQSPMDQHSQGYMSPHPSPLGAPEHVSSPMSGGGPTPPQMPP
SQPGALIPGDPQAMSQPNRGPSPFSPVQLHQLRAQILAYKMLARGQPLPE
TLQLAVQGKRTLPGLQQQQQQQQQQQQQQQQQQQQQQQPQQQPPQPQTQQ
QQQPALVNYNRPSGPGPELSGPSTPQKLPVPAPGGRPSPAPPAAAQPPAA
AVPGPSVPQPAPGQPSPVLQLQQKQSRISPIQKPQGLDPVEILQEREYRL
QARIAHRIQELENLPGSLPPDLRTKATVELKALRLLNFQRQLRQEVVACM
RRDTTLETALNSKAYKRSKRQTLREARMTEKLEKQQKIEQERKRRQKHQE
YLNSlLQHAKDFKEYHRSVAGKQKLSPCAVATWHANTEREQKKETERIEK
ERMRRLMAEDEEGYRKLIDQKKDRRIAYLLQQTDEYVANLTNLVWEHKQA
QAAKEKKKRRRRKKKAEENAEGGESALGPDGEPIDESSQMSDLPVKVTHT
ETGKVLFGPEAPKASQLDAWLEMNPGYEVAPRSDSEESDSDYEEEDEEEE
SSRQETEEKILLDPNSEEVSEKDAKQIIETAKQDVDDEYSMQYSARGSQS
YYTVAHAISERVEKQSALLINGTLKHYQLQGLEWMVSLYNNNLNGILADE
MGLGKTIQTIALITYLMEHKRLNGPYLIIVPLSTLSNWTYEFDKWAPSVV
KISYKGTPAMRRSLVPQLRSGKFNVLLTTYEYIIKDKHILAKIRWKYMIV
DEGHRMKNHHCKLTQVLNTHYVAPRRILLTGTPLQNKLPELWALLNFLLP
TIFKSCSTFEQWFNAPFAMTGERVDLNEEETILIIRRLHKVLRPFLLRRL
KKEVESQLPEKVEYVIKCDMSALQKILYRHMQAKGILLTDGSEKDKKGKG
GAKTLMNTIMQLRKICNHPYMFQHIEESFAEHLGYSNGVINGAELYRASG
KFELLDRILPKLRATNHRVLLFCQMTSLMTIMEDYFAFRNFLYLRLDGTT
KSEDRAALLKKFNEPGSQYFIFLLSTRAGGLGLNLQAADTVVIFDSDWNP
HQDLQAQDRAHRIGQQNEVRVLRLCTVNSVEEKILAAAKYKLNVDQKVIQ
AGMFDQKSSSHERRAFLQAILEHEEENEEEDEVPDDETLNQMIARREEEF
DLFMRMDMDRRREDARNPKRKPRLMEEDELPSWIIKDDAEVERLTCEEEE
EKTFGRGSRQRRDVDYSDALTEKQWLRAIEDGNLEEMEEEVRLKKRKRRR
NVDKDPAKEDVEKAKKRRGRPPAEKLSPNPPKLTKQMNATIDTVINYKDS
SGRQLSEVFIQLPSRKELPEYYELIRKPVDFKKIKERIRNHKYRSLGDLE
KDVMLLCHNAQTFNLEGSQIYEDSIVLQSVFKSARQKIAKEEESEDESNE
EEEEEDEEESESEAKSVKVKIKLNKKDDKGRDKGKGKKRPNRGKAKPWSD
FDSDEEQDEREQSEGSGTDDE.

>NP_001276326.1 probable global transcription activator SNF2L2 isoform c [Homo sapiens]

(SEQ ID NO: 7)
MSTPTDPGAMPHPGPSPGPGPSPGPILGPSPGPGPSPGSVHSMMGPSPGP
PSVSHPMPTMGSTDFPQEGMHQMHKPIDGIHDKGIVEDlHCGSMKGTGMR
PPHPGMGPPQSPMDQHSQGYMSPHPSPLGAPEHVSSPMSGGGPTPPQMPP
SQPGALIPGDPQAMSQPNRGPSPFSPVQLHQLRAQILAYKMLARGQPLPE
TLQLAVQGKRTLPGLQQQQQQQQQQQQQQQQQQQQQQQPQQQPPQPQTQQ
QQQPALVNYNRPSGPGPELSGPSTPQKLPVPAPGGRPSPAPPAAAQPPAA
AVPGPSVPQPAPGQPSPVLQLQQKQSRISPIQKPQGLDPVEILQEREYRL
QARIAHRIQELENLPGSLPPDLRTKATVELKALRLLNFQRQLRQEVVACM
RRDTTLETALNSKAYKRSKRQTLREARMTEKLEKQQKIEQERKRRQKHQE
YLNSILQHAKDFKEYHRSVAGKIQKLSKAVATWHANTEREQKKETERIEK
ERMRRLMAEDEEGYRKLIDQKKDRRLAYLLQQTDEYVANLTNLVWEHKQA
QAAKEKKKRRRRKKKAEENAEGGESALGPDGEPIDESSQMSDLPVKVTHT
ETGKVLFGPEAPKASQLDAWLEMNPGYEVAPRSDSEESDSDYEEEDEEEE
SSRQETEEKILLDPNSEEVSEKDAKQIIETAKQDVDDEYSMQYSARGSQS
YYTVAHAISERVEKQSALLINGTLKHYQLQGLEWMVSLYNNNLNGILADE
MGLGKTIQTIALITYLMEHKRLNGPYLIIVPLSTLSNWTYEFDKWAPSVV
KISYKGTPAMRRSLVPQLRSGKFNVLLTTYEYIIKDKHILAKIRWKYMIV
DEGHRMKNHHCKLTQVDLNEEETILIIRRLHKVLRPFLLRRLKKEVESQL
PEKVEYVIKCDMSALQKILYRHMQAKGILLTDGSEKDKKGKGGAKTLMNT
IMQLRKICNHPYMFQHIEESFAEHLGYSNGVINGAELYRASGKFELLDRI
LPKLRATNHRVLLFCQMTSLMTIMEDYFAFRNFLYLRLDGTTKSEDRAAL
LKKFNEPGSQYFIFLLSTRAGGLGLNLQAADTVVIFDSDWNPHQDLQAQD
RAHRIGQQNEVRVLRLCTVNSVEEKILAAAKYKLNVDQKVIQAGMFDQKS
SSHERRAFLQAILEHEEENEEEDEVPDDETLNQMIARREEEFDLFMPMDM
DRRREDARNPKRKPRLMEEDELPSWIIKDDAEVERLTCEEEEEKTFGRGS
RQRRDVDYSDALTEKQWLRAIEDGNLEEMEEEVRLKKRKRRRNVDKDPAK
EDVEKAKKRRGRPPAEKLSPNPPKLTKQMNAIIDTVINYKDSSGRQLSEV
FIQLPSRKELPEYYELIRKPVDFKKIKERIRNHKYRSLGDLEKDYMLLCH
NAQTFNLEGSQIYEDSIVLQSVFKSARQKIAKEEESEDESNEEEEEEDEE
ESESEAKSVKVKIKLNKKDDKGRDKGKGKKRPNRGKAKPVVSDFDSDEEQ
DEREQSEGSGTDDE.

>NP_001276327.1 probable global transcription activator SNF2L2 isoform d [Homo sapiens]

(SEQ ID NO: 8)
MWLAIEDGNLEEMEEEVRLKKRKRRRNVDKDPAKEDVEKAKKRRGRPPAE
KLSPNPPKLTKQMNAIIDTVINYKDSSGRQLSEVFIQLPSRKELPEYYEL
IRKPVDFKKIKERIRNHKYRSLGDLEKDVMLLCHNAQTFNLEGSQIYEDS
IVLQSVFKSARQKTAKEEESEDESNEEEEEEDEEESESEAKSVKVKIKLN
KKDDKGRDKGKGKKRPNRGKAKPWSDFDSDEEQDEREQSEGSGTDDE.

### SMARCA4

>NM_001.128849.1 Homo sapiens SWI/SNF related, matrix associated, actin dependent regulator of chromatin, subfamily a, member 4 (SMARCA4), transcript variant 1, mRNA

(SEQ ID NO: 9)
GGCGGGGGAGGCGCCGGGAAGTCGACGGCGCCGGCGGCTCCTGCAGGAGG
CCACTGTCTGCAGCTCCCGTGAAGATGTCCACTCCAGACCCACCCCTGGG
CGGAACTCCTCGGCCAGGTCCTTCCCCGGGCCCTGGCCCTTCCCCTGGAG
CCATGCTGGGCCCTAGCCCGGGTCCCTCGCCGGGCTCCGCCCACAGCATG
ATGGGGCCCAGCCCAGGGCCGCCCTCAGCAGGACACCCCATCCCCACCCA
GGGGCCTGGAGGGTACCCTCAGGACAACATGCACCAGATGCACAAGCCCA
TGGAGTCCATGCATGAGAAGGGCATGTCGGACGACCCGCGCTACAACCAG
ATGAAAGGAATGGGGATGCGGTCAGGGGGCCATGCTGGGATGGGGCCCCC
GCCCAGCCCCATGGACCAGCACTCCCAAGGTTACCCCTCGCCCCTGGGTG
GCTCTGAGCATGCCTCTAGTCCAGTTCCAGCCAGTGGCCCGTCTTCGGGG
CCCCAGATGTCTTCCGGGCCAGGAGGTGCCCCGCTGGATGGTGCTGACCC
CCAGGCCTTGGGGCAGCAGAACCGGGGCCCAACCCCATTTAACCAGAACC
AGCTGCACCAGCTCAGAGCTCAGATCATGGCCTACAAGATGCTGGCCAGG
GGGCAGCCCCTCCCCGACCACCTGCAGATGGCGGTGCAGGGCAAGCGGCC
GATGCCCGGGATGCAGCAGCAGATGCCAACGCTACCTCCACCCTCGGTGT
CCGCAACAGGACCCGGCCCTGGCCCTGGCCCTGGCCCCGGCCCGGGTCCC
GGCCCGGCACCTCCAAATTACAGCAGGCCTCATGGTATGGGAGGGCCCAA
CATGCCTCCCCCAGGACCCTCGGGCGTGCCCCCCGGGATGCCAGGCCAGC
CTCCTGGAGGGCCTCCCAAGCCCTGGCCTGAAGGACCCATGGCGAATGCT
GCTGCCCCCACGAGCACCCCTCAGAAGCTGATTCCCCCGCAGCCAACGGG
CCGCCCTTCCCCCGCGCCCCCTGCCGTCCCACCCGCCGCCTCGCCCGTGA
TGCCACCGCAGACCCAGTCCCCCGGGCAGCCGGCCCAGCCCGCGCCCATG
GTGCCACTGCACCAGAAGCAGAGCCGCATCACCCCCATCCAGAAGCCGCG
GGGCCTCGACCCTGTGGAGATCCTGCAGGAGCGCGAGTACAGGCTGCAGG
CTCGCATCGCACACCGAATTCAGGAACTTGAAAACCTTCCCGGGTCCCTG
GCCGGGGATTTGCGAACCAAAGCGACCATTGAGCTCAAGGCCCTCAGGCT
GCTGAACTTCCAGAGGCAGCTGCGCCAGGAGGTGGTGGTGTGCATGCGGA
GGGACACAGCGCTGGAGACAGCCCTCAATGCTAAGGCCTACAAGCGCAGC
AAGCGCCAGTCCCTGCGCGAGGCCCGCATCACTGAGAAGCTGGAGAAGCA
GCAGAAGATCGAGCAGGAGCGCAAGCGCCGGCAGAAGCACCAGGAATACC
TCAATAGCATTCTCCAGCATGCCAAGGATTTCAAGGAATATCACAGATCC
GTCACAGGCAAAATCCAGAAGCTGACCAAGGCAGTGGCCACGTACCATGC
CAACACGGAGCGGGAGCAGAAGAAAGAGAACGAGCGGATCGAGAAGGAGC
GCATGCGGAGGCTCATGGCTGAAGATGAGGAGGGGTACCGCAAGCTCATC
GACCAGAAGAAGGACAAGCGCCTGGCCTACCTCTTGCAGCAGACAGACGA
GTACGTGGCTAACCTCACGGAGCTGGTGCGGCAGCACAAGGCTGCCCAGG
TCGCCAAGGAGAAAAAGAAGAAAAAGAAAAAGAAGAAGGCAGAAAATGCA
GAAGGACAGACGCCTGCCATTGGGCCGGATGGCGAGCCTCTGGACGAGAC
CAGCCAGATGAGCGACCTCCCGGTGAAGGTGATCCACGTGGAGAGTGGGA
AGATCCTCACAGGCACAGATGCCCCCAAAGCCGGGCAGCTGGAGGCCTGG
CTCGAGATGAACCCGGGGTATGAAGTAGCTCCGAGGTCTGATAGTGAAGA
AAGTGGCTCAGAAGAAGAGGAAGAGGAGGAGGAGGAAGAGCAGCCGCAGG
CAGCACAGCCTCCCACCCTGCCCGTGGAGGAGAAGAAGAAGATTCCAGAT
CCAGACAGCGATGACGTCTCTGAGGTGGACGCGCGGCACATCATTGAGAA
TGCCAAGCAAGATGTCGATGATGAATATGGCGTGTCCCAGGCCCTTGCAC
GTGGCCTGCAGTCCTACTATGCCGTGGCCCATGCTGTCACTGAGAGAGTG
GACAAGCAGTCAGCGCTTATGGTCAATGGTGTCCTCAAACAGTACCAGAT
CAAAGGTTTGGAGTGGCTGGTGTCCCTGTACAACAACAACCTGAACGGCA
TCCTGGCCGACGAGATGGGCCTGGGGAAGACCATCCAGACCATCGCGCTC
ATCACGTACCTCATGGAGCACAAACGCATCAATGGGCCCTTCCTCATCAT
CGTGCCTCTCTCAACGCTGTCCAACTGGGCGTACGAGTTTGACAAGTGGG
CCCCCTCCGTGGTGAAGGTGTCTTACAAGGGATCCCCAGCAGCAAGACGG
GCCTTTGTCCCCCAGCTCCGGAGTGGGAAGTTCAACGTCTTGCTGACGAC
GTACGAGTACATCATCAAAGACAAGCACATCCTCGCCAAGATCCGTTGGA
AGTACATGATTGTGGACGAAGGTCACCGCATGAAGAACCACCACTGCAAG
CTGACGCAGGTGCTCAACACGCACTATGTGGCACCCCGCCGCCTGCTGCT
GACGGGCACACCGCTGCAGAACAAGCTTCCCGAGCTCTGGGCGCTGCTCA
ACTTCCTGCTGCCCACCATCTTCAAGAGCTGCAGCACCTTCGAGCAGTGG
TTTAACGCACCCTTTGCCATGACCGGGGAAAAGGTGGACCTGAATGAGGA
GGAAACCATTCTCATCATCCGGCGTCTCCACAAAGTGCTGCGGCCCTTCT
TGCTCCGACGACTCAAGAAGGAAGTCGAGGCCCAGTTGCCCGAAAAGGTG
GAGTACGTCATCAAGTGCGACATGTCTGCGCTGCAGCGAGTGCTCTACCG
CCACATGCAGGCCAAGGGCGTGCTGCTGACTGATGGCTCCGAGAAGGACA
AGAAGGGCAAAGGCGGCACCAAGACCCTGATGAACACCATCATGCAGCTG
CGGAAGATCTGCAACCACCCCTACATGTTCCAGCACATCGAGGAGTCCTT
TTCCGAGCACTTGGGGTTCACTGGCGGCATTGTCCAAGGGCTGGACCTGT
ACCGAGCCTCGGGTAAATTTGAGCTTCTTGATAGAATTCTTCCCAAACTC
CGAGCAACCAACCACAAAGTGCTGCTGTTCTGCCAAATGACCTCCCTCAT
GACCATCATGGAAGATTACTTTGCGTATCGCGGCTTTAAATACCTCAGGC
TTGATGGAACCACGAAGGCGGAGGACCGGGGCATGCTGCTGAAAACCTTC
AACGAGCCCGGCTCTGAGTACTTCATCTTCCTGCTCAGCACCCGGGCTGG
GGGGCTCGGCCTGAACCTCCAGTCGGCAGACACTGTGATCATTTTTGACA
GCGACTGGAATCCTCACCAGGACCTGCAAGCGCAGGACCGAGCCCACCGC
ATCGGGCAGCAGAACGAGGTGCGTGTGCTCCGCCTCTGCACCGTCAACAG
CGTGGAGGAGAAGATCCTAGCTGCAGCCAAGTACAAGCTCAACGTGGACC
AGAAGGTGATCCAGGCCGGCATGTTCGACCAGAAGTCCTCCAGCCATGAG
CGGCGCGCCTTCCTGCAGGCCATCCTGGAGCACGAGGAGCAGGATGAGAG
CAGACACTGCAGCACGGGCAGCGGCAGTGCCAGCTTCGCCCACACTGCCC
CTCCGCCAGCGGGCGTCAACCCCGACTTGGAGGAGCCACCTCTAAAGGAG
GAAGACGAGGTGCCCGACGACGAGACCGTCAACCAGATGATCGCCCGGCA
CGAGGAGGAGTTTGATCTGTTCATGCGCATGGACCTGGACCGCAGGCGCG
AGGAGGCCCGCAACCCCAAGCGGAAGCCGCGCCTCATGGAGGAGGACGAG
CTCCCCTCGTGGATCATCAAGGACGACGCGGAGGTGGAGCGGCTGACCTG
TGAGGAGGAGGAGGAGAAGATGTTCGGCCGTGGCTCCCGCCACCGCAAGG
AGGTGGACTACAGCGACTCACTGACGGAGAAGCAGTGGCTCAAGAAAATT
ACAGGAAAAGATATCCATGACACAGCCAGCAGTGTGGCACGTGGGCTACA
ATTCCAGCGTGGCCTTCAGTTCTGCACACGTGCGTCAAAGGCCATCGAGG
AGGGCACGCTGGAGGAGATCGAAGAGGAGGTCCGGCAGAAGAAATCATCA
CGGAAGCGCAAGCGAGACAGCGACGCCGGCTCCTCCACCCCGACCACCAG
CACCCGCAGCCGCGACAAGGACGACGAGAGCAAGAAGCAGAAGAAGCGCG
GGCGGCCGCCTGCCGAGAAACTCTCCCCTAACCCACCCAACCTCACCAAG
AAGATGAAGAAGATTGTGGATGCCGTGATCAAGTACAAGGACAGCAGCAG
TGGACGTCAGCTCAGCGAGGTCTTCATCCAGCTGCCCTCGCGAAAGGAGC
TGCCCGAGTACTACGAGCTCATCCGCAAGCCCGTGGACTTCAAGAAGATA
AAGGAGCGCATTCGCAACCACAAGTACCGCAGCCTCAACGACCTAGAGAA
GGACGTCATGCTCCTGTGCCAGAACGCACAGACCTTCAACCTGGAGGGCT
CCCTGATCTATGAAGACTCCATCGTCTTGCAGTCGGTCTTCACCAGCGTG
CGGCAGAAAATCGAGAAGGAGGATGACAGTGAAGGCGAGGAGAGTGAGGA
GGAGGAAGAGGGCGAGGAGGAAGGCTCCGAATCCGAATCTCGGTCCGTCA
AAGTGAAGATCAAGCTTGGCCGGAAGGAGAAGGCACAGGACCGGCTGAAG
GGCGGCCGGCGGCGGCCGAGCCGAGGGTCCCGAGCCAAGCCGGTCGTGAG
TGACGATGACAGTGAGGAGGAACAAGAGGAGGACCGCTCAGGAAGTGGCA
GCGAAGAAGACTGAGCCCCGACATTCCAGTCTCGACCCCGAGCCCCTCGT
TCCAGAGCTGAGATGGCATAGGCCTTAGCAGTAACGGGTAGCAGCAGATG
TAGTTTCAGACTTGGAGTAAAACTGTATAAACAAAAGAATCTTCCATATT
TATACAGCAGAGAAGCTGTAGGACTGTTTGTGACTGGCCCTGTCCTGGCA
TCAGTAGCATCTGTAACAGCATTAACTGTCTTAAAGAGAGAGAGAGAGAA
TTCCGAATTGGGGAACACACGATACCTGTTTTTCTTTTCCGTTGCTGGCA
GTACTGTTGCGCCGCAGTTTGGAGTCACTGTAGTTAAGTGTGGATGCATG
TGCGTCACCGTCCACTCCTCCTACTGTATTTTATTGGACAGGTCAGACTC
GCCGGGGGCCCGGCGAGGGTATGTCAGTGTCACTGGATGTCAAACAGTAA
TAAATTAAACCAACAACAAACGCACAAGCCAAAAAAAAA.

>NM_001.128844.1 Homo sapiens SWI/SNF related, matrix associated, actin dependent regulator of chromatin, subfamily a, member 4 (SMARCA4), transcript variant 2, mRNA

(SEQ ID NO: 10)
GGAGAGGCCGCCGCGGTGCTGAGGGGGAGGGGAGCCGGCGAGCGCGCGCG
CAGCGGGGGCGCGGGTGGCGCGCGTGTGTGTGAAGGGGGGGCGGTGGCCG
AGGCGGGCGGGCGCGCGCGCGAGGCTTCCCCTCGTTTGGCGGCGGCGGCG
GCTTCTTTGTTTCGTGAAGAGAAGCGAGACGCCCATTCTGCCCCCGGCCC
CGCGCGGAGGGGCGGGGGAGGCGCCGGGAAGTCGACGGCGCCGGCGGCTC
CTGCGTCTCGCCCTTTTGCCCAGGCTAGAGTGCAGTGGTGCGGTCATGGT
TCACTGCAGCCTCAACCTCCTGGACTCAGCAGGAGGCCACTGTCTGCAGC
TCCCGTGAAGATGTCCACTCCAGACCCACCCCTGGGCGGAACTCCTCGGC
CAGGTCCTTCCCCGGGCCCTGGCCCTTCCCCTGGAGCCATGCTGGGCCCT
AGCCCGGGTCCCTCGCCGGGCTCCGCCCACAGCATGATGGGGCCCAGCCC
AGGGCCGCCCTCAGCAGGACACCCCATCCCCACCCAGGGGCCTGGAGGGT
ACCCTCAGGACAACATGCACCAGATGCACAAGCCCATGGAGTCCATGCAT
GAGAAGGGCATGTCGGACGACCCGCGCTACAACCAGATGAAAGGAATGGG
GATGCGGTCAGGGGGCCATGCTGGGATGGGGCCCCCGCCCAGCCCCATGG
ACCAGCACTCCCAAGGTTACCCCTCGCCCCTGGGTGGCTCTGAGCATGCC
TCTAGTCCAGTTCCAGCCAGTGGCCCGTCTTCGGGGCCCCAGATGTCTTC
CGGGCCAGGAGGTGCCCCGCTGGATGGTGCTGACCCCCAGGCCTTGGGGC
AGCAGAACCGGGGCCCAACCCCATTTAACCAGAACCAGCTGCACCAGCTC
AGAGCTCAGATCATGGCCTACAAGATGCTGGCCAGGGGGCAGCCCCTCCC
CGACCACCTGCAGATGGCGGTGCAGGGCAAGCGGCCGATGCCCGGGATGC
AGCAGCAGATGCCAACGCTAGCTCCACCCTCGGTGTCCGCAACAGGACCC
GGCCCTGGCCCTGGCCCTGGCCCCGGCCCGGGTCCCGGCCCGGCACCTCC
AAATTACAGCAGGCCTCATGGTATGGGAGGGCCCAACATGCCTCCCCCAG
GACCCTCGGGCGTGCCCCCCGGGATGCCAGGCCAGCCTCCTGGAGGGCCT
CCCAAGCCCTGGCCTGAAGGACCCATGGCGAATGCTGCTGCCCCCACGAG
CACCCCTCAGAAGCTGATTCCCCCGCAGCCAACGGGCCGCCCTTCCCCCG
CGCCCCCTGCCGTCCCACCCGCCGCCTCGCCCGTGATGCCACCGCAGACC
CAGTCCCCCGGGCAGCCGGCCCAGCCCGCGCCCATGGTGCCACTGCACCA
GAAGCAGAGCCGCATCACCCCCATCCAGAAGCCGCGGGGCCTCGACCCTG
TGGAGATCCTGCAGGAGCGCGAGTACAGGCTGCAGGCTCGCATCGCACAC
CGAATTCAGGAACTTGAAAACCTTCCCGGGTCCCTGGCCGGGGATTTGCG
AACCAAAGCGACCATTGAGCTCAAGGCCCTCAGGCTGCTGAACTTCCAGA
GGCAGCTGCGCCAGGAGGTGGTGGTGTGCATGCGGAGGGACACAGCGCTG
GAGACAGCCCTCAATGCTAAGGCCTACAAGCGCAGCAAGCGCCAGTCCCT
GCGCGAGGCCCGCATCACTGAGAAGCTGGAGAAGCAGCAGAAGATCGAGC
AGGAGCGCAAGCGCCGGCAGAAGCACCAGGAATACCTCAATAGCATTCTC
CAGCATGCCAAGGATTTCAAGGAATATCACAGATCCGTCACAGGCAAAAT
CCAGAAGCTGACCAAGGCAGTGGCCACGTACCATGCCAACACGGAGCGGG
AGCAGAAGAAAGAGAACGAGCGGATCGAGAAGGAGCGCATGCGGAGGCTC
ATGGCTGAAGATGAGGAGGGGTACCGCAAGCTCATCGACCAGAAGAAGGA
CAAGCGCCTGGCCTAGCTCTTGCAGCAGACAGACGAGTACGTGGCTAACC
TCACGGAGCTGGTGCGGCAGCACAAGGCTGCCCAGGTCGCCAAGGAGAAA
AAGAAGAAAAAGAAAAAGAAGAAGGCAGAAAATGCAGAAGGACAGACGCC
TGCCATTGGGCCGGATGGCGAGCCTCTGGACGAGACCAGCCAGATGAGCG
ACCTCCCGGTGAAGGTGATCCACGTGGAGAGTGGGAAGATCCTCACAGGC
ACAGATGCCCCCAAAGCCGGGCAGCTGGAGGCCTGGCTCGAGATGAACCC
GGGGTATGAAGTAGCTCCGAGGTCTGATAGTGAAGAAAGTGGCTCAGAAG
AAGAGGAAGAGGAGGAGGAGGAAGAGCAGCCGCAGGCAGCACAGCCTCCC
ACCCTGCCCGTGGAGGAGAAGAAGAAGATTCCAGATCCAGACAGCGATGA
CGTCTCTGAGGTGGACGCGCGGCACATCATTGAGAATGCCAAGCAAGATG
TCGATGATGAATATGGCGTGTCCCAGGCCCTTGCACGTGGCCTGCAGTCC
TACTATGCCGTGGCCCATGCTGTCACTGAGAGAGTGGACAAGCAGTCAGC
GCTTATGGTCAATGGTGTCCTCAAACAGTACCAGATCAAAGGTTTGGAGT
GGCTGGTGTCCCTGTACAACAACAACCTGAACGGCATCCTGGCCGACGAG
ATGGGCCTGGGGAAGACCATCCAGACCATCGCGCTCATCACGTACCTCAT
GGAGCACAAACGCATCAATGGGCCCTTCCTCATCATCGTGCCTCTCTCAA
CGCTGTCCAACTGGGCGTACGAGTTTGACAAGTGGGCCCCCTCCGTGGTG
AAGGTGTCTTACAAGGGATCCCCAGCAGCAAGACGGGCCTTTGTCCCCCA
GCTCCGGAGTGGGAAGTTCAACGTCTTGCTGACGACGTACGAGTACATCA
TCAAAGACAAGCACATCCTCGCCAAGATCCGTTGGAAGTACATGATTGTG
GACGAAGGTCACCGCATGAAGAACCACCACTGCAAGCTGACGCAGGTGCT
CAACACGCACTATGTGGCACCCCGCCGCCTGCTGCTGACGGGCACACCGC
TGCAGAAGAAGCTTCCCGAGCTCTGGGCGCTGCTCAACTTCCTGCTGCCC
ACCATCTTCAAGAGCTGCAGCACCTTCGAGCAGTGGTTTAACGCACCCTT
TGCCATGACCGGGGAAAAGGTGGACCTGAATGAGGAGGAAACCATTCTCA
TCATCCGGCGTCTCCACAAAGTGCTGCGGCCCTTCTTGCTCCGACGACTC
AAGAAGGAAGTCGAGGCCCAGTTGCCCGAAAAGGTGGAGTACGTCATCAA
GTGCGACATGTCTGCGCTGCAGCGAGTGCTCTACCGCCACATGCAGGCCA
AGGGCGTGCTGCTGACTGATGGCTCCGAGAAGGACAAGAAGGGCAAAGGC
GGCACCAAGACCCTGATGAACACCATCATGCAGCTGCGGAAGATCTGCAA
CCACCCCTACATGTTCCAGCACATCGAGGAGTCCTTTTCCGAGCACTTGG
GGTTCACTGGCGGCATTGTCCAAGGGCTGGACCTGTACCGAGCCTCGGGT
AAATTTGAGCTTCTTGATAGAATTCTTCCCAAACTCCGAGCAACCAACCA
CAAAGTGCTGCTGTTCTGCCAAATGACCTCCCTCATGACCATCATGGAAG
ATTACTTTGCGTATCGCGGCTTTAAATACCTCAGGCTTGATGGAACCACG
AAGGCGGAGGACCGGGGCATGCTGCTGAAAACCTTCAACGAGCCCGGCTC
TGAGTACTTCATCTTCCTGCTCAGGACCCGGGCTGGGGGGCTCGGCCTGA
ACCTCCAGTCGGCAGACACTGTGATCATTTTTGACAGCGACTGGAATCCT
CACCAGGACCTGCAAGCGCAGGACCGAGCCCACCGCATCGGGCAGCAGAA
CGAGGTGCGTGTGCTCCGCCTCTGCACCGTCAACAGCGTGGAGGAGAAGA
TCCTAGCTGCAGCCAAGTACAAGCTCAACGTGGACCAGAAGGTGATCCAG
GCCGGCATGTTCGACCAGAAGTCCTCCAGCCATGAGCGGCGCGCCTTCCT
GCAGGCCATCCTGGAGCACGAGGAGCAGGATGAGAGCAGACACTGCAGCA
CGGGCAGCGGCAGTGCCAGCTTCGCCCACACTGCCCCTCCGCCAGCGGGC
GTCAACCCCGACTTGGAGGAGCCACCTCTAAAGGAGGAAGACGAGGTGCC
CGACGACGAGACCGTCAACCAGATGATCGCCCGGCACGAGGAGGAGTTTG
ATCTGTTCATGCGCATGGACCTGGACCGCAGGCGCGAGGAGGCCCGCAAC
CCCAAGCGGAAGCCGCGCCTCATGGAGGAGGACGAGCTCCCCTCGTGGAT
CATGAAGGACGACGCGGAGGTGGAGCGGCTGACCTGTGAGGAGGAGGAGG
AGAAGATGTTCGGCCGTGGCTCCCGCCACCGCAAGGAGGTGGACTACAGC
GACTCACTGACGGAGAAGCAGTGGCTCAAGGCCATCGAGGAGGGCACGCT
GGAGGAGATCGAAGAGGAGGTCCGGCAGAAGAAATCATCACGGAAGCGCA
AGCGAGACAGCGACGCCGGCTCCTCCACCCCGACCACCAGCACCCGCAGC
CGCGACAAGGACGACGAGAGCAAGAAGCAGAAGAAGCGCGGGCGGCCGCC
TGCCGAGAAACTCTCCCCTAACCCACCCAACCTCACCAAGAAGATGAAGA
AGATTGTGGATGCCGTGATCAAGTACAAGGACAGCAGCAGTGGACGTCAG
CTCAGCGAGGTCTTCATCCAGCTGCCCTCGCGAAAGGAGCTGCCCGAGTA
CTAGGAGCTCATCCGCAAGCCCGTGGACTTCAAGAAGATAAAGGAGCGCA
TTCGCAACCACAAGTACCGCAGCCTCAACGACCTAGAGAAGGACGTCATG
CTCCTGTGCCAGAACGCACAGACCTTCAACCTGGAGGGCTCCCTGATCTA
TGAAGACTCCATCGTCTTGCAGTCGGTCTTCACCAGCGTGCGGCAGAAAA
TCGAGAAGGAGGATGACAGTGAAGGCGAGGAGAGTGAGGAGGAGGAAGAG
GGCGAGGAGGAAGGCTCCGAATCCGAATCTCGGTCCGTCAAAGTGAAGAT
CAAGCTTGGCCGGAAGGAGAAGGCACAGGACCGGCTGAAGGGCGGCCGGC
GGCGGCCGAGCCGAGGGTCCCGAGCCAAGCCGGTCGTGAGTGACGATGAC
AGTGAGGAGGAACAAGAGGAGGACCGCTCAGGAAGTGGCAGCGAAGAAGA
CTGAGCCCCGACATTCCAGTCTCGACCCCGAGCCCCTCGTTCCAGAGCTG
AGATGGCATAGGCCTTAGCAGTAACGGGTAGCAGCAGATGTAGTTTCAGA
CTTGGAGTAAAACTGTATAAACAAAAGAATCTTCCATATTTATACAGCAG
AGAAGCTGTAGGACTGTTTGTGACTGGCCCTGTCCTGGCATCAGTAGCAT
CTGTAACAGCATTAACTGTCTTAAAGAGAGAGAGAGAGAATTCCGAATTG
GGGAACACACGATACCTGTTTTTCTTTTCCGTTGCTGGCAGTACTGTTGC
GCCGCAGTTTGGAGTCACTGTAGTTAAGTGTGGATGCATGTGCGTCACCG
TCCACTCCTCCTACTGTATTTTATTGGACAGGTCAGACTCGCCGGGGGCC
CGGCGAGGGTATGTCAGTGTCACTGGATGTCAAACAGTAATAAATTAAAC
CAACAACAAAACGCACAGCCAAAAAAAAA.

>NM_001.128845.1 Homo sapiens SWI/SNF related, matrix associated, actin dependent regulator of chromatin, subfamily a, member 4 (SMARCA4), transcript variant 4, mRNA

(SEQ ID NO: 11)
ATGTCCACTCCAGACCCACCCCTGGGCGGAACTCCTCGGCCAGGTCCTTC
CCCGGGCCCTGGCCCTTCCCCTGGAGCCATGCTGGGCCCTAGCCCGGGTC
CCTCGCCGGGCTCCGCCCACAGCATGATGGGGCCCAGCCCAGGGCCGCCC
TCAGCAGGACACCCCATCCCCACCCAGGGGCCTGGAGGGTACCCTCAGGA
CAACATGCACCAGATGCACAAGCCCATGGAGTCCATGCATGAGAAGGGCA
TGTCGGACGACCCGCGCTACAACCAGATGAAAGGAATGGGGATGCGGTCA
GGGGGCCATGCTGGGATGGGGCCCCCGCCCAGCCCCATGGACCAGCACTC
CCAAGGTTACCCCTCGCCCCTGGGTGGCTCTGAGCATGCCTCTAGTCCAG
TTCCAGCCAGTGGCCCGTCTTCGGGGCCCCAGATGTCTTCCGGGCCAGGA
GGTGCCCCGCTGGATGGTGCTGACCCCCAGGCCTTGGGGCAGCAGAACCG
GGGCCCAACCCCATTTAACCAGAACCAGCTGCACCAGCTCAGAGCTCAGA
TCATGGCCTACAAGATGCTGGCCAGGGGGCAGCCCCTCCCCGACCACCTG
CAGATGGCGGTGCAGGGCAAGCGGCCGATGCCCGGGATGCAGCAGCAGAT
GCCAACGCTAGCTCCACCCTCGGTGTCCGCAACAGGACCCGGCCCTGGCC
CTGGCCCTGGCCCCGGCCCGGGTCCCGGCCCGGCACCTCCAAATTACAGC
AGGCCTCATGGTATGGGAGGGCCCAACATGCCTCCCCCAGGACCCTCGGG
CGTGCCCCCCGGGATGCCAGGCCAGCCTCCTGGAGGGCCTCCCAAGCCCT
GGCCTGAAGGACCCATGGCGAATGCTGCTGCCCCCACGAGCACCCCTCAG
AAGCTGATTCCCCCGCAGCCAACGGGCCGCCCTTCCCCCGCGCCCCCTGC
CGTCCCACCCGCCGCCTCGCCCGTGATGCCACCGCAGACCCAGTCCCCCG
GGCAGCCGGCCCAGCCCGCGCCCATGGTGCCACTGCACCAGAAGCAGAGC
CGCATCACCCCCATCGAGAAGCCGCGGGGCCTCGACCCTGTGGAGATCCT
GCAGGAGCGCGAGTACAGGCTGCAGGCTCGCATCGCACACCGAATTCAGG
AACTTGAAAACCTTCCCGGGTCCCTGGCCGGGGATTTGCGAACCAAAGCG
ACCATTGAGCTCAAGGCCCTCAGGCTGCTGAACTTCCAGAGGCAGCTGCG
CCAGGAGGTGGTGGTGTGCATGCGGAGGGACACAGCGCTGGAGACAGCCC
TCAATGCTAAGGCCTAGAAGCGCAGCAAGCGCCAGTCCCTGCGCGAGGCC
CGCATCACTGAGAAGCTGGAGAAGCAGCAGAAGATCGAGCAGGAGCGCAA
GCGCCGGCAGAAGCACCAGGAATACCTCAATAGCATTCTCCAGCATGCCA
AGGATTTCAAGGAATATCACAGATCCGTCACAGGCAAAATCCAGAAGCTG
ACCAAGGCAGTGGCCACGTACCATGCCAACACGGAGCGGGAGCAGAAGAA
AGAGAACGAGCGGATCGAGAAGGAGCGCATGCGGAGGCTCATGGCTGAAG
ATGAGGAGGGGTACCGCAAGCTCATCGACCAGAAGAAGGACAAGCGCCTG
GCCTACCTCTTGCAGCAGACAGACGAGTACGTGGCTAACCTCACGGAGCT
GGTGCGGCAGCACAAGGCTGCCCAGGTCGCCAAGGAGAAAAAGAAGAAAA
AGAAAAAGAAGAAGGCAGAAAATGCAGAAGGACAGACGCCTGCCATTGGG
CCGGATGGCGAGCCTCTGGACGAGACCAGCCAGATGAGCGACCTCCCGGT
GAAGGTGATCCACGTGGAGAGTGGGAAGATCCTCACAGGCACAGATGCCC
CCAAAGCCGGGCAGCTGGAGGCCTGGCTCGAGATGAACCCGGGGTATGAA
GTAGCTCCGAGGTCTGATAGTGAAGAAAGTGGCTCAGAAGAAGAGGAAGA
GGAGGAGGAGGAAGAGCAGCCGCAGGCAGCACAGCCTCCCACCCTGCCCG
TGGAGGAGAAGAAGAAGATTCCAGATCCAGACAGCGATGACGTCTCTGAG
GTGGACGCGCGGCACATCATTGAGAATGCCAAGCAAGATGTCGATGATGA
ATATGGCGTGTCCCAGGCCCTTGCACGTGGCCTGCAGTCCTACTATGCCG
TGGCCCATGCTGTCACTGAGAGAGTGGACAAGCAGTCAGCGCTTATGGTC
AATGGTGTCCTCAAACAGTACCAGATCAAAGGTTTGGAGTGGCTGGTGTC
CCTGTACAACAACAACCTGAACGGCATCCTGGCCGACGAGATGGGCCTGG
GGAAGACCATCCAGACCATCGCGCTCATCACGTACCTCATGGAGCACAAA
CGCATCAATGGGCCCTTCCTCATCATCGTGCCTCTCTCAACGCTGTCCAA
CTGGGCGTACGAGTTTGACAAGTGGGCCCCCTCCGTGGTGAAGGTGTCTT
ACAAGGGATCCCCAGCAGCAAGACGGGCCTTTGTCCCCCAGCTCCGGAGT
GGGAAGTTCAACGTCTTGCTGACGACGTACGAGTACATCATCAAAGACAA
GCACATCCTCGCCAAGATCCGTTGGAAGTACATGATTGTGGACGAAGGTC
ACCGCATGAAGAACCACCACTGCAAGCTGACGCAGGTGCTCAACACGCAC
TATGTGGCACCCCGCCGCCTGCTGCTGACGGGCACACCGCTGCAGAAGAA
GCTTCCCGAGCTCTGGGCGCTGCTCAACTTCCTGCTGCCCACCATCTTCA
AGAGCTGCAGCACCTTCGAGCAGTGGTTTAACGCACCCTTTGCCATGACC
GGGGAAAAGGTGGACCTGAATGAGGAGGAAACCATTCTCATCATCCGGCG
TCTCCACAAAGTGCTGCGGCCCTTCTTGCTCCGACGACTCAAGAAGGAAG
TCGAGGCCCAGTTGCCCGAAAAGGTGGAGTACGTCATCAAGTGCGACATG
TCTGCGCTGCAGCGAGTGCTCTACCGCCACATGCAGGCCAAGGGCGTGCT
GCTGACTGATGGCTCCGAGAAGGACAAGAAGGGCAAAGGCGGCACCAAGA
CCCTGATGAACACCATCATGCAGCTGCGGAAGATCTGCAACCACCCCTAC
ATGTTCCAGCACATCGAGGAGTCCTTTTCCGAGCACTTGGGGTTCACTGG
CGGCATTGTCCAAGGGCTGGACCTGTACCGAGCCTCGGGTAAATTTGAGC
TTCTTGATAGAATTCTTCCCAAACTCCGAGCAACCAACCACAAAGTGCTG
CTGTTCTGCCAAATGACCTCCCTCATGACCATCATGGAAGATTACTTTGC
GTATCGCGGCTTTAAATACCTCAGGCTTGATGGAACCACGAAGGCGGAGG
ACCGGGGCATGCTGCTGAAAACCTTCAACGAGCCCGGCTCTGAGTACTTC
ATCTTCCTGCTCAGCACCCGGGCTGGGGGGCTCGGCCTGAACCTCCAGTC
GGCAGACACTGTGATCATTTTTGACAGCGACTGGAATCCTCACCAGGACC
TGCAAGCGCAGGACCGAGCCCACCGCATCGGGCAGCAGAACGAGGTGCGT
GTGCTCCGCCTCTGCACCGTCAACAGCGTGGAGGAGAAGATCCTAGCTGC
AGCCAAGTACAAGCTCAACGTGGACCAGAAGGTGATCCAGGCCGGCATGT
TCGACCAGAAGTCCTCCAGCCATGAGCGGCGCGCCTTCCTGCAGGCCATC
CTGGAGCACGAGGAGCAGGATGAGGAGGAAGACGAGGTGCCCGACGACGA
GACCGTCAACCAGATGATCGCCCGGCACGAGGAGGAGTTTGATCTGTTCA
TGCGCATGGACCTGGACCGCAGGCGCGAGGAGGCCCGCAACCCCAAGCGG
AAGCCGCGCCTCATGGAGGAGGACGAGCTCCCCTCGTGGATCATCAAGGA
CGACGCGGAGGTGGAGCGGCTGACCTGTGAGGAGGAGGAGGAGAAGATGT
TCGGCCGTGGCTCCCGCCACCGCAAGGAGGTGCACTACAGCGACTCACTG
ACGGAGAAGCAGTGGCTCAAGACCCTGAAGGCCATCGAGGAGGGCACGCT
GGAGGAGATCGAAGAGGAGGTCCGGCAGAAGAAATCATCACGGAAGCGCA
AGCGAGACAGCGACGCCGGCTCCTCCACCCCGACCACCAGCACCCGCAGC
CGCGACAAGGACGACGAGAGCAAGAAGCAGAAGAAGCGCGGGCGGCCGCC
TGCCGAGAAACTCTCCCCTAACCCACCCAACCTCACCAAGAAGATGAAGA
AGATTGTGGATGCCGTGATCAAGTACAAGGACAGCAGCAGTGGACGTCAG
CTCAGCGAGGTCTTCATCCAGCTGCCCTCGCGAAAGCAGCTGCCCGAGTA
CTACGAGCTCATCCGCAAGCCCGTGGACTTCAAGAAGATAAAGGAGCGCA
TTCGCAACCACAAGTACCGCAGCCTCAACGACCTAGAGAAGGACGTCATG
CTCCTGTGCCAGAACGCACAGACCTTCAACCTGGAGGGCTCCCTGATCTA
TGAAGACTCCATCGTCTTGCAGTCGGTCTTCACCAGCGTGCGGCAGAAAA
TCGAGAAGGAGGATGACAGTGAAGGCGAGGAGAGTGAGGAGGAGGAAGAG
GGCGAGGAGGAAGGCTCCGAATCCGAATCTCGGTCCGTCAAAGTGAAGAT
CAAGCTTGGCCGGAAGGAGAAGGCACAGGACCGGCTGAAGGGCGGCCGGC
GGCGGCCGAGCCGAGGGTCCCGAGCCAAGCCGGTCGTGAGTGACGATGAC
AGTGAGGAGGAACAAGAGGAGGACCGCTCAGGAAGTGGCAGCGAAGAAGA
CTGAGCCCCGAGATTCCAGTCTCGACCCCGAGCCCCTCGTTCCAGAGCTG
AGATGGCATAGGCCTTAGCAGTAACGGGTAGCAGCAGATGTAGTTTCAGA
CTTGGAGTAAAACTGTATAAACAAAAGAATCTTCCATATTTATACAGCAG
AGAAGCTGTAGGACTGTTTGTGACTGGCCCTGTCCTGGCATCAGTAGCAT
CTGTAACAGCATTAACTGTCTTAAAGAGAGAGAGAGAGAATTCCGAATTG
GGGAACACACGATACCTGTTTTTCTTTTCCGTTGCTGGCAGTACTGTTGC
GCCGCAGTTTGGAGTCACTGTAGTTAAGTGTGGATGCATGTGCGTCACCG
TCCACTCCTCCTACTGTATTTTATTGGACAGGTCAGACTCGCCGGGGGCC
CGGCGAGGGTATGTCAGTGTCACTGGATGTCAAACAGTAATAAATTAAAC
CAACAACAAAACGCACAGCCAAAAAAAAA.

>NM_001128846 Homo sapiens SWI/SNF related, matrix associated, actin dependent regulator of chromatin, subfamily a, member 4 (SMARCA4), transcript variant 5, mRNA

(SEQ ID NO: 12)
ATGTCCACTCCAGACCCACCCCTGGGCGGAACTCCTCGGCCAGGTCCTTC
CCCGGGCCCTGGCCCTTCCCCTGGAGCCATGCTGGGCCCTAGCCCGGGTC
CCTCGCCGGGCTCCGCCCACAGCATGATGGGGCCCAGCCCAGGGCCGCCC
TCAGCAGGAGACCCCATCCCCACCCAGGGGCCTGGAGGGTACGCTCAGGA
CAACATGCACCAGATGCACAAGCCCATGGAGTCCATGCATGAGAAGGGCA
TGTCGGACGACCCGCGCTAGAACCAGATGAAAGGAATGGGGATGCGGTCA
GGGGGCCATGCTGGGATGGGGCCCCCGCCCAGCCCCATGGACCAGCACTC
CCAAGGTTACCCCTCGCCCCTGGGTGGCTCTGAGCATGCCTCTAGTCCAG
TTCCAGCCAGTGGCCCGTCTTCGGGGCCCCAGATGTCTTCCGGGCCAGGA
GGTGCCCCGCTGGATGGTGCTGACCCCCAGGCCTTGGGGCAGCAGAACCG
GGGCCCAACCCCATTTAACCAGAACCAGCTGCACCAGCTCAGAGCTCAGA
TCATGGCCTACAAGATGCTGGCCAGGGGGCAGCCCCTCCCCGACCACCTG
CAGATGGCGGTGCAGGGCAAGCGGCCGATGCCCGGGATGCAGCAGCAGAT
GCCAACGCTACCTCCACCCTCGGTGTCCGCAACAGGACCCGGCCCTGGCC
CTGGCCCTGGCCCCGGCCCGGGTCCCGGCCCGGCACCTCCAAATTACAGC
AGGCCTCATGGTATGGGAGGGCCCAACATGCCTCCCCCAGGACCCTCGGG
CGTGCCCCCCGGGATGCCAGGCCAGCCTCCTGGAGGGCCTCCCAAGCCCT
GGCCTGAAGGACCCATGGCGAATGCTGCTGCCCCCACGAGCACCCCTCAG
AAGCTGATTCCCCCGCAGCCAACGGGCCGCCCTTCCCCCGCGCCCCCTGC
CGTCCCACCCGCCGCCTCGCCCGTGATGCCACCGCAGACCCAGTCCCCCG
GGCAGCCGGCCCAGCCCGCGCCCATGGTGCCACTGCACCAGAAGCAGAGC
CGCATCACCCCCATCCAGAAGCCGCGGGGCCTCGACCCTGTGGAGATCCT
GCAGGAGCGCGAGTACAGGCTGCAGGCTCGCATCGCACACCGAATTCAGG
AACTTGAAAACCTTCCCGGGTCCCTGGCCGGGGATTTGCGAACCAAAGCG
ACCATTGAGCTCAAGGCCCTCAGGCTGCTGAACTTCCAGAGGCAGCTGCG
CCAGGAGGTGGTGGTGTGCATGCGGAGGGACACAGCGCTGGAGACAGCCC
TCAATGCTAAGGCCTACAAGCGCAGCAAGCGCCAGTCCCTGCGCCAGGCC
CGCATCACTGAGAAGCTGGAGAAGCAGCAGAAGATCGAGCAGGAGCGCAA
GCGCCGGCAGAAGCACCAGGAATACCTCAATAGCATTCTCCAGCATGCCA
AGGATTTCAAGGAATATCACAGATCCGTCACAGGCAAAATCCAGAAGCTG
ACCAAGGCAGTGGCCACGTACCATGCCAACACGGAGCGGGAGCAGAAGAA
AGAGAACGAGCGGATCGAGAAGGAGCGCATGCGGAGGCTCATGGCTGAAG
ATGAGGAGGGGTAGCGCAAGCTCATCGACCAGAAGAAGGACAAGCGCCTG
GCCTAGCTCTTGCAGCAGACAGACGAGTACGTGGCTAACCTCACGGAGCT
GGTGCGGCAGCACAAGGCTGCCCAGGTCGCCAAGGAGAAAAAGAAGAAAA
AGAAAAAGAAGAAGGCAGAAAATGCAGAAGGACAGACGCCTGCCATTGGG
CCGGATGGCGAGCCTCTGGACGAGACCAGCCAGATGAGCGACCTCCCGGT
GAAGGTGATCCACGTGGAGAGTGGGAAGATCCTCACAGGCACAGATGCCC
CCAAAGCCGGGCAGCTGGAGGCCTGGCTCGAGATGAACCCGGGGTATGAA
GTAGCTCCGAGGTCTGATAGTGAAGAAAGTGGCTCAGAAGAAGAGGAAGA
GGAGGAGGAGGAAGAGCAGCCGCAGGCAGCACAGCCTCCCACCCTGCCCG
TGGAGGAGAAGAAGAAGATTCCAGATCCAGACAGCGATGACGTCTCTGAG
GTGGACGCGCGGCACATCATTGAGAATGCCAAGCAAGATGTCGATGATGA
ATATGGCGTGTCCCAGGCCCTTGCACGTGGCCTGCAGTCCTACTATGCCG
TGGCCCATGCTGTCACTGAGAGAGTGGACAAGCAGTCAGCGCTTATGGTC
AATGGTGTCCTCAAACAGTACCAGATCAAAGGTTTGGAGTGGCTGGTGTC
CCTGTACAACAACAACCTGAACGGCATCCTGGCCGACGAGATGGGCCTGG
GGAAGACCATCCAGACCATCGCGCTCATCACGTACCTCATGGAGCACAAA
CGCATCAATGGGCCCTTCCTCATCATCGTGCCTCTCTCAACGCTGTCCAA
CTGGGCGTACGAGTTTGACAAGTGGGCCCCCTCCGTGGTGAAGGTGTCTT
ACAAGGGATCCCCAGCAGCAAGACGGGCCTTTGTCCCCCAGCTCCGGAGT
GGGAAGTTCAACGTCTTGCTGACGACGTACGAGTACATCATCAAAGACAA
GCACATCCTCGCCAAGATCCGTTGGAAGTACATGATTGTGGACGAAGGTC
ACCGCATGAAGAACCACCACTGCAAGCTGACGCAGGTGCTCAACACGCAC
TATGTGGCACCCCGCCGCCTGCTGCTGACGGGCACACCGCTGCAGAACAA
GCTTCCCGAGCTCTGGGCGCTGCTCAACTTCCTGCTGCCCACCATCTTCA
AGAGCTGCAGCACCTTCGAGCAGTGGTTTAACGCACCCTTTGCCATGACC
GGGGAAAAGGTGGACCTGAATGAGGAGGAAACCATTCTCATCATCCGGCG
TCTCCACAAAGTGCTGCGGCCCTTCTTGCTCCGACGACTCAAGAAGGAAG
TCGAGGCCCAGTTGCCCGAAAAGGTGGAGTACGTCATCAAGTGCGACATG
TCTGCGCTGCAGCGAGTGCTCTAGCGCCACATGCAGGCCAAGGGCGTGCT
GCTGACTGATGGCTCCGAGAAGGACAAGAAGGGCAAAGGCGGCACCAAGA
CCCTGATGAACACCATCATGCAGCTGCGGAAGATCTGCAACCACCCCTAC
ATGTTCCAGCACATCGAGGAGTCCTTTTCCGAGCACTTGGGGTTCACTGG
CGGCATTGTCCAAGGGCTGGACCTGTACCGAGCCTCGGGTAAATTTGAGC
TTCTTGATAGAATTCTTCCCAAACTCCGAGCAACCAACCACAAAGTGCTG
CTGTTCTGCCAAATGACCTCCCTCATGACCATCATGGAAGATTAGTTTGC
GTATCGCGGCTTTAAATACCTCAGGCTTGATGGAACCACGAAGGCGGAGG
ACCGGGGCATGCTGCTGAAAACCTTCAACGAGCCCGGCTCTGAGTACTTC
ATCTTCCTGCTCAGCACCCGGGCTGGGGGGCTCGGCCTGAACCTCCAGTC
GGCAGACACTGTGATCATTTTTGACAGCGACTGGAATCCTCACCAGGACC
TGCAAGCGCAGGACCGAGCCCACCGCATCGGGCAGCAGAACGAGGTGCGT
GTGCTCCGCCTCTGCACCGTCAACAGCGTGGAGGAGAAGATCCTAGCTGC
AGCCAAGTACAAGCTCAACGTGGACCAGAAGGTGATCCAGGCCGGCATGT
TCGACCAGAAGTCCTCCAGCCATGAGCGGCGCGCCTTCCTGCAGGCCATC
CTGGAGCACGAGGAGCAGGATGAGGAGGAAGACGAGGTGCCCGACGACGA
GACCGTCAACCAGATGATCGCCCGGCACGAGGAGGAGTTTGATCTGTTCA
TGCGCATGGACCTGGACCGCAGGCGCGAGGAGGCCCGCAACCCCAAGCGG
AAGCCGCGCCTCATGGAGGAGGACGAGCTCCCCTCGTGGATCATCAAGGA
CGACGCGGAGGTGGAGCGGCTGACCTGTGAGGAGGAGGAGGAGAAGATGT
TCGGCCGTGGCTCCCGCCACCGCAAGGAGGTGGACTACAGCGACTCACTG
ACGGAGAAGCAGTGGCTCAAGACCCTGAAGGCCATCGAGGAGGGCACGCT
GGAGGAGATCGAAGAGGAGGTCCGGCAGAAGAAATCATCACGGAAGCGCA
AGCGAGACAGCGACGCCGGCTCCTCCACCCCGACCACCAGCACCCGCAGC
CGCGACAAGGACGACGAGAGCAAGAAGCAGAAGAAGCGCGGGCGGCCGCC
TGCCGAGAAACTCTCCCCTAACCCACCCAACCTCACCAAGAAGATGAAGA
AGATTGTGGATGCCGTGATCAAGTACAAGGACAGCAGTGGACGTCAGCTC
AGCGAGGTCTTCATCCAGCTGCCCTCGCGAAAGGAGCTGCCCGAGTACTA
CGAGCTCATCCGCAAGCCCGTGGACTTCAAGAAGATAAAGGAGCGCATTC
GCAACCACAAGTACCGCAGCCTCAACGACCTAGAGAAGGACGTCATGCTC
CTGTGCCAGAACGCACAGACCTTCAACCTGGAGGGCTCCCTGATCTATGA
AGACTCCATCGTCTTGCAGTCGGTCTTCACCAGCGTGCGGCAGAAAATCG
AGAAGGAGGATGACAGTGAAGGCGAGGAGAGTGAGGAGGAGGAAGAGGGC
GAGGAGGAAGGCTCCGAATCCGAATCTCGGTCCGTCAAAGTGAAGATCAA
GCTTGGCCGGAAGGAGAAGGCACAGGACCGGCTGAAGGGCGGCCGGCGGC
GGCCGAGCCGAGGGTCCCGAGCCAAGCCGGTCGTGAGTGACGATGACAGT
GAGGAGGAACAAGAGGAGGACCGCTCAGGAAGTGGCAGCGAAGAAGACTG
AGCCCCGACATTCCAGTCTCGACCCCGAGCCCCTCGTTCCAGAGCTGAGA
TGGCATAGGCCTTAGCAGTAACGGGTAGCAGCAGATGTAGTTTCAGACTT
GGAGTAAAACTGTATAAACAAAAGAATCTTCCATATTTATACAGCAGAGA
AGCTGTAGGACTGTTTGTGACTGGCCCTGTCCTGGCATCAGTAGCATCTG
TAACAGCATTAACTGTCTTAAAGAGAGAGAGAGAGAATTCCGAATTGGGG
AACACACGATACCTGTTTTTCTTTTCCGTTGCTGGCAGTACTGTTGCGCC
GCAGTTTGGAGTCACTGTAGTTAAGTGTGGATGCATGTGCGTCACCGTCC
ACTCCTCCTACTGTATTTTATTGGACAGGTCAGACTCGCCGGGGGCCCGG
CGAGGGTATGTCAGTGTCACTGGATGTCAAACAGTAATAAATTAAACCAA
CAACAAAACGCACAGCCAAAAAAAAA.

>NM_001.128847.1 Homo sapiens SWI/SNF related, matrix associated, actin dependent regulator of chromatin, subfamily a, member 4 (SMARCA4), transcript variant 6, mRNA

(SEQ ID NO: 13)
ATGTCCACTCCAGACCCACCCCTGGGCGGAACTCCTCGGCCAGGTCCTTC
CCCGGGCCCTGGCCCTTCCCCTGGAGCCATGCTGGGCCCTAGCCCGGGTC
CCTCGCCGGGCTCCGCCCACAGCATGATGGGGCCCAGCCCAGGGCCGCCC
TCAGCAGGACACCCCATCCCCACCCAGGGGCCTGGAGGGTACCCTCAGGA
CAACATGCACCAGATGCACAAGCCCATGGAGTCCATGCATGAGAAGGGCA
TGTCGGACGACCCGCGCTACAACCAGATGAAAGGAATGGGGATGCGGTCA
GGGGGCCATGCTGGGATGGGGCCCCCGCCCAGCCCCATGGACCAGCACTC
CCAAGGTTACCCCTCGCCCCTGGGTGGCTCTGAGCATGCCTCTAGTCCAG
TTCCAGCCAGTGGCCCGTCTTCGGGGCCCCAGATGTCTTCCGGGCCAGGA
GGTGCCCCGCTGGATGGTGCTGACCCCCAGGCCTTGGGGCAGCAGAACCG
GGGCCCAACCCCATTTAACCAGAACCAGCTGCACCAGCTCAGAGCTCAGA
TCATGGCCTACAAGATGCTGGCCAGGGGGCAGCCCCTCCCCGACCACCTG
CAGATGGCGGTGCAGGGCAAGCGGCCGATGCCCGGGATGCAGCAGCAGAT
GCCAACGCTAGCTCCACCCTCGGTGTCCGCAACAGGACCCGGCCCTGGCC
CTGGCCCTGGCCCCGGCCCGGGTCCCGGCCCGGCACCTCCAAATTACAGC
AGGCCTCATGGTATGGGAGGGCCCAACATGCCTCCCCCAGGACCCTCGGG
CGTGCCCCCCGGGATGCCAGGCCAGCCTCCTGGAGGGCCTCCCAAGCCCT
GGCCTGAAGGACCCATGGCGAATGCTGCTGCCCCCACGAGCACCCCTCAG
AAGCTGATTCCCCCGCAGCCAACGGGCCGCCCTTCCCCCGCGCCCCCTGC
CGTCCCACCCGCCGCCTCGCCCGTGATGCCACCGCAGACCCAGTCCCCCG
GGCAGCCGGCCCAGCCCGCGCCCATGGTGCCACTGCACCAGAAGCAGAGC
CGCATCACCCCCATCCAGAAGCCGCGGGGCCTCGACCCTGTGGAGATCCT
GCAGGAGCGCGAGTACAGGCTGCAGGCTCGCATCGCACACCGAATTCAGG
AACTTGAAAACCTTCCCGGGTCCCTGGCCGGGGATTTGCGAACCAAAGCG
ACCATTGAGCTCAAGGCCCTCAGGCTGCTGAACTTCCAGAGGCAGCTGCG
CCAGGAGGTGGTGGTGTGCATGCGGAGGGACACAGCGCTGGAGACAGCCC
TCAATGCTAAGGCCTACAAGCGCAGCAAGCGCCAGTCCCTGCGCGAGGCC
CGCATCACTGAGAAGCTGGAGAAGCAGCAGAAGATCGAGCAGGAGCGCAA
GCGCCGGCAGAAGCACCAGGAATACCTCAATAGCATTCTCCAGCATGCCA
AGGATTTCAAGGAATATCACAGATCCGTCACAGGCAAAATCCAGAAGCTG
ACCAAGGCAGTGGCCACGTACCATGCCAACACGGAGCGGGAGCAGAAGAA
AGAGAACGAGCGGATCGAGAAGGAGCGCATGCGGAGGCTCATGGCTGAAG
ATGAGGAGGGGTACCGCAAGCTCATCGACCAGAAGAAGGAGAAGCGCCTG
GCCTACCTCTTGCAGCAGACAGACGAGTACGTGGCTAACCTCACGGAGCT
GGTGCGGCAGCACAAGGCTGCCCAGGTCGCCAAGGAGAAAAAGAAGAAAA
AGAAAAAGAAGAAGGCAGAAAATGCAGAAGGACAGACGCCTGCCATTGGG
CCGGATGGCGAGCCTCTGGACGAGACCAGCCAGATGAGCGACCTCCCGGT
GAAGGTGATCCACGTGGAGAGTGGGAAGATCCTCACAGGCACAGATGCCC
CCAAAGCCGGGCAGCTGGAGGCCTGGCTCGAGATGAACCCGGGGTATGAA
GTAGCTCCGAGGTCTGATAGTGAAGAAAGTGGCTCAGAAGAAGAGGAAGA
GGAGGAGGAGGAAGAGCAGCCGCAGGCAGCACAGCCTCCCACCCTGCCCG
TGGAGGAGAAGAAGAAGATTCCAGATCCAGACAGCGATGACGTCTCTGAG
GTGGACGCGCGGCACATCATTGAGAATGCCAAGGAAGATGTCGATGATGA
ATATGGCGTGTCCCAGGCCCTTGCACGTGGCCTGCAGTCCTACTATGCCG
TGGCCCATGCTGTCACTGAGAGAGTGGACAAGCAGTCAGCGCTTATGGTC
AATGGTGTCCTCAAACAGTACCAGATCAAAGGTTTGGAGTGGCTGGTGTC
CCTGTACAACAACAACCTGAACGGCATCCTGGCCGACGAGATGGGCCTGG
GGAAGACCATCCAGACCATCGCGCTCATCACGTACCTCATGGAGCACAAA
CGCATCAATGGGCCCTTCCTCATCATCGTGCCTCTCTCAACGCTGTCCAA
CTGGGCGTACGAGTTTGACAAGTGGGCCCCCTCCGTGGTGAAGGTGTCTT
ACAAGGGATCCCCAGCAGCAAGACGGGCCTTTGTCCCCCAGCTCCGGAGT
GGGAAGTTCAACGTCTTGCTGACGACGTACGAGTACATCATCAAAGACAA
GCACATCCTCGCCAAGATCCGTTGGAAGTACATGATTGTGGACGAAGGTC
ACCGCATGAAGAACCACCACTGCAAGCTGACGCAGGTGCTCAACACGCAC
TATGTGGCACCCCGCCGCCTGCTGCTGACGGGCACACCGCTGCAGAAGAA
GCTTCCCGAGCTCTGGGCGCTGCTCAACTTCCTGCTGCCCACCATCTTCA
AGAGCTGCAGCACCTTCGAGCAGTGGTTTAACGCACCCTTTGCCATGACC
GGGGAAAAGGTGGACCTGAATGAGGAGGAAACCATTCTCATCATCCGGCG
TCTCCACAAAGTGCTGCGGCCCTTCTTGCTCCGACGACTCAAGAAGGAAG
TCGAGGCCCAGTTGCCCGAAAAGGTGGAGTACGTCATCAAGTGCGACATG
TCTGCGCTGCAGCGAGTGCTCTACCGCCACATGCAGGCCAAGGGCGTGCT
GCTGACTGATGGCTCCGAGAAGGACAAGAAGGGCAAAGGCGGCACCAAGA
CCCTGATGAACACCATCATGCAGCTGCGGAAGATCTGCAACCACCCCTAC
ATGTTCCAGCACATCGAGGAGTCCTTTTCCGAGCACTTGGGGTTCACTGG
CGGCATTGTCCAAGGGCTGGACCTGTACCGAGCCTCGGGTAAATTTGAGC
TTCTTGATAGAATTCTTCCCAAACTCCGAGCAACCAACCACAAAGTGCTG
CTGTTCTGCCAAATGACCTCCCTCATGACCATCATGGAAGATTACTTTGC
GTATCGCGGCTTTAAATACCTCAGGCTTGATGGAACCACGAAGGCGGAGG
ACCGGGGCATGCTGCTGAAAACCTTCAACGAGCCCGGCTCTGAGTACTTC
ATCTTCCTGCTCAGCACCCGGGCTGGGGGGCTCGGCCTGAACCTCCAGTC
GGCAGACACTGTGATCATTTTTGACAGCGACTGGAATCCTCACCAGGACC
TGCAAGCGCAGGACCGAGCCCACCGCATCGGGCAGCAGAACGAGGTGCGT
GTGCTCCGCCTCTGCACCGTCAACAGCGTGGAGGAGAAGATCCTAGCTGC
AGCCAAGTACAAGCTCAACGTGGACCAGAAGGTGATCCAGGCCGGCATGT
TCGACCAGAAGTCCTCCAGCCATGAGCGGCGCGCCTTCCTGCAGGCCATC
CTGGAGCACGAGGAGCAGGATGAGGAGGAAGACGAGGTGCCCGACGACGA
GACCGTCAACCAGATGATCGCCCGGCACGAGGAGGAGTTTGATCTGTTCA
TGCGCATGGACCTGGACCGCAGGCGCGAGGAGGCCCGCAACCCCAAGCGG
AAGCCGCGCCTCATGGAGGAGGACGAGCTCCCCTCGTGGATCATCAAGGA
CGACGCGGAGGTGGAGCGGCTGACCTGTGAGGAGGAGGAGGAGAAGATGT
TCGGCCGTGGCTCCCGCCACCGCAAGGAGGTGCACTACAGCGACTCACTG
ACGGAGAAGCAGTGGCTCAAGGCCATCGAGGAGGGCACGCTGGAGGAGAT
CGAAGAGGAGGTCCGGCAGAAGAAATCATCACGGAAGCGCAAGCGAGACA
GCGACGCCGGCTCCTCCACCCCGACCACCAGCACCCGCAGCCGCGACAAG
GACGACGAGAGCAAGAAGCAGAAGAAGCGCGGGCGGCCGCCTGCCGAGAA
ACTCTCCCCTAACCCACCCAACCTCACCAAGAAGATGAAGAAGATTGTGG
ATGCCGTGATCAAGTACAAGGACAGCAGCAGTGGACGTCAGCTCAGCGAG
GTCTTCATCCAGCTGCCCTCGCGAAAGGAGCTGCCCCAGTACTACGAGCT
CATCCGCAAGCCCGTGGACTTCAAGAAGATAAAGGAGCGCATTCGCAACC
ACAAGTACCGCAGCCTCAACGACCTAGAGAAGGACGTCATGCTCCTGTGC
CAGAACGCACAGACCTTCAACCTGGAGGGCTCCCTGATCTATGAAGACTC
CATCGTCTTGCAGTCGGTCTTCACCAGCGTGCGGCAGAAAATCGAGAAGG
AGGATGACAGTGAAGGCGAGGAGAGTGAGGAGGAGGAAGAGGGCGAGGAG
GAAGGCTCCGAATCCGAATCTCGGTCCGTCAAAGTGAAGATCAAGCTTGG
CCGGAAGGAGAAGGCACAGGACCGGCTGAAGGGCGGCCGGCGGCGGCCCA
GCCGAGGGTCCCGAGCCAAGCCGGTCGTGAGTGACGATGACAGTGAGGAG
GAACAAGAGGAGGACCGCTCAGGAAGTGGCAGCGAAGAAGACTGAGCCCC
GACATTCCAGTCTCGACCCCGAGCCCCTCGTTCCAGAGCTGAGATGGCAT
AGGCCTTAGCAGTAACGGGTAGCAGCAGATGTAGTTTCAGACTTGGAGTA
AAACTGTATAAACAAAAGAATCTTCCATATTTATACAGCAGAGAAGCTGT
AGGACTGTTTGTGACTGGCCCTGTCCTGGCATCAGTAGCATCTGTAACAG
CATTAACTGTCTTAAAGAGAGAGAGAGAGAATTCCGAATTGGGGAACACA
CGATACCTGTTTTTCTTTTCCGTTGCTGGCAGTACTGTTGCGCCGCAGTT
TGGAGTCACTGTAGTTAAGTGTGGATGCATGTGCGTCACCGTCCACTCCT
CCTACTGTATTTTATTGGACAGGTCAGACTCGCCGGGGGCCCGGCGAGGG
TATGTCAGTGTCACTGGATGTCAAACAGTAATAAATTAAACCAACAACAA
AACGCACAGCCAAAAAAAAA.

>NM_001128848.1 Homo sapiens SWI/SNF related, matrix associated, actin dependent regulator of chromatin, subfamily a, member 4 (SMARCA4), transcript variant 7, mRNA

(SEQ ID NO: 14)
ATGTCCACTCCAGACCCACCCCTGGGCGGAACTCCTCGGCCAGGTCCTTC
CCCGGGCCCTGGCCCTTCCCCTGGAGCCATGCTGGGCCCTAGCCCGGGTC
CCTCGCCGGGCTCCGCCCACAGCATGATGGGGCCCAGCCCAGGGCCGCCC
TCAGCAGGACACCCCATCCCCACCCAGGGGCCTGGAGGGTACCCTCAGGA
CAACATGCACCAGATGCACAAGCCCATGGAGTCCATGCATGAGAAGGGCA
TGTCGGACGACCCGCGCTACAACCAGATGAAAGGAATGGGGATGCGGTCA
GGGGGCCATGCTGGGATGGGGCCCCCGCCCAGCCCCATGGACCAGCACTC
CCAAGGTTACCCCTCGCCCCTGGGTGGCTCTGAGCATGCCTCTAGTCCAG
TTCCAGCCAGTGGCCCGTCTTCGGGGCCCCAGATGTCTTCCGGGCCAGGA
GGTGCCCCGCTGGATGGTGCTGACCCCCAGGCCTTGGGGCAGCAGAACCG
GGGCCCAACCCCATTTAACCAGAACCAGCTGCACCAGCTCAGAGCTCAGA
TCATGGCCTACAAGATGCTGGCCAGGGGGCAGCCCCTCCCCGACCACCTG
CAGATGGCGGTGCAGGGCAAGCGGCCGATGCCCGGGATGCAGCAGCAGAT
GCCAACGCTAGCTCCACCCTCGGTGTCCGCAACAGGACCCGGCCCTGGCC
CTGGCCCTGGCCCCGGCCCGGGTCCCGGCCCGGCACCTCCAAATTACAGC
AGGCCTCATGGTATGGGAGGGCCCAACATGCCTCCCCCAGGACCCTCGGG
CGTGCCCCCCGGGATGCCAGGCCAGCCTCCTGGAGGGCCTCCCAAGCCCT
GGCCTGAAGGACCCATGGCGAATGCTGCTGCCCCCACGAGCACCCCTCAG
AAGCTGATTCCCCCGCAGCCAACGGGCCGCCCTTCCCCCGCGCCCCCTGC
CGTCCCACCCGCCGCCTCGCCCGTGATGCCACCGCAGACCCAGTCCCCCG
GGCAGCCGGCCCAGCCCGCGCCCATGGTGCCACTGCACCAGAAGCAGAGC
CGCATCACCCCCATCCAGAAGCCGCGGGGCCTCGACCCTGTGGAGATCCT
GCAGGAGCGCGAGTACAGGCTGCAGGCTCGCATCGCACACCGAATTCAGG
AACTTGAAAACCTTCCCGGGTCCCTGGCCGGGGATTTGCGAACCAAAGCG
ACCATTGAGCTCAAGGCCCTCAGGCTGCTGAACTTCCAGAGGCAGCTGCG
CCAGGAGGTGGTGGTGTGCATGCGGAGGGACACAGCGCTGGAGACAGCCC
TCAATGCTAAGGCCTACAAGCGCAGCAAGCGCCAGTCCCTGCGCGAGGCC
CGCATCACTGAGAAGCTGGAGAAGCAGCAGAAGATCGAGCAGGAGCGCAA
GCGCCGGCAGAAGCACCAGGAATACCTCAATAGCATTCTCCAGCATGCCA
AGGATTTCAAGGAATATCACAGATCCGTCACAGGCAAAATCCAGAAGCTG
ACCAAGGCAGTGGCCACGTACCATGCCAACACGGAGCGGGAGCAGAAGAA
AGAGAACGAGCGGATCGAGAAGGAGCGCATGCGGAGGCTCATGGCTGAAG
ATGAGGAGGGGTACCGCAAGCTCATCGACCAGAAGAAGGACAAGCGCCTG
GCCTACCTCTTGCAGCAGACAGACGAGTACGTGGCTAACCTCACGGAGCT
GGTGCGGCAGCACAAGGCTGCCCAGGTCGCCAAGGAGAAAAAGAAGAAAA
AGAAAAAGAAGAAGGCAGAAAATGGAGAAGGACAGACGCCTGCCATTGGG
CCGGATGGCGAGCCTCTGGACGAGACCAGCCAGATGAGCGACCTCCCGGT
GAAGGTGATCCACGTGGAGAGTGGGAAGATCCTCACAGGCACAGATGCCC
CCAAAGCCGGGCAGCTGGAGGCCTGGCTCGAGATGAACCCGGGGTATGAA
GTAGCTCCGAGGTCTGATAGTGAAGAAAGTGGCTCAGAAGAAGAGGAAGA
GGAGGAGGAGGAAGAGCAGCCGCAGGCAGCACAGCCTCCCACCCTGCCCG
TGGAGGAGAAGAAGAAGATTCCAGATCCAGACAGCGATGACGTCTCTGAG
GTGGACGCGCGGCACATCATTGAGAATGCCAAGCAAGATGTCGATGATGA
ATATGGCGTGTCCCAGGCCCTTGCACGTGGCCTGCAGTCCTACTATGCCG
TGGCCCATGCTGTCACTGAGAGAGTGGACAAGCAGTCAGCGCTTATGGTC
AATGGTGTCCTCAAACAGTACCAGATCAAAGGTTTGGAGTGGCTGGTGTC
CCTGTACAACAACAACCTGAACGGCATCCTGGCCGACGAGATGGGCCTGG
GGAAGACCATCCAGACCATCGCGCTCATCACGTACCTCATGGAGCACAAA
CGCATCAATGGGCCCTTCCTCATCATCGTGCCTCTCTCAACGCTGTCCAA
CTGGGCGTACGAGTTTGACAAGTGGGCCCCCTCCGTGGTGAAGGTGTCTT
ACAAGGGATCCCCAGCAGCAAGACGGGCCTTTGTCCCCCAGCTCCGGAGT
GGGAAGTTCAACGTCTTGCTGACGACGTACGAGTACATCATCAAAGACAA
GCACATCCTCGCCAAGATCCGTTGGAAGTACATGATTGTGGACGAAGGTC
ACCGCATGAAGAACCACCACTGCAAGCTGACGCAGGTGCTCAACACGCAC
TATGTGGCACCCCGCCGCCTGCTGCTGACGGGCACACCGCTGCAGAACAA
GCTTCCCGAGCTCTGGGCGCTGCTCAACTTCCTGCTGCCCACCATCTTCA
AGAGCTGCAGCACCTTCGAGCAGTGGTTTAACGCACCCTTTGCCATGACC
GGGGAAAAGGTGGACCTGAATGAGGAGGAAACCATTCTCATCATCCGGCG
TCTCCACAAAGTGCTGCGGCCCTTCTTGCTCCGACGACTCAAGAAGGAAG
TCGAGGCCCAGTTGCCCGAAAAGGTGGAGTACGTCATCAAGTGCGACATG
TCTGCGCTGCAGCGAGTGCTCTACCGCCACATGCAGGCCAAGGGCGTGCT
GCTGACTGATGGCTCCGAGAAGGACAAGAAGGGCAAAGGCGGCACCAAGA
CCCTGATGAACACCATCATGCAGCTGCGGAAGATCTGCAACCACCCCTAC
ATGTTCCAGCACATCGAGGAGTCCTTTTCCGAGCACTTGGGGTTCACTGG
CGGCATTGTCCAAGGGCTGGACCTGTACCGAGCCTCGGGTAAATTTGAGC
TTCTTGATAGAATTCTTCCCAAACTCCGAGCAACCAACCACAAAGTGCTG
CTGTTCTGCCAAATGACCTCCCTCATGACCATCATGGAAGATTACTTTGC
GTATCGCGGCTTTAAATACCTCAGGCTTGATGGAACCACGAAGGCGGAGG
ACCGGGGCATGCTGCTGAAAACCTTCAACGAGCCCGGCTCTGAGTACTTC
ATCTTCCTGCTCAGCACCCGGGCTGGGGGGCTCGGCCTGAACCTCCAGTC
GGCAGACACTGTGATCATTTTTGACAGCGACTGGAATCCTCACCAGGACC
TGCAAGCGCAGGACCGAGCCCACCGCATCGGGCAGCAGAACGAGGTGCGT
GTGCTCCGCCTCTGCACCGTCAACAGCGTGGAGGAGAAGATCCTAGCTGC
AGCCAAGTACAAGCTCAACGTGGACCAGAAGGTGATCCAGGCCGGCATGT
TCGACCAGAAGTCCTCCAGCCATGAGCGGCGCGCCTTCCTGCAGGCCATC
CTGGAGCACGAGGAGCAGGATGAGGAGGAAGACGAGGTGCCCGACGACGA
GACCGTCAACCAGATGATCGCCCGGCACGAGGAGGAGTTTGATCTGTTCA
TGCGCATGGACCTGGACCGCAGGCGCGAGGAGGCCCGCAACCCCAAGCGG
AAGCCGCGCCTCATGGAGGAGGACGAGCTCCCCTCGTGGATCATCAAGGA
CGACGCGGAGGTGGAGCGGCTGACCTGTGAGGAGGAGGAGGAGAAGATGT
TCGGCCGTGGCTCCCGCCACCGCAAGGAGGTGGACTACAGCGACTCACTG
ACGGAGAAGCAGTGGCTCAAGGCCATCGAGGAGGGCACGCTGGAGGAGAT
CGAAGAGGAGGTCCGGCAGAAGAAATCATCACGGAAGCGCAAGCGAGACA
GCGACGCCGGCTCCTCCACCCCGACCACCAGCACCCGCAGCCGCGACAAG
GACGACGAGAGCAAGAAGCAGAAGAAGCGCGGGCGGCCGCCTGCCGAGAA
ACTCTCCCCTAACCCACCCAACCTCACCAAGAAGATGAAGAAGATTGTGG
ATGCCGTGATCAAGTACAAGGACAGCAGTGGACGTCAGCTCAGCGAGGTG
TTCATCCAGCTGCGCTCGCGAAAGGAGCTGCCCGAGTACTACGAGCTCAT
CCGCAAGCCCGTGGACTTCAAGAAGATAAAGGAGCGCATTCGCAACCACA
AGTACCGCAGCCTCAACGACCTAGAGAAGGACGTCATGCTCCTGTGCCAG
AACGCACAGACCTTCAACCTGGAGGGCTCCCTGATCTATGAAGACTCCAT
CGTCTTGCAGTCGGTCTTCACCAGCGTGCGGCAGAAAATCGAGAAGGAGG
ATGACAGTGAAGGCGAGGAGAGTGAGGAGGAGGAAGAGGGCGAGGAGGAA
GGCTCCGAATCCGAATCTCGGTCCGTCAAAGTGAAGATCAAGCTTGGCCG
GAAGGAGAAGGCACAGGACCGGCTGAAGGGCGGCCGGCGGCGGCCGAGCC
GAGGGTCCCGAGCCAAGCCGGTCGTGAGTGACGATGACAGTGAGGAGGAA
CAAGAGGAGGACCGCTCAGGAAGTGGCAGCGAAGAAGACTGAGCCCCGAC
ATTCCAGTCTCGACCCCGAGCCCCTCGTTCCAGAGCTGAGATGGCATAGG
CCTTAGCAGTAACGGGTAGCAGCAGATGTAGTTTCAGACTTGGAGTAAAA
CTGTATAAACAAAAGAATCTTCCATATTTATACAGCAGAGAAGCTGTAGG
ACTGTTTGTGACTGGCCCTGTCCTGGCATCAGTAGCATCTGTAACAGCAT
TAACTGTCTTAAAGAGAGAGAGAGAGAATTCCGAATTGGGGAACACACGA
TACCTGTTTTTCTTTTCCGTTGCTGGCAGTACTGTTGCGCCGCAGTTTGG
AGTCACTGTAGTTAAGTGTGGATGCATGTGCGTCACCGTCCACTCCTCCT
ACTGTATTTTATTGGACAGGTCAGACTCGCCGGGGGCCCGGCGAGGGTAT
GTCAGTGTCACTGGATGTCAAACAGTAATAAATTAAACCAACAACAARAC
GCACAGCCAAAAAAAAA.

>NP_001122321.1 transcription activator BRG1 isoform A [Homo sapiens]

(SEQ ID NO: 15)
MSTPDPPLGGTPRPGPSPGPGPSPGAMLGPSPGPSPGSAHSMMGPSPGPP
SAGHPIPTQGPGGYPQDNMHQMHKPMESMHEKGMSDDPRYNQMKGMGMRS
GGHAGMGPPPSPMDQHSQGYPSPLGGSEHASSPVPASGPSSGPQMSSGPG
GAPLDGADPQALGQQNRGPTPFNQNQLHQLRAQIMAYKMLARGQPLPDHL
QMAVQGKRPMPGMQQQMPTLPPPSVSATGPGPGPGPGPGPGPGPAPPNYS
RPHGMGGPNMPPPGPSGVPPGMPGQPPGGPPKPWPEGPMANAAAPTSTPQ
KLIPPQPTGRPSPAPPAVPPAASPVMPPQTQSPGQPAQPAPMVPLHQKQS
RITPIQKPRGLDPVEILQEREYRLQARIAHRIQELENLPGSLAGDLRTKA
TIELKALRLLNFQRQLRQEVVVCMRRDTALETALNAKAYKRSKRQSLREA
RITEKLEKQQKIEQERKRRQKHQEYLNSILQHAKDFKEYHRSVTGKIQKL
TKAVATYHANTEREQKKENERIEKERMRRLMAEDEEGYRKLIDQKKDKRL
AYLLQQTDEYVANLTELVRQHKAAQVAKEKKKKKKKKKAENAEGQTPAIG
PDGEPLDETSQMSDLPVKVIHVESGKILTGTDAPKAGQLEAWLEMNPGYE
VAPRSDSEESGSEEEEEEEEEEQPQAAQPPTLPVEEKKKTPDPDSDDVSE
VDARHIIENAKQDVDDEYGVSQAIARGLQSYYAVAHAVTERVDKQSALMV
NGVLKQYQIKGLEWLVSLYNNNLNGILADEMGLGKTIQTIALITYLMEHK
RINGPFLILVPLSTLSNWAYEFDKWAPSVVKVSYKGSPAARRAFVPQLRS
GKFNVLLTTYEYIIKDKHILAKIRWKYMIVDEGHRMKNHHCKLTQVLNTH
YVAPRRLLLTGTPLQNKLPELWALLNFLLPTIFKSCSTFEQWFNAPFAMT
GEKVDLNEEETILIIRRLHKVLRPFLLRRLKKEVEAQLPEKVEYVIKCDM
SALQRVLYRHMQAKGVLLTDGSEKDKKGKGGTKTLMNTIMQLRKICNHPY
MFQHIEESFSEHLGFTGGIVQGLDLYRASGKFELLDRILPKLRATNHKVL
LFCQMTSLMTIMEDYFAYRGFKYLRLDGTTKAEDRGMLLKTFNEPGSEYF
IFLLSTRAGGLGLNLQSADTVIIFDSDWNPHQDLQAQDRAHRIGQQNEVR
VLRLCTVNSVEEKILAAAKYKLNVDQKVIQAGMFDQKSSSHERRAFLQAI
LEHEEQDESRHCSTGSGSASFAHTAPPPAGVNPDLEEPPLKEEDEVPDDE
TVNQMIARHEEEFDLFMPMDLDRRREEARNPKRKPRLMEEDELPSWIIKD
DAEVERLTCEEEEEKMFGRGSRHRKEVDYSDSLTEKQWLKKITGKDIHDT
ASSVARGLQFQRGLQFCTRASKAIEEGTLEEIEEEVRQKKSSRKRKRDSD
AGSSTPTTSTRSRDKDDESKKQKKRGRPPAEKLSPNPPNLTKKMKKIVDA
VIKYKDSSSGRQLSEVFIQLPSRKELPEYYELIRKPVDFKKIKERIRNHK
YRSLNDLEKDVMLLCQNAQTFNLEGSLIYEDSIVLQSVFTSVRQKIEKED
DSEGEESEEEEEGEEEGSESESRSVKVKIKLGRKEKAQDRLKGGRRRPSR
GSRAKPVVSDDDSEEEQEEDRSGSGSEED.

>NP_001122316.1 transcription activator BRG1 isoform B [Homo sapiens]

(SEQ ID NO: 16)
MSTPDPPLGGTPRPGPSPGPGPSPGAMLGPSPGPSPGSAHSMMGPSPGPP
SAGHPIPTQGPGGYPQDNMHQMHKPMESMHEKGMSDDPRYNQMKGMGMRS
GGHAGMGPPPSPMDQHSQGYPSPLGGSEHASSPVPASGPSSGPQMSSGPG
GAPLDGADPQALGQQNRGPTPFNQNQLHQLRAQIMAYKMLARGQPLPDHL
QMAVQGKRPMPGMQQQMPTLPPPSVSATGPGPGPGPGPGPGPGPAPPNYS
RPHGMGGPNMPPPGPSGVPPGMPGQPPGGPPKPWPEGPMANAAAPTSTPQ
KLIPPQPTGRPSPAPPAVPPAASPVMPPQTQSPGQPAQPAPMVPLHQKQS
RITPIQKPRGLDPVETLQEREYRLQARIAHRTQELENLPGSLAGDLRTKA
TIELKALRLLNFQRQLRQEVVVCMRRDTALETALNAKAYKRSKRQSLREA
RITEKLEKQQKIEQERKRRQKHQEYLNSILQHAKDFKEYHRSVTGKIQKL
TKAVATYHANTEREQKKENERIEKERMRRLMAEDEEGYRKLIDQKKDKRL
AYLLQQTDEYVANLTELVRQHKAAQVAKEKKKKKKKKKAENAEGQTPAIG
PDGEPLDETSQMSDLPVKVIHVESGKILTGTDAPKAGQLEAWLEMNPGYE
VAPRSDSEESGSEEEEEEEEEEQPQAAQPPTLPVEEKKKIPDPDSDDVSE
VDARHIIENAKQDVDDEYGVSQAIARGLQSYYAVAHAVTERVDKQSALMV
NGVLKQYQIKGLEWLVSLYNNNLNGILADEMGLGKTIQTIALITYLMEHK
RINGPFLIIVPLSTLSNWAYEFDKWAPSVVKVSYKGSPAARRAFVPQLRS
GKFNVLLTTYEYTTKDKHILAKIRWKYMTVDEGHRMKNHHCKLTQVLNTH
YVAPRRLLLTGTPLQNKLPELWALLNFLLPTIFKSCSTFEQWFNAPFAMT
GEKVDLNEEETILIIRRLHKVLRPFLLRRLKKEVEAQLPEKVEYVIKCDM
SALQRVLYRHMQAKGVLLTDGSEKDKKGKGGTKTLMNTIMQLRKICNHPY
MFQHIEESFSEHLGFTGGIVQGLDLYRASGKFELLDRILPKLRATNHKVL
LFCQMTSLMTIMEDYFAYRGFKYLRLDGTTKAEDRGMLLKTFNEPGSEYF
IFLLSTRAGGLGLNLQSADTVIIFDSDWNPHQDLQAQDRAHRTGQQNEVR
VLRLCTVNSVEEKILAAAKYKLNVDQKVIQAGMFDQKSSSHERRAFLQAI
LEHEEQDESRHCSTGSGSASFAHTAPPPAGVNPDLEEPPLKEEDEVPDDE
TVNQMIARHEEEFDLFMRMDLDRRREEARNPKRKPRLMEEDELPSWIIKD
DAEVERLTCEEEEEKMFGRGSRHRKEVDYSDSLTEKQWLKAIEEGTLEEI
EEEVRQKKSSRKRKRDSDAGSSTPTTSTRSRDKDDESKKQKKRGRPPAEK
LSPNPPNLTKKMKKIVDAVIKYKDSSSGRQLSEVFIQLPSRKELPEYYEL
IRKPVDFKKIKERIRNHKYRSLNDLEKDVMLLCQNAQTFNLEGSLIYEDS
IVLQSVFTSVRQKTEKEDDSEGEESEEEEEGEEEGSESESRSVKVKIKLG
RKEKAQDRLKGGRRRPSRGSRAKPVVSDDDSEEEQEEDRSGSGSEED.

>NP_001122317.1 transcription activator BRG1 isoform C [Homo sapiens]

(SEQ ID NO: 17)
MSTPDPPLGGTPRPGPSPGPGPSPGAMLGPSPGPSPGSAHSMMGPSPGPP
SAGHPIPTQGPGGYPQDNMHQMHKPMESMHEKGMSDDPRYNQMKGMGMRS
GGHAGMGPPPSPMDQHSQGYPSPLGGSEHASSPVPASGPSSGPQMSSGPG
GAPLDGADPQALGQQNRGPTPFNQNQLHQLRAQIMAYKMLARGQPLPDHL
QMAVQGKRPMPGMQQQMPTLPPPSVSATGPGPGPGPGPGPGPGPAPPNYS
RPHGMGGPNMPPPGPSGVPPGMPGQPPGGPPKPWPEGPMANAAAPTSTPQ
KLIPPQPTGRPSPAPPAVPPAASPVMPPQTQSPGQPAQPAPMVPLHQKQS
RITPIQKPRGLDPVEILQEREYRLQARIAHRIQELENLPGSLAGDLRTKA
TIELKALRLLNFQRQLRQEVVVCMRRDTALETALNAKAYKRSKRQSLREA
RITEKLEKQQKIEQERKRRQKHQEYLNSILQHAKDFKEYHRSVTGKIQKL
TKAVATYHANTEREQKKENERIEKERMRRLMAEDEEGYRKLIDQKKDKRL
AYLLQQTDEYVANLTELVRQHKAAQVAKEKKKKKKKKKAENAEGQTPAIG
PDGEPLDETSQMSDLPVKVIHVESGKILTGTDAPKAGQLEAWLEMNPGYE
VAPRSDSEESGSEEEEEEEEEEQPQAAQPPTLPVEEKKKIPDPDSDDVSE
VDARHIIENAKQDVDDEYGVSQALARGLQSYYAVAHAVTERVDKQSALMV
NGVLKQYQIKGLEWLVSLYNNNLNGILADEMGLGKTIQTIALITYLMEHK
RINGPFLIIVPLSTLSNWAYEFDKWAPSVVKVSYKGSPAARRAFVPQLRS
GKFNVLLTTYEYIIKDKHILAKIRWKYMIVDEGHRMKNHHCKLTQVLNTH
YVAPRRLLLTGTPLQNKLPELWALLNFLLPTIFKSCSTFEQWFNAPFAMT
GEKVDLNEEETILIIRRLHKVLRPFLLRRLKKEVEAQLPEKVEYVIKCDM
SALQRVLYRHMQAKGVLLTDGSEKDKKGKGGTKTLMNTIMQLRKICNHPY
MFQHIEESFSEHLGFTGGIVQGLDLYRASGKFELLDRILPKLRATNHKVL
LFCQMTSLMTIMEDYFAYRGFKYLRLDGTTKAEDRGMLLKTFNEPGSEYF
IFLLSTRAGGLGLNLQSADTVIIFDSDWNPHQDLQAQDRAHRIGQQNEVR
VLRLCTVNSVEEKTLAAAKYKLNVDQKVIQAGMFDQKSSSHERRAFLQAI
LEHEEQDEEEDEVPDDETVNQMIARHEEEFDLFMRMDLDRRREEARNPKR
KPRLMEEDELPSWIIKDDAEVERLTCEEEEEKMFGRGSRHRKEVDYSDSL
TEKQWLKTLKAIEEGTLEEIEEEVRQKKSSRKRKRDSDAGSSTPTTSTRS
RDKDDESKKQKKRGRPPAEKLSPNPPNLTKKMKKIVDAVIKYKDSSSGRQ
LSEVFIQLPSRKELPEYYELIRKPVDFKKIKERIRNHKYRSLNDLEKDVM
LLCQNAQTFNLEGSLIYEDSIVLQSVFTSVRQKIEKEDDSEGEESEEEEE
GEEEGSESESRSVKVKTKLGRKEKAQDRLKGGRRRPSRGSRAKPVVSDDD
SEEEQEEDRSGSGSEED.

>NP_001122318.1 transcription activator BRG1 isoform D [Homo sapiens]

(SEQ ID NO: 18)
MSTPDPPLGGTPRPGPSPGPGPSPGAMLGPSPGPSPGSAHSMMGPSPGPP
SAGHPIPTQGPGGYPQDNMHQMHKPMESMHEKGMSDDPRYNQMKGMGMRS
GGHAGMGPPPSPMDQHSQGYPSPLGGSEHASSPVPASGPSSGPQMSSGPG
GAPLDGADPQALGQQNRGPTPFNQNQLHQLRAQIMAYKMLARGQPLPDHL
QMAVQGKRPMPGMQQQMPTLPPPSVSATGPGPGPGPGPGPGPGPAPPNYS
RPHGMGGPNMPPPGPSGVPPGMPGQPPGGPPKPWPEGPMANAAAPTSTPQ
KLIPPQPTGRPSPAPPAVPPAASPVMPPQTQSPGQPAQPAPMVPLHQKQS
RITPIQKPRGLDPVEILPEREYRLQARIAHRIQETENLPGSLAGDLRTKA
TIELKALRLLNFQRQLRQEVVVCMRRDTALETALNAKAYKRSKRQSLREA
RITEKLEKQQKIEQERKRRQKHQEYLNSILQHAKDFKEYHRSVTGKIQKL
TKAVATYHANTEREQKKENERIEKERMRRLMAEDEEGYRKLIDQKKDKRL
AYLLQQTDEYVANLTELVRQHKAAQVAKEKKKKKKKKKAENAEGQTPAIG
PDGEPLDETSQMSDLPVKVIHVESGKILTGTDAPKAGPLEAWLEMNPGYE
VAPRSDSEESGSEEEEEEEEEEQPQAAQPPTLPVEEKKKIPDPDSDDVSE
VDARHIIENAKQDVDDEYGVSQALARGLQSYYAVAHAVTERVDKQSALMV
NGVLKQYQIKGLEWLVSLYNNNLNGILADEMGLGKTIQTIALITYLMEHK
RINGPFLIIVPLSTLSNWAYEFDKWAPSVVKVSYKGSPAARRAFVPQLRS
GKFNVLLTTYEYTTKDKHILAKIRWKYMTVDEGHRMKNHHCKLTQVLNTH
YVAPRRLLLTGTPLQNKLPELWALLNFLLPTIFKSCSTFEQWFNAPFAMT
GEKVDLNEEETILIIRRLHKVLRPFLLRRLKKEVEAQLPEKVEYVIKCDM
SALQRVLYRHMQAKGVLLTDGSEKDKKGKGGTKTLMNTIMQLRKICNHPY
MFQHIEESFSEHLGFTGGIVQGLDLYRASGKFELLDRILPKLRATNHKVL
LECQMTSLMTIMEDYFAYRGFKYLRLDGTTKAEDRGMLLKTFNEPGSEYF
IFLLSTRAGGLGLNLQSADTVIIFDSDWNPHQDLQAQDRAHRIGQQNEVR
VLRLCTVNSVEEKTLAAAKYKLNVDQKVIQAGMFDQKSSSHERRAFLQAI
LEHEEQDEEEDEVPDDETVNQMIARHEEEFDLFMRMDLDRRREEARNPKR
KPRLMEEDELPSWIIKDDAEVERLTCEEEEEKMFGRGSRHRKEVDYSDSL
TEKQWLKTLKAIEEGTLEEIEEEVRQKKSSRKRKRDSDAGSSTPTTSTRS
RDKDDESKKQKKRGRPPAEKLSPNPPNLTKKMKKIVDAVIKYKDSSGRQL
SEVFIQLPSRKELPEYYELIRKPVDFKKIKERIRNHKYRSLNDLEKDVML
LCQNAQTFNLEGSLIYEDSIVLQSVFTSVRQKIEKEDDSEGEESEEEEEG
EEEGSESESRSVKVKIKLGRKEKAQDRLKGGRRRPSRGSRAKPVVSDDDS
EEEQEEDRSGSGSEED.

>NP_001122319.1 transcription activator BRG1 isoform E [Homo sapiens]

(SEQ ID NO: 19)
MSTPDPPLGGTPRPGPSPGPGPSPGAMLGPSPGPSPGSAHSMMGPSPGPP
SAGHPIPTQGPGGYPQDNMHQMHKPMESMHEKGMSDDPRYNQMKGMGMRS
GGHAGMGPPPSPMDQHSQGYPSPLGGSEHASSPVPASGPSSGPQMSSGPG
GAPLDGADPQALGQQNRGPTPFNQNQLHQLRAQIMAYKMLARGQPLPDHL
QMAVQGKRPMPGMQQQMPTLPPPSVSATGPGPGPGPGPGPGPGPAPPNYS
RPHGMGGPNMPPPGPSGVPPGMPGQPPGGPPKPWPEGPMANAAAPTSTPQ
KLIPPQPTGRPSPAPPAVPPAASPVMPPQTQSPGQPAQPAPMVPLHQKQS
RITPIQKPRGLDPVEILQEREYRLQARIAHRIQELENLPGSLAGDLRTKA
TTELKALRLLNFQRQLRQEVVVCMRRDTALETALNAKAYKRSKRQSLREA
RITEKLEKQQKIEQERKRRQKHQEYLNSILQHAKDFKEYHRSVTGKIQKL
TKAVATYHANTEREQKKENERIEKERMRRLMAEDEEGYRKLIDQKKDKRL
AYLLQQTDEYVANLTELVRQHKAAQVAKEKKKKKKKKKAENAEGQTPAIG
PDGEPLDETSQMSDLPVKVIHVESGKILTGTDAPKAGQLEAWLEMNPGYE
VAPRSDSEESGSEEEEEEEEEEQPQAAQPPTLPVEEKKKIPDPDSDDVSE
VDARHIIENAKQDVDDEYGVSQALARGLQSYYAVAHAVTERVDKQSALMV
NGVLKQYQIKGLEWLVSLYNNNLNGILADEMGLGKTIQTIALITYLMEHK
RINGPFLIIVPLSTLSNWAYEFDKWAPSVVKVSYKGSPAARRAFVPQLRS
GKFNVLLTTYEYIIKDKHILAKIRWKYMIVDEGHRMKNHHCKLTQVLNTH
YVAPRRLLLTGTPLQNKLPELWALLNFLLPTIFKSCSTFEQWFNAPFAMT
GEKVDLNEEETILIIRRLHKVLRPFLLRRLKKEVEAQLPEKVEYVIKCDM
SALQRVLYRHMQAKGVLLTDGSEKDKKGKGGTKTLMNTIMQLRKICNHPY
MFQHIEESFSEHLGFTGGTVQGLDLYRASGKFELLDRILPKLRATNHKVL
LFCQMTSLMTIMEDYFAYRGFKYLRLDGTTKAEDRGMLLKTFNEPGSEYF
IFLLSTRAGGLGLNLQSADTVIIFDSDWNPHQDLQAQDRAHRIGQQNEVR
VLRLCTVNSVEEKILAAAKYKLNVDQKVIQAGMFDQKSSSHERRAFLQAI
LEHEEQDEEEDEVPDDETVNQMIARHEEEFDLFMRMDLDRRREEARNPKR
KPRLMEEDELPSWIIKDDAEVERLTCEEEEEKMFGRGSRHRKEVDYSDSL
TEKQWLKAIEEGTLEEIEEEVRQKKSSRKRKRDSDAGSSTPTTSTRSRDK
DDESKKQKKRGRPPAEKLSPNPPNLTKKMKKIVDAVIKYKDSSSGRQLSE
VFIQLPSRKELPEYYELIRKPVDFKKIKERIRNHKYRSLNDLEKDVMLLC
QNAQTFNLEGSLIYEDSIVLQSVFTSVRQKIEKEDDSEGEESEEEEEGEE
EGSESESRSVKVKIKLGRKEKAQDRLKGGRRRPSRGSRAKPVVSDDDSEE
EQEEDRSGSGSEED.

>NP_001122320.1 transcription activator BRG1 isoform F [Homo sapiens]

(SEQ ID NO: 20)
MSTPDPPLGGTPRPGPSPGPGPSPGAMLGPSPGPSPGSAHSMMGPSPGPP
SAGHPIPTQGPGGYPQDNMHQMHKPMESMHEKGMSDDPRYNQMKGMGMRS
GGHAGMGPPPSPMDQHSQGYPSPLGGSEHASSPVPASGPSSGPQMSSGPG
GAPLDGADPQALGQQNRGPTPFNQNQLHQLRAQIMAYKMIARGQPLPDHL
QMAVQGKRPMPGMQQQMPTLPPPSVSATGPGPGPGPGPGPGPGPAPPNYS
RPHGMGGPNMPPPGPSGVPPGMPGQPPGGPPKPWPEGPMANAAAPTSTPQ
KLIPPQPTGRPSPAPPAVPPAASPVMPPQTQSPGQPAQPAPMVPLHQKQS
RITPIQKPRGLDPVEILQEREYRLQARIAHRIQELENLPGSLAGDLRTKA
TIELKALRLLNFQRQLRQEVVVCMRRDTALETALNAKAYKRSKRQSLREA
RITEKLEKQQKIEQERKRRQKHQEYLNSILQHAKDFKEYHRSVTGKIQKL
TKAVATYHANTEREQKKENERIEKERMRRLMAEDEEGYRKLIDQKKDKRL
AYLLQQTDEYVANLTELVRQHKAAQVAKEKKKKKKKKKAENAEGQTPAIG
PDGEPLDETSQMSDLPVKVIHVESGKILTGTDAPKAGQLEAWLEMNPGYE
VAPRSDSEESGSEEEEEEEEEEQPQAAQPPTLPVEEKKKIPDPDSDDVSE
VDARHIIENAKQDVDDEYGVSQALARGLQSYYAVAHAVTERVDKQSALMV
NGVLKQYQIKGLEWLVSLYNNNLNGILADEMGLGKTIQTIALITYLMEHK
RINGPFLIIVPLSTLSNWAYEFDKWAPSVVKVSYKGSPAARRAFVPQLRS
GKFNVLLTTYEYIIKDKHILAKIRWKYMIVDEGHRMKNHHCKLTQVLNTH
YVAPRRLLLTGTPLQNKLPELWALLNFLLPTIFKSCSTFEQWFNAPFAMT
GEKVDLNEEETILIIRRLHKVLRPFLLRRLKKEVEAQLPEKVEYVIKCDM
SALQRVLYRHMQAKGVLLTDGSEKDKKGKGGTKTLMNTIMQLRKICNHPY
MFQHIEESFSEHLGFTGGIVQGLDLYRASGKFELLDRILPKLRATNHKVL
LFCQMTSLMTIMEDYFAYRGFKYLRLDGTTKAEDRGMLLKTFNEPGSEYF
IFLLSTRAGGLGLNLQSADTVIIFDSDWNPHQDLQAQDRAHRIGQQNEVR
VLRLCTVNSVEEKTLAAAKYKLNVDQKVTQAGMFDQKSSSHERRAFLQAI
LEHEEQDEEEDEVPDDETVNQMIARHEEEFDLFMRMDLDRRREEARNPKR
KPRLMEEDELPSWIIKDDAEVERLTCEEEEEKMFGRGSRHRKEVDYSDSL
TEKQWLKAIEEGTLEEIEEEVRQKKSSRKRKRDSDAGSSTPTTSTRSRDK
DDESKKQKKRGRPPAEKLSPNPPNLTKKMKKIVDAVIKYKDSSGRQLSEV
FIQLPSRKELPEYYELIRKPVDFKKIKERIRNHKYRSLNDLEKDVMLLCQ
NAQTFNLEGSLIYEDSIVLQSVFTSVRQKIEKEDDSEGEESEEEEEGEEE
GSESESRSVKVKIKLGRKEKAQDRLKGGRRRPSRGSRAKPVVSDDDSEEE
QEEDRSGSGSEED.

### REFERENCES

All publications, patents, patent applications, patent publications, and database entries sequence database entries) mentioned herein, e.g., in the Background, Summary, Detailed Description, Examples, and/or References sections, are hereby incorporated by reference in their entireties as if each individual publication, patent, patent application, patent publication, and database entry was specifically and individually incorporated herein by reference. In case of conflict, the present application, including any definitions herein, will control.

### Equivalents and Scope

Unless otherwise defined, all technical and scientific terms used herein have the same meaning as commonly understood by one of ordinary skill in the art to which this disclosure belongs. In the specification, the singular forms also include the plural unless the context clearly dictates otherwise. All publications, patent applications, patents and other references mentioned herein are incorporated by reference. The references cited herein are not admitted to be prior art to the claimed invention. In the case of conflict, the present specification, including definitions, will control. In addition, the materials, methods and examples are illustrative only and are not intended to be limiting.

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the disclosure encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present disclosure may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects are excluded are not set forth explicitly herein.

What is claimed is:

1. A method, comprising administering an enhancer of a zeste homolog 2 (EZH2) inhibitor to a subject having or diagnosed with a cell proliferative disorder characterized by a cell or a population of cells that exhibits a loss of function of SMARCA2 and/or SMARCA4.

2. A method of treating a cell proliferative disorder in a subject in need thereof comprising administering to the subject a therapeutically effective amount of an enhancer of a zeste homolog 2 (EZH2) inhibitor, wherein the cell proliferative disorder is characterized by a cell or a population of cells that exhibits a loss of function of SMARCA2 and/or SMARCA4.

3. The method of any one of claim 1 or 2, wherein the cell proliferative disorder is a cell proliferative disorder of the lung.

4. A method of treating a cell proliferative disorder of the lung in a subject in need thereof comprising administering to the subject a therapeutically effective amount of an enhancer of a zeste homolog 2 (EZH2) inhibitor.

5. The method of claim 4, wherein the cell proliferative disorder comprises or is characterized by a cell or a population of cells that exhibits a loss of function of SMARCA2 and/or a loss of function of SMARCA4.

6. The method of any one of claim 1, 2 or 4, wherein the cell proliferative disorder comprises or is characterized by a cell or a population of cells that exhibits a loss of function of SMARCA2 and SMARCA4.

7. The method of any one of claim 1, 2 or 4, wherein the cell proliferative disorder is characterized by a stem-, stem-like, or progenitor cell of origin.

8. The method of any one of claim 1, 2 or 4, wherein the cell proliferative disorder of the lung is characterized by a malignant growth or lesion in the lung.

9. The method of claim 8, wherein the malignant growth or lesion is a primary lesion.

10. The method of claims 8, wherein the malignant growth or lesion is, or is characterized by, a secondary or metastatic lesion.

11. The method of claim 8, wherein the malignant growth is a malignant lung neoplasm, a carcinoma, or a carcinoid tumor.

12. The method of any one of claim 1, 2 or 4, wherein the cell proliferative disorder of the lung is asbestos-induced hyperplasia, squamous metaplasia, and benign reactive mesothelial metaplasia.

13. The method of any one of claim 1, 2 or 4, wherein the cell proliferative disorder of the lung is lung cancer.

14. The method of claim 13, wherein the lung cancer is small cell lung cancer.

15. The method of claim 13, wherein the lung cancer is non-small cell lung cancer.

16. The method of claim 13, wherein the lung cancer is a squamous cell carcinoma.

17. The method of claim 13, wherein the lung cancer is an adenocarcinoma.

18. The method of claim 13, wherein the lung cancer is a small cell carcinoma.

19. The method of claim 13, wherein the lung cancer is a large cell carcinoma.

20. The method of claim 13, wherein the lung cancer is an adenosquamous cell carcinoma.

21. The method of claim 13, wherein the lung cancer is mesothelioma.

22. The method of any one of claim 1, 2, or 4, wherein the cell proliferative disease is characterized by a primary tumor, wherein the primary tumor
(A) exhibits SMARCA2/SMARCA4 dual loss; and
(B) is poorly differentiated and/or exhibits epithelial to mesenchymal transition (EMT) features.

23. The method of claim 22, wherein the primary tumor exhibits low E-cadherin and high vimentin expression levels.

24. The method of any one of claim 1, 2 or 4, wherein the subject has been or is being administered an additional therapeutic agent concurrently or in temporal proximity with the administration of the EZH2 inhibitor.

25. The method of claim 24, wherein the additional therapeutic agent is a standard-of-care agent.

26. The method of claim 25, wherein the additional agent is or comprises an agent listed in Schematic 1, or is or comprises a combination of two or more agents listed in Schematic 1.

27. The method of claim 24, wherein the additional therapeutic agent is an immune checkpoint inhibitor.

28. The method of claim 27, wherein the immune checkpoint inhibitor is a CTLA4 inhibitor, a PD-1 inhibitor and/or a PD-L1 inhibitor, a LAG3 inhibitor, a B7-H3 inhibitor, or a Tim3 inhibitor.

29. The method of claim 28, wherein the immune checkpoint inhibitor comprises Ipilimumab, Ticilimumab, AGEN-1884, Nivolumab, Pembrolizumab, Atezolizumab, Durvalumab, Avelumab, BMS-936559, AMP-224, MEDI-0680, TSR-042, BGB-108, STI-1014, KY-1003, ALN-PDL, BGB-A317, KD-033, REGN-2810, PDR-001, SHR-1210, MGD-013, PF-06801591, CX-072, IMP-731, LAG-525, BMS-986016, GSK-2831781, Enoblituzumab, 1241-8H9, DS-5573, MBG-453, or a combination thereof.

30. The method of any claims 24, wherein the EZH2 inhibitor and the additional therapeutic agent are administered sequentially to the subject.

31. The method of claim 24, wherein the EZH2 inhibitor and the additional therapeutic agent are administered via different administration routes and at different intervals.

32. The method of any one of claim 1, 2 or 4, wherein the EZH2 inhibitor is administered orally twice a day.

33. The method of any one of claim 1, 2 or 4, wherein the method further comprises detecting SMARCA2 and/or SMARCA4 protein expression and/or a function of a SMARCA2 and/or of a SMARCA4 protein.

34. The method of claim 33, wherein the expression and/or function of the SMARCA2 and/or the SMARCA4 protein is evaluated by a method comprising:
(a) obtaining a biological sample from the subject;
(b) contacting the biological sample or a portion thereof with an antibody that specifically binds SMARCA2 or SMARCA4; and
(c) detecting an amount of the antibody that is bound to SMARCA2 or SMARCA4.

35. The method of any one of claim 1, 2 or 4, wherein the method further comprises detecting a genomic mutation in the gene encoding the SMARCA2 and/or the gene encoding the SMARCA4 protein in a biological sample obtained from the subject.

36. The method of claim 35, wherein the genomic mutation is detected by a method comprising:
(a) obtaining a biological sample from the subject;
(b) sequencing at least one DNA sequence encoding a SMARCA2 protein or a portion thereof, and/or at least one DNA sequence encoding a SMARCA4 protein or a portion thereof, in the biological sample; and
(c) determining if the at least one DNA sequence encoding a SMARCA2 protein or a portion thereof, and/or the at least one DNA sequence encoding a SMARCA4 protein or a portion thereof, comprises a mutation affecting the expression and/or function of the SMARCA2 protein or the SMARCA4 protein.

37. The method of any one of claim 1 or 2, wherein the EZH2 inhibitor inhibits tri-methylation of lysine 27 of histone 3 (H3K27).

38. A method, comprising detecting a SMARCA2 and/or a SMARCA4 loss of function in a sample obtained from a subject.

39. The method of claim 38, wherein the subject has cancer.

40. The method of any one of claim 38 or 39, wherein the method further comprises administering an EZH2 inhibitor to the subject, if a SMARCA2 and/or SMARCA4 loss of function is detected in the subject.

41. The method of claim 40, wherein the SMARCA2 loss of function is not associated with a genomic mutation in a gene encoding SMARCA2 protein, and/or wherein the SMARCA4 loss of function is associated with a genomic mutation in a gene encoding SMARCA4.

42. The method of claim 38, wherein the subject has NSCLC.

43. The method of any one of claim 1, 2, or 4, wherein the EZH2 inhibitor is 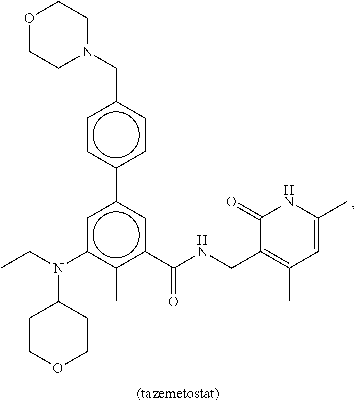
or a pharmaceutically acceptable salt thereof.

44. The method of any one of claim 1, 2, or 4, wherein the EZH2 inhibitor is 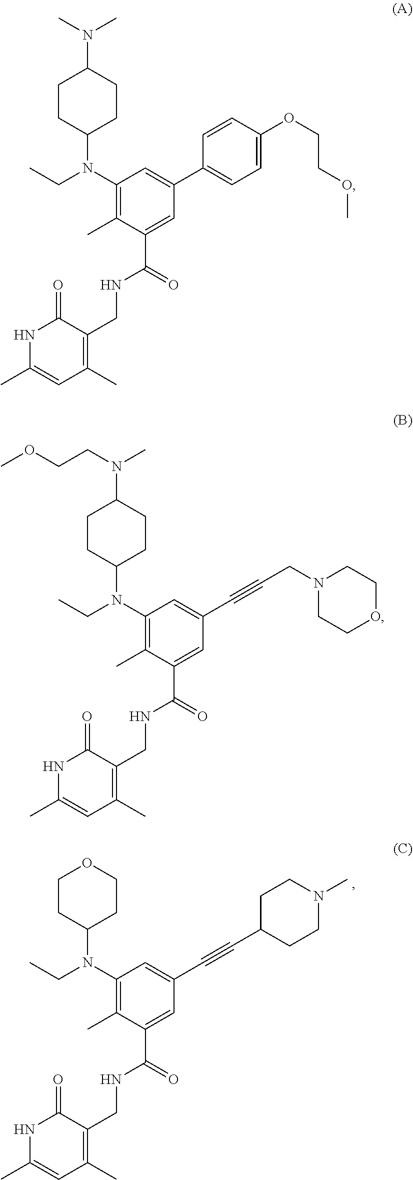 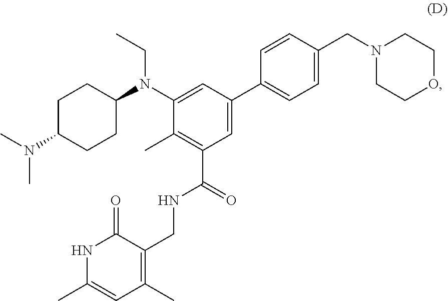 [SMILES: CCN(c1cc(-c2ccc(CN3CCOCC3)cc2)cc(C(=O)NCc2c(C)cc(C)[nH]c2=O)c1C)[C@H]1CC[C@H](N(C)C)CC1]
a stereoisomer, a pharmaceutically acceptable salt and/or a solvate thereof.

45. The method of any one of claim 1, 2, or 4, wherein the EZH2 inhibitor is 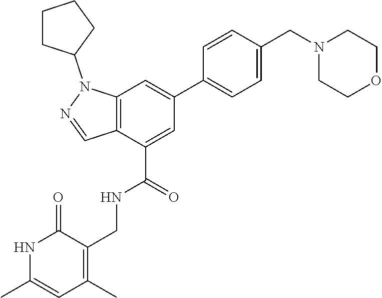 [SMILES: Cc1cc(C)c(CNC(=O)c2cc(-c3ccc(CN4CCOCC4)cc3)cc3c2cnn3C2CCCC2)c(=O)[nH]1]
or a pharmaceutically acceptable salt thereof.

46. The method of any one of claim 1, 2, or 4, wherein the EZH2 inhibitor is 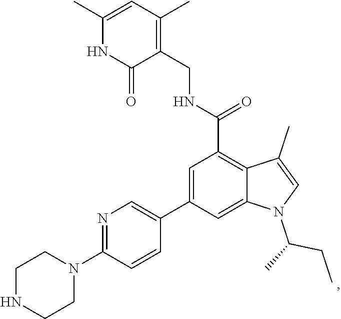 [SMILES: CC[C@H](C)n1cc(C)c2c(C(=O)NCc3c(C)cc(C)[nH]c3=O)cc(-c3ccc(N4CCNCC4)nc3)cc21]
a stereoisomer, a pharmaceutical acceptable salt and/or a solvate thereof.

47. The method of any one of claim 1, 2, or 4, wherein the EZH2 inhibitor is 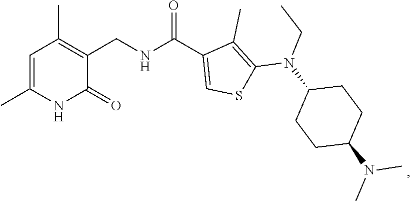
a stereoisomer, a pharmaceutically acceptable salt and/or a solvate thereof.

48. The method of any one of claim 1, 2, or 4, wherein the EZH2 inhibitor is 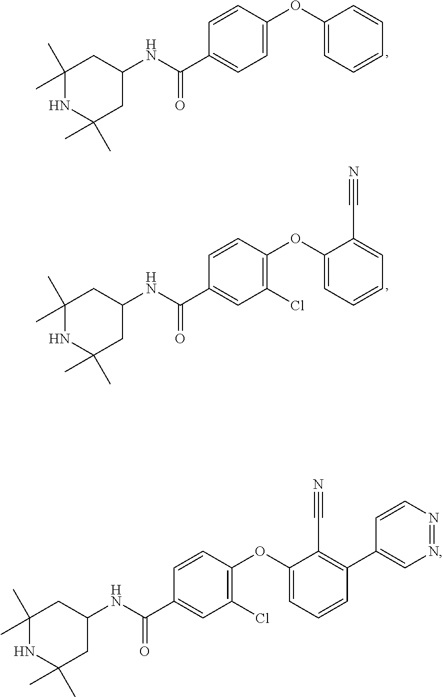 [SMILES: CC1(C)CC(NC(=O)c2ccc(Oc3ccccc3)cc2)CC(C)(C)N1 ; CC1(C)CC(NC(=O)c2ccc(Oc3ccccc3C#N)c(Cl)c2)CC(C)(C)N1 ; CC1(C)CC(NC(=O)c2ccc(Oc3cccc(-c4ccnnc4)c3C#N)c(Cl)c2)CC(C)(C)N1]
a stereoisomer, a pharmaceutically acceptable salt and/or a solvate thereof.

49. The method of any one of claim 1, 2, or 4, wherein the EZH2 inhibitor is administered orally.

50. The method of any one of claims 1, 2, or 4, wherein the EZH2 inhibitor is formulated as an oral tablet.

51. The method of any one of claim 1, 2, or 4, wherein the EZH2 inhibitor is administered at a dose of between 10 mg/kg/day and 1600 mg/kg/day.

52. The method of any one of claim 1, 2, or 4, wherein the EZH2 inhibitor is administered at a dose of about 100, 200, 400, 800, or 1600 mg.

53. The method of any one of claim 1, 2, or 4, wherein the EZH2 inhibitor is administered at a dose of about 800 mg.

54. The method of any one of claim 1, 2, or 4, wherein the EZH2 inhibitor is administered twice per day (BID).

55. Use of an enhancer of a zeste homolog 2 (EZH2) inhibitor for treating a cell proliferative disorder in a subject in need thereof, the use comprising administering to the subject a therapeutically effective amount of an enhancer of a zeste homolog 2 (EZH2.) inhibitor, wherein the cell proliferative disorder is characterized by a cell or a population of cells that exhibits a loss of function of SMARCA2 and/or SMARCA4.

56. The use of claim 55, wherein the cell proliferative disorder is a cell proliferative disorder of the lung.

57. Use of an enhancer of zeste homolog 2 (EZH2) inhibitor, for treating a cell proliferative disorder of the lung in a subject in need thereof, the use comprising administering to the subject a therapeutically effective amount of the enhancer of a zeste homolog 2 (EZH2) inhibitor.

58. The use of claim 57, wherein the cell proliferative disorder comprises or is characterized by a cell or a population of cells that exhibits a loss of function of SMARCA2 and/or a loss of function of SMARCA4.

59. The use of any one of claims 55-58, wherein the cell proliferative disorder comprises or is characterized by a cell or a population of cells that exhibits a loss of function of SMARCA2 and SMARCA4.

60. The use of any one of claims 55-59, wherein the cell proliferative disorder is characterized by a stem-, stem-like, or progenitor cell of origin.

61. The use of any one of claims 55-60, wherein the cell proliferative disorder of the lung is characterized by a malignant growth or lesion in the lung.

62. The use of any one of claims 55-61, wherein the malignant growth or lesion is a primary lesion.

63. The use of any one of claims 55-62, wherein the malignant growth or lesion is, or is characterized by, a secondary or metastatic lesion.

64. The use of any one of claims 55-63, wherein the malignant growth is a malignant lung neoplasm, a carcinoma, or a carcinoid tumor.

65. The use of any one of claims 55-64, wherein the cell proliferative disorder of the lung is asbestos-induced hyperplasia, squamous metaplasia, and benign reactive mesothelial metaplasia.

66. The use of any one of claims 55-65, wherein the cell proliferative disorder of the lung is lung cancer.

67. The use of claim 66, wherein the lung cancer is small cell lung cancer.

68. The use of claim 66, wherein the lung cancer is non-small cell lung cancer.

69. The use of claim 66, wherein the lung cancer is a squamous cell carcinoma.

70. The use of claim 66, wherein the lung cancer is an adenocarcinoma.

71. The use of claim 66, wherein the lung cancer is a small cell carcinoma.

72. The use of claim 66, wherein the lung cancer is a large cell carcinoma.

73. The use of claim 66, wherein the lung cancer is an adenosquamous cell carcinoma.

74. The use of claim 66, wherein the lung cancer is mesothelioma.

75. The use of any one of claims 55-74, wherein the cell proliferative disease is characterized by a primary tumor, wherein the primary tumor
(A) exhibits SMARCA2/SMARCA4 dual loss; and
(B) is poorly differentiated and/or exhibits epithelial to mesenchymal transition (EMT) features.

76. The use of claim 75, wherein the primary tumor exhibits low E-cadherin and high vimentin expression levels.

77. The use of any one of claims 55-76, wherein the subject has been or is being administered an additional therapeutic agent concurrently or in temporal proximity with the administration of the EZH2 inhibitor.

78. The use of claim 77, wherein the additional therapeutic agent is a standard-of-care agent.

79. The use of claim 78, wherein the additional agent is or comprises an agent listed in Schematic 1, or is or comprises a combination of two or more agents listed in Schematic 1.

80. The use of claim 79, wherein the additional therapeutic agent is an immune checkpoint inhibitor.

81. The use of claim 80, wherein the immune checkpoint inhibitor is a CTLA4 inhibitor, a PD-1 inhibitor and/or a PD-L1 inhibitor, a LAG3 inhibitor, a B7-H3 inhibitor, or a Tim3 inhibitor.

82. The use of claim 81, wherein the immune checkpoint inhibitor comprises Ipilimumab, Ticilimumab, AGEN-1884, Nivolumab, Pembrolizumab, Atezolizumab, Durvalumab, Avelumab, BMS-936559, AMP-224, MEDI-0680, TSR-042, BGB-108, STI-1014, KY-1003, ALN-PDL, BGB-A317, KD-033, REGN-2810, PDR-001, SHR-1210, MGD-013, PF-06801591, CX-072, IMP-731, LAG-525, BMS-986016, GSK-2831781 Enoblituzumab, 1241-8H9, DS-5573, MBG-453, or a combination thereof.

83. The use of any one of claims 77-82, wherein the EZH2 inhibitor and the additional therapeutic agent are administered sequentially to the subject.

84. The use of any one of claims 77-83, wherein the EZH2 inhibitor and the additional therapeutic agent are administered via different administration routes and at different intervals.

85. The use of any one of claims 55-84, wherein the EZH2 inhibitor is administered orally twice a day.

86. The use of any one of claims 55-85, wherein the use further comprises detecting SMARCA2 and/or SMARCA4 protein expression and/or a function of a SMARCA2 and/or of a SMARCA4 protein.

87. The use of claim 86, wherein the expression and/or function of the SMARCA2 and/or the SMARCA4 protein is evaluated by the steps comprising:
(a) obtaining a biological sample from the subject;
(b) contacting the biological sample or a portion thereof with an antibody that specifically binds SMARCA2 or SMARCA4; and
(c) detecting an amount of the antibody that is bound to SMARCA2 or SMARCA4.

88. The use of any one of claims 55-87, wherein the use further comprises detecting a genomic mutation in the gene encoding the SMARCA2 and/or the gene encoding the SMARCA4 protein in a biological sample obtained from the subject.

89. The use of claim 88, wherein the genomic mutation is detected by the steps comprising:
(a) obtaining a biological sample from the subject;
(b) sequencing at least one DNA sequence encoding a SMARCA2 protein or a portion thereof, and/or at least one DNA sequence encoding a SMARCA4 protein or a portion thereof, in the biological sample; and
(c) determining if the at least one DNA sequence encoding a SMARCA2 protein or a portion thereof, and/or the at least one DNA sequence encoding a SMARCA4 protein or a portion thereof, comprises a mutation affecting the expression and/or function of the SMARCA2 protein or the SMARCA4 protein.

90. The use of claim 55, wherein the EZH2 inhibitor inhibits tri-methylation of lysine 27 of histone 3 (H3K27).

91. The use of any one of claims 88-89, wherein the use further comprises detecting a SMARCA2 and/or a SMARCA4 loss of function in a sample obtained from a subject.

92. The use of claim 91, wherein the SMARCA2 loss of function is not associated with a genomic mutation in a gene encoding SMARCA2 protein, and/or wherein the SMARCA4 loss of function is associated with a genomic mutation in a gene encoding SMARCA4.

93. The use of any one of claims 91-92, wherein the subject has NSCLC.

94. The use of any one of claims 55-93, wherein the EZH2 inhibitor is 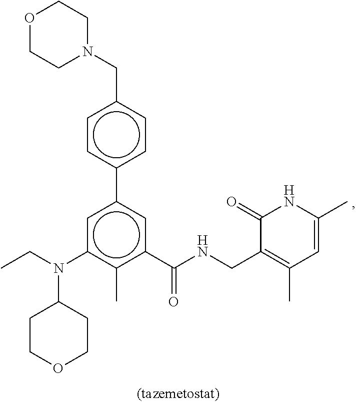
or a pharmaceutically acceptable salt thereof.

95. The use of any one of claims 55-93, wherein the EZH2 inhibitor is 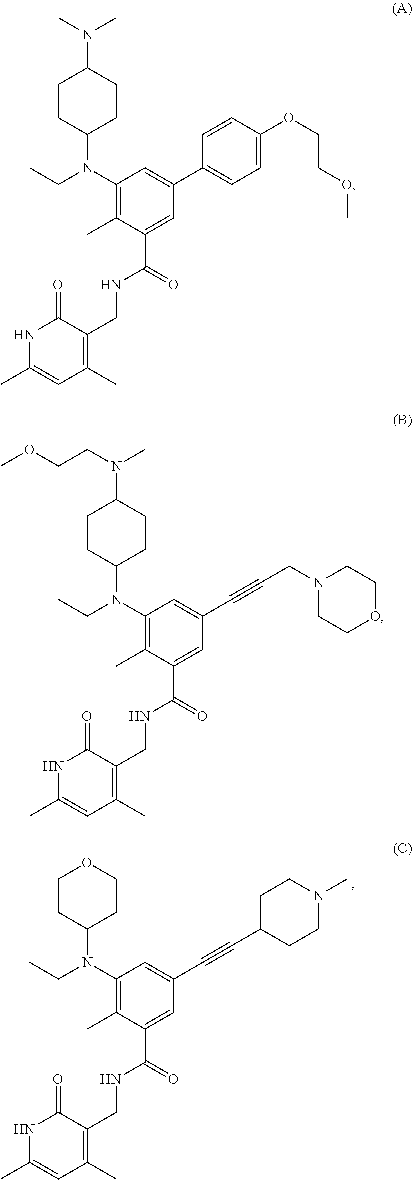 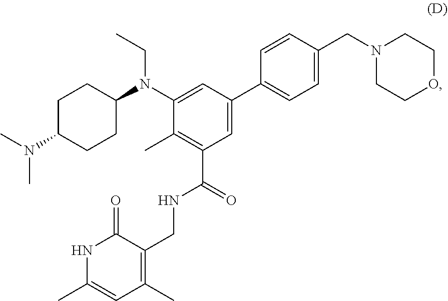 [SMILES: CCN(c1cc(-c2ccc(CN3CCOCC3)cc2)cc(C(=O)NCc2c(C)cc(C)[nH]c2=O)c1C)[C@H]1CC[C@H](N(C)C)CC1]
a stereoisomer, a pharmaceutically acceptable salt and/or a solvate thereof.

96. The use of any one of claims 55-93, wherein the EZH2 inhibitor is 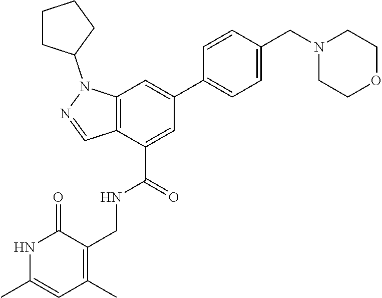 [SMILES: Cc1cc(C)c(CNC(=O)c2cc(-c3ccc(CN4CCOCC4)cc3)cc3c2cnn3C2CCCC2)c(=O)[nH]1]
or a pharmaceutically acceptable salt thereof.

97. The use of any one of claims 55-93, wherein the EZH2 inhibitor is 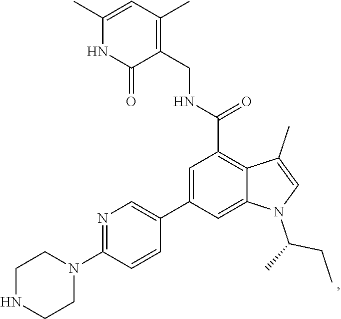 [SMILES: CC[C@H](C)n1cc(C)c2c(C(=O)NCc3c(C)cc(C)[nH]c3=O)cc(-c3ccc(N4CCNCC4)nc3)cc21]
a stereoisomer, a pharmaceutically acceptable salt and/or a solvate thereof.

98. The use of any one of claims 55-93, wherein the EZH2 inhibitor is 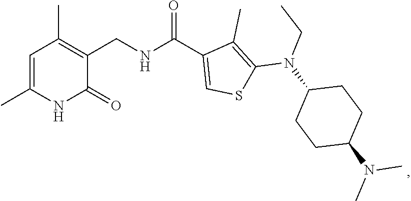
a stereoisomer, a pharmaceutically acceptable salt and/or a solvate thereof.

99. The use of any one of claims 55-93, wherein the EZH2 inhibitor is 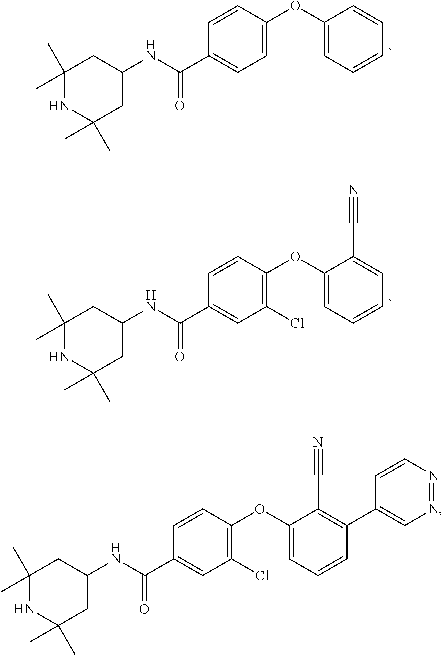 [SMILES: CC1(C)CC(NC(=O)c2ccc(Oc3ccccc3)cc2)CC(C)(C)N1 ; CC1(C)CC(NC(=O)c2ccc(Oc3ccccc3C#N)c(Cl)c2)CC(C)(C)N1 ; CC1(C)CC(NC(=O)c2ccc(Oc3cccc(-c4ccnnc4)c3C#N)c(Cl)c2)CC(C)(C)N1]
a stereoisomer, a pharmaceutically acceptable salt and/or a solvate thereof.

100. The use of any one of claims 55-93, wherein the EZH2 inhibitor is administered orally.

101. The use of any one of claims 55-93, wherein the EZH2 inhibitor is formulated as an oral tablet.

102. The use of any one of claims 55-93, wherein the EZH2 inhibitor is administered at a dose of between 10 mg/kg/day and 1600 mg/kg/day.

103. The use of any one of claims 55-93, wherein the EZH2 inhibitor is administered at a dose of about 100, 200, 400, 800, or 1600 mg.

104. The use of any one of claims 55-93, wherein the EZH2 inhibitor is administered at a dose of about 800 mg.

105. The use of any one of claims 55-93, wherein the EZH2 inhibitor is administered twice per day (BID).


SEQUENCE LISTING (20 sequences)

SEQ ID NO: 1 - DNA, 5879 bp, Homo sapiens
tcagaagaaagccccgagatcacagagacccggcgagatcacagagacccggcctgaaggaacgtggaaagaccaatgtacctgttttgaccggttgcctggagcaagaagttccagttggggagaattttcagaagataaagtcggagattgtggaaagacttgacttgcagcattactctactgactggcagagacaggagaggtagatgtccacgcccacagaccctggtgcgatgccccacccagggccttcgccggggcctgggccttcccctgggccaattcttgggcctagtccaggaccaggaccatccccaggttccgtccacagcatgatggggccaagtcctggacctccaagtgtctcccatcctatgccgacgatggggtccacagacttcccacaggaaggcatgcatcaaatgcataagcccatcgatggtatacatgacaaggggattgtagaagacatccattgtggatccatgaagggcactggtatgcgaccacctcacccaggcatgggccctccccagagtccaatggatcaacacagccaaggttatatgtcaccacacccatctccattaggagccccagagcacgtctccagccctatgtctggaggaggcccaactccacctcagatgccaccaagccagccgggggccctcatcccaggtgatccgcaggccatgagccagcccaacagaggtccctcacctttcagtcctgtccagctgcatcagcttcgagctcagattttagcttataaaatgctggcccgaggccagcccctccccgaaacgctgcagcttgcagtccaggggaaaaggacgttgcctggcttgcagcaacaacagcagcagcaacagcagcagcagcagcagcagcagcagcagcagcagcagcaacagcagccgcagcagcagccgccgcaaccacagacgcagcaacaacagcagccggcccttgttaactacaacagaccatctggcccggggccggagctgagcggcccgagcaccccgcagaagctgccggtgcccgcgcccggcggccggccctcgcccgcgccccccgcagccgcgcagccgcccgcggccgcagtgcccgggccctcagtgccgcagccggccccggggcagccctcgcccgtcctccagctgcagcagaagcagagccgcatcagccccatccagaaaccgcaaggcctggaccccgtggaaattctgcaagagcgggaatacagacttcaggcccgcatagctcataggatacaagaactggaaaatctgcctggctctttgccaccagatttaagaaccaaagcaaccgtggaactaaaagcacttcggttactcaatttccagcgtcagctgagacaggaggtggtggcctgcatgcgcagggacacgaccctggagacggctctcaactccaaagcatacaaacggagcaagcgccagactctgagagaagctcgcatgaccgagaagctggagaagcagcagaagattgagcaggagaggaaacgccgtcagaaacaccaggaatacctgaacagtattttgcaacatgcaaaagattttaaggaatatcatcggtctgtggccggaaagatccagaagctctccaaagcagtggcaacttggcatgccaacactgaaagagagcagaagaaggagacagagcggattgaaaaggagagaatgcggcgactgatggctgaagatgaggagggttatagaaaactgattgatcaaaagaaagacaggcgtttagcttaccttttgcagcagaccgatgagtatgtagccaatctgaccaatctggtttgggagcacaagcaagcccaggcagccaaagagaagaagaagaggaggaggaggaagaagaaggctgaggagaatgcagagggtggggagtctgccctgggaccggatggagagcccatagatgagagcagccagatgagtgacctccctgtcaaagtgactcacacagaaaccggcaaggttctgttcggaccagaagcacccaaagcaagtcagctggacgcctggctggaaatgaatcctggttatgaagttgcccctagatctgacagtgaagagagtgattctgattatgaggaagaggatgaggaagaagagtccagtaggcaggaaaccgaagagaaaatactcctggatccaaatagcgaagaagtttctgagaaggatgctaagcagatcattgagacagctaagcaagacgtggatgatgaatacagcatgcagtacagtgccaggggctcccagtcctactacaccgtggctcatgccatctcggagagggtggagaaacagtctgccctcctaattaatgggaccctaaagcattaccagctccagggcctggaatggatggtttccctgtataataacaacttgaacggaatcttagccgatgaaatggggcttggaaagaccatacagaccattgcactcatcacttatctgatggagcacaaaagactcaatggcccctatctcatcattgttcccctttcgactctatctaactggacatatgaatttgacaaatgggctccttctgtggtgaagatttcttacaagggtactcctgccatgcgtcgctcccttgtcccccagctacggagtggcaaattcaatgtcctcttgactacttatgagtatattataaaagacaagcacattcttgcaaagattcggtggaaatacatgatagtggacgaaggccaccgaatgaagaatcaccactgcaagctgactcaggtcttgaacactcactatgtggcccccagaaggatcctcttgactgggaccccgctgcagaataagctccctgaactctgggccctcctcaacttcctcctcccaacaatttttaagagctgcagcacatttgaacaatggttcaatgctccatttgccatgactggtgaaagggtggacttaaatgaagaagaaactatattgatcatcaggcgtctacataaggtgttaagaccatttttactaaggagactgaagaaagaagttgaatcccagcttcccgaaaaagtggaatatgtgatcaagtgtgacatgtcagctctgcagaagattctgtatcgccatatgcaagccaaggggatccttctcacagatggttctgagaaagataagaaggggaaaggaggtgctaagacacttatgaacactattatgcagttgagaaaaatctgcaaccacccatatatgtttcagcacattgaggaatcctttgctgaacacctaggctattcaaatggggtcatcaatggggctgaactgtatcgggcctcagggaagtttgagctgcttgatcgtattctgccaaaattgagagcgactaatcaccgagtgctgcttttctgccagatgacatctctcatgaccatcatggaggattattttgcttttcggaacttcctttacctacgccttgatggcaccaccaagtctgaagatcgtgctgctttgctgaagaaattcaatgaacctggatcccagtatttcattttcttgctgagcacaagagctggtggcctgggcttaaatcttcaggcagctgatacagtggtcatctttgacagcgactggaatcctcatcaggatctgcaggcccaagaccgagctcaccgcatcgggcagcagaacgaggtccgggtactgaggctctgtaccgtgaacagcgtggaggaaaagatcctcgcggccgcaaaatacaagctgaacgtggatcagaaagtgatccaggcgggcatgtttgaccaaaagtcttcaagccacgagcggagggcattcctgcaggccatcttggagcatgaggaggaaaatgaggaagaagatgaagtaccggacgatgagactctgaaccaaatgattgctcgacgagaagaagaatttgacctttttatgcggatggacatggaccggcggagggaagatgcccggaacccgaaacggaagccccgtttaatggaggaggatgagctgccctcctggatcattaaggatgacgctgaagtagaaaggctcacctgtgaagaagaggaggagaaaatatttgggagggggtcccgccagcgccgtgacgtggactacagtgacgccctcacggagaagcagtggctaagggccatcgaagacggcaatttggaggaaatggaagaggaagtacggcttaagaagcgaaaaagacgaagaaatgtggataaagatcctgcaaaagaagatgtggaaaaagctaagaagagaagaggccgccctcccgctgagaaactgtcaccaaatccccccaaactgacaaagcagatgaacgctatcatcgatactgtgataaactacaaagataggtgtaacgtggagaaggtgcccagtaattctcagttggaaatagaaggaaacagttcagggcgacagctcagtgaagtcttcattcagttaccttcaaggaaagaattaccagaatactatgaattaattaggaagccagtggatttcaaaaaaataaaggaaaggattcgtaatcataagtaccggagcctaggcgacctggagaaggatgtcatgcttctctgtcacaacgctcagacgttcaacctggagggatcccagatctatgaagactccatcgtcttacagtcagtgtttaagagtgcccggcagaaaattgccaaagaggaagagagtgaggatgaaagcaatgaagaggaggaagaggaagatgaagaagagtcagagtccgaggcaaaatcagtcaaggtgaaaattaagctcaataaaaaagatgacaaaggccgggacaaagggaaaggcaagaaaaggccaaatcgaggaaaagccaaacctgtagtgagcgattttgacagcgatgaggagcaggatgaacgtgaacagtcagaaggaagtgggacggatgatgagtgatcagtatggacctttttccttggtagaactgaattccttcctcccctgtctcatttctacccagtgagttcatttgtcatataggcactgggttgtttctatatcatcatcgtctataaactagctttaggatagtgccagacaaacatatgatatcatggtgtaaaaaacacacacatacacaaatatttgtaacatattgtgaccaaatgggcctcaaagattcagattgaaacaaacaaaaagcttttgatggaaaatatgtgggtggatagtatatttctatgggtgggtctaatttggtaacggtttgattgtgcctggttttatcacctgttcagatgagaagatttttgtcttttgtagcactgataaccaggagaagccattaaaagccactggttattttatttttcatcaggcaattttcgaggtttttatttgttcggtattgtttttttacactgtggtacatataagcaactttaataggtgataaatgtacagtagttagatttcacctgcatatacatttttccattttatgctctatgatctgaacaaaagctttttgaattgtataagatttatgtctactgtaaacattgcttaatttttttgctcttgatttaaaaaaaagttttgttgaaagcgctattgaatattgcaatctatatagtgtattggatggcttcttttgtcaccctgatctcctatgttaccaatgtgtatcgtctccttctccctaaagtgtacttaatctttgctttctttgcacaatgtctttggttgcaagtcataagcctgaggcaaataaaattccagtaatttcgaagaatgtggtgttggtgctttcctaataaagaaataatttagcttgacaaaaaaaaaaaaaaa

SEQ ID NO: 2 - DNA, 5838 bp, Homo sapiens
gcgtcttccggcgcccgcggaggaggcgagggtgggacgctgggcggagcccgagtttaggaagaggaggggacggctgtcatcaatgaagtcatattcataatctagtcctctctccctctgtttctgtactctgggtgactcagagagggaagagattcagccagcacactcctcgcgagcaagcattactctactgactggcagagacaggagaggtagatgtccacgcccacagaccctggtgcgatgccccacccagggccttcgccggggcctgggccttcccctgggccaattcttgggcctagtccaggaccaggaccatccccaggttccgtccacagcatgatggggccaagtcctggacctccaagtgtctcccatcctatgccgacgatggggtccacagacttcccacaggaaggcatgcatcaaatgcataagcccatcgatggtatacatgacaaggggattgtagaagacatccattgtggatccatgaagggcactggtatgcgaccacctcacccaggcatgggccctccccagagtccaatggatcaacacagccaaggttatatgtcaccacacccatctccattaggagccccagagcacgtctccagccctatgtctggaggaggcccaactccacctcagatgccaccaagccagccgggggccctcatcccaggtgatccgcaggccatgagccagcccaacagaggtccctcacctttcagtcctgtccagctgcatcagcttcgagctcagattttagcttataaaatgctggcccgaggccagcccctccccgaaacgctgcagcttgcagtccaggggaaaaggacgttgcctggcttgcagcaacaacagcagcagcaacagcagcagcagcagcagcagcagcagcagcagcagcagcaacagcagccgcagcagcagccgccgcaaccacagacgcagcaacaacagcagccggcccttgttaactacaacagaccatctggcccggggccggagctgagcggcccgagcaccccgcagaagctgccggtgcccgcgcccggcggccggccctcgcccgcgccccccgcagccgcgcagccgcccgcggccgcagtgcccgggccctcagtgccgcagccggccccggggcagccctcgcccgtcctccagctgcagcagaagcagagccgcatcagccccatccagaaaccgcaaggcctggaccccgtggaaattctgcaagagcgggaatacagacttcaggcccgcatagctcataggatacaagaactggaaaatctgcctggctctttgccaccagatttaagaaccaaagcaaccgtggaactaaaagcacttcggttactcaatttccagcgtcagctgagacaggaggtggtggcctgcatgcgcagggacacgaccctggagacggctctcaactccaaagcatacaaacggagcaagcgccagactctgagagaagctcgcatgaccgagaagctggagaagcagcagaagattgagcaggagaggaaacgccgtcagaaacaccaggaatacctgaacagtattttgcaacatgcaaaagattttaaggaatatcatcggtctgtggccggaaagatccagaagctctccaaagcagtggcaacttggcatgccaacactgaaagagagcagaagaaggagacagagcggattgaaaaggagagaatgcggcgactgatggctgaagatgaggagggttatagaaaactgattgatcaaaagaaagacaggcgtttagcttaccttttgcagcagaccgatgagtatgtagccaatctgaccaatctggtttgggagcacaagcaagcccaggcagccaaagagaagaagaagaggaggaggaggaagaagaaggctgaggagaatgcagagggtggggagtctgccctgggaccggatggagagcccatagatgagagcagccagatgagtgacctccctgtcaaagtgactcacacagaaaccggcaaggttctgttcggaccagaagcacccaaagcaagtcagctggacgcctggctggaaatgaatcctggttatgaagttgcccctagatctgacagtgaagagagtgattctgattatgaggaagaggatgaggaagaagagtccagtaggcaggaaaccgaagagaaaatactcctggatccaaatagcgaagaagtttctgagaaggatgctaagcagatcattgagacagctaagcaagacgtggatgatgaatacagcatgcagtacagtgccaggggctcccagtcctactacaccgtggctcatgccatctcggagagggtggagaaacagtctgccctcctaattaatgggaccctaaagcattaccagctccagggcctggaatggatggtttccctgtataataacaacttgaacggaatcttagccgatgaaatggggcttggaaagaccatacagaccattgcactcatcacttatctgatggagcacaaaagactcaatggcccctatctcatcattgttcccctttcgactctatctaactggacatatgaatttgacaaatgggctccttctgtggtgaagatttcttacaagggtactcctgccatgcgtcgctcccttgtcccccagctacggagtggcaaattcaatgtcctcttgactacttatgagtatattataaaagacaagcacattcttgcaaagattcggtggaaatacatgatagtggacgaaggccaccgaatgaagaatcaccactgcaagctgactcaggtcttgaacactcactatgtggcccccagaaggatcctcttgactgggaccccgctgcagaataagctccctgaactctgggccctcctcaacttcctcctcccaacaatttttaagagctgcagcacatttgaacaatggttcaatgctccatttgccatgactggtgaaagggtggacttaaatgaagaagaaactatattgatcatcaggcgtctacataaggtgttaagaccatttttactaaggagactgaagaaagaagttgaatcccagcttcccgaaaaagtggaatatgtgatcaagtgtgacatgtcagctctgcagaagattctgtatcgccatatgcaagccaaggggatccttctcacagatggttctgagaaagataagaaggggaaaggaggtgctaagacacttatgaacactattatgcagttgagaaaaatctgcaaccacccatatatgtttcagcacattgaggaatcctttgctgaacacctaggctattcaaatggggtcatcaatggggctgaactgtatcgggcctcagggaagtttgagctgcttgatcgtattctgccaaaattgagagcgactaatcaccgagtgctgcttttctgccagatgacatctctcatgaccatcatggaggattattttgcttttcggaacttcctttacctacgccttgatggcaccaccaagtctgaagatcgtgctgctttgctgaagaaattcaatgaacctggatcccagtatttcattttcttgctgagcacaagagctggtggcctgggcttaaatcttcaggcagctgatacagtggtcatctttgacagcgactggaatcctcatcaggatctgcaggcccaagaccgagctcaccgcatcgggcagcagaacgaggtccgggtactgaggctctgtaccgtgaacagcgtggaggaaaagatcctcgcggccgcaaaatacaagctgaacgtggatcagaaagtgatccaggcgggcatgtttgaccaaaagtcttcaagccacgagcggagggcattcctgcaggccatcttggagcatgaggaggaaaatgaggaagaagatgaagtaccggacgatgagactctgaaccaaatgattgctcgacgagaagaagaatttgacctttttatgcggatggacatggaccggcggagggaagatgcccggaacccgaaacggaagccccgtttaatggaggaggatgagctgccctcctggatcattaaggatgacgctgaagtagaaaggctcacctgtgaagaagaggaggagaaaatatttgggagggggtcccgccagcgccgtgacgtggactacagtgacgccctcacggagaagcagtggctaagggccatcgaagacggcaatttggaggaaatggaagaggaagtacggcttaagaagcgaaaaagacgaagaaatgtggataaagatcctgcaaaagaagatgtggaaaaagctaagaagagaagaggccgccctcccgctgagaaactgtcaccaaatccccccaaactgacaaagcagatgaacgctatcatcgatactgtgataaactacaaagatagttcagggcgacagctcagtgaagtcttcattcagttaccttcaaggaaagaattaccagaatactatgaattaattaggaagccagtggatttcaaaaaaataaaggaaaggattcgtaatcataagtaccggagcctaggcgacctggagaaggatgtcatgcttctctgtcacaacgctcagacgttcaacctggagggatcccagatctatgaagactccatcgtcttacagtcagtgtttaagagtgcccggcagaaaattgccaaagaggaagagagtgaggatgaaagcaatgaagaggaggaagaggaagatgaagaagagtcagagtccgaggcaaaatcagtcaaggtgaaaattaagctcaataaaaaagatgacaaaggccgggacaaagggaaaggcaagaaaaggccaaatcgaggaaaagccaaacctgtagtgagcgattttgacagcgatgaggagcaggatgaacgtgaacagtcagaaggaagtgggacggatgatgagtgatcagtatggacctttttccttggtagaactgaattccttcctcccctgtctcatttctacccagtgagttcatttgtcatataggcactgggttgtttctatatcatcatcgtctataaactagctttaggatagtgccagacaaacatatgatatcatggtgtaaaaaacacacacatacacaaatatttgtaacatattgtgaccaaatgggcctcaaagattcagattgaaacaaacaaaaagcttttgatggaaaatatgtgggtggatagtatatttctatgggtgggtctaatttggtaacggtttgattgtgcctggttttatcacctgttcagatgagaagatttttgtcttttgtagcactgataaccaggagaagccattaaaagccactggttattttatttttcatcaggcaattttcgaggtttttatttgttcggtattgtttttttacactgtggtacatataagcaactttaataggtgataaatgtacagtagttagatttcacctgcatatacatttttccattttatgctctatgatctgaacaaaagctttttgaattgtataagatttatgtctactgtaaacattgcttaatttttttgctcttgatttaaaaaaaagttttgttgaaagcgctattgaatattgcaatctatatagtgtattggatggcttcttttgtcaccctgatctcctatgttaccaatgtgtatcgtctccttctccctaaagtgtacttaatctttgctttctttgcacaatgtctttggttgcaagtcataagcctgaggcaaataaaattccagtaatttcgaagaatgtggtgttggtgctttcctaataaagaaataatttagcttgacaaaaaaaaaaaaaaa

SEQ ID NO: 3 - DNA, 5664 bp, Homo sapiens
gcgtcttccggcgcccgcggaggaggcgagggtgggacgctgggcggagcccgagtttaggaagaggaggggacggctgtcatcaatgaagtcatattcataatctagtcctctctccctctgtttctgtactctgggtgactcagagagggaagagattcagccagcacactcctcgcgagcaagcattactctactgactggcagagacaggagaggtagatgtccacgcccacagaccctggtgcgatgccccacccagggccttcgccggggcctgggccttcccctgggccaattcttgggcctagtccaggaccaggaccatccccaggttccgtccacagcatgatggggccaagtcctggacctccaagtgtctcccatcctatgccgacgatggggtccacagacttcccacaggaaggcatgcatcaaatgcataagcccatcgatggtatacatgacaaggggattgtagaagacatccattgtggatccatgaagggcactggtatgcgaccacctcacccaggcatgggccctccccagagtccaatggatcaacacagccaaggttatatgtcaccacacccatctccattaggagccccagagcacgtctccagccctatgtctggaggaggcccaactccacctcagatgccaccaagccagccgggggccctcatcccaggtgatccgcaggccatgagccagcccaacagaggtccctcacctttcagtcctgtccagctgcatcagcttcgagctcagattttagcttataaaatgctggcccgaggccagcccctccccgaaacgctgcagcttgcagtccaggggaaaaggacgttgcctggcttgcagcaacaacagcagcagcaacagcagcagcagcagcagcagcagcagcagcagcagcagcaacagcagccgcagcagcagccgccgcaaccacagacgcagcaacaacagcagccggcccttgttaactacaacagaccatctggcccggggccggagctgagcggcccgagcaccccgcagaagctgccggtgcccgcgcccggcggccggccctcgcccgcgccccccgcagccgcgcagccgcccgcggccgcagtgcccgggccctcagtgccgcagccggccccggggcagccctcgcccgtcctccagctgcagcagaagcagagccgcatcagccccatccagaaaccgcaaggcctggaccccgtggaaattctgcaagagcgggaatacagacttcaggcccgcatagctcataggatacaagaactggaaaatctgcctggctctttgccaccagatttaagaaccaaagcaaccgtggaactaaaagcacttcggttactcaatttccagcgtcagctgagacaggaggtggtggcctgcatgcgcagggacacgaccctggagacggctctcaactccaaagcatacaaacggagcaagcgccagactctgagagaagctcgcatgaccgagaagctggagaagcagcagaagattgagcaggagaggaaacgccgtcagaaacaccaggaatacctgaacagtattttgcaacatgcaaaagattttaaggaatatcatcggtctgtggccggaaagatccagaagctctccaaagcagtggcaacttggcatgccaacactgaaagagagcagaagaaggagacagagcggattgaaaaggagagaatgcggcgactgatggctgaagatgaggagggttatagaaaactgattgatcaaaagaaagacaggcgtttagcttaccttttgcagcagaccgatgagtatgtagccaatctgaccaatctggtttgggagcacaagcaagcccaggcagccaaagagaagaagaagaggaggaggaggaagaagaaggctgaggagaatgcagagggtggggagtctgccctgggaccggatggagagcccatagatgagagcagccagatgagtgacctccctgtcaaagtgactcacacagaaaccggcaaggttctgttcggaccagaagcacccaaagcaagtcagctggacgcctggctggaaatgaatcctggttatgaagttgcccctagatctgacagtgaagagagtgattctgattatgaggaagaggatgaggaagaagagtccagtaggcaggaaaccgaagagaaaatactcctggatccaaatagcgaagaagtttctgagaaggatgctaagcagatcattgagacagctaagcaagacgtggatgatgaatacagcatgcagtacagtgccaggggctcccagtcctactacaccgtggctcatgccatctcggagagggtggagaaacagtctgccctcctaattaatgggaccctaaagcattaccagctccagggcctggaatggatggtttccctgtataataacaacttgaacggaatcttagccgatgaaatggggcttggaaagaccatacagaccattgcactcatcacttatctgatggagcacaaaagactcaatggcccctatctcatcattgttcccctttcgactctatctaactggacatatgaatttgacaaatgggctccttctgtggtgaagatttcttacaagggtactcctgccatgcgtcgctcccttgtcccccagctacggagtggcaaattcaatgtcctcttgactacttatgagtatattataaaagacaagcacattcttgcaaagattcggtggaaatacatgatagtggacgaaggccaccgaatgaagaatcaccactgcaagctgactcaggtggacttaaatgaagaagaaactatattgatcatcaggcgtctacataaggtgttaagaccatttttactaaggagactgaagaaagaagttgaatcccagcttcccgaaaaagtggaatatgtgatcaagtgtgacatgtcagctctgcagaagattctgtatcgccatatgcaagccaaggggatccttctcacagatggttctgagaaagataagaaggggaaaggaggtgctaagacacttatgaacactattatgcagttgagaaaaatctgcaaccacccatatatgtttcagcacattgaggaatcctttgctgaacacctaggctattcaaatggggtcatcaatggggctgaactgtatcgggcctcagggaagtttgagctgcttgatcgtattctgccaaaattgagagcgactaatcaccgagtgctgcttttctgccagatgacatctctcatgaccatcatggaggattattttgcttttcggaacttcctttacctacgccttgatggcaccaccaagtctgaagatcgtgctgctttgctgaagaaattcaatgaacctggatcccagtatttcattttcttgctgagcacaagagctggtggcctgggcttaaatcttcaggcagctgatacagtggtcatctttgacagcgactggaatcctcatcaggatctgcaggcccaagaccgagctcaccgcatcgggcagcagaacgaggtccgggtactgaggctctgtaccgtgaacagcgtggaggaaaagatcctcgcggccgcaaaatacaagctgaacgtggatcagaaagtgatccaggcgggcatgtttgaccaaaagtcttcaagccacgagcggagggcattcctgcaggccatcttggagcatgaggaggaaaatgaggaagaagatgaagtaccggacgatgagactctgaaccaaatgattgctcgacgagaagaagaatttgacctttttatgcggatggacatggaccggcggagggaagatgcccggaacccgaaacggaagccccgtttaatggaggaggatgagctgccctcctggatcattaaggatgacgctgaagtagaaaggctcacctgtgaagaagaggaggagaaaatatttgggagggggtcccgccagcgccgtgacgtggactacagtgacgccctcacggagaagcagtggctaagggccatcgaagacggcaatttggaggaaatggaagaggaagtacggcttaagaagcgaaaaagacgaagaaatgtggataaagatcctgcaaaagaagatgtggaaaaagctaagaagagaagaggccgccctcccgctgagaaactgtcaccaaatccccccaaactgacaaagcagatgaacgctatcatcgatactgtgataaactacaaagatagttcagggcgacagctcagtgaagtcttcattcagttaccttcaaggaaagaattaccagaatactatgaattaattaggaagccagtggatttcaaaaaaataaaggaaaggattcgtaatcataagtaccggagcctaggcgacctggagaaggatgtcatgcttctctgtcacaacgctcagacgttcaacctggagggatcccagatctatgaagactccatcgtcttacagtcagtgtttaagagtgcccggcagaaaattgccaaagaggaagagagtgaggatgaaagcaatgaagaggaggaagaggaagatgaagaagagtcagagtccgaggcaaaatcagtcaaggtgaaaattaagctcaataaaaaagatgacaaaggccgggacaaagggaaaggcaagaaaaggccaaatcgaggaaaagccaaacctgtagtgagcgattttgacagcgatgaggagcaggatgaacgtgaacagtcagaaggaagtgggacggatgatgagtgatcagtatggacctttttccttggtagaactgaattccttcctcccctgtctcatttctacccagtgagttcatttgtcatataggcactgggttgtttctatatcatcatcgtctataaactagctttaggatagtgccagacaaacatatgatatcatggtgtaaaaaacacacacatacacaaatatttgtaacatattgtgaccaaatgggcctcaaagattcagattgaaacaaacaaaaagcttttgatggaaaatatgtgggtggatagtatatttctatgggtgggtctaatttggtaacggtttgattgtgcctggttttatcacctgttcagatgagaagatttttgtcttttgtagcactgataaccaggagaagccattaaaagccactggttattttatttttcatcaggcaattttcgaggtttttatttgttcggtattgtttttttacactgtggtacatataagcaactttaataggtgataaatgtacagtagttagatttcacctgcatatacatttttccattttatgctctatgatctgaacaaaagctttttgaattgtataagatttatgtctactgtaaacattgcttaatttttttgctcttgatttaaaaaaaagttttgttgaaagcgctattgaatattgcaatctatatagtgtattggatggcttcttttgtcaccctgatctcctatgttaccaatgtgtatcgtctccttctccctaaagtgtacttaatctttgctttctttgcacaatgtctttggttgcaagtcataagcctgaggcaaataaaattccagtaatttcgaagaatgtggtgttggtgctttcctaataaagaaataatttagcttgacaaaaaaaaaaaaaaa

SEQ ID NO: 4 - DNA, 1846 bp, Homo sapiens
cttggagaggcggaggtggaaacgatgcgcaggagttggcttggggctttttgtttgcgtgtccctgtttacctattcataatcatggatcccctctgctttgtgatactgtgaaccacgcataacagcaattctttacaccaccgggttgagaagaaggcgcctgaggctgactttctggacctgccgtcacgcagtaaagatgtggttggccatcgaagacggcaatttggaggaaatggaagaggaagtacggcttaagaagcgaaaaagacgaagaaatgtggataaagatcctgcaaaagaagatgtggaaaaagctaagaagagaagaggccgccctcccgctgagaaactgtcaccaaatccccccaaactgacaaagcagatgaacgctatcatcgatactgtgataaactacaaagatagttcagggcgacagctcagtgaagtcttcattcagttaccttcaaggaaagaattaccagaatactatgaattaattaggaagccagtggatttcaaaaaaataaaggaaaggattcgtaatcataagtaccggagcctaggcgacctggagaaggatgtcatgcttctctgtcacaacgctcagacgttcaacctggagggatcccagatctatgaagactccatcgtcttacagtcagtgtttaagagtgcccggcagaaaattgccaaagaggaagagagtgaggatgaaagcaatgaagaggaggaagaggaagatgaagaagagtcagagtccgaggcaaaatcagtcaaggtgaaaattaagctcaataaaaaagatgacaaaggccgggacaaagggaaaggcaagaaaaggccaaatcgaggaaaagccaaacctgtagtgagcgattttgacagcgatgaggagcaggatgaacgtgaacagtcagaaggaagtgggacggatgatgagtgatcagtatggacctttttccttggtagaactgaattccttcctcccctgtctcatttctacccagtgagttcatttgtcatataggcactgggttgtttctatatcatcatcgtctataaactagctttaggatagtgccagacaaacatatgatatcatggtgtaaaaaacacacacatacacaaatatttgtaacatattgtgaccaaatgggcctcaaagattcagattgaaacaaacaaaaagcttttgatggaaaatatgtgggtggatagtatatttctatgggtgggtctaatttggtaacggtttgattgtgcctggttttatcacctgttcagatgagaagatttttgtcttttgtagcactgataaccaggagaagccattaaaagccactggttattttatttttcatcaggcaattttcgaggtttttatttgttcggtattgtttttttacactgtggtacatataagcaactttaataggtgataaatgtacagtagttagatttcacctgcatatacatttttccattttatgctctatgatctgaacaaaagctttttgaattgtataagatttatgtctactgtaaacattgcttaatttttttgctcttgatttaaaaaaaagttttgttgaaagcgctattgaatattgcaatctatatagtgtattggatggcttcttttgtcaccctgatctcctatgttaccaatgtgtatcgtctccttctccctaaagtgtacttaatctttgctttctttgcacaatgtctttggttgcaagtcataagcctgaggcaaataaaattccagtaatttcgaagaatgtggtgttggtgctttcctaataaagaaataatttagcttgacaaaaaaaaaaaaaaa

SEQ ID NO: 5 - protein, 1590 aa, Homo sapiens
MSTPTDPGAMPHPGPSPGPGPSPGPILGPSPGPGPSPGSVHSMMGPSPGPPSVSHPMPTMGSTDFPQEGMHQMHKPIDGIHDKGIVEDIHCGSMKGTGMRPPHPGMGPPQSPMDQHSQGYMSPHPSPLGAPEHVSSPMSGGGPTPPQMPPSQPGALIPGDPQAMSQPNRGPSPFSPVQLHQLRAQILAYKMLARGQPLPETLQLAVQGKRTLPGLQQQQQQQQQQQQQQQQQQQQQQQPQQQPPQPQTQQQQQPALVNYNRPSGPGPELSGPSTPQKLPVPAPGGRPSPAPPAAAQPPAAAVPGPSVPQPAPGQPSPVLQLQQKQSRISPIQKPQGLDPVEILQEREYRLQARIAHRIQELENLPGSLPPDLRTKATVELKALRLLNFQRQLRQEVVACMRRDTTLETALNSKAYKRSKRQTLREARMTEKLEKQQKIEQERKRRQKHQEYLNSILQHAKDFKEYHRSVAGKIQKLSKAVATWHANTEREQKKETERIEKERMRRLMAEDEEGYRKLIDQKKDRRLAYLLQQTDEYVANLTNLVWEHKQAQAAKEKKKRRRRKKKAEENAEGGESALGPDGEPIDESSQMSDLPVKVTHTETGKVLFGPEAPKASQLDAWLEMNPGYEVAPRSDSEESDSDYEEEDEEEESSRQETEEKILLDPNSEEVSEKDAKQIIETAKQDVDDEYSMQYSARGSQSYYTVAHAISERVEKQSALLINGTLKHYQLQGLEWMVSLYNNNLNGILADEMGLGKTIQTIALITYLMEHKRLNGPYLIIVPLSTLSNWTYEFDKWAPSVVKISYKGTPAMRRSLVPQLRSGKFNVLLTTYEYIIKDKHILAKIRWKYMIVDEGHRMKNHHCKLTQVLNTHYVAPRRILLTGTPLQNKLPELWALLNFLLPTIFKSCSTFEQWFNAPFAMTGERVDLNEEETILIIRRLHKVLRPFLLRRLKKEVESQLPEKVEYVIKCDMSALQKILYRHMQAKGILLTDGSEKDKKGKGGAKTLMNTIMQLRKICNHPYMFQHIEESFAEHLGYSNGVINGAELYRASGKFELLDRILPKLRATNHRVLLFCQMTSLMTIMEDYFAFRNFLYLRLDGTTKSEDRAALLKKFNEPGSQYFIFLLSTRAGGLGLNLQAADTVVIFDSDWNPHQDLQAQDRAHRIGQQNEVRVLRLCTVNSVEEKILAAAKYKLNVDQKVIQAGMFDQKSSSHERRAFLQAILEHEEENEEEDEVPDDETLNQMIARREEEFDLFMRMDMDRRREDARNPKRKPRLMEEDELPSWIIKDDAEVERLTCEEEEEKIFGRGSRQRRDVDYSDALTEKQWLRAIEDGNLEEMEEEVRLKKRKRRRNVDKDPAKEDVEKAKKRRGRPPAEKLSPNPPKLTKQMNAIIDTVINYKDRCNVEKVPSNSQLEIEGNSSGRQLSEVFIQLPSRKELPEYYELIRKPVDFKKIKERIRNHKYRSLGDLEKDVMLLCHNAQTFNLEGSQIYEDSIVLQSVFKSARQKIAKEEESEDESNEEEEEEDEEESESEAKSVKVKIKLNKKDDKGRDKGKGKKRPNRGKAKPVVSDFDSDEEQDEREQSEGSGTDDE

SEQ ID NO: 6 - protein, 1572 aa, Homo sapiens
MSTPTDPGAMPHPGPSPGPGPSPGPILGPSPGPGPSPGSVHSMMGPSPGPPSVSHPMPTMGSTDFPQEGMHQMHKPIDGIHDKGIVEDIHCGSMKGTGMRPPHPGMGPPQSPMDQHSQGYMSPHPSPLGAPEHVSSPMSGGGPTPPQMPPSQPGALIPGDPQAMSQPNRGPSPFSPVQLHQLRAQILAYKMLARGQPLPETLQLAVQGKRTLPGLQQQQQQQQQQQQQQQQQQQQQQQPQQQPPQPQTQQQQQPALVNYNRPSGPGPELSGPSTPQKLPVPAPGGRPSPAPPAAAQPPAAAVPGPSVPQPAPGQPSPVLQLQQKQSRISPIQKPQGLDPVEILQEREYRLQARIAHRIQELENLPGSLPPDLRTKATVELKALRLLNFQRQLRQEVVACMRRDTTLETALNSKAYKRSKRQTLREARMTEKLEKQQKIEQERKRRQKHQEYLNSILQHAKDFKEYHRSVAGKIQKLSKAVATWHANTEREQKKETERIEKERMRRLMAEDEEGYRKLIDQKKDRRLAYLLQQTDEYVANLTNLVWEHKQAQAAKEKKKRRRRKKKAEENAEGGESALGPDGEPIDESSQMSDLPVKVTHTETGKVLFGPEAPKASQLDAWLEMNPGYEVAPRSDSEESDSDYEEEDEEEESSRQETEEKILLDPNSEEVSEKDAKQIIETAKQDVDDEYSMQYSARGSQSYYTVAHAISERVEKQSALLINGTLKHYQLQGLEWMVSLYNNNLNGILADEMGLGKTIQTIALITYLMEHKRLNGPYLIIVPLSTLSNWTYEFDKWAPSVVKISYKGTPAMRRSLVPQLRSGKFNVLLTTYEYIIKDKHILAKIRWKYMIVDEGHRMKNHHCKLTQVLNTHYVAPRRILLTGTPLQNKLPELWALLNFLLPTIFKSCSTFEQWFNAPFAMTGERVDLNEEETILIIRRLHKVLRPFLLRRLKKEVESQLPEKVEYVIKCDMSALQKILYRHMQAKGILLTDGSEKDKKGKGGAKTLMNTIMQLRKICNHPYMFQHIEESFAEHLGYSNGVINGAELYRASGKFELLDRILPKLRATNHRVLLFCQMTSLMTIMEDYFAFRNFLYLRLDGTTKSEDRAALLKKFNEPGSQYFIFLLSTRAGGLGLNLQAADTVVIFDSDWNPHQDLQAQDRAHRIGQQNEVRVLRLCTVNSVEEKILAAAKYKLNVDQKVIQAGMFDQKSSSHERRAFLQAILEHEEENEEEDEVPDDETLNQMIARREEEFDLFMRMDMDRRREDARNPKRKPRLMEEDELPSWIIKDDAEVERLTCEEEEEKIFGRGSRQRRDVDYSDALTEKQWLRAIEDGNLEEMEEEVRLKKRKRRRNVDKDPAKEDVEKAKKRRGRPPAEKLSPNPPKLTKQMNAIIDTVINYKDSSGRQLSEVFIQLPSRKELPEYYELIRKPVDFKKIKERIRNHKYRSLGDLEKDVMLLCHNAQTFNLEGSQIYEDSIVLQSVFKSARQKIAKEEESEDESNEEEEEEDEEESESEAKSVKVKIKLNKKDDKGRDKGKGKKRPNRGKAKPVVSDFDSDEEQDEREQSEGSGTDDE

SEQ ID NO: 7 - protein, 1514 aa, Homo sapiens
MSTPTDPGAMPHPGPSPGPGPSPGPILGPSPGPGPSPGSVHSMMGPSPGPPSVSHPMPTMGSTDFPQEGMHQMHKPIDGIHDKGIVEDIHCGSMKGTGMRPPHPGMGPPQSPMDQHSQGYMSPHPSPLGAPEHVSSPMSGGGPTPPQMPPSQPGALIPGDPQAMSQPNRGPSPFSPVQLHQLRAQILAYKMLARGQPLPETLQLAVQGKRTLPGLQQQQQQQQQQQQQQQQQQQQQQQPQQQPPQPQTQQQQQPALVNYNRPSGPGPELSGPSTPQKLPVPAPGGRPSPAPPAAAQPPAAAVPGPSVPQPAPGQPSPVLQLQQKQSRISPIQKPQGLDPVEILQEREYRLQARIAHRIQELENLPGSLPPDLRTKATVELKALRLLNFQRQLRQEVVACMRRDTTLETALNSKAYKRSKRQTLREARMTEKLEKQQKIEQERKRRQKHQEYLNSILQHAKDFKEYHRSVAGKIQKLSKAVATWHANTEREQKKETERIEKERMRRLMAEDEEGYRKLIDQKKDRRLAYLLQQTDEYVANLTNLVWEHKQAQAAKEKKKRRRRKKKAEENAEGGESALGPDGEPIDESSQMSDLPVKVTHTETGKVLFGPEAPKASQLDAWLEMNPGYEVAPRSDSEESDSDYEEEDEEEESSRQETEEKILLDPNSEEVSEKDAKQIIETAKQDVDDEYSMQYSARGSQSYYTVAHAISERVEKQSALLINGTLKHYQLQGLEWMVSLYNNNLNGILADEMGLGKTIQTIALITYLMEHKRLNGPYLIIVPLSTLSNWTYEFDKWAPSVVKISYKGTPAMRRSLVPQLRSGKFNVLLTTYEYIIKDKHILAKIRWKYMIVDEGHRMKNHHCKLTQVDLNEEETILIIRRLHKVLRPFLLRRLKKEVESQLPEKVEYVIKCDMSALQKILYRHMQAKGILLTDGSEKDKKGKGGAKTLMNTIMQLRKICNHPYMFQHIEESFAEHLGYSNGVINGAELYRASGKFELLDRILPKLRATNHRVLLFCQMTSLMTIMEDYFAFRNFLYLRLDGTTKSEDRAALLKKFNEPGSQYFIFLLSTRAGGLGLNLQAADTVVIFDSDWNPHQDLQAQDRAHRIGQQNEVRVLRLCTVNSVEEKILAAAKYKLNVDQKVIQAGMFDQKSSSHERRAFLQAILEHEEENEEEDEVPDDETLNQMIARREEEFDLFMRMDMDRRREDARNPKRKPRLMEEDELPSWIIKDDAEVERLTCEEEEEKIFGRGSRQRRDVDYSDALTEKQWLRAIEDGNLEEMEEEVRLKKRKRRRNVDKDPAKEDVEKAKKRRGRPPAEKLSPNPPKLTKQMNAIIDTVINYKDSSGRQLSEVFIQLPSRKELPEYYELIRKPVDFKKIKERIRNHKYRSLGDLEKDVMLLCHNAQTFNLEGSQIYEDSIVLQSVFKSARQKIAKEEESEDESNEEEEEEDEEESESEAKSVKVKIKLNKKDDKGRDKGKGKKRPNRGKAKPVVSDFDSDEEQDEREQSEGSGTDDE

SEQ ID NO: 8 - protein, 248 aa, Homo sapiens
MWLAIEDGNLEEMEEEVRLKKRKRRRNVDKDPAKEDVEKAKKRRGRPPAEKLSPNPPKLTKQMNAIIDTVINYKDSSGRQLSEVFIQLPSRKELPEYYELIRKPVDFKKIKERIRNHKYRSLGDLEKDVMLLCHNAQTFNLEGSQIYEDSIVLQSVFKSARQKIAKEEESEDESNEEEEEEDEEESESEAKSVKVKIKLNKKDDKGRDKGKGKKRPNRGKAKPVVSDFDSDEEQDEREQSEGSGTDDE

SEQ ID NO: 9 - DNA, 5589 bp, Homo sapiens
ggcgggggaggcgccgggaagtcgacggcgccggcggctcctgcaggaggccactgtctgcagctcccgtgaagatgtccactccagacccacccctgggcggaactcctcggccaggtccttccccgggccctggcccttcccctggagccatgctgggccctagcccgggtccctcgccgggctccgcccacagcatgatggggcccagcccagggccgccctcagcaggacaccccatccccacccaggggcctggagggtaccctcaggacaacatgcaccagatgcacaagcccatggagtccatgcatgagaagggcatgtcggacgacccgcgctacaaccagatgaaaggaatggggatgcggtcagggggccatgctgggatggggcccccgcccagccccatggaccagcactcccaaggttacccctcgcccctgggtggctctgagcatgcctctagtccagttccagccagtggcccgtcttcggggccccagatgtcttccgggccaggaggtgccccgctggatggtgctgacccccaggccttggggcagcagaaccggggcccaaccccatttaaccagaaccagctgcaccagctcagagctcagatcatggcctacaagatgctggccagggggcagcccctccccgaccacctgcagatggcggtgcagggcaagcggccgatgcccgggatgcagcagcagatgccaacgctacctccaccctcggtgtccgcaacaggacccggccctggccctggccctggccccggcccgggtcccggcccggcacctccaaattacagcaggcctcatggtatgggagggcccaacatgcctcccccaggaccctcgggcgtgccccccgggatgccaggccagcctcctggagggcctcccaagccctggcctgaaggacccatggcgaatgctgctgcccccacgagcacccctcagaagctgattcccccgcagccaacgggccgcccttcccccgcgccccctgccgtcccacccgccgcctcgcccgtgatgccaccgcagacccagtcccccgggcagccggcccagcccgcgcccatggtgccactgcaccagaagcagagccgcatcacccccatccagaagccgcggggcctcgaccctgtggagatcctgcaggagcgcgagtacaggctgcaggctcgcatcgcacaccgaattcaggaacttgaaaaccttcccgggtccctggccggggatttgcgaaccaaagcgaccattgagctcaaggccctcaggctgctgaacttccagaggcagctgcgccaggaggtggtggtgtgcatgcggagggacacagcgctggagacagccctcaatgctaaggcctacaagcgcagcaagcgccagtccctgcgcgaggcccgcatcactgagaagctggagaagcagcagaagatcgagcaggagcgcaagcgccggcagaagcaccaggaatacctcaatagcattctccagcatgccaaggatttcaaggaatatcacagatccgtcacaggcaaaatccagaagctgaccaaggcagtggccacgtaccatgccaacacggagcgggagcagaagaaagagaacgagcggatcgagaaggagcgcatgcggaggctcatggctgaagatgaggaggggtaccgcaagctcatcgaccagaagaaggacaagcgcctggcctacctcttgcagcagacagacgagtacgtggctaacctcacggagctggtgcggcagcacaaggctgcccaggtcgccaaggagaaaaagaagaaaaagaaaaagaagaaggcagaaaatgcagaaggacagacgcctgccattgggccggatggcgagcctctggacgagaccagccagatgagcgacctcccggtgaaggtgatccacgtggagagtgggaagatcctcacaggcacagatgcccccaaagccgggcagctggaggcctggctcgagatgaacccggggtatgaagtagctccgaggtctgatagtgaagaaagtggctcagaagaagaggaagaggaggaggaggaagagcagccgcaggcagcacagcctcccaccctgcccgtggaggagaagaagaagattccagatccagacagcgatgacgtctctgaggtggacgcgcggcacatcattgagaatgccaagcaagatgtcgatgatgaatatggcgtgtcccaggcccttgcacgtggcctgcagtcctactatgccgtggcccatgctgtcactgagagagtggacaagcagtcagcgcttatggtcaatggtgtcctcaaacagtaccagatcaaaggtttggagtggctggtgtccctgtacaacaacaacctgaacggcatcctggccgacgagatgggcctggggaagaccatccagaccatcgcgctcatcacgtacctcatggagcacaaacgcatcaatgggcccttcctcatcatcgtgcctctctcaacgctgtccaactgggcgtacgagtttgacaagtgggccccctccgtggtgaaggtgtcttacaagggatccccagcagcaagacgggcctttgtcccccagctccggagtgggaagttcaacgtcttgctgacgacgtacgagtacatcatcaaagacaagcacatcctcgccaagatccgttggaagtacatgattgtggacgaaggtcaccgcatgaagaaccaccactgcaagctgacgcaggtgctcaacacgcactatgtggcaccccgccgcctgctgctgacgggcacaccgctgcagaacaagcttcccgagctctgggcgctgctcaacttcctgctgcccaccatcttcaagagctgcagcaccttcgagcagtggtttaacgcaccctttgccatgaccggggaaaaggtggacctgaatgaggaggaaaccattctcatcatccggcgtctccacaaagtgctgcggcccttcttgctccgacgactcaagaaggaagtcgaggcccagttgcccgaaaaggtggagtacgtcatcaagtgcgacatgtctgcgctgcagcgagtgctctaccgccacatgcaggccaagggcgtgctgctgactgatggctccgagaaggacaagaagggcaaaggcggcaccaagaccctgatgaacaccatcatgcagctgcggaagatctgcaaccacccctacatgttccagcacatcgaggagtccttttccgagcacttggggttcactggcggcattgtccaagggctggacctgtaccgagcctcgggtaaatttgagcttcttgatagaattcttcccaaactccgagcaaccaaccacaaagtgctgctgttctgccaaatgacctccctcatgaccatcatggaagattactttgcgtatcgcggctttaaatacctcaggcttgatggaaccacgaaggcggaggaccggggcatgctgctgaaaaccttcaacgagcccggctctgagtacttcatcttcctgctcagcacccgggctggggggctcggcctgaacctccagtcggcagacactgtgatcatttttgacagcgactggaatcctcaccaggacctgcaagcgcaggaccgagcccaccgcatcgggcagcagaacgaggtgcgtgtgctccgcctctgcaccgtcaacagcgtggaggagaagatcctagctgcagccaagtacaagctcaacgtggaccagaaggtgatccaggccggcatgttcgaccagaagtcctccagccatgagcggcgcgccttcctgcaggccatcctggagcacgaggagcaggatgagagcagacactgcagcacgggcagcggcagtgccagcttcgcccacactgcccctccgccagcgggcgtcaaccccgacttggaggagccacctctaaaggaggaagacgaggtgcccgacgacgagaccgtcaaccagatgatcgcccggcacgaggaggagtttgatctgttcatgcgcatggacctggaccgcaggcgcgaggaggcccgcaaccccaagcggaagccgcgcctcatggaggaggacgagctcccctcgtggatcatcaaggacgacgcggaggtggagcggctgacctgtgaggaggaggaggagaagatgttcggccgtggctcccgccaccgcaaggaggtggactacagcgactcactgacggagaagcagtggctcaagaaaattacaggaaaagatatccatgacacagccagcagtgtggcacgtgggctacaattccagcgtggccttcagttctgcacacgtgcgtcaaaggccatcgaggagggcacgctggaggagatcgaagaggaggtccggcagaagaaatcatcacggaagcgcaagcgagacagcgacgccggctcctccaccccgaccaccagcacccgcagccgcgacaaggacgacgagagcaagaagcagaagaagcgcgggcggccgcctgccgagaaactctcccctaacccacccaacctcaccaagaagatgaagaagattgtggatgccgtgatcaagtacaaggacagcagcagtggacgtcagctcagcgaggtcttcatccagctgccctcgcgaaaggagctgcccgagtactacgagctcatccgcaagcccgtggacttcaagaagataaaggagcgcattcgcaaccacaagtaccgcagcctcaacgacctagagaaggacgtcatgctcctgtgccagaacgcacagaccttcaacctggagggctccctgatctatgaagactccatcgtcttgcagtcggtcttcaccagcgtgcggcagaaaatcgagaaggaggatgacagtgaaggcgaggagagtgaggaggaggaagagggcgaggaggaaggctccgaatccgaatctcggtccgtcaaagtgaagatcaagcttggccggaaggagaaggcacaggaccggctgaagggcggccggcggcggccgagccgagggtcccgagccaagccggtcgtgagtgacgatgacagtgaggaggaacaagaggaggaccgctcaggaagtggcagcgaagaagactgagccccgacattccagtctcgaccccgagcccctcgttccagagctgagatggcataggccttagcagtaacgggtagcagcagatgtagtttcagacttggagtaaaactgtataaacaaaagaatcttccatatttatacagcagagaagctgtaggactgtttgtgactggccctgtcctggcatcagtagcatctgtaacagcattaactgtcttaaagagagagagagagaattccgaattggggaacacacgatacctgtttttcttttccgttgctggcagtactgttgcgccgcagtttggagtcactgtagttaagtgtggatgcatgtgcgtcaccgtccactcctcctactgtattttattggacaggtcagactcgccgggggcccggcgagggtatgtcagtgtcactggatgtcaaacagtaataaattaaaccaacaacaaaacgcacagccaaaaaaaaa

SEQ ID NO: 10 - DNA, 5779 bp, Homo sapiens
ggagaggccgccgcggtgctgagggggaggggagccggcgagcgcgcgcgcagcgggggcgcgggtggcgcgcgtgtgtgtgaagggggggcggtggccgaggcgggcgggcgcgcgcgcgaggcttcccctcgtttggcggcggcggcggcttctttgtttcgtgaagagaagcgagacgcccattctgcccccggccccgcgcggaggggcgggggaggcgccgggaagtcgacggcgccggcggctcctgcgtctcgcccttttgcccaggctagagtgcagtggtgcggtcatggttcactgcagcctcaacctcctggactcagcaggaggccactgtctgcagctcccgtgaagatgtccactccagacccacccctgggcggaactcctcggccaggtccttccccgggccctggcccttcccctggagccatgctgggccctagcccgggtccctcgccgggctccgcccacagcatgatggggcccagcccagggccgccctcagcaggacaccccatccccacccaggggcctggagggtaccctcaggacaacatgcaccagatgcacaagcccatggagtccatgcatgagaagggcatgtcggacgacccgcgctacaaccagatgaaaggaatggggatgcggtcagggggccatgctgggatggggcccccgcccagccccatggaccagcactcccaaggttacccctcgcccctgggtggctctgagcatgcctctagtccagttccagccagtggcccgtcttcggggccccagatgtcttccgggccaggaggtgccccgctggatggtgctgacccccaggccttggggcagcagaaccggggcccaaccccatttaaccagaaccagctgcaccagctcagagctcagatcatggcctacaagatgctggccagggggcagcccctccccgaccacctgcagatggcggtgcagggcaagcggccgatgcccgggatgcagcagcagatgccaacgctacctccaccctcggtgtccgcaacaggacccggccctggccctggccctggccccggcccgggtcccggcccggcacctccaaattacagcaggcctcatggtatgggagggcccaacatgcctcccccaggaccctcgggcgtgccccccgggatgccaggccagcctcctggagggcctcccaagccctggcctgaaggacccatggcgaatgctgctgcccccacgagcacccctcagaagctgattcccccgcagccaacgggccgcccttcccccgcgccccctgccgtcccacccgccgcctcgcccgtgatgccaccgcagacccagtcccccgggcagccggcccagcccgcgcccatggtgccactgcaccagaagcagagccgcatcacccccatccagaagccgcggggcctcgaccctgtggagatcctgcaggagcgcgagtacaggctgcaggctcgcatcgcacaccgaattcaggaacttgaaaaccttcccgggtccctggccggggatttgcgaaccaaagcgaccattgagctcaaggccctcaggctgctgaacttccagaggcagctgcgccaggaggtggtggtgtgcatgcggagggacacagcgctggagacagccctcaatgctaaggcctacaagcgcagcaagcgccagtccctgcgcgaggcccgcatcactgagaagctggagaagcagcagaagatcgagcaggagcgcaagcgccggcagaagcaccaggaatacctcaatagcattctccagcatgccaaggatttcaaggaatatcacagatccgtcacaggcaaaatccagaagctgaccaaggcagtggccacgtaccatgccaacacggagcgggagcagaagaaagagaacgagcggatcgagaaggagcgcatgcggaggctcatggctgaagatgaggaggggtaccgcaagctcatcgaccagaagaaggacaagcgcctggcctacctcttgcagcagacagacgagtacgtggctaacctcacggagctggtgcggcagcacaaggctgcccaggtcgccaaggagaaaaagaagaaaaagaaaaagaagaaggcagaaaatgcagaaggacagacgcctgccattgggccggatggcgagcctctggacgagaccagccagatgagcgacctcccggtgaaggtgatccacgtggagagtgggaagatcctcacaggcacagatgcccccaaagccgggcagctggaggcctggctcgagatgaacccggggtatgaagtagctccgaggtctgatagtgaagaaagtggctcagaagaagaggaagaggaggaggaggaagagcagccgcaggcagcacagcctcccaccctgcccgtggaggagaagaagaagattccagatccagacagcgatgacgtctctgaggtggacgcgcggcacatcattgagaatgccaagcaagatgtcgatgatgaatatggcgtgtcccaggcccttgcacgtggcctgcagtcctactatgccgtggcccatgctgtcactgagagagtggacaagcagtcagcgcttatggtcaatggtgtcctcaaacagtaccagatcaaaggtttggagtggctggtgtccctgtacaacaacaacctgaacggcatcctggccgacgagatgggcctggggaagaccatccagaccatcgcgctcatcacgtacctcatggagcacaaacgcatcaatgggcccttcctcatcatcgtgcctctctcaacgctgtccaactgggcgtacgagtttgacaagtgggccccctccgtggtgaaggtgtcttacaagggatccccagcagcaagacgggcctttgtcccccagctccggagtgggaagttcaacgtcttgctgacgacgtacgagtacatcatcaaagacaagcacatcctcgccaagatccgttggaagtacatgattgtggacgaaggtcaccgcatgaagaaccaccactgcaagctgacgcaggtgctcaacacgcactatgtggcaccccgccgcctgctgctgacgggcacaccgctgcagaacaagcttcccgagctctgggcgctgctcaacttcctgctgcccaccatcttcaagagctgcagcaccttcgagcagtggtttaacgcaccctttgccatgaccggggaaaaggtggacctgaatgaggaggaaaccattctcatcatccggcgtctccacaaagtgctgcggcccttcttgctccgacgactcaagaaggaagtcgaggcccagttgcccgaaaaggtggagtacgtcatcaagtgcgacatgtctgcgctgcagcgagtgctctaccgccacatgcaggccaagggcgtgctgctgactgatggctccgagaaggacaagaagggcaaaggcggcaccaagaccctgatgaacaccatcatgcagctgcggaagatctgcaaccacccctacatgttccagcacatcgaggagtccttttccgagcacttggggttcactggcggcattgtccaagggctggacctgtaccgagcctcgggtaaatttgagcttcttgatagaattcttcccaaactccgagcaaccaaccacaaagtgctgctgttctgccaaatgacctccctcatgaccatcatggaagattactttgcgtatcgcggctttaaatacctcaggcttgatggaaccacgaaggcggaggaccggggcatgctgctgaaaaccttcaacgagcccggctctgagtacttcatcttcctgctcagcacccgggctggggggctcggcctgaacctccagtcggcagacactgtgatcatttttgacagcgactggaatcctcaccaggacctgcaagcgcaggaccgagcccaccgcatcgggcagcagaacgaggtgcgtgtgctccgcctctgcaccgtcaacagcgtggaggagaagatcctagctgcagccaagtacaagctcaacgtggaccagaaggtgatccaggccggcatgttcgaccagaagtcctccagccatgagcggcgcgccttcctgcaggccatcctggagcacgaggagcaggatgagagcagacactgcagcacgggcagcggcagtgccagcttcgcccacactgcccctccgccagcgggcgtcaaccccgacttggaggagccacctctaaaggaggaagacgaggtgcccgacgacgagaccgtcaaccagatgatcgcccggcacgaggaggagtttgatctgttcatgcgcatggacctggaccgcaggcgcgaggaggcccgcaaccccaagcggaagccgcgcctcatggaggaggacgagctcccctcgtggatcatcaaggacgacgcggaggtggagcggctgacctgtgaggaggaggaggagaagatgttcggccgtggctcccgccaccgcaaggaggtggactacagcgactcactgacggagaagcagtggctcaaggccatcgaggagggcacgctggaggagatcgaagaggaggtccggcagaagaaatcatcacggaagcgcaagcgagacagcgacgccggctcctccaccccgaccaccagcacccgcagccgcgacaaggacgacgagagcaagaagcagaagaagcgcgggcggccgcctgccgagaaactctcccctaacccacccaacctcaccaagaagatgaagaagattgtggatgccgtgatcaagtacaaggacagcagcagtggacgtcagctcagcgaggtcttcatccagctgccctcgcgaaaggagctgcccgagtactacgagctcatccgcaagcccgtggacttcaagaagataaaggagcgcattcgcaaccacaagtaccgcagcctcaacgacctagagaaggacgtcatgctcctgtgccagaacgcacagaccttcaacctggagggctccctgatctatgaagactccatcgtcttgcagtcggtcttcaccagcgtgcggcagaaaatcgagaaggaggatgacagtgaaggcgaggagagtgaggaggaggaagagggcgaggaggaaggctccgaatccgaatctcggtccgtcaaagtgaagatcaagcttggccggaaggagaaggcacaggaccggctgaagggcggccggcggcggccgagccgagggtcccgagccaagccggtcgtgagtgacgatgacagtgaggaggaacaagaggaggaccgctcaggaagtggcagcgaagaagactgagccccgacattccagtctcgaccccgagcccctcgttccagagctgagatggcataggccttagcagtaacgggtagcagcagatgtagtttcagacttggagtaaaactgtataaacaaaagaatcttccatatttatacagcagagaagctgtaggactgtttgtgactggccctgtcctggcatcagtagcatctgtaacagcattaactgtcttaaagagagagagagagaattccgaattggggaacacacgatacctgtttttcttttccgttgctggcagtactgttgcgccgcagtttggagtcactgtagttaagtgtggatgcatgtgcgtcaccgtccactcctcctactgtattttattggacaggtcagactcgccgggggcccggcgagggtatgtcagtgtcactggatgtcaaacagtaataaattaaaccaacaacaaaacgcacagccaaaaaaaaa

SEQ ID NO: 11 - DNA, 5329 bp, Homo sapiens
atgtccactccagacccacccctgggcggaactcctcggccaggtccttccccgggccctggcccttcccctggagccatgctgggccctagcccgggtccctcgccgggctccgcccacagcatgatggggcccagcccagggccgccctcagcaggacaccccatccccacccaggggcctggagggtaccctcaggacaacatgcaccagatgcacaagcccatggagtccatgcatgagaagggcatgtcggacgacccgcgctacaaccagatgaaaggaatggggatgcggtcagggggccatgctgggatggggcccccgcccagccccatggaccagcactcccaaggttacccctcgcccctgggtggctctgagcatgcctctagtccagttccagccagtggcccgtcttcggggccccagatgtcttccgggccaggaggtgccccgctggatggtgctgacccccaggccttggggcagcagaaccggggcccaaccccatttaaccagaaccagctgcaccagctcagagctcagatcatggcctacaagatgctggccagggggcagcccctccccgaccacctgcagatggcggtgcagggcaagcggccgatgcccgggatgcagcagcagatgccaacgctacctccaccctcggtgtccgcaacaggacccggccctggccctggccctggccccggcccgggtcccggcccggcacctccaaattacagcaggcctcatggtatgggagggcccaacatgcctcccccaggaccctcgggcgtgccccccgggatgccaggccagcctcctggagggcctcccaagccctggcctgaaggacccatggcgaatgctgctgcccccacgagcacccctcagaagctgattcccccgcagccaacgggccgcccttcccccgcgccccctgccgtcccacccgccgcctcgcccgtgatgccaccgcagacccagtcccccgggcagccggcccagcccgcgcccatggtgccactgcaccagaagcagagccgcatcacccccatccagaagccgcggggcctcgaccctgtggagatcctgcaggagcgcgagtacaggctgcaggctcgcatcgcacaccgaattcaggaacttgaaaaccttcccgggtccctggccggggatttgcgaaccaaagcgaccattgagctcaaggccctcaggctgctgaacttccagaggcagctgcgccaggaggtggtggtgtgcatgcggagggacacagcgctggagacagccctcaatgctaaggcctacaagcgcagcaagcgccagtccctgcgcgaggcccgcatcactgagaagctggagaagcagcagaagatcgagcaggagcgcaagcgccggcagaagcaccaggaatacctcaatagcattctccagcatgccaaggatttcaaggaatatcacagatccgtcacaggcaaaatccagaagctgaccaaggcagtggccacgtaccatgccaacacggagcgggagcagaagaaagagaacgagcggatcgagaaggagcgcatgcggaggctcatggctgaagatgaggaggggtaccgcaagctcatcgaccagaagaaggacaagcgcctggcctacctcttgcagcagacagacgagtacgtggctaacctcacggagctggtgcggcagcacaaggctgcccaggtcgccaaggagaaaaagaagaaaaagaaaaagaagaaggcagaaaatgcagaaggacagacgcctgccattgggccggatggcgagcctctggacgagaccagccagatgagcgacctcccggtgaaggtgatccacgtggagagtgggaagatcctcacaggcacagatgcccccaaagccgggcagctggaggcctggctcgagatgaacccggggtatgaagtagctccgaggtctgatagtgaagaaagtggctcagaagaagaggaagaggaggaggaggaagagcagccgcaggcagcacagcctcccaccctgcccgtggaggagaagaagaagattccagatccagacagcgatgacgtctctgaggtggacgcgcggcacatcattgagaatgccaagcaagatgtcgatgatgaatatggcgtgtcccaggcccttgcacgtggcctgcagtcctactatgccgtggcccatgctgtcactgagagagtggacaagcagtcagcgcttatggtcaatggtgtcctcaaacagtaccagatcaaaggtttggagtggctggtgtccctgtacaacaacaacctgaacggcatcctggccgacgagatgggcctggggaagaccatccagaccatcgcgctcatcacgtacctcatggagcacaaacgcatcaatgggcccttcctcatcatcgtgcctctctcaacgctgtccaactgggcgtacgagtttgacaagtgggccccctccgtggtgaaggtgtcttacaagggatccccagcagcaagacgggcctttgtcccccagctccggagtgggaagttcaacgtcttgctgacgacgtacgagtacatcatcaaagacaagcacatcctcgccaagatccgttggaagtacatgattgtggacgaaggtcaccgcatgaagaaccaccactgcaagctgacgcaggtgctcaacacgcactatgtggcaccccgccgcctgctgctgacgggcacaccgctgcagaacaagcttcccgagctctgggcgctgctcaacttcctgctgcccaccatcttcaagagctgcagcaccttcgagcagtggtttaacgcaccctttgccatgaccggggaaaaggtggacctgaatgaggaggaaaccattctcatcatccggcgtctccacaaagtgctgcggcccttcttgctccgacgactcaagaaggaagtcgaggcccagttgcccgaaaaggtggagtacgtcatcaagtgcgacatgtctgcgctgcagcgagtgctctaccgccacatgcaggccaagggcgtgctgctgactgatggctccgagaaggacaagaagggcaaaggcggcaccaagaccctgatgaacaccatcatgcagctgcggaagatctgcaaccacccctacatgttccagcacatcgaggagtccttttccgagcacttggggttcactggcggcattgtccaagggctggacctgtaccgagcctcgggtaaatttgagcttcttgatagaattcttcccaaactccgagcaaccaaccacaaagtgctgctgttctgccaaatgacctccctcatgaccatcatggaagattactttgcgtatcgcggctttaaatacctcaggcttgatggaaccacgaaggcggaggaccggggcatgctgctgaaaaccttcaacgagcccggctctgagtacttcatcttcctgctcagcacccgggctggggggctcggcctgaacctccagtcggcagacactgtgatcatttttgacagcgactggaatcctcaccaggacctgcaagcgcaggaccgagcccaccgcatcgggcagcagaacgaggtgcgtgtgctccgcctctgcaccgtcaacagcgtggaggagaagatcctagctgcagccaagtacaagctcaacgtggaccagaaggtgatccaggccggcatgttcgaccagaagtcctccagccatgagcggcgcgccttcctgcaggccatcctggagcacgaggagcaggatgaggaggaagacgaggtgcccgacgacgagaccgtcaaccagatgatcgcccggcacgaggaggagtttgatctgttcatgcgcatggacctggaccgcaggcgcgaggaggcccgcaaccccaagcggaagccgcgcctcatggaggaggacgagctcccctcgtggatcatcaaggacgacgcggaggtggagcggctgacctgtgaggaggaggaggagaagatgttcggccgtggctcccgccaccgcaaggaggtggactacagcgactcactgacggagaagcagtggctcaagaccctgaaggccatcgaggagggcacgctggaggagatcgaagaggaggtccggcagaagaaatcatcacggaagcgcaagcgagacagcgacgccggctcctccaccccgaccaccagcacccgcagccgcgacaaggacgacgagagcaagaagcagaagaagcgcgggcggccgcctgccgagaaactctcccctaacccacccaacctcaccaagaagatgaagaagattgtggatgccgtgatcaagtacaaggacagcagcagtggacgtcagctcagcgaggtcttcatccagctgccctcgcgaaaggagctgcccgagtactacgagctcatccgcaagcccgtggacttcaagaagataaaggagcgcattcgcaaccacaagtaccgcagcctcaacgacctagagaaggacgtcatgctcctgtgccagaacgcacagaccttcaacctggagggctccctgatctatgaagactccatcgtcttgcagtcggtcttcaccagcgtgcggcagaaaatcgagaaggaggatgacagtgaaggcgaggagagtgaggaggaggaagagggcgaggaggaaggctccgaatccgaatctcggtccgtcaaagtgaagatcaagcttggccggaaggagaaggcacaggaccggctgaagggcggccggcggcggccgagccgagggtcccgagccaagccggtcgtgagtgacgatgacagtgaggaggaacaagaggaggaccgctcaggaagtggcagcgaagaagactgagccccgacattccagtctcgaccccgagcccctcgttccagagctgagatggcataggccttagcagtaacgggtagcagcagatgtagtttcagacttggagtaaaactgtataaacaaaagaatcttccatatttatacagcagagaagctgtaggactgtttgtgactggccctgtcctggcatcagtagcatctgtaacagcattaactgtcttaaagagagagagagagaattccgaattggggaacacacgatacctgtttttcttttccgttgctggcagtactgttgcgccgcagtttggagtcactgtagttaagtgtggatgcatgtgcgtcaccgtccactcctcctactgtattttattggacaggtcagactcgccgggggcccggcgagggtatgtcagtgtcactggatgtcaaacagtaataaattaaaccaacaacaaaacgcacagccaaaaaaaaa

SEQ ID NO: 12 - DNA, 5326 bp, Homo sapiens
atgtccactccagacccacccctgggcggaactcctcggccaggtccttccccgggccctggcccttcccctggagccatgctgggccctagcccgggtccctcgccgggctccgcccacagcatgatggggcccagcccagggccgccctcagcaggacaccccatccccacccaggggcctggagggtaccctcaggacaacatgcaccagatgcacaagcccatggagtccatgcatgagaagggcatgtcggacgacccgcgctacaaccagatgaaaggaatggggatgcggtcagggggccatgctgggatggggcccccgcccagccccatggaccagcactcccaaggttacccctcgcccctgggtggctctgagcatgcctctagtccagttccagccagtggcccgtcttcggggccccagatgtcttccgggccaggaggtgccccgctggatggtgctgacccccaggccttggggcagcagaaccggggcccaaccccatttaaccagaaccagctgcaccagctcagagctcagatcatggcctacaagatgctggccagggggcagcccctccccgaccacctgcagatggcggtgcagggcaagcggccgatgcccgggatgcagcagcagatgccaacgctacctccaccctcggtgtccgcaacaggacccggccctggccctggccctggccccggcccgggtcccggcccggcacctccaaattacagcaggcctcatggtatgggagggcccaacatgcctcccccaggaccctcgggcgtgccccccgggatgccaggccagcctcctggagggcctcccaagccctggcctgaaggacccatggcgaatgctgctgcccccacgagcacccctcagaagctgattcccccgcagccaacgggccgcccttcccccgcgccccctgccgtcccacccgccgcctcgcccgtgatgccaccgcagacccagtcccccgggcagccggcccagcccgcgcccatggtgccactgcaccagaagcagagccgcatcacccccatccagaagccgcggggcctcgaccctgtggagatcctgcaggagcgcgagtacaggctgcaggctcgcatcgcacaccgaattcaggaacttgaaaaccttcccgggtccctggccggggatttgcgaaccaaagcgaccattgagctcaaggccctcaggctgctgaacttccagaggcagctgcgccaggaggtggtggtgtgcatgcggagggacacagcgctggagacagccctcaatgctaaggcctacaagcgcagcaagcgccagtccctgcgcgaggcccgcatcactgagaagctggagaagcagcagaagatcgagcaggagcgcaagcgccggcagaagcaccaggaatacctcaatagcattctccagcatgccaaggatttcaaggaatatcacagatccgtcacaggcaaaatccagaagctgaccaaggcagtggccacgtaccatgccaacacggagcgggagcagaagaaagagaacgagcggatcgagaaggagcgcatgcggaggctcatggctgaagatgaggaggggtaccgcaagctcatcgaccagaagaaggacaagcgcctggcctacctcttgcagcagacagacgagtacgtggctaacctcacggagctggtgcggcagcacaaggctgcccaggtcgccaaggagaaaaagaagaaaaagaaaaagaagaaggcagaaaatgcagaaggacagacgcctgccattgggccggatggcgagcctctggacgagaccagccagatgagcgacctcccggtgaaggtgatccacgtggagagtgggaagatcctcacaggcacagatgcccccaaagccgggcagctggaggcctggctcgagatgaacccggggtatgaagtagctccgaggtctgatagtgaagaaagtggctcagaagaagaggaagaggaggaggaggaagagcagccgcaggcagcacagcctcccaccctgcccgtggaggagaagaagaagattccagatccagacagcgatgacgtctctgaggtggacgcgcggcacatcattgagaatgccaagcaagatgtcgatgatgaatatggcgtgtcccaggcccttgcacgtggcctgcagtcctactatgccgtggcccatgctgtcactgagagagtggacaagcagtcagcgcttatggtcaatggtgtcctcaaacagtaccagatcaaaggtttggagtggctggtgtccctgtacaacaacaacctgaacggcatcctggccgacgagatgggcctggggaagaccatccagaccatcgcgctcatcacgtacctcatggagcacaaacgcatcaatgggcccttcctcatcatcgtgcctctctcaacgctgtccaactgggcgtacgagtttgacaagtgggccccctccgtggtgaaggtgtcttacaagggatccccagcagcaagacgggcctttgtcccccagctccggagtgggaagttcaacgtcttgctgacgacgtacgagtacatcatcaaagacaagcacatcctcgccaagatccgttggaagtacatgattgtggacgaaggtcaccgcatgaagaaccaccactgcaagctgacgcaggtgctcaacacgcactatgtggcaccccgccgcctgctgctgacgggcacaccgctgcagaacaagcttcccgagctctgggcgctgctcaacttcctgctgcccaccatcttcaagagctgcagcaccttcgagcagtggtttaacgcaccctttgccatgaccggggaaaaggtggacctgaatgaggaggaaaccattctcatcatccggcgtctccacaaagtgctgcggcccttcttgctccgacgactcaagaaggaagtcgaggcccagttgcccgaaaaggtggagtacgtcatcaagtgcgacatgtctgcgctgcagcgagtgctctaccgccacatgcaggccaagggcgtgctgctgactgatggctccgagaaggacaagaagggcaaaggcggcaccaagaccctgatgaacaccatcatgcagctgcggaagatctgcaaccacccctacatgttccagcacatcgaggagtccttttccgagcacttggggttcactggcggcattgtccaagggctggacctgtaccgagcctcgggtaaatttgagcttcttgatagaattcttcccaaactccgagcaaccaaccacaaagtgctgctgttctgccaaatgacctccctcatgaccatcatggaagattactttgcgtatcgcggctttaaatacctcaggcttgatggaaccacgaaggcggaggaccggggcatgctgctgaaaaccttcaacgagcccggctctgagtacttcatcttcctgctcagcacccgggctggggggctcggcctgaacctccagtcggcagacactgtgatcatttttgacagcgactggaatcctcaccaggacctgcaagcgcaggaccgagcccaccgcatcgggcagcagaacgaggtgcgtgtgctccgcctctgcaccgtcaacagcgtggaggagaagatcctagctgcagccaagtacaagctcaacgtggaccagaaggtgatccaggccggcatgttcgaccagaagtcctccagccatgagcggcgcgccttcctgcaggccatcctggagcacgaggagcaggatgaggaggaagacgaggtgcccgacgacgagaccgtcaaccagatgatcgcccggcacgaggaggagtttgatctgttcatgcgcatggacctggaccgcaggcgcgaggaggcccgcaaccccaagcggaagccgcgcctcatggaggaggacgagctcccctcgtggatcatcaaggacgacgcggaggtggagcggctgacctgtgaggaggaggaggagaagatgttcggccgtggctcccgccaccgcaaggaggtggactacagcgactcactgacggagaagcagtggctcaagaccctgaaggccatcgaggagggcacgctggaggagatcgaagaggaggtccggcagaagaaatcatcacggaagcgcaagcgagacagcgacgccggctcctccaccccgaccaccagcacccgcagccgcgacaaggacgacgagagcaagaagcagaagaagcgcgggcggccgcctgccgagaaactctcccctaacccacccaacctcaccaagaagatgaagaagattgtggatgccgtgatcaagtacaaggacagcagtggacgtcagctcagcgaggtcttcatccagctgccctcgcgaaaggagctgcccgagtactacgagctcatccgcaagcccgtggacttcaagaagataaaggagcgcattcgcaaccacaagtaccgcagcctcaacgacctagagaaggacgtcatgctcctgtgccagaacgcacagaccttcaacctggagggctccctgatctatgaagactccatcgtcttgcagtcggtcttcaccagcgtgcggcagaaaatcgagaaggaggatgacagtgaaggcgaggagagtgaggaggaggaagagggcgaggaggaaggctccgaatccgaatctcggtccgtcaaagtgaagatcaagcttggccggaaggagaaggcacaggaccggctgaagggcggccggcggcggccgagccgagggtcccgagccaagccggtcgtgagtgacgatgacagtgaggaggaacaagaggaggaccgctcaggaagtggcagcgaagaagactgagccccgacattccagtctcgaccccgagcccctcgttccagagctgagatggcataggccttagcagtaacgggtagcagcagatgtagtttcagacttggagtaaaactgtataaacaaaagaatcttccatatttatacagcagagaagctgtaggactgtttgtgactggccctgtcctggcatcagtagcatctgtaacagcattaactgtcttaaagagagagagagagaattccgaattggggaacacacgatacctgtttttcttttccgttgctggcagtactgttgcgccgcagtttggagtcactgtagttaagtgtggatgcatgtgcgtcaccgtccactcctcctactgtattttattggacaggtcagactcgccgggggcccggcgagggtatgtcagtgtcactggatgtcaaacagtaataaattaaaccaacaacaaaacgcacagccaaaaaaaaa

SEQ ID NO: 13 - DNA, 5320 bp, Homo sapiens
atgtccactccagacccacccctgggcggaactcctcggccaggtccttccccgggccctggcccttcccctggagccatgctgggccctagcccgggtccctcgccgggctccgcccacagcatgatggggcccagcccagggccgccctcagcaggacaccccatccccacccaggggcctggagggtaccctcaggacaacatgcaccagatgcacaagcccatggagtccatgcatgagaagggcatgtcggacgacccgcgctacaaccagatgaaaggaatggggatgcggtcagggggccatgctgggatggggcccccgcccagccccatggaccagcactcccaaggttacccctcgcccctgggtggctctgagcatgcctctagtccagttccagccagtggcccgtcttcggggccccagatgtcttccgggccaggaggtgccccgctggatggtgctgacccccaggccttggggcagcagaaccggggcccaaccccatttaaccagaaccagctgcaccagctcagagctcagatcatggcctacaagatgctggccagggggcagcccctccccgaccacctgcagatggcggtgcagggcaagcggccgatgcccgggatgcagcagcagatgccaacgctacctccaccctcggtgtccgcaacaggacccggccctggccctggccctggccccggcccgggtcccggcccggcacctccaaattacagcaggcctcatggtatgggagggcccaacatgcctcccccaggaccctcgggcgtgccccccgggatgccaggccagcctcctggagggcctcccaagccctggcctgaaggacccatggcgaatgctgctgcccccacgagcacccctcagaagctgattcccccgcagccaacgggccgcccttcccccgcgccccctgccgtcccacccgccgcctcgcccgtgatgccaccgcagacccagtcccccgggcagccggcccagcccgcgcccatggtgccactgcaccagaagcagagccgcatcacccccatccagaagccgcggggcctcgaccctgtggagatcctgcaggagcgcgagtacaggctgcaggctcgcatcgcacaccgaattcaggaacttgaaaaccttcccgggtccctggccggggatttgcgaaccaaagcgaccattgagctcaaggccctcaggctgctgaacttccagaggcagctgcgccaggaggtggtggtgtgcatgcggagggacacagcgctggagacagccctcaatgctaaggcctacaagcgcagcaagcgccagtccctgcgcgaggcccgcatcactgagaagctggagaagcagcagaagatcgagcaggagcgcaagcgccggcagaagcaccaggaatacctcaatagcattctccagcatgccaaggatttcaaggaatatcacagatccgtcacaggcaaaatccagaagctgaccaaggcagtggccacgtaccatgccaacacggagcgggagcagaagaaagagaacgagcggatcgagaaggagcgcatgcggaggctcatggctgaagatgaggaggggtaccgcaagctcatcgaccagaagaaggacaagcgcctggcctacctcttgcagcagacagacgagtacgtggctaacctcacggagctggtgcggcagcacaaggctgcccaggtcgccaaggagaaaaagaagaaaaagaaaaagaagaaggcagaaaatgcagaaggacagacgcctgccattgggccggatggcgagcctctggacgagaccagccagatgagcgacctcccggtgaaggtgatccacgtggagagtgggaagatcctcacaggcacagatgcccccaaagccgggcagctggaggcctggctcgagatgaacccggggtatgaagtagctccgaggtctgatagtgaagaaagtggctcagaagaagaggaagaggaggaggaggaagagcagccgcaggcagcacagcctcccaccctgcccgtggaggagaagaagaagattccagatccagacagcgatgacgtctctgaggtggacgcgcggcacatcattgagaatgccaagcaagatgtcgatgatgaatatggcgtgtcccaggcccttgcacgtggcctgcagtcctactatgccgtggcccatgctgtcactgagagagtggacaagcagtcagcgcttatggtcaatggtgtcctcaaacagtaccagatcaaaggtttggagtggctggtgtccctgtacaacaacaacctgaacggcatcctggccgacgagatgggcctggggaagaccatccagaccatcgcgctcatcacgtacctcatggagcacaaacgcatcaatgggcccttcctcatcatcgtgcctctctcaacgctgtccaactgggcgtacgagtttgacaagtgggccccctccgtggtgaaggtgtcttacaagggatccccagcagcaagacgggcctttgtcccccagctccggagtgggaagttcaacgtcttgctgacgacgtacgagtacatcatcaaagacaagcacatcctcgccaagatccgttggaagtacatgattgtggacgaaggtcaccgcatgaagaaccaccactgcaagctgacgcaggtgctcaacacgcactatgtggcaccccgccgcctgctgctgacgggcacaccgctgcagaacaagcttcccgagctctgggcgctgctcaacttcctgctgcccaccatcttcaagagctgcagcaccttcgagcagtggtttaacgcaccctttgccatgaccggggaaaaggtggacctgaatgaggaggaaaccattctcatcatccggcgtctccacaaagtgctgcggcccttcttgctccgacgactcaagaaggaagtcgaggcccagttgcccgaaaaggtggagtacgtcatcaagtgcgacatgtctgcgctgcagcgagtgctctaccgccacatgcaggccaagggcgtgctgctgactgatggctccgagaaggacaagaagggcaaaggcggcaccaagaccctgatgaacaccatcatgcagctgcggaagatctgcaaccacccctacatgttccagcacatcgaggagtccttttccgagcacttggggttcactggcggcattgtccaagggctggacctgtaccgagcctcgggtaaatttgagcttcttgatagaattcttcccaaactccgagcaaccaaccacaaagtgctgctgttctgccaaatgacctccctcatgaccatcatggaagattactttgcgtatcgcggctttaaatacctcaggcttgatggaaccacgaaggcggaggaccggggcatgctgctgaaaaccttcaacgagcccggctctgagtacttcatcttcctgctcagcacccgggctggggggctcggcctgaacctccagtcggcagacactgtgatcatttttgacagcgactggaatcctcaccaggacctgcaagcgcaggaccgagcccaccgcatcgggcagcagaacgaggtgcgtgtgctccgcctctgcaccgtcaacagcgtggaggagaagatcctagctgcagccaagtacaagctcaacgtggaccagaaggtgatccaggccggcatgttcgaccagaagtcctccagccatgagcggcgcgccttcctgcaggccatcctggagcacgaggagcaggatgaggaggaagacgaggtgcccgacgacgagaccgtcaaccagatgatcgcccggcacgaggaggagtttgatctgttcatgcgcatggacctggaccgcaggcgcgaggaggcccgcaaccccaagcggaagccgcgcctcatggaggaggacgagctcccctcgtggatcatcaaggacgacgcggaggtggagcggctgacctgtgaggaggaggaggagaagatgttcggccgtggctcccgccaccgcaaggaggtggactacagcgactcactgacggagaagcagtggctcaaggccatcgaggagggcacgctggaggagatcgaagaggaggtccggcagaagaaatcatcacggaagcgcaagcgagacagcgacgccggctcctccaccccgaccaccagcacccgcagccgcgacaaggacgacgagagcaagaagcagaagaagcgcgggcggccgcctgccgagaaactctcccctaacccacccaacctcaccaagaagatgaagaagattgtggatgccgtgatcaagtacaaggacagcagcagtggacgtcagctcagcgaggtcttcatccagctgccctcgcgaaaggagctgcccgagtactacgagctcatccgcaagcccgtggacttcaagaagataaaggagcgcattcgcaaccacaagtaccgcagcctcaacgacctagagaaggacgtcatgctcctgtgccagaacgcacagaccttcaacctggagggctccctgatctatgaagactccatcgtcttgcagtcggtcttcaccagcgtgcggcagaaaatcgagaaggaggatgacagtgaaggcgaggagagtgaggaggaggaagagggcgaggaggaaggctccgaatccgaatctcggtccgtcaaagtgaagatcaagcttggccggaaggagaaggcacaggaccggctgaagggcggccggcggcggccgagccgagggtcccgagccaagccggtcgtgagtgacgatgacagtgaggaggaacaagaggaggaccgctcaggaagtggcagcgaagaagactgagccccgacattccagtctcgaccccgagcccctcgttccagagctgagatggcataggccttagcagtaacgggtagcagcagatgtagtttcagacttggagtaaaactgtataaacaaaagaatcttccatatttatacagcagagaagctgtaggactgtttgtgactggccctgtcctggcatcagtagcatctgtaacagcattaactgtcttaaagagagagagagagaattccgaattggggaacacacgatacctgtttttcttttccgttgctggcagtactgttgcgccgcagtttggagtcactgtagttaagtgtggatgcatgtgcgtcaccgtccactcctcctactgtattttattggacaggtcagactcgccgggggcccggcgagggtatgtcagtgtcactggatgtcaaacagtaataaattaaaccaacaacaaaacgcacagccaaaaaaaaa

SEQ ID NO: 14 - DNA, 5317 bp, Homo sapiens
atgtccactccagacccacccctgggcggaactcctcggccaggtccttccccgggccctggcccttcccctggagccatgctgggccctagcccgggtccctcgccgggctccgcccacagcatgatggggcccagcccagggccgccctcagcaggacaccccatccccacccaggggcctggagggtaccctcaggacaacatgcaccagatgcacaagcccatggagtccatgcatgagaagggcatgtcggacgacccgcgctacaaccagatgaaaggaatggggatgcggtcagggggccatgctgggatggggcccccgcccagccccatggaccagcactcccaaggttacccctcgcccctgggtggctctgagcatgcctctagtccagttccagccagtggcccgtcttcggggccccagatgtcttccgggccaggaggtgccccgctggatggtgctgacccccaggccttggggcagcagaaccggggcccaaccccatttaaccagaaccagctgcaccagctcagagctcagatcatggcctacaagatgctggccagggggcagcccctccccgaccacctgcagatggcggtgcagggcaagcggccgatgcccgggatgcagcagcagatgccaacgctacctccaccctcggtgtccgcaacaggacccggccctggccctggccctggccccggcccgggtcccggcccggcacctccaaattacagcaggcctcatggtatgggagggcccaacatgcctcccccaggaccctcgggcgtgccccccgggatgccaggccagcctcctggagggcctcccaagccctggcctgaaggacccatggcgaatgctgctgcccccacgagcacccctcagaagctgattcccccgcagccaacgggccgcccttcccccgcgccccctgccgtcccacccgccgcctcgcccgtgatgccaccgcagacccagtcccccgggcagccggcccagcccgcgcccatggtgccactgcaccagaagcagagccgcatcacccccatccagaagccgcggggcctcgaccctgtggagatcctgcaggagcgcgagtacaggctgcaggctcgcatcgcacaccgaattcaggaacttgaaaaccttcccgggtccctggccggggatttgcgaaccaaagcgaccattgagctcaaggccctcaggctgctgaacttccagaggcagctgcgccaggaggtggtggtgtgcatgcggagggacacagcgctggagacagccctcaatgctaaggcctacaagcgcagcaagcgccagtccctgcgcgaggcccgcatcactgagaagctggagaagcagcagaagatcgagcaggagcgcaagcgccggcagaagcaccaggaatacctcaatagcattctccagcatgccaaggatttcaaggaatatcacagatccgtcacaggcaaaatccagaagctgaccaaggcagtggccacgtaccatgccaacacggagcgggagcagaagaaagagaacgagcggatcgagaaggagcgcatgcggaggctcatggctgaagatgaggaggggtaccgcaagctcatcgaccagaagaaggacaagcgcctggcctacctcttgcagcagacagacgagtacgtggctaacctcacggagctggtgcggcagcacaaggctgcccaggtcgccaaggagaaaaagaagaaaaagaaaaagaagaaggcagaaaatgcagaaggacagacgcctgccattgggccggatggcgagcctctggacgagaccagccagatgagcgacctcccggtgaaggtgatccacgtggagagtgggaagatcctcacaggcacagatgcccccaaagccgggcagctggaggcctggctcgagatgaacccggggtatgaagtagctccgaggtctgatagtgaagaaagtggctcagaagaagaggaagaggaggaggaggaagagcagccgcaggcagcacagcctcccaccctgcccgtggaggagaagaagaagattccagatccagacagcgatgacgtctctgaggtggacgcgcggcacatcattgagaatgccaagcaagatgtcgatgatgaatatggcgtgtcccaggcccttgcacgtggcctgcagtcctactatgccgtggcccatgctgtcactgagagagtggacaagcagtcagcgcttatggtcaatggtgtcctcaaacagtaccagatcaaaggtttggagtggctggtgtccctgtacaacaacaacctgaacggcatcctggccgacgagatgggcctggggaagaccatccagaccatcgcgctcatcacgtacctcatggagcacaaacgcatcaatgggcccttcctcatcatcgtgcctctctcaacgctgtccaactgggcgtacgagtttgacaagtgggccccctccgtggtgaaggtgtcttacaagggatccccagcagcaagacgggcctttgtcccccagctccggagtgggaagttcaacgtcttgctgacgacgtacgagtacatcatcaaagacaagcacatcctcgccaagatccgttggaagtacatgattgtggacgaaggtcaccgcatgaagaaccaccactgcaagctgacgcaggtgctcaacacgcactatgtggcaccccgccgcctgctgctgacgggcacaccgctgcagaacaagcttcccgagctctgggcgctgctcaacttcctgctgcccaccatcttcaagagctgcagcaccttcgagcagtggtttaacgcaccctttgccatgaccggggaaaaggtggacctgaatgaggaggaaaccattctcatcatccggcgtctccacaaagtgctgcggcccttcttgctccgacgactcaagaaggaagtcgaggcccagttgcccgaaaaggtggagtacgtcatcaagtgcgacatgtctgcgctgcagcgagtgctctaccgccacatgcaggccaagggcgtgctgctgactgatggctccgagaaggacaagaagggcaaaggcggcaccaagaccctgatgaacaccatcatgcagctgcggaagatctgcaaccacccctacatgttccagcacatcgaggagtccttttccgagcacttggggttcactggcggcattgtccaagggctggacctgtaccgagcctcgggtaaatttgagcttcttgatagaattcttcccaaactccgagcaaccaaccacaaagtgctgctgttctgccaaatgacctccctcatgaccatcatggaagattactttgcgtatcgcggctttaaatacctcaggcttgatggaaccacgaaggcggaggaccggggcatgctgctgaaaaccttcaacgagcccggctctgagtacttcatcttcctgctcagcacccgggctggggggctcggcctgaacctccagtcggcagacactgtgatcatttttgacagcgactggaatcctcaccaggacctgcaagcgcaggaccgagcccaccgcatcgggcagcagaacgaggtgcgtgtgctccgcctctgcaccgtcaacagcgtggaggagaagatcctagctgcagccaagtacaagctcaacgtggaccagaaggtgatccaggccggcatgttcgaccagaagtcctccagccatgagcggcgcgccttcctgcaggccatcctggagcacgaggagcaggatgaggaggaagacgaggtgcccgacgacgagaccgtcaaccagatgatcgcccggcacgaggaggagtttgatctgttcatgcgcatggacctggaccgcaggcgcgaggaggcccgcaaccccaagcggaagccgcgcctcatggaggaggacgagctcccctcgtggatcatcaaggacgacgcggaggtggagcggctgacctgtgaggaggaggaggagaagatgttcggccgtggctcccgccaccgcaaggaggtggactacagcgactcactgacggagaagcagtggctcaaggccatcgaggagggcacgctggaggagatcgaagaggaggtccggcagaagaaatcatcacggaagcgcaagcgagacagcgacgccggctcctccaccccgaccaccagcacccgcagccgcgacaaggacgacgagagcaagaagcagaagaagcgcgggcggccgcctgccgagaaactctcccctaacccacccaacctcaccaagaagatgaagaagattgtggatgccgtgatcaagtacaaggacagcagtggacgtcagctcagcgaggtcttcatccagctgccctcgcgaaaggagctgcccgagtactacgagctcatccgcaagcccgtggacttcaagaagataaaggagcgcattcgcaaccacaagtaccgcagcctcaacgacctagagaaggacgtcatgctcctgtgccagaacgcacagaccttcaacctggagggctccctgatctatgaagactccatcgtcttgcagtcggtcttcaccagcgtgcggcagaaaatcgagaaggaggatgacagtgaaggcgaggagagtgaggaggaggaagagggcgaggaggaaggctccgaatccgaatctcggtccgtcaaagtgaagatcaagcttggccggaaggagaaggcacaggaccggctgaagggcggccggcggcggccgagccgagggtcccgagccaagccggtcgtgagtgacgatgacagtgaggaggaacaagaggaggaccgctcaggaagtggcagcgaagaagactgagccccgacattccagtctcgaccccgagcccctcgttccagagctgagatggcataggccttagcagtaacgggtagcagcagatgtagtttcagacttggagtaaaactgtataaacaaaagaatcttccatatttatacagcagagaagctgtaggactgtttgtgactggccctgtcctggcatcagtagcatctgtaacagcattaactgtcttaaagagagagagagagaattccgaattggggaacacacgatacctgtttttcttttccgttgctggcagtactgttgcgccgcagtttggagtcactgtagttaagtgtggatgcatgtgcgtcaccgtccactcctcctactgtattttattggacaggtcagactcgccgggggcccggcgagggtatgtcagtgtcactggatgtcaaacagtaataaattaaaccaacaacaaaacgcacagccaaaaaaaaa

SEQ ID NO: 15 - protein, 1679 aa, Homo sapiens
MSTPDPPLGGTPRPGPSPGPGPSPGAMLGPSPGPSPGSAHSMMGPSPGPPSAGHPIPTQGPGGYPQDNMHQMHKPMESMHEKGMSDDPRYNQMKGMGMRSGGHAGMGPPPSPMDQHSQGYPSPLGGSEHASSPVPASGPSSGPQMSSGPGGAPLDGADPQALGQQNRGPTPFNQNQLHQLRAQIMAYKMLARGQPLPDHLQMAVQGKRPMPGMQQQMPTLPPPSVSATGPGPGPGPGPGPGPGPAPPNYSRPHGMGGPNMPPPGPSGVPPGMPGQPPGGPPKPWPEGPMANAAAPTSTPQKLIPPQPTGRPSPAPPAVPPAASPVMPPQTQSPGQPAQPAPMVPLHQKQSRITPIQKPRGLDPVEILQEREYRLQARIAHRIQELENLPGSLAGDLRTKATIELKALRLLNFQRQLRQEVVVCMRRDTALETALNAKAYKRSKRQSLREARITEKLEKQQKIEQERKRRQKHQEYLNSILQHAKDFKEYHRSVTGKIQKLTKAVATYHANTEREQKKENERIEKERMRRLMAEDEEGYRKLIDQKKDKRLAYLLQQTDEYVANLTELVRQHKAAQVAKEKKKKKKKKKAENAEGQTPAIGPDGEPLDETSQMSDLPVKVIHVESGKILTGTDAPKAGQLEAWLEMNPGYEVAPRSDSEESGSEEEEEEEEEEQPQAAQPPTLPVEEKKKIPDPDSDDVSEVDARHIIENAKQDVDDEYGVSQALARGLQSYYAVAHAVTERVDKQSALMVNGVLKQYQIKGLEWLVSLYNNNLNGILADEMGLGKTIQTIALITYLMEHKRINGPFLIIVPLSTLSNWAYEFDKWAPSVVKVSYKGSPAARRAFVPQLRSGKFNVLLTTYEYIIKDKHILAKIRWKYMIVDEGHRMKNHHCKLTQVLNTHYVAPRRLLLTGTPLQNKLPELWALLNFLLPTIFKSCSTFEQWFNAPFAMTGEKVDLNEEETILIIRRLHKVLRPFLLRRLKKEVEAQLPEKVEYVIKCDMSALQRVLYRHMQAKGVLLTDGSEKDKKGKGGTKTLMNTIMQLRKICNHPYMFQHIEESFSEHLGFTGGIVQGLDLYRASGKFELLDRILPKLRATNHKVLLFCQMTSLMTIMEDYFAYRGFKYLRLDGTTKAEDRGMLLKTFNEPGSEYFIFLLSTRAGGLGLNLQSADTVIIFDSDWNPHQDLQAQDRAHRIGQQNEVRVLRLCTVNSVEEKILAAAKYKLNVDQKVIQAGMFDQKSSSHERRAFLQAILEHEEQDESRHCSTGSGSASFAHTAPPPAGVNPDLEEPPLKEEDEVPDDETVNQMIARHEEEFDLFMRMDLDRRREEARNPKRKPRLMEEDELPSWIIKDDAEVERLTCEEEEEKMFGRGSRHRKEVDYSDSLTEKQWLKKITGKDIHDTASSVARGLQFQRGLQFCTRASKAIEEGTLEEIEEEVRQKKSSRKRKRDSDAGSSTPTTSTRSRDKDDESKKQKKRGRPPAEKLSPNPPNLTKKMKKIVDAVIKYKDSSSGRQLSEVFIQLPSRKELPEYYELIRKPVDFKKIKERIRNHKYRSLNDLEKDVMLLCQNAQTFNLEGSLIYEDSIVLQSVFTSVRQKIEKEDDSEGEESEEEEEGEEEGSESESRSVKVKIKLGRKEKAQDRLKGGRRRPSRGSRAKPVVSDDDSEEEQEEDRSGSGSEED

SEQ ID NO: 16 - protein, 1647 aa, Homo sapiens
MSTPDPPLGGTPRPGPSPGPGPSPGAMLGPSPGPSPGSAHSMMGPSPGPPSAGHPIPTQGPGGYPQDNMHQMHKPMESMHEKGMSDDPRYNQMKGMGMRSGGHAGMGPPPSPMDQHSQGYPSPLGGSEHASSPVPASGPSSGPQMSSGPGGAPLDGADPQALGQQNRGPTPFNQNQLHQLRAQIMAYKMLARGQPLPDHLQMAVQGKRPMPGMQQQMPTLPPPSVSATGPGPGPGPGPGPGPGPAPPNYSRPHGMGGPNMPPPGPSGVPPGMPGQPPGGPPKPWPEGPMANAAAPTSTPQKLIPPQPTGRPSPAPPAVPPAASPVMPPQTQSPGQPAQPAPMVPLHQKQSRITPIQKPRGLDPVEILQEREYRLQARIAHRIQELENLPGSLAGDLRTKATIELKALRLLNFQRQLRQEVVVCMRRDTALETALNAKAYKRSKRQSLREARITEKLEKQQKIEQERKRRQKHQEYLNSILQHAKDFKEYHRSVTGKIQKLTKAVATYHANTEREQKKENERIEKERMRRLMAEDEEGYRKLIDQKKDKRLAYLLQQTDEYVANLTELVRQHKAAQVAKEKKKKKKKKKAENAEGQTPAIGPDGEPLDETSQMSDLPVKVIHVESGKILTGTDAPKAGQLEAWLEMNPGYEVAPRSDSEESGSEEEEEEEEEEQPQAAQPPTLPVEEKKKIPDPDSDDVSEVDARHIIENAKQDVDDEYGVSQALARGLQSYYAVAHAVTERVDKQSALMVNGVLKQYQIKGLEWLVSLYNNNLNGILADEMGLGKTIQTIALITYLMEHKRINGPFLIIVPLSTLSNWAYEFDKWAPSVVKVSYKGSPAARRAFVPQLRSGKFNVLLTTYEYIIKDKHILAKIRWKYMIVDEGHRMKNHHCKLTQVLNTHYVAPRRLLLTGTPLQNKLPELWALLNFLLPTIFKSCSTFEQWFNAPFAMTGEKVDLNEEETILIIRRLHKVLRPFLLRRLKKEVEAQLPEKVEYVIKCDMSALQRVLYRHMQAKGVLLTDGSEKDKKGKGGTKTLMNTIMQLRKICNHPYMFQHIEESFSEHLGFTGGIVQGLDLYRASGKFELLDRILPKLRATNHKVLLFCQMTSLMTIMEDYFAYRGFKYLRLDGTTKAEDRGMLLKTFNEPGSEYFIFLLSTRAGGLGLNLQSADTVIIFDSDWNPHQDLQAQDRAHRIGQQNEVRVLRLCTVNSVEEKILAAAKYKLNVDQKVIQAGMFDQKSSSHERRAFLQAILEHEEQDESRHCSTGSGSASFAHTAPPPAGVNPDLEEPPLKEEDEVPDDETVNQMIARHEEEFDLFMRMDLDRRREEARNPKRKPRLMEEDELPSWIIKDDAEVERLTCEEEEEKMFGRGSRHRKEVDYSDSLTEKQWLKAIEEGTLEEIEEEVRQKKSSRKRKRDSDAGSSTPTTSTRSRDKDDESKKQKKRGRPPAEKLSPNPPNLTKKMKKIVDAVIKYKDSSSGRQLSEVFIQLPSRKELPEYYELIRKPVDFKKIKERIRNHKYRSLNDLEKDVMLLCQNAQTFNLEGSLIYEDSIVLQSVFTSVRQKIEKEDDSEGEESEEEEEGEEEGSESESRSVKVKIKLGRKEKAQDRLKGGRRRPSRGSRAKPVVSDDDSEEEQEEDRSGSGSEED

SEQ ID NO: 17 - protein, 1617 aa, Homo sapiens
MSTPDPPLGGTPRPGPSPGPGPSPGAMLGPSPGPSPGSAHSMMGPSPGPPSAGHPIPTQGPGGYPQDNMHQMHKPMESMHEKGMSDDPRYNQMKGMGMRSGGHAGMGPPPSPMDQHSQGYPSPLGGSEHASSPVPASGPSSGPQMSSGPGGAPLDGADPQALGQQNRGPTPFNQNQLHQLRAQIMAYKMLARGQPLPDHLQMAVQGKRPMPGMQQQMPTLPPPSVSATGPGPGPGPGPGPGPGPAPPNYSRPHGMGGPNMPPPGPSGVPPGMPGQPPGGPPKPWPEGPMANAAAPTSTPQKLIPPQPTGRPSPAPPAVPPAASPVMPPQTQSPGQPAQPAPMVPLHQKQSRITPIQKPRGLDPVEILQEREYRLQARIAHRIQELENLPGSLAGDLRTKATIELKALRLLNFQRQLRQEVVVCMRRDTALETALNAKAYKRSKRQSLREARITEKLEKQQKIEQERKRRQKHQEYLNSILQHAKDFKEYHRSVTGKIQKLTKAVATYHANTEREQKKENERIEKERMRRLMAEDEEGYRKLIDQKKDKRLAYLLQQTDEYVANLTELVRQHKAAQVAKEKKKKKKKKKAENAEGQTPAIGPDGEPLDETSQMSDLPVKVIHVESGKILTGTDAPKAGQLEAWLEMNPGYEVAPRSDSEESGSEEEEEEEEEEQPQAAQPPTLPVEEKKKIPDPDSDDVSEVDARHIIENAKQDVDDEYGVSQALARGLQSYYAVAHAVTERVDKQSALMVNGVLKQYQIKGLEWLVSLYNNNLNGILADEMGLGKTIQTIALITYLMEHKRINGPFLIIVPLSTLSNWAYEFDKWAPSVVKVSYKGSPAARRAFVPQLRSGKFNVLLTTYEYIIKDKHILAKIRWKYMIVDEGHRMKNHHCKLTQVLNTHYVAPRRLLLTGTPLQNKLPELWALLNFLLPTIFKSCSTFEQWFNAPFAMTGEKVDLNEEETILIIRRLHKVLRPFLLRRLKKEVEAQLPEKVEYVIKCDMSALQRVLYRHMQAKGVLLTDGSEKDKKGKGGTKTLMNTIMQLRKICNHPYMFQHIEESFSEHLGFTGGIVQGLDLYRASGKFELLDRILPKLRATNHKVLLFCQMTSLMTIMEDYFAYRGFKYLRLDGTTKAEDRGMLLKTFNEPGSEYFIFLLSTRAGGLGLNLQSADTVIIFDSDWNPHQDLQAQDRAHRIGQQNEVRVLRLCTVNSVEEKILAAAKYKLNVDQKVIQAGMFDQKSSSHERRAFLQAILEHEEQDEEEDEVPDDETVNQMIARHEEEFDLFMRMDLDRRREEARNPKRKPRLMEEDELPSWIIKDDAEVERLTCEEEEEKMFGRGSRHRKEVDYSDSLTEKQWLKTLKAIEEGTLEEIEEEVRQKKSSRKRKRDSDAGSSTPTTSTRSRDKDDESKKQKKRGRPPAEKLSPNPPNLTKKMKKIVDAVIKYKDSSSGRQLSEVFIQLPSRKELPEYYELIRKPVDFKKIKERIRNHKYRSLNDLEKDVMLLCQNAQTFNLEGSLIYEDSIVLQSVFTSVRQKIEKEDDSEGEESEEEEEGEEEGSESESRSVKVKIKLGRKEKAQDRLKGGRRRPSRGSRAKPVVSDDDSEEEQEEDRSGSGSEED

SEQ ID NO: 18 - protein, 1616 aa, Homo sapiens
MSTPDPPLGGTPRPGPSPGPGPSPGAMLGPSPGPSPGSAHSMMGPSPGPPSAGHPIPTQGPGGYPQDNMHQMHKPMESMHEKGMSDDPRYNQMKGMGMRSGGHAGMGPPPSPMDQHSQGYPSPLGGSEHASSPVPASGPSSGPQMSSGPGGAPLDGADPQALGQQNRGPTPFNQNQLHQLRAQIMAYKMLARGQPLPDHLQMAVQGKRPMPGMQQQMPTLPPPSVSATGPGPGPGPGPGPGPGPAPPNYSRPHGMGGPNMPPPGPSGVPPGMPGQPPGGPPKPWPEGPMANAAAPTSTPQKLIPPQPTGRPSPAPPAVPPAASPVMPPQTQSPGQPAQPAPMVPLHQKQSRITPIQKPRGLDPVEILQEREYRLQARIAHRIQELENLPGSLAGDLRTKATIELKALRLLNFQRQLRQEVVVCMRRDTALETALNAKAYKRSKRQSLREARITEKLEKQQKIEQERKRRQKHQEYLNSILQHAKDFKEYHRSVTGKIQKLTKAVATYHANTEREQKKENERIEKERMRRLMAEDEEGYRKLIDQKKDKRLAYLLQQTDEYVANLTELVRQHKAAQVAKEKKKKKKKKKAENAEGQTPAIGPDGEPLDETSQMSDLPVKVIHVESGKILTGTDAPKAGQLEAWLEMNPGYEVAPRSDSEESGSEEEEEEEEEEQPQAAQPPTLPVEEKKKIPDPDSDDVSEVDARHIIENAKQDVDDEYGVSQALARGLQSYYAVAHAVTERVDKQSALMVNGVLKQYQIKGLEWLVSLYNNNLNGILADEMGLGKTIQTIALITYLMEHKRINGPFLIIVPLSTLSNWAYEFDKWAPSVVKVSYKGSPAARRAFVPQLRSGKFNVLLTTYEYIIKDKHILAKIRWKYMIVDEGHRMKNHHCKLTQVLNTHYVAPRRLLLTGTPLQNKLPELWALLNFLLPTIFKSCSTFEQWFNAPFAMTGEKVDLNEEETILIIRRLHKVLRPFLLRRLKKEVEAQLPEKVEYVIKCDMSALQRVLYRHMQAKGVLLTDGSEKDKKGKGGTKTLMNTIMQLRKICNHPYMFQHIEESFSEHLGFTGGIVQGLDLYRASGKFELLDRILPKLRATNHKVLLFCQMTSLMTIMEDYFAYRGFKYLRLDGTTKAEDRGMLLKTFNEPGSEYFIFLLSTRAGGLGLNLQSADTVIIFDSDWNPHQDLQAQDRAHRIGQQNEVRVLRLCTVNSVEEKILAAAKYKLNVDQKVIQAGMFDQKSSSHERRAFLQAILEHEEQDEEEDEVPDDETVNQMIARHEEEFDLFMRMDLDRRREEARNPKRKPRLMEEDELPSWIIKDDAEVERLTCEEEEEKMFGRGSRHRKEVDYSDSLTEKQWLKTLKAIEEGTLEEIEEEVRQKKSSRKRKRDSDAGSSTPTTSTRSRDKDDESKKQKKRGRPPAEKLSPNPPNLTKKMKKIVDAVIKYKDSSGRQLSEVFIQLPSRKELPEYYELIRKPVDFKKIKERIRNHKYRSLNDLEKDVMLLCQNAQTFNLEGSLIYEDSIVLQSVFTSVRQKIEKEDDSEGEESEEEEEGEEEGSESESRSVKVKIKLGRKEKAQDRLKGGRRRPSRGSRAKPVVSDDDSEEEQEEDRSGSGSEED

SEQ ID NO: 19 - protein, 1614 aa, Homo sapiens
MSTPDPPLGGTPRPGPSPGPGPSPGAMLGPSPGPSPGSAHSMMGPSPGPPSAGHPIPTQGPGGYPQDNMHQMHKPMESMHEKGMSDDPRYNQMKGMGMRSGGHAGMGPPPSPMDQHSQGYPSPLGGSEHASSPVPASGPSSGPQMSSGPGGAPLDGADPQALGQQNRGPTPFNQNQLHQLRAQIMAYKMLARGQPLPDHLQMAVQGKRPMPGMQQQMPTLPPPSVSATGPGPGPGPGPGPGPGPAPPNYSRPHGMGGPNMPPPGPSGVPPGMPGQPPGGPPKPWPEGPMANAAAPTSTPQKLIPPQPTGRPSPAPPAVPPAASPVMPPQTQSPGQPAQPAPMVPLHQKQSRITPIQKPRGLDPVEILQEREYRLQARIAHRIQELENLPGSLAGDLRTKATIELKALRLLNFQRQLRQEVVVCMRRDTALETALNAKAYKRSKRQSLREARITEKLEKQQKIEQERKRRQKHQEYLNSILQHAKDFKEYHRSVTGKIQKLTKAVATYHANTEREQKKENERIEKERMRRLMAEDEEGYRKLIDQKKDKRLAYLLQQTDEYVANLTELVRQHKAAQVAKEKKKKKKKKKAENAEGQTPAIGPDGEPLDETSQMSDLPVKVIHVESGKILTGTDAPKAGQLEAWLEMNPGYEVAPRSDSEESGSEEEEEEEEEEQPQAAQPPTLPVEEKKKIPDPDSDDVSEVDARHIIENAKQDVDDEYGVSQALARGLQSYYAVAHAVTERVDKQSALMVNGVLKQYQIKGLEWLVSLYNNNLNGILADEMGLGKTIQTIALITYLMEHKRINGPFLIIVPLSTLSNWAYEFDKWAPSVVKVSYKGSPAARRAFVPQLRSGKFNVLLTTYEYIIKDKHILAKIRWKYMIVDEGHRMKNHHCKLTQVLNTHYVAPRRLLLTGTPLQNKLPELWALLNFLLPTIFKSCSTFEQWFNAPFAMTGEKVDLNEEETILIIRRLHKVLRPFLLRRLKKEVEAQLPEKVEYVIKCDMSALQRVLYRHMQAKGVLLTDGSEKDKKGKGGTKTLMNTIMQLRKICNHPYMFQHIEESFSEHLGFTGGIVQGLDLYRASGKFELLDRILPKLRATNHKVLLFCQMTSLMTIMEDYFAYRGFKYLRLDGTTKAEDRGMLLKTFNEPGSEYFIFLLSTRAGGLGLNLQSADTVIIFDSDWNPHQDLQAQDRAHRIGQQNEVRVLRLCTVNSVEEKILAAAKYKLNVDQKVIQAGMFDQKSSSHERRAFLQAILEHEEQDEEEDEVPDDETVNQMIARHEEEFDLFMRMDLDRRREEARNPKRKPRLMEEDELPSWIIKDDAEVERLTCEEEEEKMFGRGSRHRKEVDYSDSLTEKQWLKAIEEGTLEEIEEEVRQKKSSRKRKRDSDAGSSTPTTSTRSRDKDDESKKQKKRGRPPAEKLSPNPPNLTKKMKKIVDAVIKYKDSSSGRQLSEVFIQLPSRKELPEYYELIRKPVDFKKIKERIRNHKYRSLNDLEKDVMLLCQNAQTFNLEGSLIYEDSIVLQSVFTSVRQKIEKEDDSEGEESEEEEEGEEEGSESESRSVKVKIKLGRKEKAQDRLKGGRRRPSRGSRAKPVVSDDDSEEEQEEDRSGSGSEED

SEQ ID NO: 20 - protein, 1613 aa, Homo sapiens
MSTPDPPLGGTPRPGPSPGPGPSPGAMLGPSPGPSPGSAHSMMGPSPGPPSAGHPIPTQGPGGYPQDNMHQMHKPMESMHEKGMSDDPRYNQMKGMGMRSGGHAGMGPPPSPMDQHSQGYPSPLGGSEHASSPVPASGPSSGPQMSSGPGGAPLDGADPQALGQQNRGPTPFNQNQLHQLRAQIMAYKMLARGQPLPDHLQMAVQGKRPMPGMQQQMPTLPPPSVSATGPGPGPGPGPGPGPGPAPPNYSRPHGMGGPNMPPPGPSGVPPGMPGQPPGGPPKPWPEGPMANAAAPTSTPQKLIPPQPTGRPSPAPPAVPPAASPVMPPQTQSPGQPAQPAPMVPLHQKQSRITPIQKPRGLDPVEILQEREYRLQARIAHRIQELENLPGSLAGDLRTKATIELKALRLLNFQRQLRQEVVVCMRRDTALETALNAKAYKRSKRQSLREARITEKLEKQQKIEQERKRRQKHQEYLNSILQHAKDFKEYHRSVTGKIQKLTKAVATYHANTEREQKKENERIEKERMRRLMAEDEEGYRKLIDQKKDKRLAYLLQQTDEYVANLTELVRQHKAAQVAKEKKKKKKKKKAENAEGQTPAIGPDGEPLDETSQMSDLPVKVIHVESGKILTGTDAPKAGQLEAWLEMNPGYEVAPRSDSEESGSEEEEEEEEEEQPQAAQPPTLPVEEKKKIPDPDSDDVSEVDARHIIENAKQDVDDEYGVSQALARGLQSYYAVAHAVTERVDKQSALMVNGVLKQYQIKGLEWLVSLYNNNLNGILADEMGLGKTIQTIALITYLMEHKRINGPFLIIVPLSTLSNWAYEFDKWAPSVVKVSYKGSPAARRAFVPQLRSGKFNVLLTTYEYIIKDKHILAKIRWKYMIVDEGHRMKNHHCKLTQVLNTHYVAPRRLLLTGTPLQNKLPELWALLNFLLPTIFKSCSTFEQWFNAPFAMTGEKVDLNEEETILIIRRLHKVLRPFLLRRLKKEVEAQLPEKVEYVIKCDMSALQRVLYRHMQAKGVLLTDGSEKDKKGKGGTKTLMNTIMQLRKICNHPYMFQHIEESFSEHLGFTGGIVQGLDLYRASGKFELLDRILPKLRATNHKVLLFCQMTSLMTIMEDYFAYRGFKYLRLDGTTKAEDRGMLLKTFNEPGSEYFIFLLSTRAGGLGLNLQSADTVIIFDSDWNPHQDLQAQDRAHRIGQQNEVRVLRLCTVNSVEEKILAAAKYKLNVDQKVIQAGMFDQKSSSHERRAFLQAILEHEEQDEEEDEVPDDETVNQMIARHEEEFDLFMRMDLDRRREEARNPKRKPRLMEEDELPSWIIKDDAEVERLTCEEEEEKMFGRGSRHRKEVDYSDSLTEKQWLKAIEEGTLEEIEEEVRQKKSSRKRKRDSDAGSSTPTTSTRSRDKDDESKKQKKRGRPPAEKLSPNPPNLTKKMKKIVDAVIKYKDSSGRQLSEVFIQLPSRKELPEYYELIRKPVDFKKIKERIRNHKYRSLNDLEKDVMLLCQNAQTFNLEGSLIYEDSIVLQSVFTSVRQKIEKEDDSEGEESEEEEEGEEEGSESESRSVKVKIKLGRKEKAQDRLKGGRRRPSRGSRAKPVVSDDDSEEEQEEDRSGSGSEED